(19) United States
(12) Patent Application Publication
(10) Pub. No.: US 2022/0175735 A1
(43) Pub. Date: Jun. 9, 2022

(54) METHODS OF TREATING PULMONARY DISEASES AND DISORDERS

(71) Applicant: Proteostasis Therapeutics, Inc., Boston, MA (US)

(72) Inventors: John Miller, Worcester, MA (US); Cecilia M. Bastos, South Grafton, MA (US); Benito Munoz, Newtonville, MA (US)

(21) Appl. No.: 17/354,751

(22) Filed: Jun. 22, 2021

Related U.S. Application Data
(63) Continuation of application No. 16/065,384, filed on Jun. 22, 2018, now abandoned.
(60) Provisional application No. 62/271,804, filed on Dec. 28, 2015.
(60) Provisional application No. 62/271,191, filed on Dec. 22, 2015.

(51) Int. Cl. A61K 31/422; A61K 31/404; A61K 31/4245; A61K 31/47; A61K 31/7036; A61K 45/06; A61K 31/443; A61P 11/06; A61P 11/08; A61K 9/00; A61K 31/341; A61K 31/4192; A61K 31/433; A61K 31/454; A61K 31/5377

(52) U.S. Cl. CPC A61K 31/422; A61K 31/191; A61K 31/4245; A61K 31/47; A61K 31/7036; A61K 45/06; A61K 31/443; A61P 11/06; A61P 11/08; A61K 9/0053; A61K 9/0073; A61K 31/341; A61K 31/4192; A61K 31/433; A61K 31/454; A61K 31/5377; A61K 31/404

(57) ABSTRACT

The present disclosure features disclosed method of treating disorders such as COPD, bronchitis and/or asthma using disclosed compounds, optionally together with one or more additional active agents. Contemplated methods include administrating orally or by inhalation to a patient one or more disclosed compounds.

### CROSS REFERENCE TO RELATED APPLICATIONS

This application is a continuation of U.S. patent application Ser. No. 16/065,384, filed Jun. 22, 2018, which is a national stage filing under 35 U.S.C. § 371 of PCT/US2016/068266, filed Dec. 22, 2016, which claims the benefit of, and priority to, U.S. Provisional Patent Application Nos. 62/271,191, filed Dec. 22, 2015; and 62/271,804, filed Dec. 28, 2015; the contents of each of which are hereby incorporated by reference herein in their entirety.

### BACKGROUND

Chronic obstructive pulmonary disease (COPD) is a progressive lung condition that affects 329 million people, or nearly 5% of the worldwide population and is characterized by, for example, chronically reduced airflow in the lungs, shortness of breath, cough, and sputum production. Smoking is a major risk factor for COPD. Prevalence of COPD is higher among smokers and ex-smokers compared to those who have never smoked, and increases with the number of years for which an individual has smoked. Other important risk factors are: male sex, old age, genetics, air pollution, and occupational exposures such as workplace dusts. The causes and symptoms associated with COPD have been associated with related pulmonary diseases and disorders, including for example, chronic bronchitis and emphysema. These symptoms are present for a prolonged period of time and typically worsen over time. There is no known cure for COPD, but the symptoms are treatable and its progression can be delayed. The major goals of therapeutic intervention are to alleviate symptoms, the reduction of severity and frequency of acute exacerbations, and the overall improvement in the health status of the patients.

For example, a decrease in activity of the cystic fibrosis transmembrane conductance regulator (CFTR) ion transport channel has been implicated in COPD pathogenesis. The cystic fibrosis transmembrane conductance regulator (CFTR) gene encodes a multi-membrane spanning epithelial chloride channel (Riordan et al., Annu Rev Biochem 77, 701-26 (2008)). Mutations of the CFTR gene affecting chloride ion channel function and/or activity of the CFTR channel may lead to dysregulation of epithelial fluid transport in the lung, pancreas, and other organs. Smokers with COPD have decreased CFTR activity in both the upper and lower airways, suggesting that decreased CFTR activity may play a role in the pathogenesis of COPD. Decreased CFTR activity has also been associated with the development of chronic bronchitis.

Further, individuals with smoking-induced lung disease, and in particular those with COPD-associated chronic bronchitis exhibit pathologic features similar to CF including mucus stasis, or lack of mucus clearing or transport, and accumulation. Mucus stasis in COPD may be associated with lung function decline, exacerbation frequency, and early mortality. At present, no therapies definitively address mucus accumulation in COPD. Experimental evidence has confirmed that cigarette smoking reduces CFTR activity and is causally related to reduced mucus transport in smokers due to inhibition of CFTR dependent fluid transport. CFTR modulators may be a strategy for the treatment of COPD given their ability to increase CFTR protein activity which can improve airway hydration and restore normal mucus function.

In addition to COPD, mutations in the CFTR gene and/or the activity of the CFTR channel has also been implicated in other conditions, including for example, cystic fibrosis, congenital bilateral absence of vas deferens (CBAVD), acute, recurrent, or chronic pancreatitis, disseminated bronchiectasis, asthma, allergic pulmonary aspergillosis, dry eye disease, Sjogren's syndrome and chronic sinusitis.

Despite the availability of a number of different pharmacological and medical options currently approved or recommended for COPD treatment, there are still significant unmet medical needs perceived by both patients and treating physicians such as exacerbation and symptom control, improving health status and slowing the decline of lung function and disease progression. Therefore, there remains a need in the art for compounds, compositions and methods of increasing CFTR activity as well as for methods of treating COPD, associated pulmonary diseases and disorders, and other conditions related to CFTR activity.

### SUMMARY

Provided herein in part is a method of treating chronic obstructive pulmonary disease, bronchitis, or asthma in a patient in need thereof, or in a patient at risk of developing chronic obstructive pulmonary disease, comprising a) administering an effective amount of a disclosed compound (e.g., represented by Formula III or IV) and b) optionally administering an effective amount of one or more of an additional active agent, wherein Formulas III and IV are:

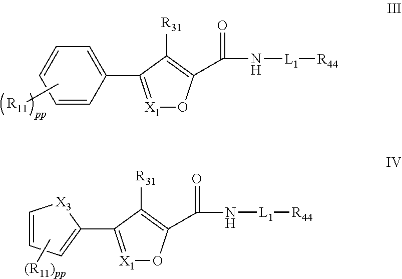

and pharmaceutically acceptable salts, stereoisomers, and prodrugs thereof, wherein X₁, X₃, R₁₁, pp, R₃₁, L₁ and R₄₄ are defined below.

### DETAILED DESCRIPTION

As used herein, the words “a” and “an” are meant to include one or more unless otherwise specified. For example, the term “an agent” encompasses both a single agent and a combination of two or more agents.

As discussed above, the present disclosure is directed in part to compounds as described herein having a pharmaceutically acceptable salt, prodrug or solvate thereof, pharmaceutical compositions, and methods of treating pulmonary disorders, e.g., COPD.

For example, provided herein in an embodiment, is a method of treating COPD, bronchitis, or asthma in a patient in need thereof, or in a patient at risk of developing chronic obstructive pulmonary disease, comprising a) administering an effective amount of a disclosed compound (e.g., a compound represented by Formula III or IV) and b) optionally administering an effective amount of one or more of an additional active agent. For example, provided herein is a method of treating emphysema, a form of COPD, in a patient in need thereof, comprising a) administering an effective amount of a disclosed compound (e.g., a compound represented by Formula III or IV) and b) optionally administering an effective amount of one or more of an additional active agent.

A method for treating mucus stasis in a patient suffering from lack of mucus clearing and/or limited mucus transport comprising administering to the patient an effective amount of a disclosed compound (e.g. a compound of Formula III or IV), and optionally administering an effective amount of one or more of an additional active agents is also provided herein. For example, provided herein is a method of improving airway hydration and/or restoring normal mucus function in a patient in need thereof comprising administering an effective amount of a provided compound and optionally an effective amount of one or more.

For example, provided herein is a method of treating chronic bronchitis, a form of COPD, in a patient in need thereof, comprising a) administering an effective amount of a disclosed compound (e.g., a compound represented by Formula III or IV) and b) optionally administering an effective amount of one or more of an additional active agent.

Contemplated herein is a method of treating a patient at risk for developing COPD, wherein for example, the risk factor for developing COPD is a history of smoking and/or air pollution and/or be at risk for or suffer from mesothelioma.

In some embodiments, contemplated methods (e.g., of treating COPD) include administering by inhalation or orally an effective amount of a disclosed compound or composition, and optionally administering an effective amount of another active agent is oral or inhalation administration. In other embodiments, contemplated methods (e.g., of treating COPD) include administering orally an effective amount of a disclosed compound or composition, and optionally administering an effective amount of another active agent is oral or inhalation administration

In some embodiments, a disclosed compound has the following Formula III or IV:

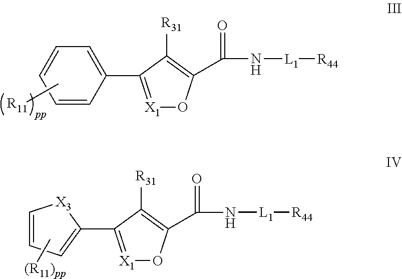

and pharmaceutically acceptable salts, stereoisomers, and prodrugs thereof, wherein:

X₁ is CR₃₃ or N;

X₃ is selected from the group consisting of O, S, and NRₕₕ;

pp is 1, 2, or 3;

R₁₁ is independently selected for each occurrence from the group consisting of hydrogen, halogen, C₁₋₄ alkyl (optionally substituted by one, two or three halogens);

R₃₁ is selected from the group consisting of hydrogen, halogen, and C₁₋₄ alkyl;

R₃₃ is selected from the group consisting of H, halogen, C₁₋₄ alkyl, and —NR′R″ wherein R′ and R″ are each independently selected for each occurrence from H and C₁₋₄ alkyl or taken together with the nitrogen to which they are attached form a heterocyclic ring;

L₁ is selected from the group consisting of C₁₋₆ alkylene, C₃₋₆ cycloalkylene, C₃₋₆ cycloalkylene-C₁₋₄ alkylene, C₁₋₃ alkylene-NRₕₕ—S(O)_{w}, —C₁₋₃ alkylene-S(O)_{w}—NRₕₕ—, C₃₋₆ cycloalkylene-C₀₋₂ alkylene-S(O)_{w}—NRₕₕ, and C₃₋₆ cycloalkylene-C₀₋₂ alkylene NRₕₕ—S(O)_{w}—, wherein L₁ may be optionally substituted by one, two or three substituents selected from the group consisting of halogen, hydroxyl, and C₁₋₃ alkyl (optionally substituted by one, two or three substituents each selected independently from R_{ff});

R₄₄ is selected from the group consisting of H, halogen, hydroxyl, C₁₋₃ alkoxy, phenyl, —O-phenyl, —NR′-phenyl, heterocycle, and a 5-6 membered monocyclic or 8-10 membered bicyclic heteroaryl having one, two or three heteroatoms each selected from O, N, and S; wherein phenyl, —O-phenyl, —NR′-phenyl, heterocycle and heteroaryl may be optionally substituted by one or two substituents each selected independently from R_{gg};

R_{ff} is selected for each occurrence from group consisting of halogen, hydroxyl, C₁₋₄ alkyl, C₁₋₄ alkyoxy, C₂₋₄ alkenyl, C₃₋₆ cycloalkyl, —NR′R″, —NR′—S(O)_{w}—C₁₋₃ alkyl, S(O)_{w}—NR′R″, and —S(O)_{w}—C₁₋₃ alkyl, where w is 0, 1, or 2, wherein C₁₋₄ alkyl, C₁₋₄ alkyoxy, C₂₋₄ alkenyl and C₃₋₆ cycloalkyl may be optionally substituted by one, two or three substituents each independently selected from the group consisting of halogen, hydroxyl, —NR′R″, —NR′—S(O)_{w}—C₁₋₃ alkyl, S(O)_{w}—NR′R″, and —S(O)_{w}—C₁₋₃ alkyl;

R_{gg} is selected for each occurrence from the group consisting of:
a) halogen, hydroxyl, cyano, —NR′R″, —NR′—S(O)_{w}—C₁₋₃ alkyl, —S(O)_{w}—NR′R″, and —S(O)_{w}—C₁₋₃ alkyl, where w is 0, 1, or 2;
b) C₁₋₆ alkyl, C₃₋₆ cycloalkyl, and C₁₋₆ alkenyl, wherein C₁₋₆ alkyl, C₃₋₆ cycloalkyl, and C₁₋₆ alkenyl are optionally substituted by one, two, or three substituents each independently selected from Rⱼⱼ; and
c) heterocycle, optionally substituted by one, two, or three substituents each independently selected from Rₗₗ;

Rⱼⱼ is selected for each occurrence from the group consisting of halogen, hydroxyl, C₃₋₆ cycloalkyl, C₃₋₆ cycloalkoxy, C₁₋₆ alkoxy (optionally substituted by one, two, or three substituents each independently selected from Rₖₖ), heterocycle, C(O)OH, —C(O)OC₁₋₆ alkyl, —NR′R″, —NR′—S(O)_{w}—C₁₋₃ alkyl, —S(O)_{w}—NR′R″, and —S(O)_{w}—C₁₋₃ alkyl, where w is 0, 1, or 2; Rₖₖ is selected for each occurrence from the group consisting of halogen, hydroxyl, C₁₋₆ alkyl (optionally substituted by one, two, or three substituents each independently selected from halogen, hydroxyl, C₃₋₆ cycloalkyl, and heterocycle (optionally substituted by C₁₋₆ alkyl)), C₃₋₆ cycloalkyl (optionally substituted by one, two, or three substituents each independently selected from halogen, hydroxyl, and C₁₋₆ alkyl), phenyl, heterocycle (optionally substituted by one, two or three substituents independently selected from halogen, hydroxyl, and C₁₋₆ alkyl), and heteroaryl;

Rₗₗ is selected for each occurrence from the group consisting of halogen, hydroxyl, C₁₋₆ alkyl (optionally substituted by one, two, or three substituents each independently selected from halogen, hydroxyl, and C₃₋₆ cycloalkyl) and heterocycle (optionally substituted by one, two or three substituents independently selected from halogen, hydroxyl, and C₁₋₆ alkyl);

R′ and R″ are each independently selected for each occurrence from H and C₁₋₄ alkyl;

w is 0, 1 or 2; and

Rₕₕ is selected for each occurrence from the group consisting of H, C₁₋₆ alkyl and C₃₋₆ cycloalkyl.

For example, in certain of these embodiments, L₁ of one or more of the above formulas is C₁₋₃ alkylene, C₃₋₅ cycloalkylene, or C₃₋₆ cycloalkylene-C₁₋₄ alkylene and/or R₃₁ is H or F.

In certain embodiments, R_{gg} is selected from the group consisting of:

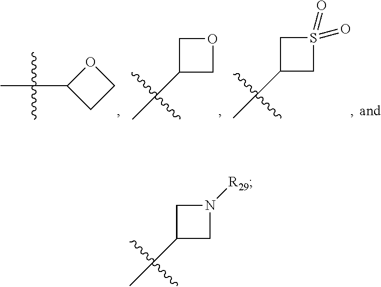

wherein R₂₉ is selected from C₁₋₆ alkyl (optionally substituted by one, two or three substituents each independently selected from the group consisting of halogen, hydroxyl, C₁₋₆ alkoxy, and cycloalkyl) and heterocycle (optionally substituted by one, two or three substituents each independently selected from the group consisting of halogen, hydroxyl, C₁₋₆ alkyl and C₁₋₆ alkoxy). For example, R₂₉ may be selected from the group consisting of:

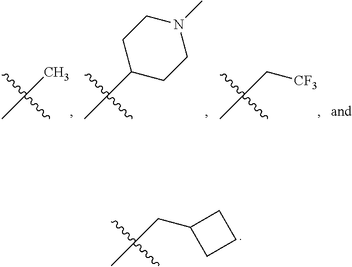

In an embodiment, a disclosed compound has the formula:

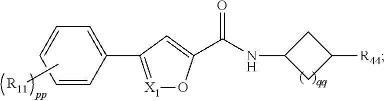

wherein qq is 0 or 1.

For example, a disclosed compound may have, in certain embodiments the following formula:

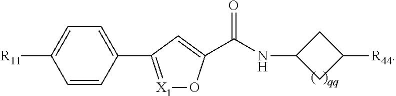

For example, R₄₄ as in the above formulas may be selected from the group consisting of: pyrrolidinyl, piperidinyl, tetrahydropyranyl, and tetrahydofuranyl. In other embodiments, R₄₄ is selected from the group consisting of:

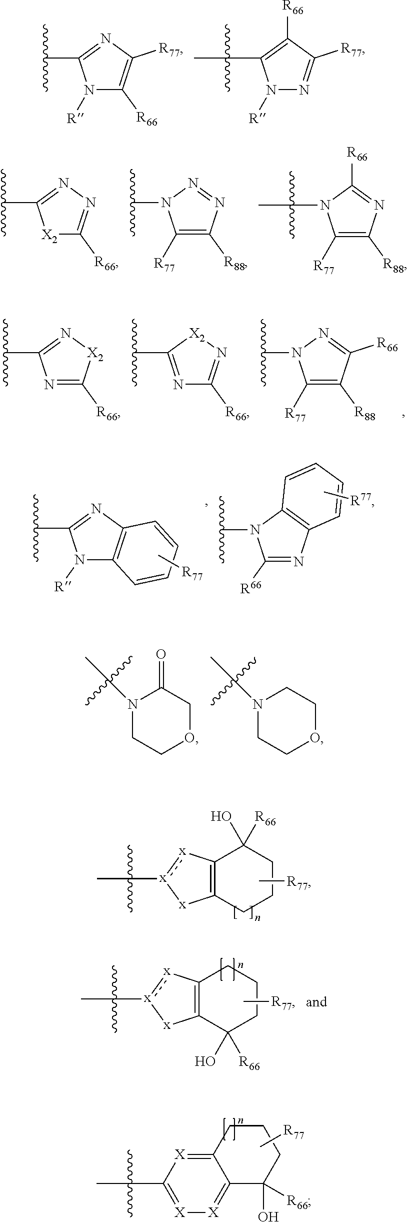

wherein X independently for each occurrence is selected from the group consisting of O, S, NRₕₕ, C, C(R₈₈), and C(R₈₈)(R₉₉); X₂ independently for each occurrence is selected from the group consisting of O, S and NRₕₕ; R″ is H or C₁₋₄alkyl, each R₆₆, R₇₇, R₈₈ and R₉₉ is independently selected for each occurrence from H and R_{gg}, and n is 0, 1, 2, or 3.

In certain embodiments, each of R₆₆, R₇₇, R₈₈ and R₉₉ is independently selected for each occurrence from the group consisting of hydrogen, halogen, hydroxyl, C₁₋₆ alkyl, C₃₋₆ cycloalkyl, and heterocycle, wherein C₁₋₆ alkyl, C₃₋₆ cycloalkyl, and heterocycle are optionally substituted by one, two or three substituents each independently selected from the group consisting of hydroxyl, C₁₋₆ alkyl, C₁₋₆ alkoxy, —S(O)_{w}—C₁₋₃ alkyl (w is 0, 1, or 2) and —NR′S(O)₂C₁₋₆ alkyl. In some embodiments, R′ is H or C₁₋₄ alkyl. In certain embodiments, R₆₆, R₇₇ and R₈₈ may be selected from the group consisting of H, halogen, methyl (optionally substituted by one, two or three substituents each selected from halogen, hydroxyl, methoxy and ethoxy), ethyl (optionally substituted by one, two or three substituents each selected from halogen, hydroxyl, methoxy and ethoxy), propyl ((optionally substituted by one, two or three substituents each selected from halogen, hydroxyl, methoxy and ethoxy), isopropyl ((optionally substituted by one, two or three substituents each selected from halogen, hydroxyl, methoxy and ethoxy), n-butyl (optionally substituted by one, two or three substituents each selected from halogen, hydroxyl, methoxy and ethoxy), t-butyl (optionally substituted by one, two or three substituents each selected from halogen, hydroxyl, methoxy and ethoxy), s-butyl (optionally substituted by one, two or three substituents each selected from halogen, hydroxyl, methoxy and ethoxy) and isobutyl (optionally substituted by one, two or three substituents each selected from halogen, hydroxyl, methoxy and ethoxy).

In certain embodiments, pp is 0, 1 or 2, and R₁₁ is selected from H, F, or methyl.

For example, a disclosed compound may be represented by:

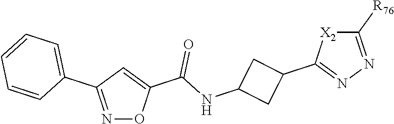

wherein X₂ is selected from the group consisting of O, S or NRₕₕ (defined above);

R₇₆ is selected from the group consisting of C₁₋₆alkyl (optionally interrupted by one or more oxygen atoms or NR″, and optionally substituted by one or more substituents selected from the group consisting of halogen, hydroxyl, S(O)_{w}—C₁₋₃ alkyl (w is 0, 1, or 2), C₃₋₆cycloalkyl (optionally substituted by one or more substituents selected from heterocycle, C₁₋₆alkyl, and halogen) and heterocycle (optionally substituted by one or more substituents selected from heterocycle, C₁₋₆alkyl, and halogen)); and heterocycle (optionally substituted by one or more substituents selected from the group consisting of halogen, hydroxyl, S(O)_{w}—C₁₋₃ alkyl (w is 0, 1, or 2), C₃₋₆cycloalkyl (optionally substituted by one or more substituents selected from heterocycle, C₁₋₆alkyl, and halogen) and heterocycle (optionally substituted by one or more substituents selected from heterocycle, C₁₋₆alkyl, and halogen).

In some embodiments, a disclosed compound has the Formula (Ia) or the Formula (IIa):

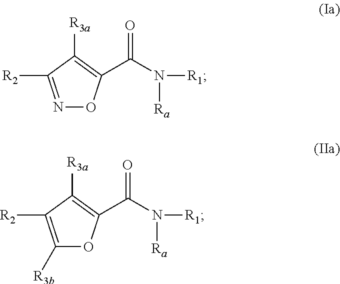

or a pharmaceutically acceptable salt, prodrug or solvate thereof, wherein:

R₁ is selected from the group consisting of:

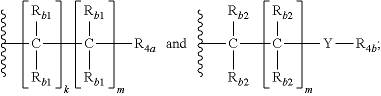

R₂ is selected from the group consisting of optionally substituted aryl and optionally substituted heteroaryl;

R₃ₐ and R_{3b} are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, N(R_{d})(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c}, (C═NR_{d})R_{c}, optionally substituted heterocyclic and optionally substituted heteroaryl;

R₄ₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, halo, OR_{c}, S(O)ₙR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)R_{c}, N(R_{d})(COOR_{c}), NR_{d}C(O)C(O)R_{c}, NR_{d}C(O)NR_{d}R_{d}, NR_{d}S(O)ₙR_{d}R_{d}, NR_{d}S(O)ₙR_{c}, S(O)NR_{d}R_{d}, OC(O)OR_{c}, (C═NR_{d})R_{c}, optionally substituted heterocyclic and optionally substituted heteroaryl;

R_{4b} is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂cycloalkenyl, optionally substituted aryl, optionally substituted heterocyclic and optionally substituted heteroaryl;

Rₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂cycloalkenyl, optionally substituted heterocyclic, optionally substituted aryl, optionally substituted heteroaryl, C(O)OR_{c}, C(O)R_{c}, C(O)C(O)R_{c} and S(O)ₙR_{c};

or alternatively, Rₐ and the nitrogen atom to which it is attached is taken together with an adjacent C(R_{b1})(R_{b1}) or C(R_{b2})(R_{b2}) to form an optionally substituted, 4- to 12-membered heterocyclic ring containing one or more ring nitrogen atoms, wherein said heterocyclic ring optionally contains one or more ring heteroatoms selected from oxygen and sulfur;

each R_{b1} and R_{b2} is independently selected for each occurrence from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, N(R_{d})(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c} and (C═NR_{d})R_{c}; or alternatively, two geminal R_{b1} groups or two geminal R_{b2} groups and the carbon to which they are attached are taken together to form a C(O) group, or yet alternatively, two geminal R_{b1} groups or two geminal R_{b2} groups are taken together with the carbon atom to which they are attached to form a spiro C₃-C₁₂ cycloalkyl, a spiro C₃-C₁₂ cycloalkenyl, a spiro heterocyclic, a spiro aryl or spiro heteroaryl, each optionally substituted; Y is selected from the group consisting of S(O)ₙ, NR_{d}, NR_{d}S(O)ₙ, NR_{d}S(O)ₙNR_{d}, NR_{d}C(O), NR_{d}C(O)O, NR_{d}C(O)C(O), NR_{d}C(O)NR_{d}, S(O)ₙNR_{d}, and O;

each R_{c} is independently selected for each occurrence from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;

each R_{d} is independently selected for each occurrence from the group consisting of hydrogen, optionally substituted C₁-C₁₀ alkyl, optionally substituted C₂-C₁₀ alkenyl, optionally substituted C₂-C₁₀ alkynyl, optionally substituted C₁-C₁₀ alkoxy, optionally substituted C₃-C₁₂ cycloalkyl, optionally substituted C₃-C₁₂ cycloalkenyl, optionally substituted heterocyclic, optionally substituted aryl and optionally substituted heteroaryl; or two geminal R_{d} groups are taken together with the nitrogen atom to which they are attached to form an optionally substituted heterocyclic or an optionally substituted heteroaryl;

k is 0 or 1;

m is 0, 1, 2, 3, 4, or 5;

each n is independently 0, 1 or 2.

In some embodiments, m is 0, 1 or 2. In some embodiments, k is 0. In some embodiments, m is 0, 1 or 2, k is 0.

In some embodiments, each of R₃ₐ and R_{3b} is hydrogen.

In some embodiments, Rₐ is hydrogen or C₁-C₄ alkyl (optionally substituted by 1, 2 or 3 halogens).

In some embodiments, R_{b1} and R_{b2} are each independently selected from the group consisting of hydrogen, hydroxyl, C₁₋₄ alkoxy (optionally substituted by one, two or three substituents independently selected from halogen and hydroxyl) and C₁-C₄ alkyl (optionally substituted by one, two or three substituents independently selected from halogen and hydroxyl). In certain embodiments, R_{b1} and R_{b2} for each occurrence are hydrogen.

In some embodiments, R₂ is selected from the group consisting of phenyl and a 5-6 membered heteroaryl having one or two heteroatoms each selected from N, S, and O, wherein R₂ is optionally substituted by one or two substituents each independently selected from the group consisting of halogen, and C₁-C₄ alkyl (optionally substituted by one, two or three halogens).

In certain embodiments, R₂ is phenyl.

In other embodiments, R₂ is selected from the group consisting of: optionally substituted thienyl, optionally substituted furanyl and optionally substituted pyridinyl.

In some embodiments, R₄ₐ is selected from the group consisting of optionally substituted C₁-C₆ alkyl, optionally substituted C₃-C₇ cycloalkyl, phenyl, OR_{c}, C(O)OR_{c}, C(O)R_{c}, optionally substituted heterocycle and optionally substituted heteroaryl, wherein R_{c} is selected, independently for each occurrence, from the group consisting of H and C₁₋₆ alkyl.

In certain embodiments, R₄ₐ is heterocycle, or a 5-6 membered monocyclic or a 8-10 membered bicyclic heteroaryl having one, two or three heteroatoms selected from N, S or O, wherein the heterocycle or heteroaryl are optionally substituted by one, two or three substituents independently selected for each occurrence from the group consisting of halogen, C₁₋₆ alkyl (optionally substituted by one, two or three substituents each independently selected from halogen and hydroxyl), C₁₋₆ alkoxy (optionally substituted by one, two or three halogens), hydroxyl, and NR_{d}R_{d} wherein R_{d} is independently for each occurrence selected from H and C₁₋₄ alkyl, or the two R_{d}s taken together with the N to which they are attached form a heterocyclic ring). For example, R₄ₐ can be selected from the group consisting of tetrahydropyranyl, thiadiazolyl, tetrahydrofuranyl, and morpholinyl. As another example, R₄ₐ can be a monocyclic heteroaryl containing one, two or three ring nitrogen atoms. As a further example, R₄ₐ can be selected from the group consisting of furanyl, pyridinyl, pyrazinyl, pyrazolyl, imidazolyl, isoxazolyl, triazolyl, thiazolyl, oxadiazolyl, thiadiazolyl, thienyl, piperazinyl, and benzimidazolyl, each optionally substituted.

In certain embodiments, R₄ₐ is selected from the group consisting of:

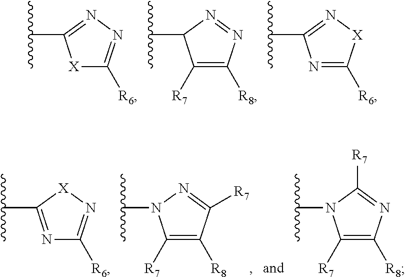

wherein each X is independently O, S or NR_{g};

each R_{g} is independently selected from the group consisting of hydrogen, C₁-C₄ alkyl, C₃-C₆ cycloalkyl, and

each R₆, R₇ and R₈ is independently selected for each occurrence from the group consisting of hydrogen, C₁-C₆ alkyl, C₂-C₆ alkenyl, C₂-C₁₆ alkynyl, C₃-C₇ cycloalkyl, C₃-C₇ cycloalkenyl, phenyl, heterocycle, heteroaryl, halo, hydroxyl, carboxyl, OR_{c}, NR_{d}R_{d}, C(O)OR_{c}, CN, C(O)R_{c}, wherein the C₁₋₆ alkyl, C₂-C₆ alkenyl, C₂-C₁₆ alkynyl, C₃-C₇ cycloalkyl, C₃-C₇ cycloalkenyl, phenyl, heterocycle, and heteroaryl of R₆, R₇ and R₈ may each be optionally substituted by one, two or three substituents selected from halo, hydroxyl, C₁₋₆ alkyl and C₁₋₆ alkoxy;

R_{c} is C₁₋₄ alkyl; and

R_{d} is independently for each occurrence selected from the group consisting of H and C₁₋₄ alkyl, or the two R_{d}s taken together with the N to which they are attached form a heterocyclic ring.

In some embodiments, a disclosed compound has the Formula (Ib) or the Formula (IIb):

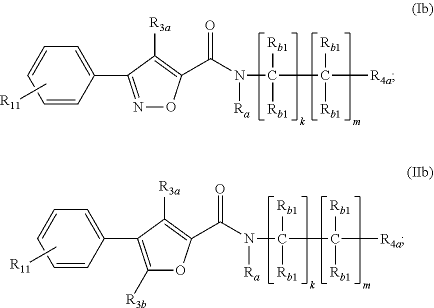

wherein, R₁₁ is selected from the group consisting of hydrogen, optionally substituted C₁-C₁₀ alkyl, optionally substituted C₂-C₁₀ alkenyl, optionally substituted C₂-C₁₀ alkynyl, and halo. In certain embodiments, R₄ₐ is an optionally substituted C₃-C₇ cycloalkyl (e.g., optionally substituted cyclopropyl or an optionally substituted cyclobutyl).

In certain of these embodiments, R₄ₐ is substituted with a substituent having the formula:

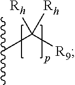

wherein each Rₕ is independently selected for each occurrence from the group consisting of hydrogen, halo, hydroxyl, C₁-C₆ alkyl, and C₃-C₆ cycloalkyl, or two geminal Rₕ groups are independently taken together with the carbon atom to which they are attached to form an optionally substituted carbocyclic or heterocycle;

R₉ is selected from the group consisting of hydrogen, halo, CN, hydroxyl, methyl (optionally substituted by one, two or three substituents selected from halogen and hydroxyl), C₂-C₄ alkenyl, C₂-C₄ alkynyl, C₃-C₆ cycloalkyl, C₁₋₆ alkoxy, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, NR_{d}(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c}, (C═NR_{d})R_{c};

R_{c} is independently selected for each occurrence from the group consisting of H, C₁-C₆ alkyl, C₃₋₆ cycloalkyl, heterocycle, and heteroaryl;

R_{d} is independently selected for each occurrence from H and C₁₋₄ alkyl, or the two R_{d}s taken together with the N to which they are attached form a heterocyclic ring; and p is 0, 1, or 2.

For example, R₄ₐ can be selected from the group consisting of:

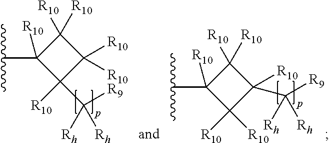

wherein

each R₁₀ is independently selected from the group consisting of hydrogen, optionally substituted C₁-C₆ alkyl, optionally substituted C₂-C₆ alkenyl, optionally substituted C₂-C₆ alkynyl, optionally substituted C₃-C₆ cycloalkyl, optionally substituted C₃-C₆ cycloalkenyl, optionally substituted aryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, NR_{d}(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c}, (C═NR_{d})R_{c}, optionally substituted heterocyclic and optionally substituted heteroaryl; alternatively, two geminal R₁₀ groups are taken together with the carbon atom to which they are attached to form a spiro C₃-C₇ cycloalkyl, a spiro C₃-C₇ cycloalkenyl, a spiro heterocyclic, a spiro aryl or spiro heteroaryl, each optionally substituted; or yet alternatively, two vicinal R₁₀ groups are taken together with the carbon atoms to which they are attached to form a fused, optionally substituted cyclic group selected from the group consisting of C₄-C₈ cycloalkyl, C₄-C₈ cycloalkenyl, 4- to 8-membered heterocyclic, aryl and heteroaryl, each optionally substituted; or further alternatively, two R₁₀ groups attached to non-adjacent carbon atoms are taken together with the carbon atoms to which they are attached to form a bridged cyclic group selected from the group consisting of C₃-C₈ cycloalkyl, C₃-C₈ cycloalkenyl, and 4- to 8-membered heterocyclic, each optionally substituted;

each Rₕ is independently selected from the group consisting of hydrogen, halo, optionally substituted C₁-C₁₀ alkyl, and optionally substituted C₃-C₆ cycloalkyl, or two geminal R_{b} groups are independently taken together with the carbon atom to which they are attached to form an optionally substituted heterocyclic or an optionally substituted heteroaryl;

R₉ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂cycloalkenyl, optionally substituted aryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, NR_{d}(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c}, (C═NR_{d})R_{c}, optionally substituted heterocyclic and optionally substituted heteroaryl; and p is 0, 1, or 2.

In some embodiments, Y is S, S(O)₂ or S(O)₂NR_{d}.

In some embodiments, R_{4b} is heterocycle or a 5-6 membered monocyclic or a 8-10 membered bicyclic heteroaryl having one, two or three heteroatoms selected from N, S or O, wherein the heterocycle or heteroaryl are optionally substituted by one, two or three substituents independently selected for each occurrence from the group consisting of halogen, C₁₋₆ alkyl (optionally substituted by one, two or three substituents each independently selected from halogen and hydroxyl), C₁₋₆ alkoxy (optionally substituted by one, two or three halogens), hydroxyl, and NR_{d}R_{d} wherein R_{d} is independently for each occurrence selected from H and C₁₋₄ alkyl, or the two R_{d}s taken together with the N to which they are attached form a heterocyclic ring). For example, R_{4b} can be selected from the group consisting of furanyl, pyridinyl, pyrazinyl, pyrazolyl, imidazolyl, isoxazolyl, triazolyl, thiazolyl, oxadiazolyl, thiadiazolyl, thienyl, piperazinyl, and benzimidazolyl, each optionally substituted.

Exemplary compounds are shown below in Table 1:

TABLE 1

| # | Structure |
|---|---|
| 1 | 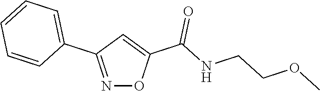 [SMILES: COCCNC(=O)c1cc(-c2ccccc2)no1] |
| 2 | 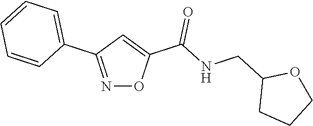 [SMILES: O=C(NCC1CCCO1)c1cc(-c2ccccc2)no1] |
| 3 | 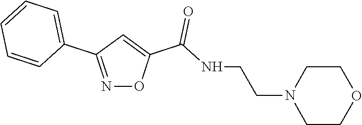 [SMILES: O=C(NCCN1CCOCC1)c1cc(-c2ccccc2)no1] |
| 4 | 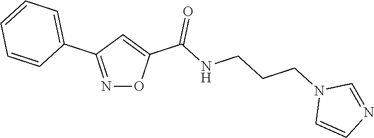 [SMILES: O=C(NCCCn1ccnc1)c1cc(-c2ccccc2)no1] |
| 5 | 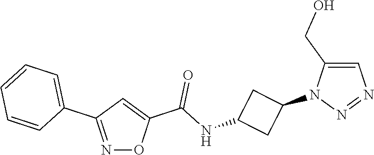 |
| 6 | 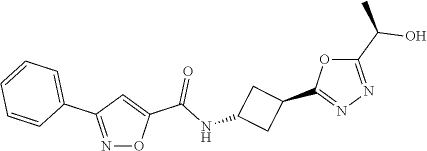 |
| 7 | 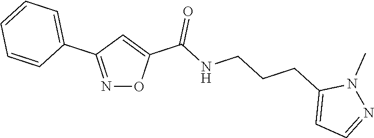 [SMILES: Cn1nccc1CCCNC(=O)c1cc(-c2ccccc2)no1] |
| 8 | 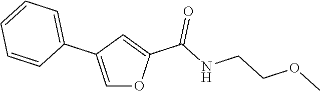 [SMILES: COCCNC(=O)c1cc(-c2ccccc2)co1] |
| 9 | 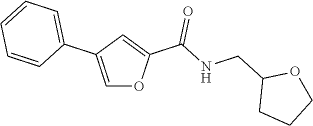 [SMILES: O=C(NCC1CCCO1)c1cc(-c2ccccc2)co1] |
| 10 | 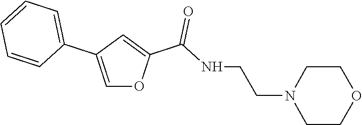 [SMILES: O=C(NCCN1CCOCC1)c1cc(-c2ccccc2)co1] |
| 11 | 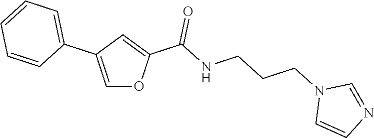 [SMILES: O=C(NCCCn1ccnc1)c1cc(-c2ccccc2)co1] |
| 12 | 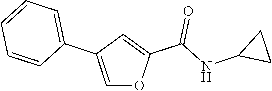 [SMILES: O=C(NC1CC1)c1cc(-c2ccccc2)co1] |
| 13 | 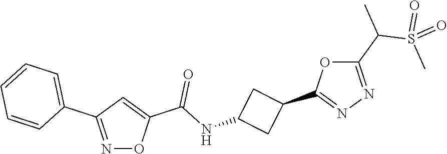 |
| 14 | 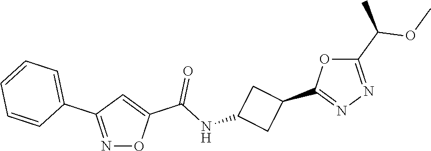 |
| 15 | 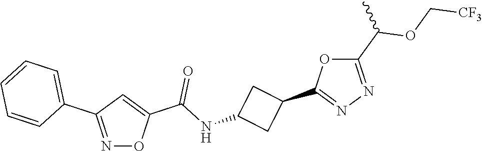 |
| 16 | 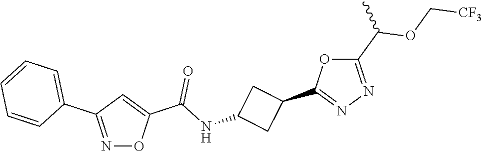 |
| 17 | 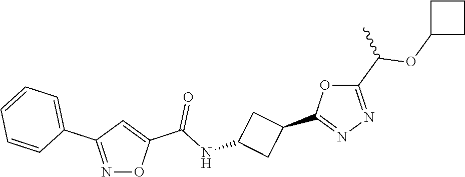 |
| 18 | 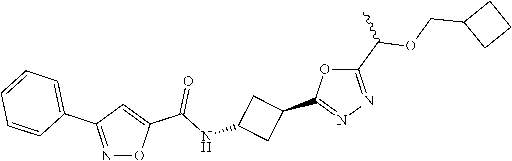 |
| 19 | 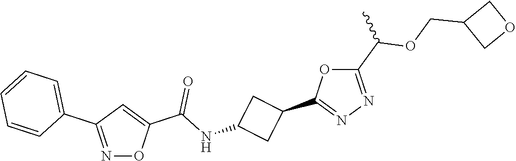 |
| 20 | 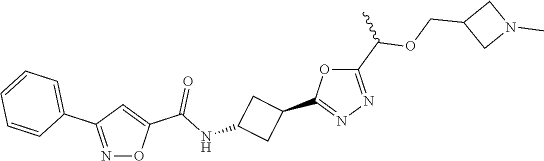 |
| 21 | 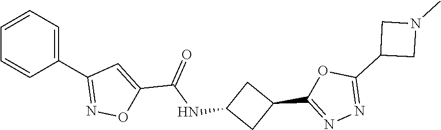 [SMILES: CN1CC(c2nnc([C@H]3C[C@H](NC(=O)c4cc(-c5ccccc5)no4)C3)o2)C1] |
| 22 | 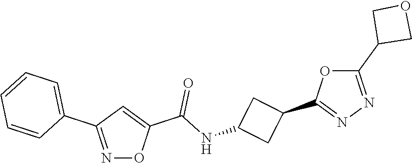 |
| 23 | 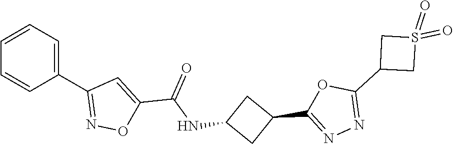 [SMILES: O=C(N[C@H]1C[C@H](c2nnc(C3CS(=O)(=O)C3)o2)C1)c1cc(-c2ccccc2)no1] |
| 24 | 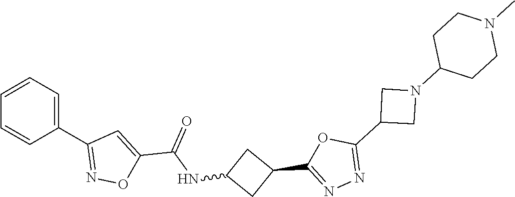 |
| 25 | 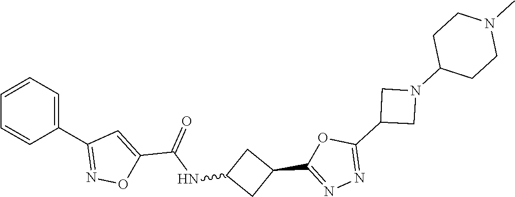 |
| 26 | 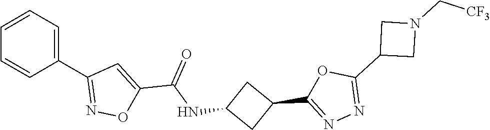 [SMILES: O=C(N[C@H]1C[C@H](c2nnc(C3CN(CC(F)(F)F)C3)o2)C1)c1cc(-c2ccccc2)no1] |
| 27 | 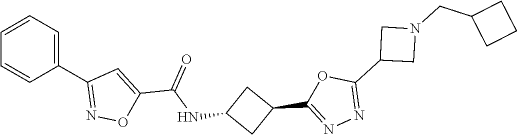 [SMILES: O=C(N[C@H]1C[C@H](c2nnc(C3CN(CC4CCC4)C3)o2)C1)c1cc(-c2ccccc2)no1] |
| 28 | 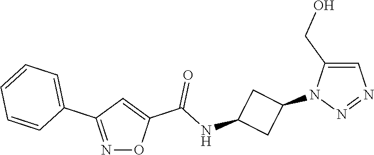 [SMILES: O=C(N[C@H]1C[C@@H](n2nncc2CO)C1)c1cc(-c2ccccc2)no1] |
| 29 | 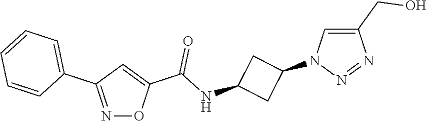 [SMILES: O=C(N[C@H]1C[C@@H](n2cc(CO)nn2)C1)c1cc(-c2ccccc2)no1] |
| 30 | 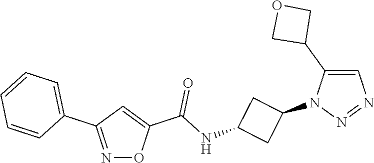 [SMILES: O=C(N[C@H]1C[C@H](n2nncc2C2COC2)C1)c1cc(-c2ccccc2)no1] |
| 31 | 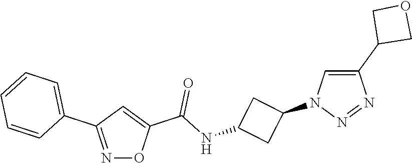 |
| 32 | 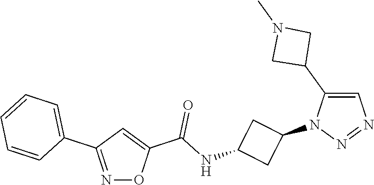 [SMILES: CN1CC(c2cnnn2[C@H]2C[C@H](NC(=O)c3cc(-c4ccccc4)no3)C2)C1] |
| 33 | 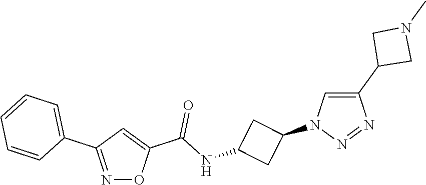 |
| 34 | 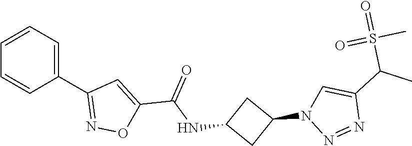 [SMILES: CC(c1cn([C@H]2C[C@H](NC(=O)c3cc(-c4ccccc4)no3)C2)nn1)S(C)(=O)=O] |
| 35 | 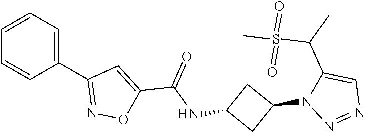 [SMILES: CC(c1cnnn1[C@H]1C[C@H](NC(=O)c2cc(-c3ccccc3)no2)C1)S(C)(=O)=O] |
| 36 | 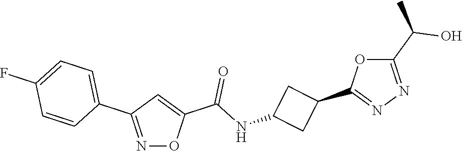 |
| 37 | 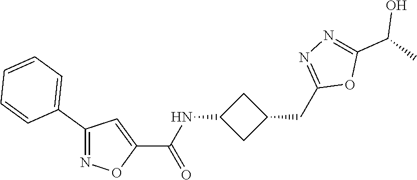 |
| 38 | 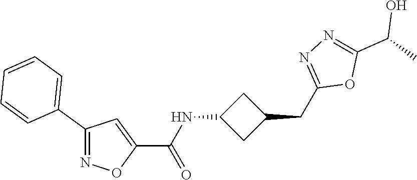 |
| 39 | 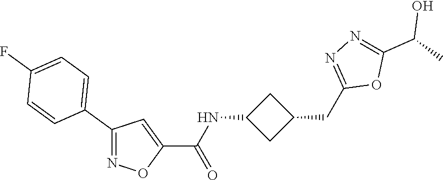 |
| 40 | 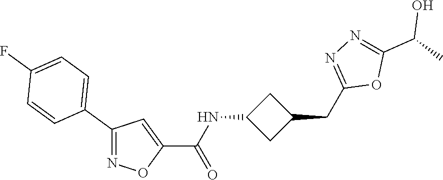 |
| 41 | 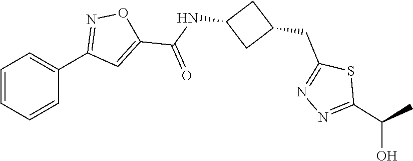 [SMILES: C[C@@H](O)c1nnc(C[C@H]2C[C@@H](NC(=O)c3cc(-c4ccccc4)no3)C2)s1] |
| 42 | 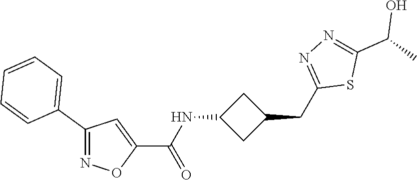 |
| 43 | 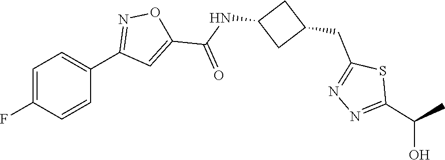 [SMILES: C[C@@H](O)c1nnc(C[C@H]2C[C@@H](NC(=O)c3cc(-c4ccc(F)cc4)no3)C2)s1] |
| 44 | 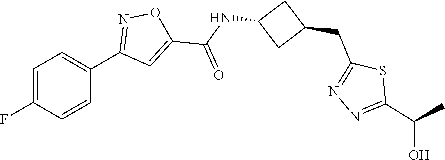 [SMILES: C[C@@H](O)c1nnc(C[C@H]2C[C@H](NC(=O)c3cc(-c4ccc(F)cc4)no3)C2)s1] |
| 45 | 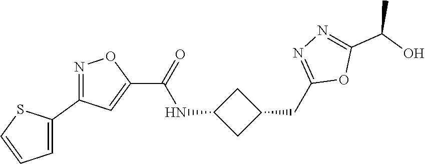 [SMILES: C[C@@H](O)c1nnc(C[C@H]2C[C@@H](NC(=O)c3cc(-c4cccs4)no3)C2)o1] |
| 46 | 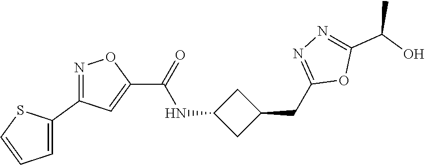 [SMILES: C[C@@H](O)c1nnc(C[C@H]2C[C@H](NC(=O)c3cc(-c4cccs4)no3)C2)o1] |
| 47 | 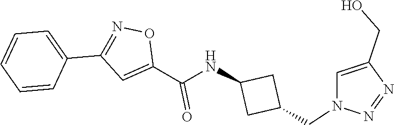 [SMILES: O=C(N[C@H]1C[C@H](Cn2cc(CO)nn2)C1)c1cc(-c2ccccc2)no1] |
| 48 | 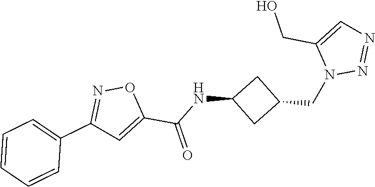 [SMILES: O=C(N[C@H]1C[C@H](Cn2nncc2CO)C1)c1cc(-c2ccccc2)no1] |
| 49 | 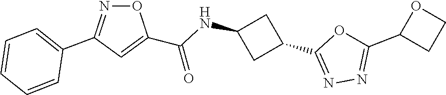 [SMILES: O=C(N[C@H]1C[C@H](c2nnc(C3CCO3)o2)C1)c1cc(-c2ccccc2)no1] |
| 50 | 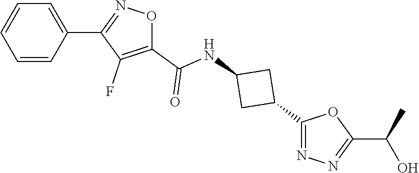 [SMILES: C[C@@H](O)c1nnc([C@H]2C[C@H](NC(=O)c3onc(-c4ccccc4)c3F)C2)o1] |
| 51 | 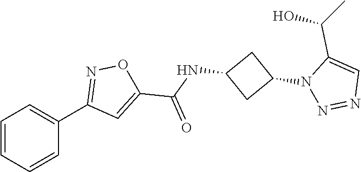 [SMILES: C[C@@H](O)c1cnnn1[C@H]1C[C@@H](NC(=O)c2cc(-c3ccccc3)no2)C1] |
| 52 | 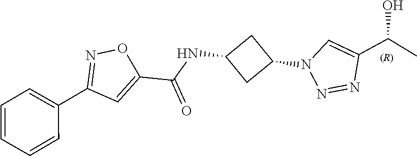 |
| 53 | 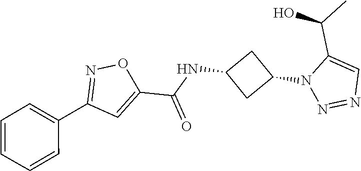 [SMILES: C[C@H](O)c1cnnn1[C@H]1C[C@@H](NC(=O)c2cc(-c3ccccc3)no2)C1] |
| 54 | 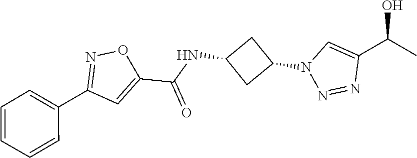 [SMILES: C[C@H](O)c1cn([C@H]2C[C@@H](NC(=O)c3cc(-c4ccccc4)no3)C2)nn1] |
| 55 | 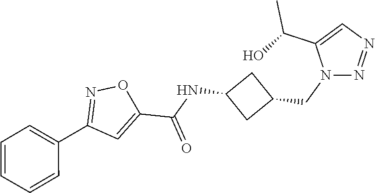 [SMILES: C[C@@H](O)c1cnnn1C[C@H]1C[C@@H](NC(=O)c2cc(-c3ccccc3)no2)C1] |
| 56 | 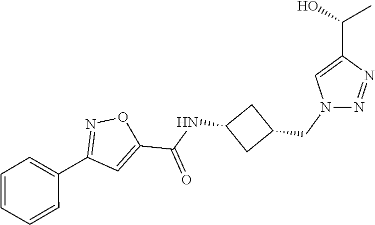 [SMILES: C[C@@H](O)c1cn(C[C@H]2C[C@@H](NC(=O)c3cc(-c4ccccc4)no3)C2)nn1] |
| 57 | 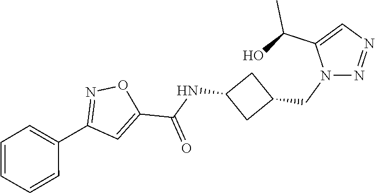 [SMILES: C[C@H](O)c1cnnn1C[C@H]1C[C@@H](NC(=O)c2cc(-c3ccccc3)no2)C1] |
| 58 | 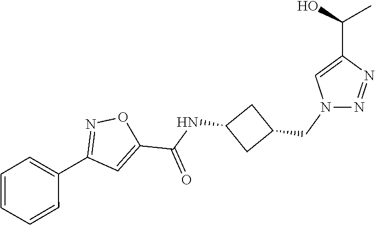 [SMILES: C[C@H](O)c1cn(C[C@H]2C[C@@H](NC(=O)c3cc(-c4ccccc4)no3)C2)nn1] |
| 59 | 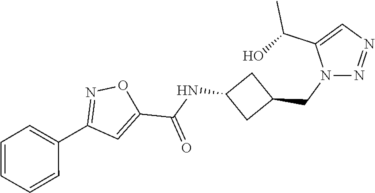 [SMILES: C[C@@H](O)c1cnnn1C[C@H]1C[C@H](NC(=O)c2cc(-c3ccccc3)no2)C1] |
| 60 | 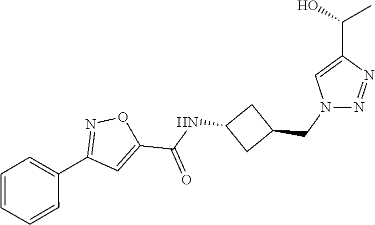 [SMILES: C[C@@H](O)c1cn(C[C@H]2C[C@H](NC(=O)c3cc(-c4ccccc4)no3)C2)nn1] |
| 61 | 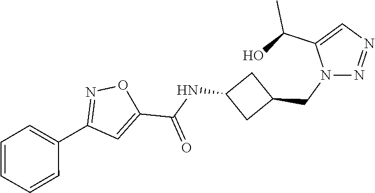 [SMILES: C[C@H](O)c1cnnn1C[C@H]1C[C@H](NC(=O)c2cc(-c3ccccc3)no2)C1] |
| 62 | 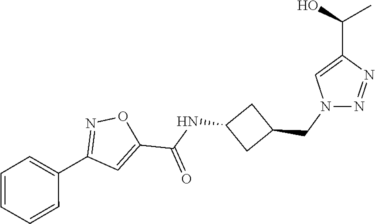 [SMILES: C[C@H](O)c1cn(C[C@H]2C[C@H](NC(=O)c3cc(-c4ccccc4)no3)C2)nn1] |
| 63 | 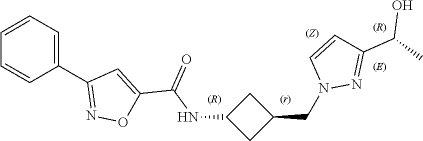 |
| 64 | 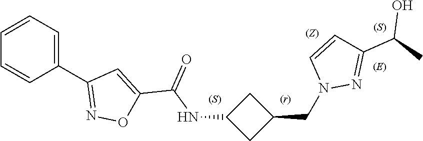 |
| 65 | 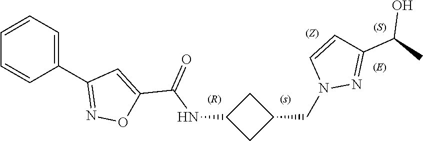 |
| 66 | 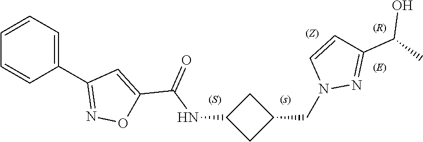 |
| 67 | 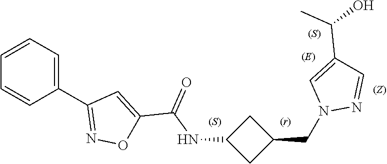 |
| 68 | 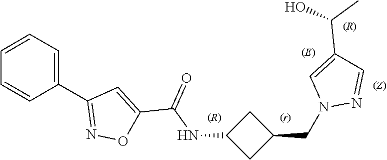 |
| 69 | 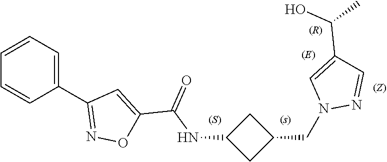 |
| 70 | 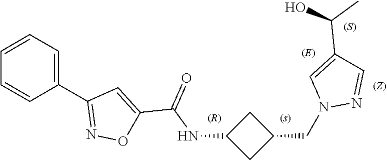 |
| 71 | 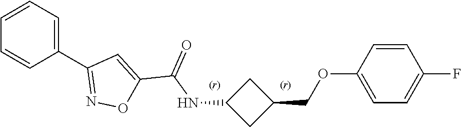 |
| 72 | 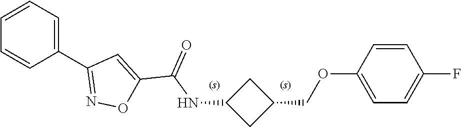 |
| 73 | 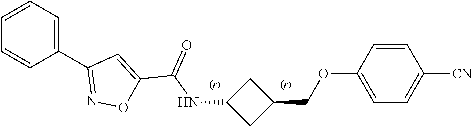 |
| 74 | 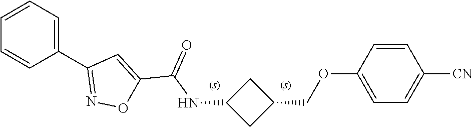 |
| 75 | 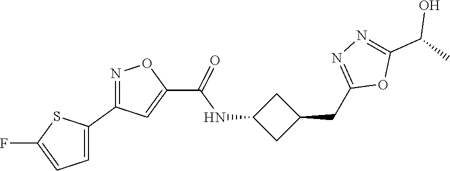 [SMILES: C[C@@H](O)c1nnc(C[C@H]2C[C@H](NC(=O)c3cc(-c4ccc(F)s4)no3)C2)o1] |
| 76 | 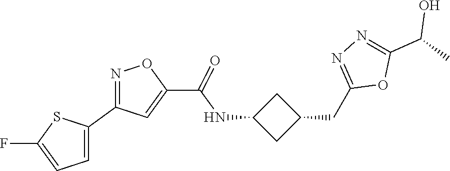 [SMILES: C[C@@H](O)c1nnc(C[C@H]2C[C@@H](NC(=O)c3cc(-c4ccc(F)s4)no3)C2)o1] |

Also contemplated herein are pharmaceutical compositions that include a disclosed compound and a pharmaceutically acceptable carrier or excipient. In certain embodiments, the compositions can include at least one additional CFTR modulator as described anywhere herein or at least two additional CFTR modulators, each independently as described anywhere herein.

It is to be understood that the specific embodiments described herein can be taken in combination with other specific embodiments delineated herein.

The features and other details of the disclosure will now be more particularly described. Before further description of the present invention, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

It will be appreciated that the description of the disclosure should be construed in congruity with the laws and principals of chemical bonding.

The term “alkyl”, as used herein, unless otherwise indicated, refers to both branched and straight-chain saturated aliphatic hydrocarbon groups having the specified number of carbon atoms; for example, “C₁-C₁₀ alkyl” denotes alkyl having 1 to 10 carbon atoms, and straight or branched hydrocarbons of 1-6, 1-4, or 1-3 carbon atoms, referred to herein as C₁₋₆alkyl, C₁₋₄alkyl, and C₁₋₃alkyl, respectively. Examples of alkyl include, but are not limited to, methyl, ethyl, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl.

The term, “alkenyl”, as used herein, refers to both straight and branched-chain moieties having the specified number of carbon atoms and having at least one carbon-carbon double bond. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

The term, “alkynyl”, as used herein, refers to both straight and branched-chain moieties having the specified number or carbon atoms and having at least one carbon-carbon triple bond.

The term “cycloalkyl,” as used herein, refers to saturated cyclic alkyl moieties having 3 or more carbon atoms, for example, 3-10, 3-8, 3-6, or 4-6 carbons, referred to herein as C₃₋₁₀cycloalkyl, C₃₋₆cycloalkyl or C₄₋₆cycloalkyl, respectively. The term cycloalkyl also includes bridged or fused cycloalkyls. Examples of cycloalkyl include, but are not limited to, cyclopropyl, cyclobutyl, cyclopentyl, cyclohexyl, cycloheptyl, adamantyl, bicyclo[1.1.1]pentane-, bicyclo[2.2.1]heptane, and bicyclo[3.2.1]octane. The term “cycloalkenyl,” as used herein, refers to cyclic alkenyl moieties having 3 or more carbon atoms.

The term “cycloalkoxy” as used herein refers to a cycloalkyl group attached to oxygen (cycloalkyl-O—). Exemplary cycloalkoxy groups include, but are not limited to, cycloalkoxy groups of 3-6 carbon atoms, referred to herein as C₃₋₆cycloalkoxy groups. Exemplary cycloalkoxy groups include, but are not limited to, cyclopropoxy, cyclobutoxy, cyclohexyloxy, etc.

The term “cycloalkynyl,” as used herein, refers to cyclic alkynyl moieties having 5 or more carbon atoms.

“Alkylene” means a straight or branched, saturated aliphatic divalent radical having the number of carbons indicated. “Cycloalkylene” refers to a divalent radical of carbocyclic saturated hydrocarbon group having the number of carbons indicated.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, etc.

The term “heterocyclic” or “heterocycle” encompasses heterocycloalkyl, heterocycloalkenyl, heterobicycloalkyl, heterobicycloalkenyl, heteropolycycloalkyl, heteropolycycloalkenyl, and the like unless indicated otherwise. Heterocycloalkyl refers to cycloalkyl groups containing one two, or three heteroatoms within the ring (O, S(O)_{w}, or NR where w is 0, 1, or 2 and R is e.g., H, C₁₋₃alkyl, phenyl) and for example 3, 4, or 5 carbons within the ring. Heterocycloalkenyl as used herein refers to cycloalkenyl groups containing one or more heteroatoms (O, S or N) within the ring. Heterobicycloalkyl refers to bicycloalkyl groups containing one or more heteroatoms (O, S(O)_{w} or NR) within a ring. Heterobicycloalkenyl as used herein refers to bicycloalkenyl groups containing one or more heteroatoms (O, S or N) within a ring. A heterocycle can refer to, for example, a saturated or partially unsaturated 4- to 12 or 4-10-membered ring structure, including bridged or fused rings, and whose ring structures include one to three heteroatoms, such as nitrogen, oxygen, and sulfur. Where possible, heterocyclic rings may be linked to the adjacent radical through carbon or nitrogen. Examples of heterocyclic groups include, but are not limited to, pyrrolidine, piperidine, morpholine, morpholine-one, thiomorpholine, piperazine, oxetane, azetidine, thietane dioxide, tetrahydrofuran or dihydrofuran etc.

Cycloalkyl, cycloalkenyl, heterocyclic, groups also include groups similar to those described above for each of these respective categories, but which are substituted with one or more oxo moieties.

The term “aryl”, as used herein, refers to mono- or polycyclic aromatic carbocyclic ring systems. A polycyclic aryl is a polycyclic ring system that comprises at least one aromatic ring. Polycyclic aryls can comprise fused rings, covalently attached rings or a combination thereof. The term “aryl” embraces aromatic radicals, such as, phenyl, naphthyl, indenyl, tetrahydronaphthyl, and indanyl. An aryl group may be substituted or unsubstituted. In some embodiments, the aryl is a C₄-C₁₀ aryl. Examples of optionally substituted aryl are phenyl, substituted phenyl, naphthyl and substituted naphthyl.

The term “heteroaryl”, as used herein, refers to aromatic carbocyclic groups containing one or more heteroatoms (O, S, or N) within a ring. A heteroaryl group, unless indicated otherwise, can be monocyclic or polycyclic. A heteroaryl group may additionally be substituted or unsubstituted. The heteroaryl groups of this disclosure can also include ring systems substituted with one or more oxo moieties. A polycyclic heteroaryl can comprise fused rings, covalently attached rings or a combination thereof. A polycyclic heteroaryl is a polycyclic ring system that comprises at least one aromatic ring containing one or more heteroatoms within a ring. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, thiazolopyridinyl, oxazolopyridinyl and azaindolyl. The foregoing heteroaryl groups may be C-attached or heteroatom-attached (where such is possible). For instance, a group derived from pyrrole may be pyrrol-1-yl (N-attached) or pyrrol-3-yl (C-attached). In some embodiments, the heteroaryl is 4- to 12-membered heteroaryl. In yet other embodiments, the heteroaryl is a mono or bicyclic 4- to 10-membered heteroaryl.

The term “substituted” refers to substitution by independent replacement of one, two, or three or more of the hydrogen atoms with substituents including, but not limited to, and unless indicated otherwise, —C₁-C₁₂ alkyl, —C₂-C₁₂ alkenyl, —C₂-C₁₂ alkynyl, —C₃-C₁₂ cycloalkyl, —C₃-C₁₂ cycloalkenyl, C₃-C₁₂ cycloalkynyl, -heterocyclic, —F, —Cl, —Br, —I, —OH, —NO₂, —N₃, —CN, —NH₂, oxo, thioxo, —NHRₓ, —NRₓRₓ, dialkylamino, -diarylamino, -diheteroarylamino, —ORₓ, —C(O)R_{y}, —C(O)C(O)R_{y}, —OCO₂R_{y}, —OC(O)R_{y}, OC(O)C(O)R_{y}, —NHC(O)R_{y}, —NHCO₂R_{y}, —NHC(O)C(O)R_{y}, NHC(S)NH₂, —NHC(S)NHRₓ, —NHC(NH)NH₂, —NHC(NH)NHRₓ, —NHC(NH)Rₓ, —C(NH)NHRₓ, and (C═NRₓ)Rₓ; —NRₓC(O)Rₓ, —NRₓC(O)N(Rₓ)₂, —NRₓCO₂R_{y}, —NRₓC(O)C(O)R_{y}, —NRₓC(S)NH₂, —NRₓC(S)NHRₓ, —NRₓC(NH)NH₂, —NRₓC(NH)NHRₓ, —NRₓC(NH)Rₓ, —C(NRₓ)NHRₓ—S(O)R_{y}, —NHSO₂Rₓ, —CH₂NH₂, —CH₂SO₂CH₃, -aryl, -arylalkyl, -heteroaryl, -heteroarylalkyl, -heterocycloalkyl, —C₃-C₁₂-cycloalkyl, -polyalkoxyalkyl, -polyalkoxy, -methoxymethoxy, -methoxyethoxy, —SH, —S—Rₓ, or -methylthiomethyl, wherein Rₓ is selected from the group consisting of hydrogen, —C₁-C₁₂ alkyl, —C₂-C₁₂ alkenyl, —C₂-C₁₂ alkynyl, —C₃-C₁₂ cycloalkyl, -aryl, -heteroaryl and -heterocyclic and —R_{y} is selected from the group consisting of hydrogen, —C₁-C₁₂ alkyl, —C₂-C₁₂ alkenyl, —C₂-C₁₂ alkynyl, —C₃-C₁₂ cycloalkyl, -aryl, -heteroaryl, -heterocyclic, —NH₂, —NH—C₁-C₁₂ alkyl, —NH—C₂-C₁₂ alkenyl, —NH—C₂-C₁₂-alkynyl, —NH—C₃-C₁₂ cycloalkyl, —NH-aryl, —NH-heteroaryl and —NH-heterocyclic. It is understood that the aryls, heteroaryls, alkyls, and the like can be further substituted.

The terms “halo” or “halogen” as used herein refer to F, Cl, Br, or I.

The term “haloalkyl” as used herein refers to an alkyl group having 1 to (2n+1) substituent(s) independently selected from F, Cl, Br or I, where n is the maximum number of carbon atoms in the alkyl group. It will be understood that haloalkyl is a specific example of an optionally substituted alkyl.

The terms “hydroxy” and “hydroxyl” as used herein refers to the radical —OH.

As will be understood by the skilled artisan, “H” is the symbol for hydrogen, “N” is the symbol for nitrogen, “S” is the symbol for sulfur, and “O” is the symbol for oxygen. “Me” is an abbreviation for methyl.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009. Where a particular compound is described or depicted, it is intended to encompass that chemical structure as well as tautomers of that structure.

The term “enantiomerically pure” means a stereomerically pure composition of a compound. For example, a stereochemically pure composition is a composition that is free or substantially free of other stereoisomers of that compound. In another example, for a compound having one chiral center, an enantiomerically pure composition of the compound is free or substantially free of the other enantiomer. In yet another example, for a compound having two chiral centers, an enantiomerically pure composition is free or substantially free of the other diastereomers.

Where a particular stereochemistry is described or depicted it is intended to mean that a particular enantiomer is present in excess relative to the other enantiomer. A compound has an R-configuration at a specific position when it is present in excess compared to the compound having an S-configuration at that position. A compound has an S-configuration at a specific position when it is present in excess compared to the compound having an R-configuration at that position.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent. The term “pharmaceutically acceptable salt(s)” as used herein refers to salts of acidic or basic groups that may be present in a disclosed compounds used in disclosed compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

Also included in the present disclosure are methods that include administering prodrugs of the compounds described herein, or a pharmaceutical composition thereof or method of use of the prodrug.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al, Nature Reviews Drug Discovery 2008, 7, 255). For example, if a compound of the disclosure or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino(C₂₋₃)alkyl (such as β-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a compound of the disclosure contains an alcohol functional group, a prodrug can be formed by the replacement of the hydrogen atom of the alcohol group with a group such as (C₁₋₆)alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxymethyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl-α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound of the disclosure incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. For examples, see Simplicio, et al., Molecules 2008, 13, 519 and references therein.

The disclosure additionally encompasses embodiments wherein one or more of the nitrogen atoms in a disclosed compound are oxidized to N-oxide.

Representative and exemplary synthetic routes for the preparation of compounds described herein are shown in the schemes below and throughout the Examples section.

### Methods of Use

The disclosure in part is directed to method of treating chronic obstructive pulmonary disease, bronchitis, or asthma in a patient in need thereof, or in a patient at risk of developing chronic obstructive pulmonary disease, comprising a) administering an effective amount of a disclosed compound (e.g. a compound of Formula (Ia), (IIa), (Ib), (IIb), (III), or (IV)) and b) optionally administering an effective amount of one or more of an additional active agent.

The disclosure is in part directed to a method of enhancing (e.g., increasing) CFTR activity in a subject (e.g., a subject suffering from any one or more of the conditions described herein) comprising administering a compound of the disclosure in an effective amount. The disclosure also encompasses a method of treating a patient suffering from a condition associated with CFTR activity comprising administering to said patient an effective amount of a compound described herein. In certain embodiments, the disease is COPD.

“Treating” or “treatment” includes preventing or delaying the onset of the symptoms, complications, or biochemical indicia of a disease, alleviating or ameliorating the symptoms or arresting or inhibiting further development of the disease, condition, or disorder. A “subject” is an animal to be treated or in need of treatment. A “patient” is a human subject in need of treatment.

An “effective amount” refers to that amount of an agent that is sufficient to achieve a desired and/or recited effect. In the context of a method of treatment, an “effective amount” of a therapeutic or active agent that is sufficient to ameliorate of one or more symptoms of a disorder and/or prevent advancement of a disorder, cause regression of the disorder and/or to achieve a desired effect.

The term “modulating” encompasses increasing, enhancing, inhibiting, decreasing, suppressing, and the like. The terms “increasing” and “enhancing” mean to cause a net gain by either direct or indirect means. As used herein, the terms “inhibiting” and “decreasing” encompass causing a net decrease by either direct or indirect means.

For example, CFTR activity in a patient may be enhanced after administration of a compound described herein when there is an increase in the CFTR activity as compared to that in the absence of the administration of the compound. CFTR activity encompasses, for example, chloride channel activity of the CFTR, and/or other ion transport activity (for example, HCO₃⁻ transport). In certain of these embodiments, the activity of one or more (e.g., one or two) mutant CFTRs (e.g., ΔF508, S549N, G542X mutations, Class IV CFTR mutations, Class V CFTR mutations, and Class VI mutations. Contemplated subject, G551D, R117H, N1303K, W1282X, R553X, 621+1G>T, 1717-1G>A, 3849+10kbC>T, 2789+5G>A, 3120+1G>A, 1507del, R1162X, 1898+1G>A, 3659delC, G85E, D1152H, R560T, R347P, 2184insA, A455E, R334W, Q493X, and 2184delA CFTR) is enhanced (e.g., increased). In certain embodiments, contemplated patients treated by disclosed methods, e.g, for treating COPD, do not have a CFTR mutation.

In certain embodiments a patient may have a Class I mutation, e.g., a G542X; a Class II/I mutation, e.g., a ΔF508/G542X compound heterozygous mutation. In other embodiments, the mutation is a Class III mutation, e.g., a G551D; a Class II/Class III mutation, e.g., a ΔF508/G551D compound heterozygous mutation. In still other embodiments, the mutation is a Class V mutation, e.g., a A455E; Class II/Class V mutation, e.g., a ΔF508/A455E compound heterozygous mutation. Of the more than 1000 known mutations of the CFTR gene, ΔF508 is the most prevalent mutation of CFTR which results in misfolding of the protein and impaired trafficking from the endoplasmic reticulum to the apical membrane (Dormer et al. (2001). J Cell Sci 114, 4073-4081; http://www.genet.sickkids.on.ca/app). In certain embodiments, ΔF508 CFTR activity is enhanced (e.g., increased). In certain embodiments, ΔF508 CFTR activity and/or G542X CFTR activity and/or G551D CFTR activity and/or A455E CFTR activity is enhanced (e.g., increased protein C deficiency, Aβ-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome). An enhancement of CFTR activity can be measured, for example, using literature described methods, including for example, Ussing chamber assays, patch clamp assays, and hBE Ieq assay (Devor et al. (2000), Am J Physiol Cell Physiol 279(2): C461-79; Dousmanis et al. (2002), J Gen Physiol 119(6): 545-59; Bruscia et al. (2005), PNAS 103(8): 2965-2971).

In some embodiments, disclosed methods of treatment that include administering a disclosed compound to a patient may further comprise administering an additional therapeutic or active agent. For example, in an embodiment, provided herein is a method of administering a disclosed compound and at least one additional therapeutic or active agent. In certain aspects, the disclosure is directed to a method comprising administering a disclosed compound, and at least two additional therapeutic agents. Additional therapeutic agents include, for example, those selected from the group consisting of: β₂ agonists, muscarinic antagonists, anticholinergics, corticosteroids, methylxanthine compounds, antihistamines, decongestants, anti-tussive drug substances, PDE I-VI inhibitors, prostacycline analogs, and calcium blockers. Exemplary additional active agents contemplated herein (e.g., for use in stable COPD) include bronchodilators such as β2 agonists and anticholinergics, and in certain embodiments, theophylline. Long-acting β2 agonists, or LABA, or long-acting muscarinic antagonists, or LAMA with or without inhaled corticosteroids, or ICS, may be used concomitantly or in combination for those with moderate to severe COPD. PDE-4 inhibitors, may be example, may be co-administered for example, in patients having severe COPD. Additional agents may include supplemental therapies, such as oxygen, pulmonary rehabilitation and physiotherapy, immunizations, as well as modified or additional nutrition and exercise plans.

Additional therapeutic or active agents include corticosteroids, for example, selected from the group consisting of: dexamethasone, budesonide, beclomethasone, triamcinolone, dexamethasone, mometasone, ciclesonide, fluticasone, flunisolide, dexamethasone sodium phosphate and pharmaceutically acceptable salts and esters thereof. For example, a corticosteroid may be selected from budesonide or beclomethasone dipropionate.

In another embodiment, an additional active agent is selected from the group consisting of interferon γ1β; bosentan, entanercept, and imatinib mesylate.

Contemplated therapeutic agents that may be administered in the disclosed methods include β-agonists such as a long acting β-agonist. Contemplated β-agonists may be selected from the group consisting of: albuterol, formoterol, pirbuterol, metapoterenol, salmeterol, arformoterol, indacaterol, levalbuterol, terbutaline and pharmaceutically acceptable salts thereof.

Additional active agents contemplated for use in one or more disclosed methods include long acting muscarinic antagonists, such as those selected from the group consisting of tiotropium, glycopyrronium, aclidinium and pharmaceutically acceptable salts thereof.

Further contemplated active agents for use in one or more disclosed methods include CFTR modulators known as CFTR potentiators, such as those selected from the group consisting of ivacaftor, isotopes of ivacaftor, GLPG1837/ABBV-974, FDL169, modulators that increase hydration and mucus (e.g., lancovutide, denufusol, sildenafil, miglustat, buphenyl), mucolytic agents, bronchodilators, antibiotics, anti-infective agents, anti-inflammatory agents, ion channel modulating agents (e.g., ENaC inhibitors), therapeutic agents used in gene therapy, CFTR correctors, and CFTR potentiators, or other agents that modulates CFTR activity. In some embodiments, at least one additional therapeutic agent is selected from the group consisting of a CFTR corrector and a CFTR potentiator. Non-limiting examples of CFTR correctors and potentiators include VX-770 (Ivacaftor), VX-809 (3-(6-(1-(2,2-difluorobenzo[d][1,3]dioxol-5-yl)cyclopropanecarboxamido)-3-methylpyridin-2-yl)benzoic acid, VX-661 (1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide), VX-983, VX-152, VX-440, and Ataluren (PTC124) (3-[5-(2-fluorophenyl)-1,2,4-oxadiazol-3-yl]benzoic acid), FDL169, GLPG1837/ABBV-974 (for example, a CFTR potentiator), GLPG 2665, GLPG2222 (for example, a corrector); and compounds described in, e.g., WO2014/144860 and 2014/176553, hereby incorporated by reference. Non-limiting examples of modulators include QBW-251, QR-010, NB-124, and compounds described in, e.g., WO2014/045283; WO2014/081821, WO2014/081820, WO2014/152213; WO2014/160440, WO2014/160478, US2014027933; WO2014/0228376, WO2013/038390, WO2011/113894, WO2013/038386; and WO2014/180562, of which the disclosed modulators in those publications are contemplated as an additional therapeutic agents and incorporated by reference. Non-limiting examples of anti-inflammatory agents include N6022 (3-(5-(4-(1H-imidazol-1-yl) phenyl)-1-(4-carbamoyl-2-methylphenyl)-1H-pyrrol-2-yl) propanoic acid), CTX-4430, N1861, N1785, and N91115. In some embodiments, the methods described herein can further include administering an additional therapeutic agent or administering at least two additional therapeutic agents. For example, two additional active agents may be administered where each selected from the group consisting of vilanterol, umeclidine, formoterol, salmeterol, budesone, fluticasone and pharmaceutically acceptable salts thereof.

In some embodiments, the methods described herein can further include administering an additional CFTR modulator or administering at least two additional CFTR modulators. In certain embodiments, at least one CFTR modulator is a CFTR corrector (e.g., VX-809, VX-661, VX-983, VX-152, VX-440, GLPG2665, and GLPG2222) or potentiator (e.g., ivacaftor, genistein and GLPG1837). In certain of these embodiments, one of the at least two additional therapeutic agents is a CFTR corrector (e.g., VX-809, VX-661, VX-983, VX-152, and VX-440) and the other is a CFTR potentiator (e.g., ivacaftor and genistein). In certain of these embodiments, one of the at least two additional therapeutic agents is a CFTR corrector (e.g., GLPG2222 or GLPG2665) and the other is a CFTR potentiator (e.g., GLPG1837). In certain of these embodiments, one of the at least two additional therapeutic agents is a CFTR corrector (e.g., VX-809 or VX-661) and the other is a CFTR potentiator (e.g., ivacaftor). In certain of these embodiments, at least one CFTR modulator is an agent that enhances read-through of stop codons (e.g., NB124 or ataluren). Administration of disclosed therapeutic agents in combination typically is carried out over a defined time period (usually a day, days, weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, inhalational routes, intravenous routes, intramuscular routes, and direct absorption through mucous membrane tissues. The therapeutic agents can be administered by the same route or by different routes. For example, a first therapeutic agent of the combination selected may be administered by intravenous injection or inhalation or nebulizer while the other therapeutic agents of the combination may be administered orally. Alternatively, for example, all therapeutic agents may be administered orally or all therapeutic agents may be administered by intravenous injection, inhalation or nebulization.

Combination therapy also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by a day, days or even weeks.

The components of a disclosed combination may be administered to a patient simultaneously or sequentially. It will be appreciated that the components may be present in the same pharmaceutically acceptable carrier and, therefore, are administered simultaneously. Alternatively, the active ingredients may be present in separate pharmaceutical carriers, such as, conventional oral dosage forms, that can be administered either simultaneously or sequentially.

### Compositions

Provided herein in an embodiment, are pharmaceutical compositions comprising a pharmaceutically acceptable carrier or excipient and a compound described herein, and methods of administering such compositions. For example, a disclosed compound, or a pharmaceutically acceptable salt, solvate, clathrate or prodrug thereof, can be administered in e.g., a disclosed method, in pharmaceutical compositions comprising a pharmaceutically acceptable carrier or excipient. The excipient can be chosen based on the expected route of administration of the composition in therapeutic applications. The route of administration of the composition depends on the condition to be treated. For example, intravenous injection may be preferred for treatment of a systemic disorder and oral administration may be preferred to treat a gastrointestinal disorder. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. A pharmaceutical composition comprising a disclosed compound or a pharmaceutically acceptable salt, solvate, clathrate or prodrug, can be administered by a variety of routes including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

The pharmaceutical composition can also be administered by nasal administration or inhalation. As used herein, nasally administering or nasal administration includes administering the composition to the mucus membranes of the nasal passage or nasal cavity of the patient. As used herein, pharmaceutical compositions for nasal administration of a composition include therapeutically effective amounts of the compounds prepared by well-known methods to be administered, for example, as a nasal spray, nasal drop, suspension, gel, ointment, cream or powder. Administration of the composition may also take place using a nasal tampon or nasal sponge.

In addition to the usual meaning of administering the formulations described herein to any part, tissue or organ whose primary function is gas exchange with the external environment, for purposes of the present disclosure, “pulmonary” will also mean to include a tissue or cavity that is contingent to the respiratory tract, in particular, the sinuses. For pulmonary (e.g., inhalation) administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the compound intended for pulmonary administration is dissolved, suspended or emulsified in a mixture of a solvent, surfactant and propellant. The mixture is maintained under pressure in a canister that has been sealed with a metering valve.

The disclosure is illustrated by the following examples which are not meant to be limiting in any way.

### EXEMPLIFICATION

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. At least some of the compounds identified as “intermediates” herein are contemplated as compounds of the invention.

### Preparation of tert-butyl (trans-3-azidocyclobutyl) carbamate

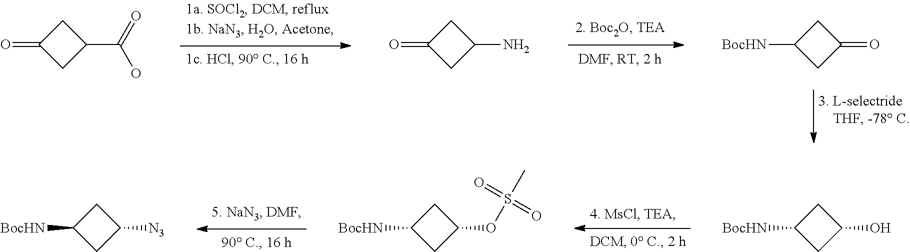 [SMILES: O=C1CC(C(=O)O)C1 ; NC1CC(=O)C1 ; CC(C)(C)OC(=O)NC1CC(=O)C1 ; CC(C)(C)OC(=O)N[C@H]1C[C@H](N=[N+]=[N-])C1 ; CC(C)(C)OC(=O)N[C@H]1C[C@@H](OS(C)(=O)=O)C1 ; CC(C)(C)OC(=O)N[C@H]1C[C@@H](O)C1]

Step 1: 3-Amino-cyclobutan-1-one: SOCl₂ (15.6 g, 131.46 mmol) was added dropwise to an ice-cooled solution of 3-oxocyclobutane carboxylic acid (5.0 g, 43.82 mmol) in dry DCM (30 mL) and the reaction mixture was refluxed for 3 h. The reaction mixture was cooled to room temperature and the volatiles were removed under reduced pressure to get the crude compound which was azeotropically distilled with toluene (20 mL×2) to remove acidic traces. The crude compound was dissolved in dry acetone (15 mL) and to the resulting solution was added a solution of NaN₃ (5.69 g, 87.64 mmol) in water (20 mL) at 0° C. over 30 min. The reaction mixture was stirred for 1 h at 0° C. and crushed ice was added to the reaction mixture. The aq. phase was extracted with ether (3×50 mL), dried over sodium sulfate and concentrated to ˜¼th volume. Then the reaction mixture was added to toluene (70 mL) and heated to 90° C., until evolution of N₂ ceased (˜30 min). To the resulting reaction mixture was added 20% HCl (50 mL) at 0° C. and the reaction mixture was gently heated to 90° C. for 16 h. The organic layer was separated and washed with water (50 mL). The aqueous layer was concentrated under vacuum to afford the product (5 g, crude) as a brown solid. ¹H-NMR (400 MHz, CDCl₃) δ 8.75 (br, 3H), 3.92-3.86 (m obscured by solvent signal, 2H), 3.38-3.31 (m, 3H).

Step 2: tert-butyl (3-oxocyclobutyl) carbamate: TEA (29.72 g, 293.73 mmol) was added dropwise to a solution of 3-aminocyclobutan-1-one (5.0 g, 58.74 mmol) and Boc₂O (25.64 g, 117.49 mmol) in DMF (80 mL) and the reaction mixture was stirred at room temperature for 2 h. After complete consumption of starting material as indicated by TLC, the reaction mixture was diluted with water (100 mL) and extracted with diethyl ether (6×70 mL). The combined organic layer was washed with brine (2×100 mL) and dried over Na₂SO₄. The solvent was removed under reduced pressure to get the crude compound which was purified by silica gel (100-200) column chromatography using 30% ethyl acetate in n-hexane to afford the product (5.3 g, 65% after two steps) as an off-white solid. ¹H-NMR (400 MHz, CDCl₃) δ 4.91 (br, 1H), 4.25 (br, 1H), 3.41-3.34 (m, 2H), 3.07-3.00 (m, 2H), 1.44 (s, 9H).

Step 3: tert-butyl cis-3-hydroxycyclobutyl)carbamate: a solution of L-Selectride (1M solution in THF) (8.053 mL, 8.05 mmol) was added dropwise over a period of 20 min to a solution of tert-butyl (3-oxocyclobutyl)carbamate (1.0 g, 5.40 mmol) in THF (25 mL) under N₂ atmosphere at −78° C. and the reaction mixture was stirred for 1h at −78° C. To the resulting reaction mixture was added a solution of NaOH (3.25 g) in water (4 mL) over a period of 10 min followed by 30% aqueous H₂O₂ (3 mL) over a period of 20 min. The reaction mixture was allowed to warm to room temperature and diluted with ethyl acetate (100 mL). The organic layer was separated off and washed with 10% aq. Na₂SO₃ (40 mL) followed by brine (40 mL). The organic layer dried over Na₂SO₄ and concentrated under reduced pressure to get the crude compound which was further purified by neutral alumina column chromatography using 50% ethyl acetate in n-hexane as eluent to afford the desired compound. The compound was washed with n-hexane to afford the product (0.750 g, 74%) as white solid. m. p. 119° C. (lit. value 117° C.); ¹H NMR (400 MHz, CDCl₃) δ 4.63 (br, 1H), 4.03-3.96 (m, 1H), 3.66-3.64 (m, 1H), 2.76-2.72 (m, 2H), 1.91 (br, 1H), 1.79-1.76 (m, 2H), 1.42 (s, 9H).

Step 4: cis-3-((tert-butoxycarbonyl)amino)cyclobutyl methanesulfonate: triethylamine (1.0 g, 9.93 mmol) was added to a cold (−10° C.) solution of tert-butyl (cis-3-hydroxycyclobutyl)carbamate (0.62 g, 3.31 mmol) in DCM (30 mL) followed by dropwise addition of methanesulfonyl chloride (0.45 g, 3.97 mmol) and the reaction mixture was stirred at −10° C. for 2 h. The reaction mixture was diluted with DCM (100 mL) and washed with water (5 mL) followed by dilute citric acid (30 mL) and brine (30 mL). The organic layer was dried over Na₂SO₄, concentrated under reduced pressure to afford the product (0.800 g, crude) as white solid which was used as such in next step without further purification. ¹H NMR (400 MHz, CDCl₃) δ 4.73-4.66 (m, 2H), 3.85-3.80 (m, 1H), 2.98 (s, 3H), 2.93-2.86 (m, 2H), 2.20-2.13 (m, 2H), 1.42 (s, 9H).

Step 5: tert-butyl (trans-3-azidocyclobutyl) carbamate: NaN₃ (0.49 g, 7.54 mmol) was added to a solution of cis-3-((tert-butoxycarbonyl) amino)cyclobutyl methanesulfonate (0.8 g, 3.01 mmol) in dry DMF (20 mL) and the mixture was heated at 85° C. for 16 h. The reaction mixture was diluted with water (40 mL) and the aqueous phase was extracted with ethyl acetate (50 mL×3). The combined organic layer was washed with brine (50 mL×4) and dried over Na₂SO₄. The solvent was removed under reduced pressure to get the crude product (0.73 g) as an off-white solid.

### Preparation of tert-butyl (cis-3-azidocyclobutyl) carbamate

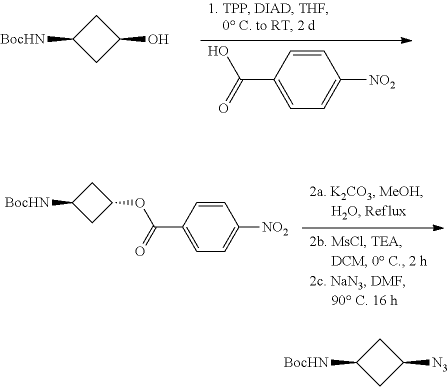 [SMILES: CC(C)(C)OC(=O)N[C@H]1C[C@@H](O)C1 ; O=C(O)c1ccc([N+](=O)[O-])cc1 ; CC(C)(C)OC(=O)N[C@H]1C[C@H](OC(=O)c2ccc([N+](=O)[O-])cc2)C1 ; CC(C)(C)OC(=O)N[C@H]1C[C@@H](N=[N+]=[N-])C1]

Step 1: trans-3-((tert-butoxycarbonyl)amino)cyclobutyl 4-nitrobenzoate: To an ice-cooled solution of tert-butyl (cis-3-hydroxycyclobutyl)carbamate (1.5 g, 80.11 mmol) and 4-nitrobenzoic acid (1.47 g, 88.12 mmol) in dry THF (60 mL) was added triphenylphosphine (3.15 g, 12.01 mmol) followed by dropwise addition of DIAD (8.09 g, 40.05 mmol) and the reaction mixture was stirred at room temperature for 2 days. The solvent was removed under reduced pressure to afford the crude compound which was purified by silica gel (100-200 mesh) column chromatography. Elution with 50% ethyl acetate in n-hexane followed by washing with diethyl ether (4 mL×2) gave the product (2.3 g, 85%) as a white solid. ¹H-NMR (400 MHz, CDCl₃) δ 8.29-8.27 (q, 2H, J=8.92 Hz), 8.21-8.19 (q, 2H, J=8.92 Hz), 5.37-5.32 (m, 1H), 4.77 (br, 1H), 4.41-4.38 (m, 1H), 2.64-2.58 (m, 2H), 2.47-2.40 (m, 2H), 1.44 (s, 9H); LC-MS (ES, M/Z): [M+H]⁺=336.8.

Step 2a: Trans-tert-butyl-3-hydroxycyclobutyl carbamate: trans-3-((tert-butoxycarbonyl) amino) cyclobutyl 4-nitrobenzoate was added (2.3 g, 68.38 mmol) to a suspension of K₂CO₃ (1.41 g, 10.25 mmol) in MeOH (50 mL) and water (10 mL) and the reaction mixture was heated at reflux for 2 h. The reaction mixture was cooled and filtered through celite bed. The filtrate was concentrated under reduced pressure to afford the crude product (4.2 g, crude) as an off-white solid which was used as such without further purification.

Step 2b: trans-3-((tert-butoxycarbonyl)amino)cyclobutyl methanesulfonate: triethylamine (6.8 g, 67.29 mmol) was added to a suspension of trans-tert-butyl-3-hydroxycyclobutyl carbamate (4.2 g, 22.43 mmol) in DCM (100 mL) followed by dropwise addition of methanesulfonyl chloride (3.08 g, 26.91 mmol) at −10° C. and the reaction mixture was stirred at −10° C. for 2 h. The reaction mixture was diluted with DCM (100 mL) and washed with water (50 mL) followed by brine (30 mL). The organic layer was dried over sodium sulfate and concentrated under reduced pressure to obtain the crude product (3.4 g, crude) as a yellow solid which was used as such in next step without purification.

Step 2c: cis-tert-butyl (3-azidocyclobutyl)carbamate: sodium azide (2.08 g, 32.035 mmol) was added to a solution of trans-3-((tert-butoxycarbonyl)amino)cyclobutyl methanesulfonate (3.4 g, 12.81 mmol) in dry DMF (20 mL) at room temperature and the reaction mixture was heated at 85° C. for 16 h. The crude reaction mixture was diluted with water (50 mL) and the aqueous phase was extracted with ethyl acetate (50 mL×3). The combined organic layer was washed with brine (50 mL×4) and dried over Na₂SO₄. The solvent was removed under reduced pressure to give the crude compound which was purified by neutral alumina column chromatography using 10% MeOH in DCM as eluent to afford the product (1.0 g, 68% after two steps) as a white solid. ¹H NMR (400 MHz, CDCl₃) δ 4.66 (br, 1H), 3.86-3.84 (m, 1H), 3.57-3.53 (m, 1H), 2.76-2.69 (m, 2H), 1.92-1.85 (m, 2H), 1.42 (s, 9H).

### Preparation of trans-3-(3-phenylisoxazole-5-carboxamido)cyclobutane-1-carboxylic acid

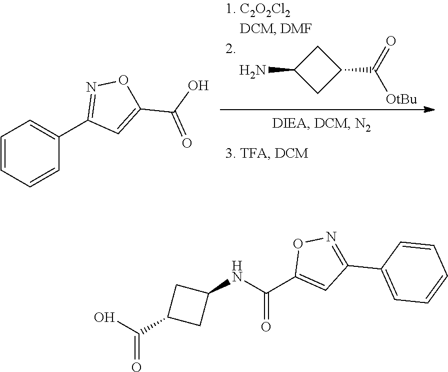 [SMILES: O=C(O)c1cc(-c2ccccc2)no1 ; CC(C)(C)OC(=O)[C@H]1C[C@H](N)C1 ; O=C(N[C@H]1C[C@H](C(=O)O)C1)c1cc(-c2ccccc2)no1]

Step 1: 3-phenylisoxazole-5-carbonyl chloride: DMF (0.5 mL) was added to a solution of 3-phenylisoxazole-5-carboxylic acid (10 g, 52.86 mmol, 1.00 eq.) and oxalyl chloride (8.74 g, 68.86 mmol, 1.30 eq.) in dichloromethane (200 mL) and the solution was stirred for 1 h at 0° C. The resulting mixture was concentrated under vacuum to give 11.265 g (crude) of 3-phenylisoxazole-5-carbonyl chloride as a yellow solid.

Step 2: tert-butyl 3-trans-(3-phenylisoxazole-5-amido)cyclobutane-1-carboxylate: a solution of 3-phenylisoxazole-5-carbonyl chloride (8.21 g, 39.54 mmol, 1.50 eq.) in dichloromethane (60 mL) was added dropwise to a solution of tert-butyl 3-trans-aminocyclobutane-1-carboxylate (4.5 g, 26.28 mmol, 1.00 eq.) and DIEA (6.79 g, 52.54 mmol, 2.00 eq.) in dichloromethane (30 mL) under N₂. The resulting solution was stirred for 2 h at 0° C. and then quenched with 100 mL of 5% K₂CO₃ aqueous. The resulting solution was extracted with dichloromethane and the organic layers combined, dried and concentrated under vacuum to give 9.7 g (crude) of tert-butyl 3-(3-phenylisoxazole-5-amido)cyclobutane-1-carboxylate as a light yellow solid. LC-MS (ES, m/z): [M+1]⁺=343.1.

Step 3: -3-trans-(3-phenylisoxazole-5-amido)cyclobutane-1-carboxylic acid: a solution of tert-butyl 3-trans-(3-phenylisoxazole-5-amido)cyclobutane-1-carboxylate (9.7 g, 28.33 mmol, 1.00 eq.) and trifluoroacetic acid (30 mL) in dichloromethane (100 mL) was stirred for 6 h at room temperature. The resulting mixture was concentrated under vacuum, dissolved in 20 mL of toluene and the solids were collected by filtration to obtain 5.116 g (63%) of 3-trans-(3-phenylisoxazole-5-amido)cyclobutane-1-carboxylic acid as a light yellow solid. LC-MS (ES, m/z): [M+1]⁺=287.0.

### Preparation of (R)-2-methoxypropanehydrazide

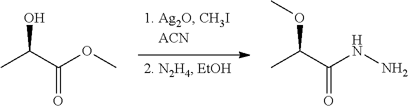

Step 1: methyl (2R)-2-methoxypropanoate: Ag₂O (6.1 g, 26.4 mmol, 1.10 eq.) was added to a solution of iodomethane (27.3 g, 192 mmol, 8.00 eq.) and methyl (2R)-2-hydroxypropanoate (2.5 g, 24 mmol, 1.00 eq.) in acetonitrile (30 mL) and the solution was stirred for 16 h at 85° C. in an oil bath. The solids were filtered and the mixture was diluted with DCM (100 mL). The resulting mixture was washed with water (3×50 mL), dried over anhydrous sodium sulfate and concentrated under vacuum to obtain 2 g (70%) of methyl (2R)-2-methoxypropanoate as colorless oil. ¹H NMR (400 MHz, CDCl₃): δ 3.92-3.87 (m, 1H), 3.76 (s, 3H), 3.40 (s, 3H), 1.42-1.40 (d, J=6.8 Hz, 3H).

Step 2: (2R)-2-methoxypropanehydrazide: a solution of methyl (2R)-2-methoxypropanoate (2 g, 16.93 mmol, 1.00 eq.) and hydrazine hydrate (5.3 g, 84.70 mmol, 5.00 eq.) in ethanol (50 mL) was stirred for 16 h at 70° C. in an oil bath. The resulting mixture was concentrated under vacuum to obtain 2 g (crude) of (2R)-2-methoxypropanehydrazide as light yellow oil. LC-MS (ES, m/z): [M+1]⁺=119.

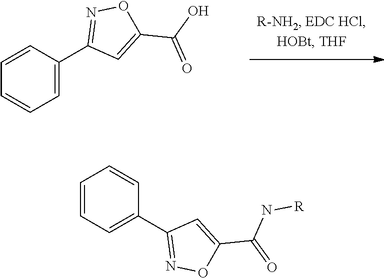

General procedure (1) for amide coupling: EDC HCl (1.98 mmol), HOBt.H₂O (1.32 mmol) and the appropriate amine (1.45 mmol) were added to a solution of 3-phenylisoxazole-5-carboxylic acid (1.32 mmol) in THF (10 mL) at room temperature. Reaction mixture was stirred for 15 h at room temperature and the reaction mixture was concentrated to dryness. The crude solid was extracted with EtOAc (3×10 mL) and washed with water. The combined organic layers were dried over Na₂SO₄ and concentrated. The crude compound was purified by Combiflash chromatography to give the corresponding amide.

### Example 1: N-(2-methoxyethyl)-3-phenylisoxazole-5-carboxamide

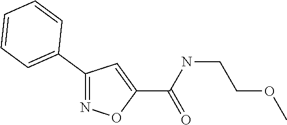 [SMILES: COCCNC(=O)c1cc(-c2ccccc2)no1]

Compound 1 was obtained as an off white solid using the general procedure 1 (0.120 g, 37.0%); ¹H-NMR (400 MHz, CDCl₃) δ 7.82-7.79 (m, 2H), 7.50-7.45 (m, 3H), 7.21 (s, 1H), 6.98-6.97 (br, 1H), 3.68-3.64 (m, 2H), 3.57-3.55 (t, 2H), 3.40 (s, 3H); LC-MS (ES, m/z): [M+H]⁺=247.2; HPLC purity: 99.76% at 220 nm and 99.64% at 254 nm.

### Example 2: 3-phenyl-N-((tetrahydrofuran-2-yl)methyl)isoxazole-5-carboxamide

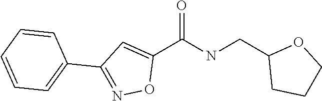 [SMILES: O=C(NCC1CCCO1)c1cc(-c2ccccc2)no1]

Compound 2 was obtained as a white solid using the general procedure 1 (0.110 g, 30.6%); ¹H NMR (400 MHz, CDCl₃) δ 7.82-7.80 (m, 2H), 7.49-7.45 (m, 3H), 7.25-7.21 (d, J=14.9 Hz, 1H), 6.95 (br, 1H), 4.08-4.06 (m, 1H), 3.92-3.89 (m, 1H), 3.81-3.71 (m, 2H), 3.44-3.39 (m, 1H), 2.06-1.99 (m, 1H), 1.96-1.91 (m, 2H), 1.63-1.58 (m, 1H); LC-MS (ES, m/z): [M+H]⁺=273.2; HPLC purity: 99.78% at 220 nm and 99.79% at 254 nm.

### Example 3: N-(2-morpholinoethyl)-3-phenylisoxazole-5-carboxamide

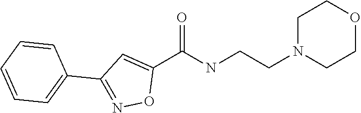 [SMILES: O=C(NCCN1CCOCC1)c1cc(-c2ccccc2)no1]

Compound 3 was obtained as a white solid using the general procedure 1 (0.125 g, 31.5%); ¹H NMR (400 MHz, CDCl₃) δ 7.82-7.80 (m, 2H), 7.49-7.46 (m, 3H), 7.21 (s, 2H), 3.75-3.72 (t, 4H), 3.58-3.53 (q, 2H), 2.61-2.58 (t, 2H), 2.51-2.50 (m, 4H); LC-MS (ES, m/z): [M+H]⁺=302.1; HPLC purity: 99.81% at 220 nm and 99.87% at 254 nm.

### Example 4: N-(3-(1H-imidazol-1-yl)propyl)-3-phenylisoxazole-5-carboxamide

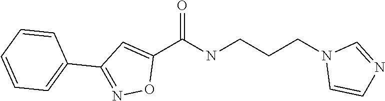 [SMILES: O=C(NCCCn1ccnc1)c1cc(-c2ccccc2)no1]

Compound 4 was obtained as a white solid using the general procedure 1 (0.127 g, 32.6%); ¹H NMR (400 MHz, CDCl₃) δ 7.82-7.79 (m, 2H), 7.52 (s, 1H), 7.50-7.46 (m, 3H), 7.22 (s, 1H), 7.08 (s, 1H), 6.98-6.97 (m, 1H), 6.79-6.76 (m, 1H), 4.08-4.04 (t, 2H), 3.52-3.47 (m, 2H), 2.18-2.11 (m, 2H); LC-MS (ES, m/z): [M+H]⁺=297.2; HPLC purity: 98.05% at 220 nm and 97.78% at 254 nm.

### Example 5: N-trans-3-(5-(hydroxymethyl)-1H-1,2,3-triazol-1-yl)cyclobutyl)-3-phenylisoxazole-5-carboxamide

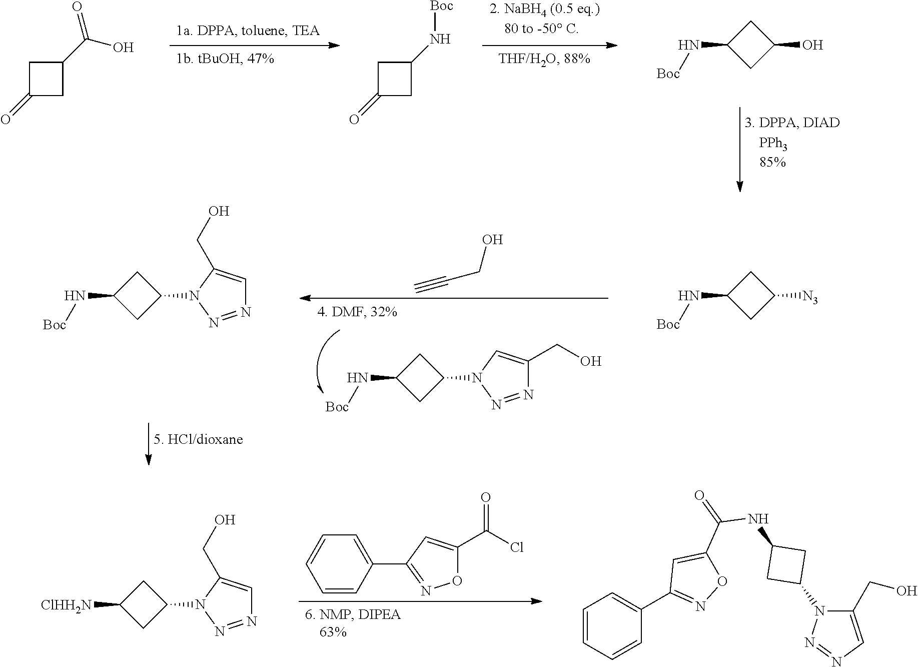

Step 1: tert-butyl (3-oxocyclobutyl)carbamate: DPPA (4.0 g, 1.1 eq.) was added dropwise to a cold (−5˜5° C.) solution of 3-oxocyclobutanecarboxylic acid (1.5 g, 1.0 eq.) and TEA (1.5 g, 1.1 eq.) in toluene (30 mL), and the mixture was stirred at −5-0° C. for 16 h. The reaction mixture was washed with NaHCO₃ (2×9 mL), water (1×9 mL) and NaCl aq. (1×4.5 mL) at 0˜10° C. The combined organic layer was dried over Na₂SO₄, filtered, and t-BuOH (7.5 mL) added to the filtrate. The reaction mixture was heated at 90˜100° C. for 16 h. The mixture was concentrated under vacuum at 60˜70° C., suspended in TBME (4.5 mL), filtered, and the solid dried over air to give 1.15 g (purity: 98.5%, yield: 47.2%) of product as a white solid.

Step 2: tert-butyl (cis-3-hydroxycyclobutyl)carbamate: a solution of tert-butyl (3-oxocyclobutyl)carbamate (200 mg, 1.0 eq.) in THF (1 mL) was added dropwise to a cold (below −70° C.) solution of NaBH₄ (20.4 mg, 0.5 eq.) in THF (1.8 mL) and water (2 mL), maintaining the temperature at −80˜−70° C. (ca. for 2 h for completion of addition). The mixture was stirred at −60˜−50° C. for 3 h, water (2 mL) was added to the reaction mixture and allowed to warm up to 15° C. The reaction mixture was then extracted with ethyl acetate (2 mL, 2×1 mL) and the combined organic layers were washed with brine (1 mL). The organic layer was concentrated under vacuum at 35˜40° C., the solid dissolved in toluene (1 mL, 80˜90° C.) and gradually cooled to 25-30° C. for 2.5 h. The mixture was stirred for 2 h at 25-30° C., filtered, and the solid dried in the air to give the product (177 mg with ratio of cis:trans (96.4:3.6), yield: 87.6%) as an off-white solid.

Step 3: tert-butyl (trans-3-azidocyclobutyl)carbamate: a solution of PPh₃ (315 mg) and DIAD (243 mg) in THF (3 mL) was stirred for 20 min at 0-10° C. A solution of tert-butyl (cis-3-hydroxycyclobutyl)carbamate (150 mg, 1.0 eq.) and DPPA (265 mg, 1.2 eq.) in THF (1 ml) was added dropwise and mixture was then warmed to 25-30° C. and stirred for 2 h. Brine (3 mL) was added to the reaction mixture, extracted with ethyl acetate (3 mL) and then concentrated under vacuum to give the crude oil. The mixture was purified by SiO₂ column chromatography and eluted with ethyl acetate/petroleum ether (0%˜10%) gradually. The product was suspended in n-heptane (0.3 mL) and stirred for 0.5 h at 20˜25° C. The mixture was filtered and the solid dried in air to give the product in 85% yield and ratio of cis/trans=4:96 checked by ¹H NMR.

Step 4: tert-butyl (trans-3-(5-(hydroxymethyl)-1H-1,2,3-triazol-1-yl)cyclobutyl)carbamate: a solution of tert-butyl (trans-3-azidocyclobutyl)carbamate (246 mg, 1.0 eq.) and prop-2-yn-1-ol (326 mg, 5.0 eq.) in DMF (1.2 mL) was heated at 90˜95° C. for 20 h. The mixture was concentrated under vacuum at 65° C. to give a ˜1:1 mixture of 4 and 5 regioisomers (353 mg). The mixture was purified by SFC to give tert-butyl (trans-3-(5-(hydroxymethyl)-1H-1,2,3-triazol-1-yl)cyclobutyl)carbamate (101 mg 32% yield, purity: 99.9% (205 nm)) as a solid.

Step 5: (1-(trans-3-aminocyclobutyl)-1H-1,2,3-triazol-5-yl)methanol hydrochloride: tert-butyl (trans-3-(5-(hydroxymethyl)-1H-1,2,3-triazol-1-yl)cyclobutyl)carbamate (101 mg, 1.0 eq.) was added slowly (5 portions) to a solution of HCl/dioxane (3.5 mol/L, 2 mL) at 20-˜30° C., and then stirred for 18 h at 20˜30° C. The reaction mixture was concentrated under vacuum at 55° C. to give the product (93.4 mg, assay 67% based on free base, Y: 100%) as a solid.

Step 6: N-(trans-3-(5-(hydroxymethyl)-1H-1,2,3-triazol-1-yl)cyclobutyl)-3-phenylisoxazole-5-carboxamide: DIPEA (388 mg, 3.00 mmol, 3.00 eq.) was added dropwise to a 0° C. solution of lithio 3-phenylisoxazole-5-carboxylate (190 mg, 0.97 mmol, 1.00 eq.), [1-[trans-3-aminocyclobutyl]-1H-1,2,3-triazol-5-yl]methanol hydrochloride (204 mg, 1.00 mmol, 1.00 eq.) and HATU (684 mg, 1.80 mmol, 1.80 eq.) in DMF (5 mL). The resulting solution was stirred for 1 hour at room temperature and then diluted with 50 mL of water/ice. The resulting solution was extracted with ethyl acetate (3×50 mL) and the organic layers combined. The resulting mixture was washed with brine (2×30 mL), dried over anhydrous sodium sulfate and concentrated under vacuum. The crude product was purified by Flash-Prep-HPLC with the following conditions (IntelFlash-1): Column, C18; mobile phase, H₂O/CH₃CN=100:1 increasing to H₂O/CH₃CN=1:100 within 30 min; Detector, UV 254 nm to afford 100 mg (30%) of 3-phenyl-N-[trans-3-[5-(hydroxymethyl)-1H-1,2,3-triazol-1-yl]cyclobutyl]isoxazole-5-carboxamide as a white solid. LC-MS (ES, m/z): [M+1]⁺=340; ¹H NMR (400 MHz, DMSO-d₆): δ 9.54-9.52 (d, J=7.2 Hz, 1H), 7.96-7.94 (m, 2H), 7.69-7.63 (m, 2H), 7.56-7.54 (m, 3H), 5.45-5.42 (t, J=5.6 Hz, 1H), 5.27-5.20 (m, 1H), 4.80-4.71 (m, 1H), 4.56-4.55 (d, J=5.6 Hz, 2H), 2.93-2.87 (m, 2H), 2.81-2.75 (m, 2H).

### Example 6: N-trans-3-(5-((R)-1-hydroxyethyl)-1,3,4-oxadiazol-2-yl)cyclobutyl)-3-phenylisoxazole-5-carboxamide

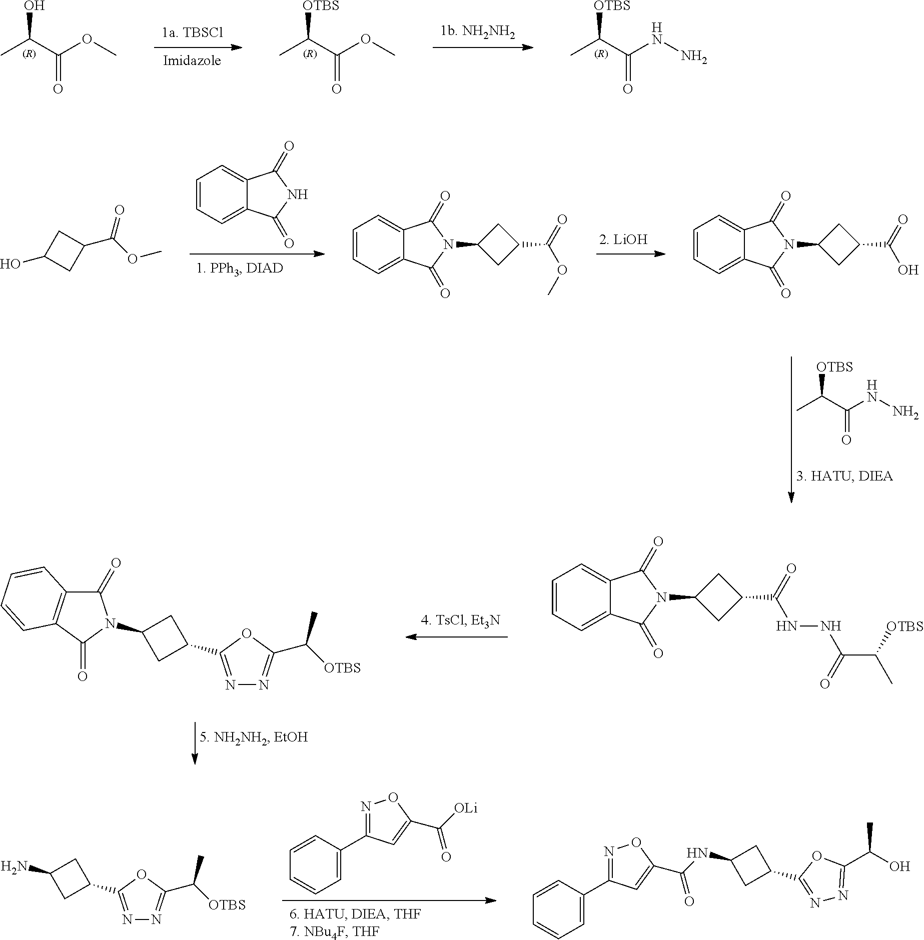

Step 1a: methyl (2R)-2-[(tert-butyldimethylsilyl)oxy]propanoate: into a 250-mL round-bottom flask was placed a solution of methyl (2R)-2-hydroxypropanoate (5 g, 48.03 mmol, 1.00 eq.) and imidazole (6.5 g, 95.59 mmol, 2.00 eq.) in dichloromethane (100 mL), followed by the dropwise addition of a solution of tert-butyl(chloro)dimethylsilane (8.7 g, 57.72 mmol, 1.20 eq.) in dichloromethane (50 mL) at 0° C. The resulting solution was stirred for 2 h at room temperature. The reaction was quenched by the addition of 100 mL of water/ice. The resulting solution was extracted with dichloromethane (3×100 mL) and the organic layers combined. The resulting mixture was washed with brine (3×50 mL), dried over anhydrous sodium sulfate and concentrated under vacuum to afford 7 g (67%) of methyl (2R)-2-[(tert-butyldimethylsilyl)oxy]propanoate as a colorless oil.

Step 1b: (2R)-2-[(tert-butyldimethylsilyl)oxy]propanehydrazide: into a 250-mL round-bottom flask was placed a solution of methyl (2R)-2-[(tert-butyldimethylsilyl)oxy]propanoate (7 g, 32.06 mmol, 1.00 eq.) in ethanol (100 mL). To the solution was added hydrazine (10 g, 159.81 mmol, 5.00 eq., 80%). The resulting solution was stirred for 15 h at 90° C. in an oil bath. The resulting solution was quenched by the addition of water/ice. The resulting solution was extracted with ethyl acetate (3×100 mL) and the organic layers combined. The resulting mixture was washed with brine (2×100 mL), dried over anhydrous sodium sulfate and concentrated under vacuum to afford 6.5 g (93%) of (2R)-2-[(tert-butyldimethylsilyl)oxy]propanehydrazide as a colorless oil. LC-MS (ES, m/z): [M+1]⁺=219.

Step 1: methyl (trans-3-(1,3-dioxo-2,3-dihydro-1H-isoindol-2-yl)cyclobutane-1-carboxylate: into a 250-mL round-bottom flask, under nitrogen was placed a solution of methyl 3-cis-hydroxycyclobutane-1-carboxylate (8 g, 61.47 mmol, 1.00 eq.), 2,3-dihydro-1H-isoindole-1,3-dione (18.1 g, 123.02 mmol, 2.00 eq.) and triphenylphosphine (32.3 g, 123.15 mmol, 2.00 eq.) in THF (100 mL), followed by addition of DIAD (24.9 g, 123.14 mmol, 2.00 eq.) dropwise with stirring at 0° C. The resulting solution was stirred for 2.5 hours at room temperature. The resulting mixture was concentrated under vacuum. The residue was applied onto a silica gel column with ethyl acetate/petroleum ether (1:5). The crude product was re-crystallized from petroleum ether/ethyl acetate in the ratio of 10:1 to afford 7.2 g (45%) of methyl trans-3-(1,3-dioxo-2,3-dihydro-1H-isoindol-2-yl)cyclobutane-1-carboxylate as a white solid. LC-MS (ES, m/z): [M+1]⁺=260. ¹H-NMR (400 MHz, CDCl₃): δ 7.85-7.82 (m, 2H), 7.74-7.71 (m, 2H), 5.08-5.04 (m, 1H), 3.75 (s, 3H), 3.34-3.32 (m, 1H), 3.20-3.12 (m, 2H), 2.66-2.60 (m, 2H).

Step 2: trans-3-(1,3-dioxo-2,3-dihydro-1H-isoindol-2-yl)cyclobutane-1-carboxylic acid: into a 100-mL round-bottom flask, was placed a solution of methyl trans-3-(1,3-dioxo-2,3-dihydro-1H-isoindol-2-yl)cyclobutane-1-carboxylate (7.2 g, 27.77 mmol, 1.00 eq.) in 1,4-dioxane (100 mL). To the solution was added 5M hydrogen chloride aqueous (10 mL). The resulting solution was stirred for 4 hours at 80° C. in an oil bath. The resulting mixture was concentrated under vacuum to afford 6.2 g (91%) of trans-3-(1,3-dioxo-2,3-dihydro-1H-isoindol-2-yl)cyclobutane-1-carboxylic acid as a white solid. LC-MS (ES, m/z): [M−1]⁻=244.

Step 3: (2R)-2-[(tert-butyldimethylsilyl)oxy]-N-[trans-3-(1,3-dioxo-2,3-dihydro-1H-isoindol-2-yl)cyclobutyl]carbonyl]propanehydrazide: into a 250-mL round-bottom flask, was placed a solution of trans-3-(1,3-dioxo-2,3-dihydro-1H-isoindol-2-yl)cyclobutane-1-carboxylic acid (6.2 g, 25.28 mmol, 1.00 eq.), (2R)-2-[(tert-butyldimethylsilyl)oxy]propanehydrazide (6.61 g, 30.27 mmol, 1.20 eq.) and HATU (14.4 g, 37.89 mmol, 1.50 eq.) in THF (100 mL), followed by the addition of DIEA (9.81 g, 75.91 mmol, 3.00 eq.) dropwise with stirring at 0° C. The resulting solution was stirred for 1 hour at room temperature. The reaction was then quenched by the addition of 100 mL of water/ice. The resulting solution was extracted with ethyl acetate (3×50 mL) and the organic layers combined. The resulting mixture was washed with brine (2×50 mL), dried over anhydrous sodium sulfate and concentrated under vacuum. The residue was applied onto a silica gel column with ethyl acetate/petroleum ether (1:4) to afford 7 g (62%) of (2R)-2-[(tert-butyldimethylsilyl)oxy]-N-[trans-3-(1,3-dioxo-2,3-dihydro-1H-isoindol-2-yl)cyclobutyl]carbonyl]propanehydrazide as colorless oil. LC-MS (ES, m/z): [M+1]⁺=446.

Step 4: 2-[trans-3-[5-[(1R)-1-[(tert-butyldimethylsilyl)oxy]ethyl]-1,3,4-oxadiazol-2-yl]cyclobutyl]-2,3-dihydro-1H-isoindole-1,3-dione: into a 250-mL round-bottom flask was placed a solution of (2R)-2-[(tert-butyldimethylsilyl)oxy]-N-[[trans-3-(1,3-dioxo-2,3-dihydro-1H-isoindol-2-yl)cyclobutyl]carbonyl]propanehydrazide (6.95 g, 15.60 mmol, 1.00 eq.) and TEA (7.89 g, 77.97 mmol, 5.00 eq.) in dichloromethane (100 mL), followed by addition of a solution of 4-methylbenzene-1-sulfonyl chloride (8.92 g, 46.79 mmol, 3.00 eq.) in dichloromethane (50 mL) dropwise with stirring at 0° C. The resulting solution was stirred for 15 hours at room temperature. The reaction was then quenched by the addition of 100 mL of water/ice. The resulting solution was extracted with dichloromethane (2×50 mL) and the organic layers combined. The resulting mixture was washed with brine (2×50 mL), dried over anhydrous sodium sulfate and concentrated under vacuum. The crude product was purified by Flash-Prep-HPLC with the following conditions (IntelFlash-1): Column, C18; mobile phase, H₂O/CH₃CN=100:1 increasing to H₂O/CH₃CN=1:100 within 30 min; Detector, UV 254 nm to afford 3.28 g (49%) of 2-[trans-3-[5-[(1R)-1-[(tert-butyldimethylsilyl)oxy]ethyl]-1,3,4-oxadiazol-2-yl]cyclobutyl]-2,3-dihydro-1H-isoindole-1,3-dione as colorless oil. LC-MS (ES, m/z): [M+1]⁺=428. ¹H-NMR (400 MHz, CDCl₃): δ 7.72-7.70 (m, 2H), 7.60-7.58 (m, 2H), 5.04-4.96 (m, 2H), 3.83-3.78 (m, 1H), 3.26-3.24 (m, 2H), 2.67-2.62 (m, 2H), 1.49-1.48 (d, J=6.8 Hz, 3H), 0.76 (s, 9H), 0.01 (s, 3H), 0.00 (s, 3H).

Step 5: trans-3-[5-[(1R)-1-[(tert-butyldimethylsilyl)oxy]ethyl]-1,3,4-oxadiazol-2-yl]cyclobutan-1-amine: into a 250-mL round-bottom flask, was placed a solution of 2-[trans-3-[5-[(1R)-1-[(tert-butyldimethylsilyl)oxy]ethyl]-1,3,4-oxadiazol-2-yl]cyclobutyl]-2,3-dihydro-1H-isoindole-1,3-dione (1.18 g, 2.76 mmol, 1.00 eq.) in ethanol (100 mL). To the solution was added hydrazine hydrate (3.45 g, 55.13 mmol, 20.00 eq., 80%). The resulting solution was stirred for 3 hours at room temperature. The solids were filtered. The resulting mixture was concentrated under vacuum to afford 760 mg (crude) of trans-3-[5-[(1R)-1-[(tert-butyldimethylsilyl)oxy]ethyl]-1,3,4-oxadiazol-2-yl]cyclobutan-1-amine as colorless oil. LC-MS (ES, m/z): [M+1]⁺=298.

Step 6: N-(trans-3-[5-[(1R)-1-[(tert-butyldimethylsilyl)oxy]ethyl]-1,3,4-oxadiazol-2-yl]cyclobutyl)-3-phenylisoxazole-5-carboxamide: into a 100-mL round-bottom flask, was placed a solution of lithio 3-phenylisoxazole-5-carboxylate (300 mg, 1.54 mmol, 1.20 eq.), 3-[5-[(1R)-1-[(tert-butyldimethylsilyl)oxy]ethyl]-1,3,4-oxadiazol-2-yl]cyclobutan-1-amine (380 mg, 1.28 mmol, 1.00 eq.) and HATU (728 mg, 1.92 mmol, 1.50 eq.) in THF (50 mL). This was followed by the addition of DIEA (500 mg, 3.87 mmol, 3.00 eq.) dropwise with stirring at 0° C. The resulting solution was stirred for 1 hour at room temperature. The resulting solution was diluted with 50 mL of water/ice. The resulting solution was extracted with ethyl acetate (3×50 mL) and the organic layers combined. The resulting mixture was washed with brine (2×30 mL), dried over anhydrous sodium sulfate and concentrated under vacuum to afford 300 mg (50%) of N-(trans-3-[5-[(1R)-1-[(tert-butyldimethylsilyl)oxy]ethyl]-1,3,4-oxadiazol-2-yl]cyclobutyl)-3-phenylisoxazole-5-carboxamide as an off-white crude solid. LC-MS (ES, m/z): [M+1]⁺=469.

Step 7: N-(trans-3-[5-[(1R)-1-hydroxyethyl]-1,3,4-oxadiazol-2-yl]cyclobutyl)-3-phenylisoxazole-5-carboxamide: into a 50-mL round-bottom flask, was placed a solution of N-(3-[trans-5-[(1R)-1-[(tert-butyldimethylsilyl)oxy]ethyl]-1,3,4-oxadiazol-2-yl]cyclobutyl)-3-phenylisoxazole-5-carboxamide (300 mg, 0.64 mmol, 1.00 eq.) and TBAF (1 mol/L in tetrahydrofuran, 1 mL) in THF (5 mL). The resulting solution was stirred for 3 hours at room temperature and diluted with 20 mL of water. The resulting solution was extracted with ethyl acetate (3×30 mL) and the organic layers combined. The resulting mixture was washed with brine (2×10 mL), dried over anhydrous sodium sulfate and concentrated under vacuum. The residue was applied onto a silica gel column with dichloromethane/methanol (20:1). The crude product was purified by Flash-Prep-HPLC with the following conditions (IntelFlash-1): Column, C18; mobile phase, H₂O/CH₃CN=100:1 increasing to H₂O/CH₃CN=1:100 within 30 min; Detector, UV 254 nm to afford 149.2 mg (66%) of N-(trans-3-[5-[(1R)-1-hydroxyethyl]-1,3,4-oxadiazol-2-yl]cyclobutyl)-3-phenylisoxazole-5-carboxamide (Compound A) as a white solid. LC-MS (ES, m/z): [M+1]⁺=355; ¹H NMR (400 MHz, DMSO-d₆): δ 9.48-9.46 (d, J=7.6 Hz, 1H), 7.96-7.93 (m, 2H), 7.67 (s, 1H), 7.56-7.54 (m, 3H), 5.95-5.94 (d, J=5.6 Hz, 1H), 4.95-4.89 (m, 1H), 4.73-4.63 (m, 1H), 3.77-3.71 (m, 1H), 2.73-2.50 (m, 4H), 1.50-1.48 (d, J=6.8 Hz, 3H).

### Example 7: N-(3-(1-methyl-1H-pyrazol-5-yl)propyl)-3-phenylisoxazole-5-carboxamide

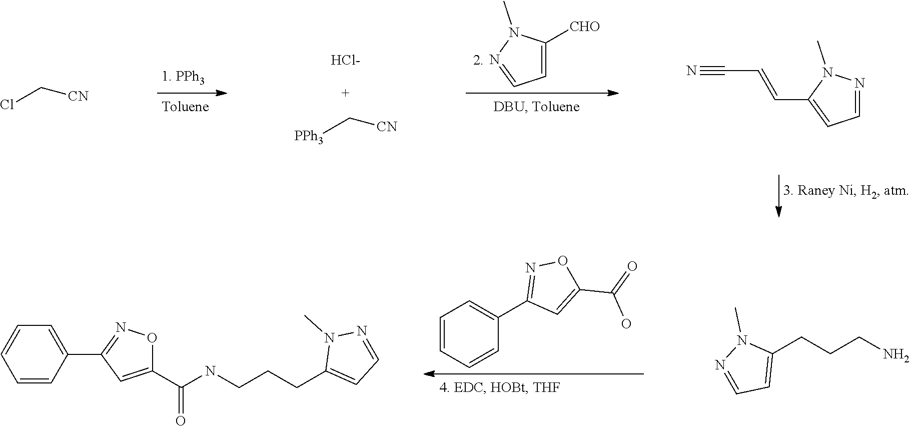

Step 1: Cyanomethyl triphenylphosphonium chloride: chloroacetonitrile (10 g, 0.132 mol) was added dropwise to a solution of triphenylphosphine (23.5 g, 0.0895 mol) in (120 mL) toluene and heated at reflux for 6 h. The reaction mixture was cooled to room temperature, the solids filtered and washed with (2×20 mL) diethyl ether. Compound (15 g, 49.58%) was obtained as a white solid. ¹H-NMR (400 MHz, DMSO) δ 8.02-7.97 (m, 3H), 7.90-7.79 (m, 12H), 5.94 (s, 1H), 5.90 (s, 1H); LC-MS (ES, m/z): [M+H]⁺=301.7

Step 2: 3-(2-Methyl-2H-pyrazol-3-yl)-acrylonitrile (4): To a stirred solution of 2-methyl-2H-pyrazole-3-carbaldehyde 3 (3.8 g, 0.0345 mol) in toluene (50 mL) was added cyanomethyl triphenylphosphonium chloride (12.8 g, 0.0389 mol) at room temperature. DBU (1.52 mL, 0.0099 mol) was then added dropwise and heated to reflux for 3 h. After completion of the reaction, the toluene was distilled off completely under vacuum. The resultant crude product was purified on combi flash, with the desired product eluted in 15% EtOAc:Hexane to afford the product (1.1 g, 24.01% yield) as a white solid. ¹H NMR (400 MHz, CDCl₃) δ 7.46-7.45 (d, J=176 Hz, 1H), 7.3-7.25 (m, 1H), 6.56 (s, 1H), 5.79-5.75 (d, J=16.34 Hz, 1H), 3.93 (s, 3H). LC-MS (ES, m/z): [M+H]⁺=134.1.

Step 3: 3-(1-methyl-1H-pyrazol-5-yl)propan-1-amine: Raney Ni (1 g, 50% in water suspension) was added to a solution of 3-(2-methyl-2H-pyrazol-3-yl)-acrylonitrile (1.0 g, 0.0075 mol) in ethanol (10 mL) at room temperature. The reaction mixture was then stirred under a hydrogen atmosphere for 16 h, filtered through a celite bed and washed with ethanol (2×10 mL). The filtrate was evaporated under vacuum to afford the compound (0.9 g, 86.53% yield) as a yellow oil. The crude product was used directly for amide coupling.

Step 4: N-(3-(1-methyl-1H-pyrazol-5-yl)propyl)-3-phenylisoxazole-5-carboxamide: EDC HCl (0.220 g, 0.00115 mole) and HOBt.H₂O (0.129 g, 0.00084 mole) were added to a solution of 3-phenylisoxazole-5-carboxylic acid (0.150 g, 0.00076 mol) in THF (5 mL) and stirred at room temperature for 20 minutes. To this reaction mixture was added 3-(1-methyl-1H-pyrazol-5-yl)propan-1-amine (0.16 g, 0.00115 mol) and DIPEA (0.590 mL, 0.0023 mole) and stirred for 16 h. The reaction mixture was concentrated on a rotary evaporator and the mixture was purified using combiflash, desired product eluted in 35% EtOAc:hexane (0.115 g, 47.23%) as an off white solid. ¹H NMR (400 MHz, CDCl₃) δ 7.54-7.53 (m, 1H), 7.50-7.48 (m, 1H), 7.38-7.37 (d, J=1.84 Hz, 1H), 7.15-7.14 (m, 1H), 6.88 (br, 1H), 6.81 (s, 1H), 3.79 (s, 3H), 3.56-3.51 (q, 2H), 2.71-2.67 (t, 2H), 2.02-1.95 (m, 2H); LC-MS (ES, m/z): [M+H]⁺=316.9; HPLC purity: 95.83% at 220 nm and 98.85% at 254 nm.

### Example 8: N-(2-methoxyethyl)-4-phenylfuran-2-carboxamide

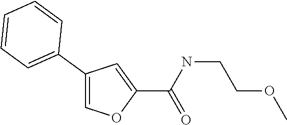 [SMILES: COCCNC(=O)c1cc(-c2ccccc2)co1]

Compound 8 was obtained as an off white solid using the general procedure 1. Yield: 57%; ¹H-NMR (400 MHz, DMSO-d₆) δ 8.42 (br, 1H), 8.35 (s, 1H), 7.66 (d, J=7.6 Hz, 2H), 7.57 (s, 1H), 7.42 (t, J=7.6 Hz, 2H), 7.31 (t, J=7.3 Hz, 1H), 3.44-3.39 (m, 4H), 3.26 (s, 3H); LC-MS (ES, m/z): [M+H]⁺=246.0; HPLC purity 99.32% at 220 nm and 99.35% at 254 nm.

### Example 9: 4-phenyl-N-((tetrahydrofuran-2-yl)methyl)furan-2-carboxamide

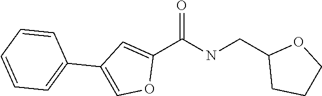 [SMILES: O=C(NCC1CCCO1)c1cc(-c2ccccc2)co1]

Compound 9 was obtained as an off white solid using the general procedure 1. Yield: 46%; ¹H-NMR (400 MHz, DMSO-d₆) δ 8.42 (br, 1H), 8.35 (s, 1H), 7.66 (d, J=7.6 Hz, 2H), 7.59 (s, 1H), 7.42 (t, J=7.2 Hz, 2H), 7.31 (t, J=7.2 Hz, 1H), 3.97 (m, 1H), 3.79 (m, 1H), 3.64 (m, 1H), 3.27 (s, 2H), 1.90-1.78 (m, 3H), 1.61 (m, 1H); LC-MS (ES, m/z): [M+H]⁺=271.9; HPLC purity 98.21% at 220 nm and 98.35% at 254 nm.

### Example 10: N-(2-morpholinoethyl)-4-phenylfuran-2-carboxamide

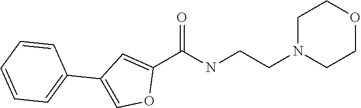 [SMILES: O=C(NCCN1CCOCC1)c1cc(-c2ccccc2)co1]

Compound 10 was obtained as an off white solid using the general procedure 1. Yield: 42%; ¹H-NMR (400 MHz, DMSO-d₆) δ 8.35 (m, 2H), 7.67 (d, J=7.6 Hz, 2H), 7.54 (s, 1H), 7.42 (t, J=7.2 Hz, 2H), 7.31 (t, J=7.2 Hz, 1H), 3.56 (s, 4H), 3.36 (s, 2H), 2.46-2.40 (m, 6H); LC-MS (ES, m/z): [M+H]⁺=300.7; HPLC purity 99.42% at 220 nm and 99.36% at 254 nm.

### Example 11: N-(3-(1H-imidazol-1-yl)propyl)-4-phenylfuran-2-carboxamide

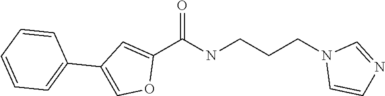 [SMILES: O=C(NCCCn1ccnc1)c1cc(-c2ccccc2)co1]

Compound 11 was obtained as an off white solid using the general procedure 1. Yield: 33%; ¹H-NMR (400 MHz, DMSO-d₆) δ 8.54 (t, J=5.6 Hz, 1H), 8.36 (s, 1H), 7.67 (d, J=7.2 Hz, 2H), 7.56 (s, 1H), 7.43 (t, J=7.2 Hz, 2H), 7.31 (t, J=7.2 Hz, 1H), 7.21 (s, 1H), 6.89 (s, 1H), 4.02 (t, J=6.8 Hz, 2H), 3.23 (q, J=6.8 Hz, 2H), 1.97 (quintet, J=6.8 Hz, 2H); LC-MS (ES, m/z): [M+H]⁺=296.1; HPLC purity 99.51% at 220 nm and 99.21% at 254 nm.

### Example 12: N-cyclopropyl-4-phenylfuran-2-carboxamide

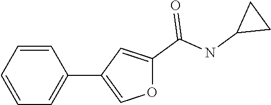 [SMILES: O=C(NC1CC1)c1cc(-c2ccccc2)co1]

Compound 12 was obtained as an off white solid using the general procedure 1 (0.032 g, 19.04%); ¹H NMR (400 MHz, CDCl₃) δ 7.66 (s, 1H), 7.48-7.46 (m, 2H), 7.41-7.36 (m, 3H), 7.31-7.24 (m, 1H), 6.44 (s, 1H), 2.89-2.85 (m, 1H), 0.89-0.84 (m, 2H), 0.65-0.61 (m, 2H); LC-MS (ES, m/z): [M+H]⁺=228.1; HPLC purity: 99.57% at 220 nm and 99.02% at 254 nm.

### Example 13: N-(trans-3-(5-(1-(methylsulfonyl)ethyl)-1,3,4-oxadiazol-2-yl)cyclobutyl)-3-phenylisoxazole-5-carboxamide

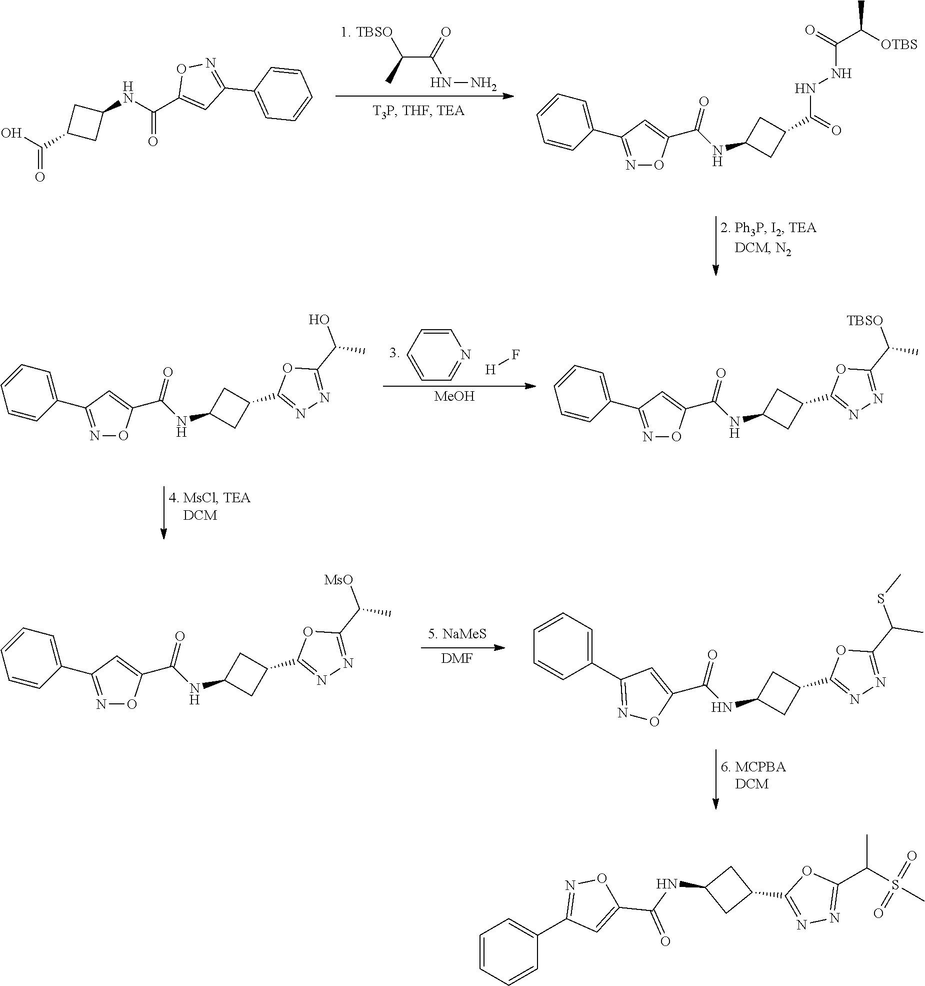

Step 1: N-trans-(3-[[(2R)-2-[(tert-butyldimethylsilyl)oxy]propanehydrazido]carbonyl]cyclobutyl)-3-phenylisoxazole-5-carboxamide: T₃P (50%) (55.6 g, 5.00 eq.), TEA (8.83 g, 87.26 mmol, 5.00 eq.) and (2R)-2-[(tert-butyldimethylsilyl)oxy]propanehydrazide (4.95 g, 22.67 mmol, 1.30 eq.) were added to a solution of 3-(3-phenylisoxazole-5-amido)cyclobutane-1-carboxylic acid (5 g, 17.47 mmol, 1.00 eq.) in tetrahydrofuran (50 mL) and the solution was stirred for 1.5 hours at 30° C. The reaction was then quenched by the addition of water, extracted with dichloromethane and the organic layers combined, dried and concentrated under vacuum. The residue was applied onto a silica gel column with ethyl acetate/petroleum ether (1:1) to give 8.45 g (crude) of N-trans-(3-[[(2R)-2-[(tert-butyldimethylsilyl)oxy]propanehydrazido]carbonyl]cyclobutyl)-3-phenylisoxazole-5-carboxamide as a light yellow solid; LC-MS (ES, m/z): [M+1]⁺=487.1.

Step 2: N-trans-(3-[5-[(R)-1-[(tert-butyldimethylsilyl)oxy]ethyl]-1,3,4-oxadiazol-2-yl]cyclobutyl)-3-phenylisoxazole-5-carboxamide: I₂ (20.74 g, 5.00 eq.) and TEA (9.98 g, 98.63 mmol, 6.00 eq.) were added to a solution of Ph₃P (21.56 g, 5.00 eq.) in dichloromethane (50 mL), followed by the dropwise addition of a solution of N-trans-(3-[[(2R)-2-[(tert-butyldimethylsilyl)oxy]propanehydrazido]carbonyl]cyclobutyl)-3-phenylisoxazole-5-carboxamide (8 g, 16.44 mmol, 1.00 eq.) in dichloromethane (50 mL). The resulting solution was stirred for 2.5 hours at 0° C., then quenched by the addition of water, and the solution was extracted with dichloromethane and the organic layers combined, dried and concentrated under vacuum to afford 3.19 g (41%) of N-trans-(3-[5-[(R)-1-[(tert-butyldimethylsilyl)oxy]ethyl]-1,3,4-oxadiazol-2-yl]cyclobutyl)-3-phenylisoxazole-5-carboxamide as a brown solid; LC-MS (ES, m/z): [M+1]⁺=469.1.

Step 3: N-trans-(3-[5-[(1R)-1-hydroxyethyl]-1,3,4-oxadiazol-2-yl]cyclobutyl)-3-phenylisoxazole-5-carboxamide: a solution of N-trans-(3-[5-[(1R)-1-[(tert-butyldimethylsilyl)oxy]ethyl]-1,3,4-oxadiazol-2-yl]cyclobutyl)-3-phenylisoxazole-5-carboxamide (25.3 g, 53.99 mmol, 1.00 eq.) and pyridine hydrofluoride (15 g, 151.35 mmol, 2.80 eq.) in methanol (50 mL) was stirred for 5 hours at room temperature. The reaction was then quenched by the addition of water, extracted with dichloromethane and the organic layers combined, dried and concentrated under vacuum. The residue was dissolved in 50 mL of toluene and the solids were collected by filtration to give 1.85 g (10%) of N-trans-(3-[5-[(1R)-1-hydroxyethyl]-1,3,4-oxadiazol-2-yl]cyclobutyl)-3-phenylisoxazole-5-carboxamide as a yellow solid; LC-MS (ES, m/z): [M+1]⁺=355.0.

Step 4: (R)-1-[5-trans-[3-(3-phenylisoxazole-5-amido)cyclobutyl]-1,3,4-oxadiazol-2-yl]ethyl methanesulfonate: TEA (1.28 g, 12.65 mmol, 3.00 eq.) and MsCl (0.725 g, 1.50 eq.) were added to a solution of N-trans-(3-[5-[(R)-1-hydroxyethyl]-1,3,4-oxadiazol-2-yl]cyclobutyl)-3-phenylisoxazole-5-carboxamide (1.5 g, 4.23 mmol, 1.00 eq.) in dichloromethane (50 mL) and the solution was stirred for 3 hours at 0° C. The reaction was then quenched by the addition of 200 mL of saturation NH₄Cl, extracted with dichloromethane and the organic layers combined, dried and concentrated under vacuum to give 1.72 g (94%) of (R)-1-[5-trans-[3-(3-phenylisoxazole-5-amido)cyclobutyl]-1,3,4-oxadiazol-2-yl]ethyl methanesulfonate as a yellow solid; LC-MS (ES, m/z): [M+1]⁺=433.0.

Step 5: N-trans-(3-[5-[1-(methylsulfanyl)ethyl]-1,3,4-oxadiazol-2-yl]cyclobutyl)-3-phenylisoxazole-5-carboxamide: a solution of (R)-1-[5-trans-[3-(3-phenylisoxazole-5-amido)cyclobutyl]-1,3,4-oxadiazol-2-yl]ethyl methanesulfonate (400 mg, 0.92 mmol, 1.00 eq.) and NaMeS (132 mg, 2.00 eq.) in DMF (3 mL) was stirred for 5 hours at 100° C. The resulting mixture was concentrated under vacuum and the residue was applied onto a silica gel column with ethyl acetate/petroleum ether (4:5) to give 254 mg (71%) of N-trans-(3-[5-[1-(methylsulfanyl)ethyl]-1,3,4-oxadiazol-2-yl]cyclobutyl)-3-phenylisoxazole-5-carboxamide as a yellow solid; LC-MS (ES, m/z): [M+1]⁺=385.0.

Step 6: N-(3-[5-trans-[1-methanesulfonylethyl]-1,3,4-oxadiazol-2-yl]cyclobutyl)-3-phenylisoxazole-5-carboxamide: a solution of N-(3-[5-trans-[1-(methylsulfanyl)ethyl]-1,3,4-oxadiazol-2-yl]cyclobutyl)-3-phenylisoxazole-5-carboxamide (230 mg, 0.60 mmol, 1.00 eq.) and MCPBA (0.42 g, 4.00 eq.) in dichloromethane (5 mL) was stirred for 2 hours at room temperature. The resulting mixture was concentrated under vacuum and the residue was applied onto a silica gel column with dichloromethane/methanol (25:1) to give 80 mg (32%) of a racemic mixture of N-(3-[5-trans-[1-methanesulfonylethyl]-1,3,4-oxadiazol-2-yl]cyclobutyl)-3-phenylisoxazole-5-carboxamide as a yellow solid; LC-MS (ES, m/z): [M+1]⁺=417.0 ¹H NMR (DMSO-d₆, 400 MHz, ppm): δ 9.44 (s, 1H), 7.93-7.91 (m, 2H), 7.65 (s, 1H), 7.54-7.52 (m, 3H), 5.16-5.11 (m, 1H), 4.69-4.63 (m, 1H), 3.78-3.75 (m, 1H), 3.14 (s, 3H), 2.72-2.65 (m, 4H), 1.74-1.70 (m, 3H); HPLC purity: 97.1% at 254 nm.

### Example 14: N-(trans-3-(5-((R)-1-methoxyethyl)-1,3,4-oxadiazol-2-yl)cyclobutyl)-3-phenylisoxazole-5-carboxamide

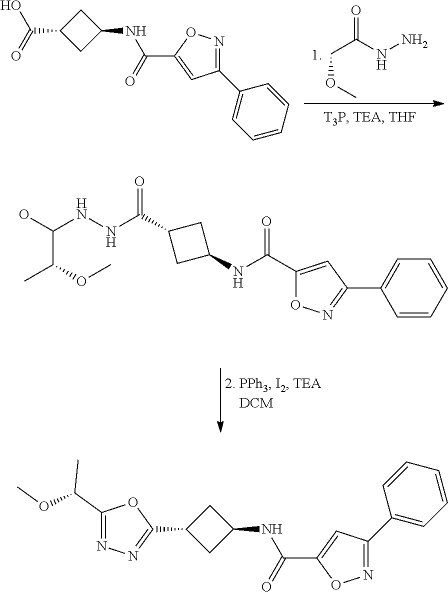

Step 1: 3-phenyl-N-[trans-3-[N-[(2R)-2-methoxypropanoyl]hydrazinecarbonyl]cyclobutyl]-1,2-oxazole-5-carboxamide: TEA (315 mg, 3.11 mmol, 2.97 eq.) and T₃P (667 mg) were added to a solution of trans-3-(3-phenylisoxazole-5-amido)cyclobutane-1-carboxylic acid (300 mg, 1.05 mmol, 1.00 eq.) and (2R)-2-methoxypropanehydrazide (185 mg, 1.57 mmol, 1.49 eq.) in tetrahydrofuran (5 mL) and the mixture was stirred for 2 hours at room temperature. The resulting mixture was concentrated under vacuum, diluted with 5 mL of methanol. The solids were collected by filtration and dried in an oven under reduced pressure to give 200 mg (49%) of 3-phenyl-N-[trans-3-[N-[(2R)-2-methoxypropanoyl]hydrazinecarbonyl]cyclobutyl]-1,2-oxazole-5-carboxamide as a white solid. LC-MS (ES, m/z): [M+1]⁺=387.2.

Step 2: 3-phenyl-N-[trans-3-[5-[(1S)-1-methoxyethyl]-1,3,4-oxadiazol-2-yl]cyclobutyl]-1,2-oxazole-5-carboxamide: 3-phenyl-N-[trans-3-[N-[(2R)-2-methoxypropanoyl]hydrazinecarbonyl]cyclobutyl]-1,2-oxazole-5-carboxamide (150 mg, 0.39 mmol, 1.00 eq.) was added to a solution of PPh₃ (150 mg, 0.57 mmol, 1.47 eq.), I₂ (150 mg) and TEA (120 mg, 1.19 mmol, 3.05 eq.) in dichloromethane (5 mL) and the mixture was stirred for 2 hours at 0° C. The resulting mixture was washed with water (2×5 mL) and concentrated under vacuum. The crude product was purified by Prep-HPLC with the following conditions: (Waters): Column: XBridge C18 OBD Prep Column 10 μm, 19 mm×250 mm; mobile phase, water (0.5% NH₄HCO₃) and CH₃CN; Gradient; 40% of CH₃CN to 45% of CH₃CN in 10 min; Detector, UV 254 nm to give 101.8 mg (71%) of 3-phenyl-N-[trans-3-[5-[(1S)-1-methoxyethyl]-1,3,4-oxadiazol-2-yl]cyclobutyl]-1,2-oxazole-5-carboxamide as a light yellow solid. LC-MS (ES, m/z): [M+1]⁺=369.0; ¹H NMR (DMSO-d₆, 300 MHz, ppm): δ 9.46-9.44 (d, J=7.2 Hz, 1H), 7.94-7.93 (m, 2H), 7.66 (s, 1H), 7.55-7.54 (m, 3H), 4.72-4.64 (m, 2H), 3.78-3.73 (m, 1H), 3.29 (s, 3H), 2.73-2.61 (m, 4H), 1.51-1.49 (d, J=6.8 Hz, 3H); HPLC purity: 99.1% at 254 nm.

### Example 15 and 16: 3-phenyl-N-(trans-3-(5-((S)-1-(2,2,2-trifluoroethoxy)ethyl)-1,3,4-oxadiazol-2-yl)cyclobutyl)isoxazole-5-carboxamide and 3-phenyl-N-(trans-3-(5-((R)-1-(2,2,2-trifluoroethoxy)ethyl)-1,3,4-oxadiazol-2-yl)cyclobutyl)isoxazole-5-carboxamide

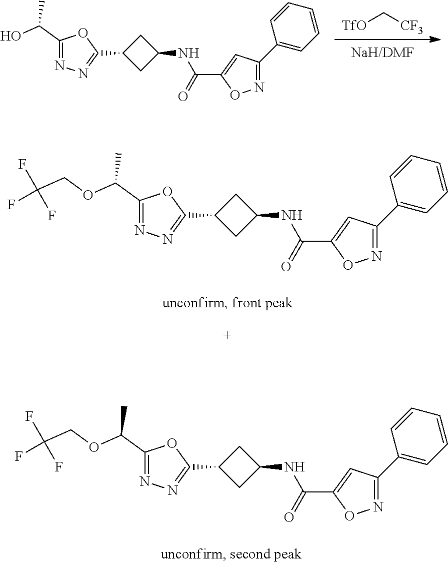 [SMILES: C[C@@H](O)c1nnc([C@H]2C[C@H](NC(=O)c3cc(-c4ccccc4)no3)C2)o1 ; O=S(=O)(OCC(F)(F)F)C(F)(F)F ; C[C@@H](OCC(F)(F)F)c1nnc([C@H]2C[C@H](NC(=O)c3cc(-c4ccccc4)no3)C2)o1 ; C[C@H](OCC(F)(F)F)c1nnc([C@H]2C[C@H](NC(=O)c3cc(-c4ccccc4)no3)C2)o1]

2,2,2-trifluoroethyl trifluoromethanesulfonate (491 mg, 2.12 mmol, 1.50 eq.) was added to a solution of 3-phenyl-N-[trans-3-[5-[(1R)-1-hydroxyethyl]-1,3,4-oxadiazol-2-yl]cyclobutyl]-isoxazole-5-carboxamide (500 mg, 1.41 mmol, 1.00 eq.) and sodium hydride (85 mg, 2.12 mmol, 1.50 eq.) in DMF (10 mL) and the solution was stirred for 2 hours at room temperature. The reaction mixture was diluted with water (30 mL), extracted with ethyl acetate (3×30 mL) and the organic layers were combined, dried over anhydrous sodium sulfate and concentrated under vacuum. The crude product was purified by Prep-HPLC with the following conditions (Waters): Column: XBridge C18 OBD Prep Column 10 μm, 19 mm×250 mm; Mobile Phase A: water (10 mmol/L NH₄HCO₃), Mobile Phase B: ACN; Flow rate: 25 mL/min; Gradient: 15% B to 65% B in 8 min; 254/220 nm. The isomers were purified by Chiral-Prep-HPLC with the following conditions: Column: Chiralpak IA 2*25 cm, 5 um; Mobile Phase A: Hexane; HPLC, Mobile Phase B: EtOH, HPLC Flow rate: 18 mL/min; Gradient: 40 B to 40 B in 15 min; 254/220 nm; RT1: 9.505; RT2: 11.208. This resulted in 19.1 mg (3%) of front peak as a white solid and 16.8 mg of second peak as a white solid.

Front Peak: LC-MS (ES, m/z): [M+1]⁺=437.1. ¹H-NMR (DMSO-d₆, 300 MHz, ppm): δ 7.87-7.86 (m, 2H), 7.49-7.47 (m, 3H), 7.37 (s, 1H), 5.00-4.94 (m, 1H), 4.11-4.02 (m, 2H), 3.81-3.74 (m, 1H), 2.78-2.68 (m, 4H), 1.64-1.62 (d, J=6.6 Hz, 3H); HPLC purity: 98.6% at 254 nm.

Second Peak: LC-MS (ES, m/z): [M+1]⁺=437.1; ¹H NMR (DMSO-d₆, 300 MHz, ppm): δ 7.86 (br, 2H), 7.48 (br, 3H), 7.37 (s, 1H), 5.00-4.94 (m, 1H), 4.10-4.02 (m, 2H), 3.79-3.77 (m, 1H), 2.78-2.69 (m, 4H), 1.64-1.62 (d, J=6.6 Hz, 3H); HPLC purity: 98.9% at 254 nm.

### Example 17: N-(trans-3-(5-(1-cyclobutoxyethyl)-1,3,4-oxadiazol-2-yl)cyclobutyl)-3-phenylisoxazole-5-carboxamide

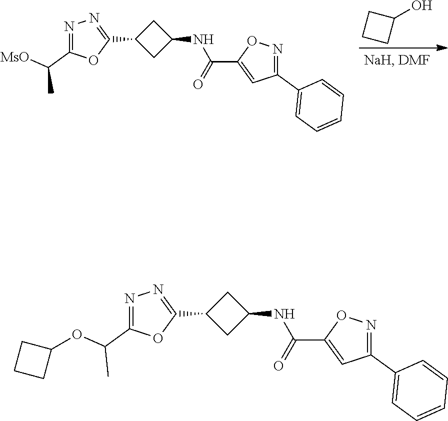 [SMILES: C[C@@H](OS(C)(=O)=O)c1nnc([C@H]2C[C@H](NC(=O)c3cc(-c4ccccc4)no3)C2)o1 ; OC1CCC1 ; CC(OC1CCC1)c1nnc([C@H]2C[C@H](NC(=O)c3cc(-c4ccccc4)no3)C2)o1]

Sodium hydride (84 mg, 2.10 mmol, 3.00 eq.) was added in portions to a cold (0° C.) solution of cyclobutanol (150 mg, 2.08 mmol, 3.00 eq.) in DMF (10 mL) and the resulting solution was stirred for 30 min at 0° C. (R)-1-[5-trans-[3-(3-phenylisoxazole-5-amido)cyclobutyl]-1,3,4-oxadiazol-2-yl]ethyl methanesulfonate (300 mg, 0.69 mmol, 1.00 eq.) was added to the mixture and stirred for an additional 2 hours at 25° C. The reaction was then quenched by the addition of 100 mL of water, extracted with ethyl acetate (2×100 mL) and the organic layers combined. The resulting mixture was washed with brine (2×100 mL), dried over anhydrous sodium sulfate and concentrated under vacuum. The crude product was purified by Prep-TLC (petroleum ether:ethyl acetate=1:1) to give 50.2 mg (18%) of 3-phenyl-N-[trans-3-[5-(1-cyclobutoxyethyl)-1,3,4-oxadiazol-2-yl]cyclobutyl]isoxazole-5-carboxamide as a white solid; LC-MS (ES, m/z): [M+H]⁺=409.4; ¹H NMR (300 MHz, DMSO-d₆) δ 9.46-9.43 (d, J=7.2 Hz, 1H), 7.95-7.92 (m, 2H), 7.65 (s, 1H), 7.56-7.54 (m, 3H), 4.78-4.64 (m, 2H), 4.04-3.99 (m, 1H), 3.77-3.74 (m, 1H), 2.71-2.50 (m, 4H), 2.18-2.14 (m, 1H), 1.97-1.85 (m, 2H), 1.75-1.57 (m, 2H), 1.49-1.47 (d, J=6.6 Hz, 3H), 1.47-1.40 (m, 1H); HPLC purity: 98.0% at 254 nm.

### Example 18: N-(trans-3-(5-(1-(cyclobutylmethoxy)ethyl)-1,3,4-oxadiazol-2-yl)cyclobutyl)-3-phenylisoxazole-5-carboxamide

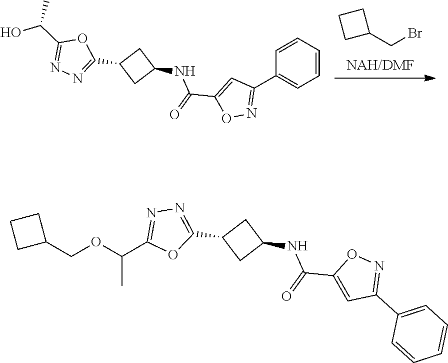

(Bromomethyl)cyclobutane (83 mg, 0.56 mmol, 2.00 eq.) was added to a solution of 3-phenyl-N-[trans-3-[5-(1-hydroxyethyl)-1,3,4-oxadiazol-2-yl]cyclobutyl]isoxazole-5-carboxamide (100 mg, 0.28 mmol, 1.00 eq.) and sodium hydride (17 mg, 0.42 mmol, 1.50 eq.) in DMF (2 mL). The resulting solution was stirred for 2 hours at room temperature, the reaction mixture was quenched by the addition of water (20 mL) and the solution was extracted with ethyl acetate (3×10 mL). The organic layers were combined and dried over anhydrous sodium sulfate and concentrated under vacuum. The crude product was purified by Prep-HPLC with the following conditions (Waters): Column: XBridge Prep C18 OBD Column 19×150 mm, Sum; Mobile Phase A: water (10 mmol/L NH₄HCO₃), Mobile Phase B: ACN; Flow rate: 20 mL/min; Gradient: 40% B to 80% B in 8 min; 254 nm to give 21.2 mg (18%) of 3-phenyl-N-[trans-3-[5-[1-(cyclobutylmethoxy)ethyl]-1,3,4-oxadiazol-2-yl]cyclobutyl]isoxazole-5-carboxamide as a white solid; LC-MS (ES, m/z): [M+1]⁺=421.0; ¹H NMR (DMSO-d₆, 300 MHz, ppm): δ 9.46-9.43 (d, J=7.2 Hz, 1H), 7.94-7.93 (m, 2H), 7.66 (s, 1H), 7.57-7.54 (m, 3H), 4.81-4.74 (m, 1H), 4.72-4.64 (m, 1H), 3.77-3.74 (m, 1H), 3.49-3.36 (m, 2H), 2.70-2.65 (m, 4H), 1.96-1.91 (m, 2H), 1.88-1.80 (m, 2H), 1.75-1.67 (m, 2H), 1.50-1.48 (d, J=6.6 Hz, 3H); HPLC purity: 99.8% at 254 nm.

### Example 19: N-(trans-3-(5-(1-(oxetan-3-ylmethoxy)ethyl)-1,3,4-oxadiazol-2-yl)cyclobutyl)-3-phenylisoxazole-5-carboxamide

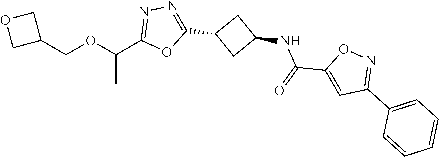 [SMILES: CC(OCC1COC1)c1nnc([C@H]2C[C@H](NC(=O)c3cc(-c4ccccc4)no3)C2)o1]

The title compound was prepared using the method shown in example 18.

### Example 20: N-(trans-3-(5-((R)-1-((1-methylazetidin-3-yl)methoxy)ethyl)-1,3,4-oxadiazol-2-yl)cyclobutyl)-3-phenylisoxazole-5-carboxamide

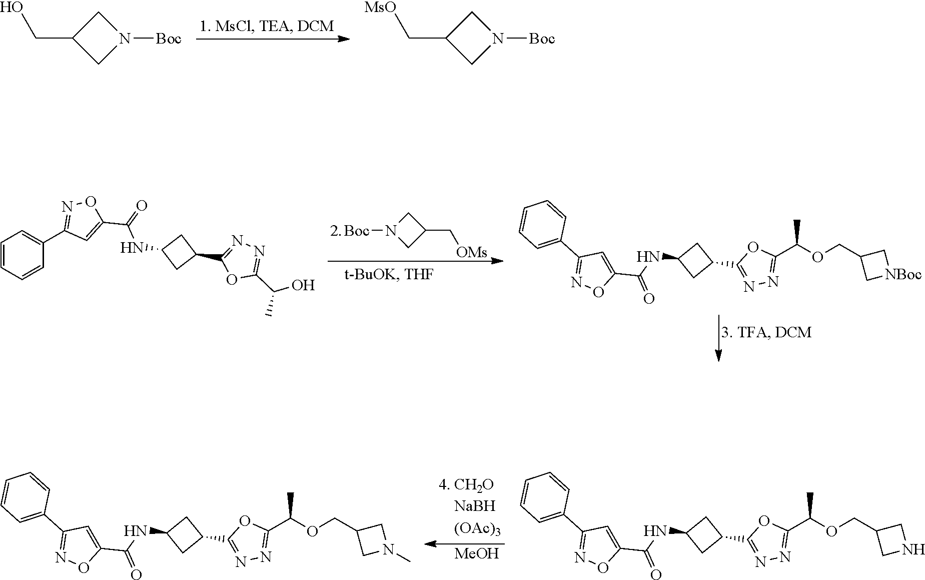

Step 1: tert-butyl 3-[(methanesulfonyloxy)methyl]azetidine-1-carboxylate: MsCl (549 mg, 4.82 mmol, 1.20 eq.) and TEA (606 mg, 6.00 mmol, 1.50 eq.) were added to a solution of tert-butyl 3-(hydroxymethyl)azetidine-1-carboxylate (750 mg, 4.01 mmol, 1.00 eq.) in dichloromethane (20 mL) and the solution was stirred for 3 hours at room temperature. The resulting solution was diluted with ethyl acetate (50 mL), washed with saturated sodium carbonate aq. (1×30 mL), water (1×30 mL), dried over anhydrous sodium sulfate and concentrated under vacuum to give 980 mg (92%) of tert-butyl 3-[(methanesulfonyloxy)methyl]azetidine-1-carboxylate as colorless oil.

Step 2: tert-butyl 3-[(1-[5-[trans-3-(3-phenylisoxazole-5-amido)cyclobutyl]-1,3,4-oxadiazol-2-yl]ethoxy)methyl]azetidine-1-carboxylate: tert-butyl 3-[(methanesulfonyloxy)methyl]azetidine-1-carboxylate (670 mg, 2.53 mmol, 1.50 eq.) was added to a solution of 3-phenyl-N-[trans-3-[5-[(1R)-1-hydroxyethyl]-1,3,4-oxadiazol-2-yl]cyclobutyl]-isoxazole-5-carboxamide (600 mg, 1.69 mmol, 1.00 eq.) and t-BuOK (570 mg, 5.08 mmol, 3.00 eq.) in THF (15 mL). The reaction was stirred for 16 hours at 80° C. in an oil bath then diluted with ethyl acetate (100 mL). The resulting solution was washed with water (2×30 mL), brine (1×30 mL), dried over anhydrous sodium sulfate and concentrated under vacuum. The residue was applied onto a silica gel column with ethyl acetate/petroleum ether (1:10 up to 1:2) to give 287 mg (32%) of tert-butyl 3-[(1-[5-[trans-3-(3-phenylisoxazole-5-amido)cyclobutyl]-1,3,4-oxadiazol-2-yl]ethoxy)methyl]azetidine-1-carboxylate as a light yellow solid; LC-MS (ES, m/z): [M+H]⁺=524.2.

Step 3: 3-phenyl-N-[trans-3-[5-[1-(azetidin-3-ylmethoxy)ethyl]-1,3,4-oxadiazol-2-yl]cyclobutyl]isoxazole-5-carboxamide: a solution of tert-butyl 3-[(1-[5-[trans-3-(3-phenylisoxazole-5-amido)cyclobutyl]-1,3,4-oxadiazol-2-yl]ethoxy)methyl]azetidine-1-carboxylate (237 mg, 0.45 mmol, 1.00 eq.) and TFA (1.5 mL) in DCM (4 mL) was stirred for 2 hours at room temperature. The reaction was quenched by addition of 20 mL of saturated sodium carbonate aqueous and extracted with ethyl acetate (2×50 mL). The combined organic layer was washed with water (1×10 mL), brine (1×10 mL), dried over anhydrous sodium sulfate and concentrated under vacuum to give 150 mg (78%) of 3-phenyl-N-[trans-3-[5-[1-(azetidin-3-ylmethoxy)ethyl]-1,3,4-oxadiazol-2-yl]cyclobutyl]isoxazole-5-carboxamide as a yellow solid; LC-MS (ES, m/z): [M+H]⁺=424.2.

Step 4: 3-phenyl-N-[trans3-(5-[1-[(1-methylazetidin-3-yl)methoxy]ethyl]-1,3,4-oxadiazol-2-yl)cyclobutyl]-isoxazole-5-carboxamide: HCHO (57 mg, 0.70 mmol, 1.50 eq.) was added to a solution of 3-phenyl-N-[trans-3-[5-[1-(azetidin-3-ylmethoxy)ethyl]-1,3,4-oxadiazol-2-yl]cyclobutyl]isoxazole-5-carboxamide (150 mg, 0.35 mmol, 1.00 eq.) in methanol (3 mL) and stirred for 30 min. NaBH(OAc)₃ (150 mg, 0.71 mmol, 2.00 eq.) was added to the reaction mixture and stirred 16 hours at room temperature. After removing the solid by filtration, the crude product (3 mL) was purified by Prep-HPLC with the following conditions (Waters): Column: XBridge C18 OBD Prep Column 10 μm, 19 mm×250 mm; Mobile Phase A: water (10 mmol/L NH₄HCO₃), Mobile Phase B: ACN; Flow rate: 20 mL/min; Gradient: 15% B to 45% B in 8 min; 220/254 nm to give 68.6 mg (44%) of 3-phenyl-N-[trans3-(5-[1-[(1-methylazetidin-3-yl)methoxy]ethyl]-1,3,4-oxadiazol-2-yl)cyclobutyl]-isoxazole-5-carboxamide as a white solid; LC-MS (ES, m/z): [M+H]⁺=438.2; ¹H NMR (CDOD, 400 MHz): δ 7.89-7.87 (m, 2H), 7.51-7.50 (m, 3H), 7.39 (s, 1H), 4.85-4.78 (m, 2H), 3.85-3.59 (m, 3H), 3.48-3.43 (m, 2H), 3.16-3.11 (m, 2H), 2.87-2.73 (m, 4H), 2.60-2.57 (m, 1H), 2.35-2.33 (m, 3H), 1.61-1.58 (m, 3H); HPLC purity: 97% at 254 nm.

### Example 21: N-(trans-3-(5-(1-methylazetidin-3-yl)-1,3,4-oxadiazol-2-yl)cyclobutyl)-3-phenylisoxazole-5-carboxamide trifluoroacetate

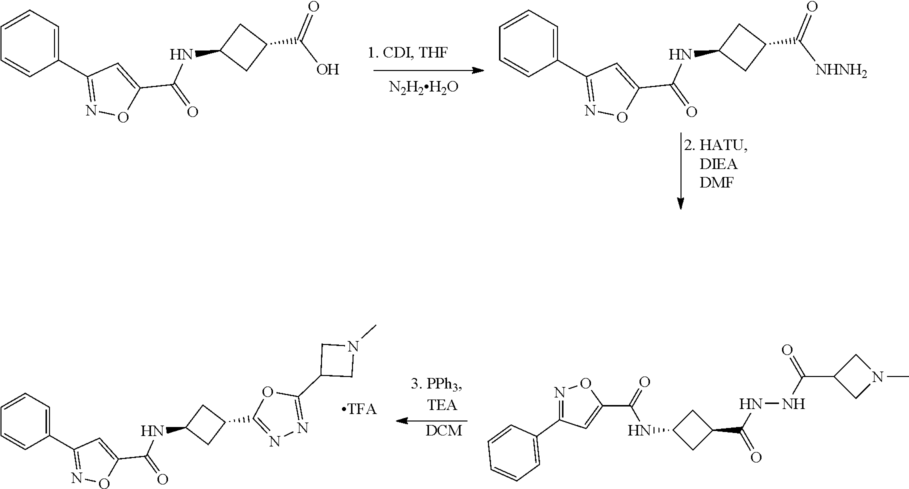

Step 1: 3-phenyl-N-[trans-3-(hydrazinecarbonyl)cyclobutyl]isoxazole-5-carboxamide: a solution of trans-3-(3-phenylisoxazole-5-amido)cyclobutane-1-carboxylic acid (1.706 g, 5.96 mmol, 1.00 eq.) and CDI (1.933 g, 11.92 mmol, 2.00 eq.) in tetrahydrofuran (30 mL) was stirred for 0.5 hour at room temperature. Hydrazine hydrate (1.118 g, 22.33 mmol, 3.75 eq.) was added to the reaction mixture and stirred for 2 hours at room temperature. The product was precipitated by the addition of water and collected by filtration to give 780 mg (44%) of 3-phenyl-N-[trans-3-(hydrazinecarbonyl)cyclobutyl]isoxazole-5-carboxamide as a white solid; LC-MS (ES, m/z): [M+H]⁺=301.2.

Step 2: 3-phenyl-N-[trans-3-[[(1-methylazetidin-3-yl)formohydrazido]carbonyl]cyclobutyl]-isoxazole-5-carboxamide: 1-methylazetidine-3-carboxylic acid (172.5 mg, 1.50 mmol, 1.50 eq.), HATU (570 mg, 1.50 mmol, 1.50 eq.) and DIEA (387 mg, 2.99 mmol, 3.00 eq.) were added to a solution of 3-phenyl-N-[trans-3-(hydrazinecarbonyl)cyclobutyl]-isoxazole-5-carboxamide (300 mg, 1.00 mmol, 1.00 eq.) in DMF (10 mL) and then stirred for 2 hours at room temperature. The crude product was purified by Flash-Prep-HPLC with the following conditions (IntelFlash-1): Column, C18; mobile phase, MeCN/H₂O=5:95 increasing to MeCN/H₂O=95:5 within 30 min; Detector, UV 254 nm to give 200 mg (50%) of 3-phenyl-N-[trans-3-[[(1-methylazetidin-3-yl)formohydrazido]carbonyl]cyclobutyl]-isoxazole-5-carboxamide as an off-white solid; LC-MS (ES, m/z): [M+H]⁺=398.0.

Step 3: 3-phenyl-N-[trans-3-[5-(1-methylazetidin-3-yl)-1,3,4-oxadiazol-2-yl]cyclobutyl]-isoxazole-5-carboxamide: I₂ (232 mg) and TEA (276 mg, 2.73 mmol, 5.99 eq.) were added to a cold (0° C.) solution of PPh₃ (239 mg, 0.91 mmol, 2.00 eq.) in DCM (20 mL). To the mixture was added 3-phenyl-N-[trans-3-[[(1-methylazetidin-3-yl)formohydrazido]carbonyl]cyclobutyl]-isoxazole-5-carboxamide (181 mg, 0.46 mmol, 1.00 eq.) at 0° C. The resulting solution was stirred for 3 hours at room temperature, diluted with 50 mL of DCM, washed with NaHSO₃ aqueous (2×50 mL) and concentrated under vacuum. The residue was applied onto a Prep-TLC with ethyl acetate/petroleum ether (1:1). The resulting crude product was purified by Prep-HPLC with the following conditions (HPLC-10): Column: XBridge C18 OBD Prep Column 100 Å, 10 μm, 19 mm×250 mm; Mobile Phase A: water (10 mmol/L NH₄HCO₃), Mobile Phase B: ACN; Flow rate: 20 mL/min; Gradient: 20% B to 30% B in 10 min; 254&220 nm to give 50 mg (29%) of 3-phenyl-N-[trans-3-[5-(1-methylazetidin-3-yl)-1,3,4-oxadiazol-2-yl]cyclobutyl]-isoxazole-5-carboxamide as a yellow solid; LC-MS (ES, m/z): [M−TFA+H]⁺=380.1; ¹H NMR (300 MHz, DMSO-d₆, ppm): δ 10.19-10.12 (m, 1H), 9.49-9.47 (d, J=7.5 Hz, 1H), 7.95-7.92 (m, 2H), 7.66-7.64 (d, J=8.1 Hz, 1H), 7.56-7.54 (t, J=3.3 Hz, 3H), 4.75-4.62 (m, 6H), 3.78-3.69 (m, 1H), 2.94 (s, 3H), 2.44-2.72 (m, 4H); HPLC purity: 97.1% at 254 nm.

### Example 22: N-trans-3-(5-(oxetan-3-yl)-1,3,4-oxadiazol-2-yl)cyclobutyl)-3-phenylisoxazole-5-carboxamide

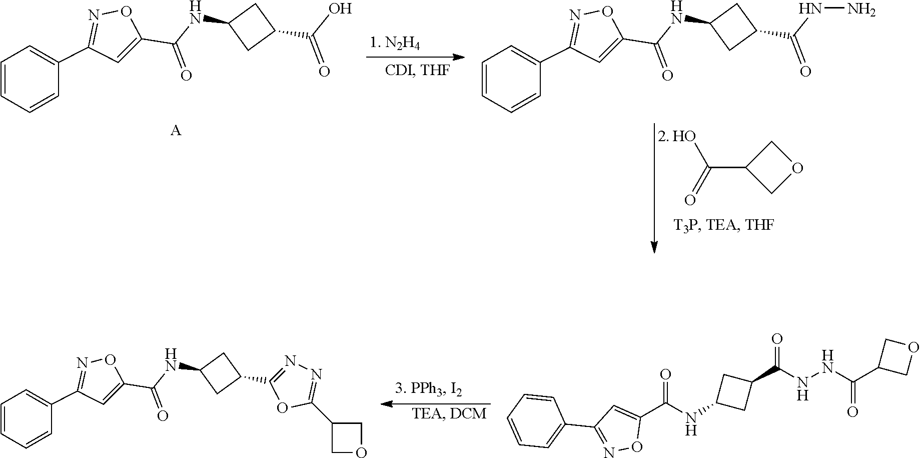

Step 1: 3-phenyl-N-[trans-3-(hydrazinecarbonyl)cyclobutyl]isoxazole-5-carboxamide: CDI (2.26 g, 13.94 mmol, 2.00 eq.) was added to a solution of N-trans-3-(3-phenylisoxazole-5-amido)cyclobutane-1-carboxylic acid (prepared according to procedure shown in example 13, 2 g, 6.99 mmol 1.00 eq.) in THF (3 mL) and the solution was stirred for 1 hour at room temperature, followed by the addition of hydrazine hydrate (1.33 g, 21.25 mmol, 3.00 eq., 80%). The resulting solution was stirred for additional 1 hour at room temperature and then quenched with water. After removing the solids by filtration, the resulting mixture was concentrated under vacuum and the residue was washed with 10 mL of methanol to give 960 mg (46%) of 3-phenyl-N-[trans-3-(hydrazinecarbonyl)cyclobutyl]isoxazole-5-carboxamide as a white solid; LC-MS (ES, m/z): [M+H]⁺=301.1.

Step 2: 3-phenyl-N-[trans-3-[(oxetan-3-ylformohydrazido)carbonyl]cyclobutyl]isoxazole-5-carboxamide: oxetane-3-carboxylic acid (170 mg, 1.67 mmol, 1.00 eq.), T₃P (5.3 g, 8.33 mmol, 5.00 eq., 50%) and TEA (838 mg, 8.3 mmol, 5.00 eq.) were added to a solution of 3-phenyl-N-[trans-3-(hydrazinecarbonyl)cyclobutyl]isoxazole-5-carboxamide (500 mg, 1.66 mmol, 1.00 eq.) in THF (50 mL). The resulting solution was stirred for 20 min at room temperature, then quenched by the addition of 200 mL of water. The resulting solution was extracted with dichloromethane (3×200 mL) and the organic layers combined. The resulting mixture was washed with brine, dried over anhydrous sodium sulfate and concentrated under vacuum. The residue solid was washed with 2 mL of methanol to afford 420 mg (66%) of 3-phenyl-N-[trans-3-[(oxetan-3-ylformohydrazido)carbonyl]cyclobutyl]isoxazole-5-carboxamide as an off-white solid; LC-MS (ES, m/z): [M+H]⁺=385.0.

Step 3: 3-phenyl-N-[trans-3-[5-(oxetan-3-yl)-1,3,4-oxadiazol-2-yl]cyclobutyl]isoxazole-5-carboxamide: I₂ (579 mg, 2.28 mmol, 2.50 eq.), TEA (598 mg, 5.91 mmol, 6.50 eq.) and 3-phenyl-N-[trans-3-[(oxetan-3-ylformohydrazido)carbonyl]cyclobutyl]isoxazole-5-carboxamide (350 mg, 0.91 mmol, 1.00 eq.) were added to a cold solution of PPh₃ (597 mg, 2.28 mmol, 2.50 eq.) in dichloromethane (30 mL) at 0° C. The resulting solution was stirred for 1 hour at room temperature, then quenched by the addition of water. The resulting solution was extracted with ethyl acetate and the organic layers combined. The resulting mixture was washed with brine, dried over anhydrous sodium sulfate and concentrated under vacuum. The residue was applied onto a silica gel column with dichloromethane/methanol (10:1) to afford 100.4 mg (30%) of 3-phenyl-N-[trans-3-[5-(oxetan-3-yl)-1,3,4-oxadiazol-2-yl]cyclobutyl]isoxazole-5-carboxamide as a white solid; LC-MS (ES, m/z): [M+H]⁺=367.1; ¹H NMR (300 MHz, DMSO-d₆, ppm): δ 9.46-9.44 (d, 1H, J=7.5 Hz), 7.95-7.92 (m, 2H), 7.66 (s, 1H), 7.56-7.54 (m, 3H), 4.95-4.90 (m, 2H), 4.83-4.79 (m, 2H), 4.75-4.51 (m, 2H), 3.78-3.71 (m, 1H), 2.70-2.65 (m, 4H); HPLC purity: 96.5% at 254 nm.

### Example 23: N-(trans-3-(5-(1,1-dioxidothietan-3-yl)-1,3,4-oxadiazol-2-yl)cyclobutyl)-3-phenylisoxazole-5-carboxamide

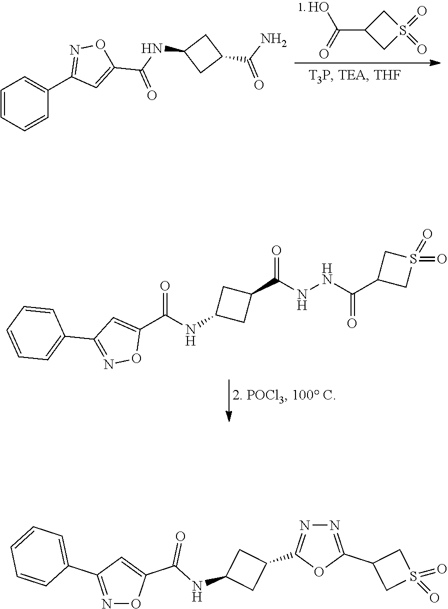 [SMILES: NC(=O)[C@H]1C[C@H](NC(=O)c2cc(-c3ccccc3)no2)C1 ; O=C(O)C1CS(=O)(=O)C1 ; O=C(N[C@H]1C[C@H](C(=O)NNC(=O)C2CS(=O)(=O)C2)C1)c1cc(-c2ccccc2)no1 ; O=C(N[C@H]1C[C@H](c2nnc(C3CS(=O)(=O)C3)o2)C1)c1cc(-c2ccccc2)no1]

Step 1: N-(trans-3-(2-(1,1-dioxidothietane-3-carbonyl)hydrazine-1-carbonyl)cyclobutyl)-3-phenylisoxazole-5-carboxamide: a solution of thietane-3-carboxylic acid 1,1-dioxide (500 mg, 3.4 mmol, 1.00 eq.), 3-phenyl-N-[trans-3-(hydrazinecarbonyl)cyclobutyl]-isoxazole-5-carboxamide (1.0 g, 3.4 mmol, 1.00 eq.), T₃P (10 mL) and TEA (4 mL) in tetrahydrofuran (20 mL) was stirred for 1 hour at room temperature. The reaction was then quenched by the addition of water and the solids were collected by filtration to afford 30 mg (42%) of N-(trans-3-(2-(1,1-dioxidothietane-3-carbonyl)hydrazine-1-carbonyl)cyclobutyl)-3-phenylisoxazole-5-carboxamide as a light yellow solid. LC-MS (ES, m/z): [M+H]⁺=433.1.

Step 2: N-(trans-3-(5-(1,1-dioxidothietan-3-yl)-1,3,4-oxadiazol-2-yl)cyclobutyl)-3-phenylisoxazole-5-carboxamide: a solution of N-(trans-3-(2-(1,1-dioxidothietane-3-carbonyl)hydrazine-1-carbonyl)cyclobutyl)-3-phenylisoxazole-5-carboxamide (400 mg, 0.92 mmol, 1.00 eq.) in POCl₃ (8 mL) was stirred for 3 hours at 100° C. in an oil bath. The reaction was then quenched by the addition of sodium bicarbonate aqueous/ice, extracted with ethyl acetate and the organic layers combined. The resulting mixture was washed with water, dried over anhydrous sodium sulfate and concentrated under vacuum to give 105.8 mg (28%) of N-(trans-3-(5-(1,1-dioxidothietan-3-yl)-1,3,4-oxadiazol-2-yl)cyclobutyl)-3-phenylisoxazole-5-carboxamide as a white solid; LC-MS (ES, m/z): [M+H]⁺=415.2; ¹H NMR (DMSO-d₆, 400 MHz): δ 9.46-9.42 (m, 1H), 7.95-7.91 (m, 2H), 7.66-7.65 (m, 1H), 7.55-7.54 (m, 3H), 4.75-4.57 (m, 5H), 4.23-4.14 (m, 1H), 3.73-3.52 (m, 1H), 2.70-2.66 (m, 4H); HPLC purity: 99.2% at 254 nm.

### Examples 24 and 25: N-cis-(3-(5-(1-(1-methylpiperidin-4-yl)azetidin-3-yl)-1,3,4-oxadiazol-2-yl)cyclobutyl)-3-phenylisoxazole-5-carboxamide and N-trans-(3-(5-(1-(1-methylpiperidin-4-yl)azetidin-3-yl)-1,3,4-oxadiazol-2-yl)cyclobutyl)-3-phenylisoxazole-5-carboxamide

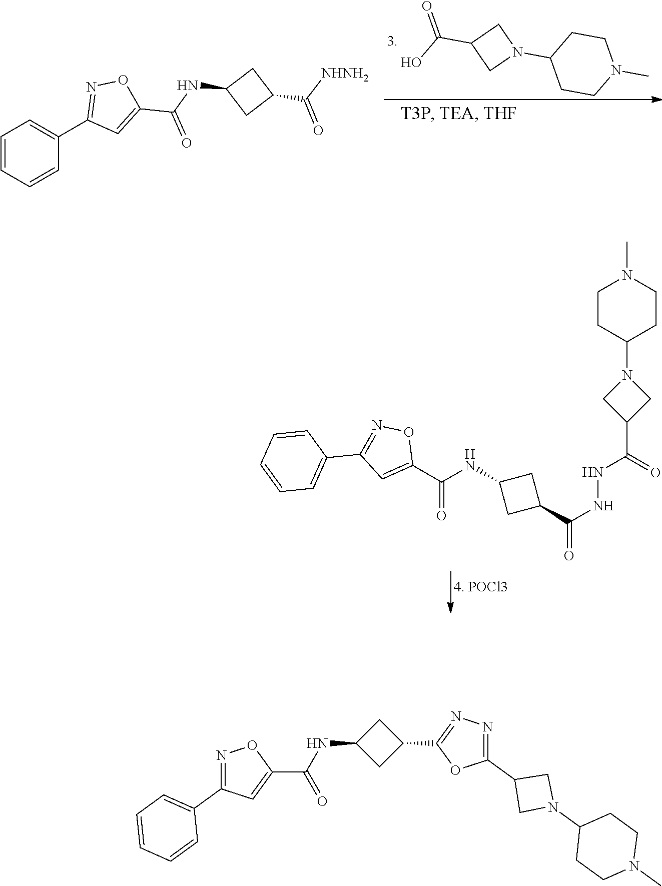 [SMILES: NNC(=O)[C@H]1C[C@H](NC(=O)c2cc(-c3ccccc3)no2)C1 ; CN1CCC(N2CC(C(=O)O)C2)CC1 ; CN1CCC(N2CC(C(=O)NNC(=O)[C@H]3C[C@H](NC(=O)c4cc(-c5ccccc5)no4)C3)C2)CC1 ; CN1CCC(N2CC(c3nnc([C@H]4C[C@H](NC(=O)c5cc(-c6ccccc6)no5)C4)o3)C2)CC1]

Step 1: benzyl 1-(1-methylpiperidin-4-yl)azetidine-3-carboxylate: a solution of trifluoroacetic acid benzyl azetidine-3-carboxylate (1.3 g, 4.26 mmol, 1.00 eq.), 1-methylpiperidin-4-one (482 mg, 4.26 mmol, 1.10 eq.) and acetic acid (255 mg, 4.25 mmol, 1.00 eq.) in DCE (20 mL) was stirred for 30 min, followed by the addition of NaBH(OAc)₃ (1.44 g, 6.79 mmol, 1.60 eq.). The resulting solution was stirred for 16 hours at room temperature. The reaction was then quenched by the addition of water, extracted with dichloromethane and the organic layers combined. The organic layer was washed with brine, dried over anhydrous sodium sulfate and concentrated under vacuum. The residue was applied onto a silica gel column with DCM/MeOH (10:1) to give 830 mg (68%) of benzyl 1-(1-methylpiperidin-4-yl)azetidine-3-carboxylate as yellow oil; LC-MS (ES, m/z): [M+H]⁺=289.2.

Step 2: 1-(1-methylpiperidin-4-yl)azetidine-3-carboxylic acid: Palladium on carbon (100 mg) was added to a solution of benzyl 1-(1-methylpiperidin-4-yl)azetidine-3-carboxylate (830 mg, 2.88 mmol, 1.00 eq.) in methanol (20 mL), the solution was degassed and back filled with hydrogen. The resulting solution was stirred for 2 hours at room temperature, and the solids were filtered out. The resulting mixture was concentrated under vacuum to give 570 mg (crude) of 1-(1-methylpiperidin-4-yl)azetidine-3-carboxylic acid as light yellow oil; LC-MS (ES, m/z): [M+H]⁺=199.1.

Step 3: 3-phenyl-N-[trans-3-([[1-(1-methylpiperidin-4-yl)azetidin-3-yl]formohydrazido]carbonyl)cyclobutyl]-isoxazole-5-carboxamide: a solution of 3-phenyl-N-[trans-3-(hydrazinecarbonyl)cyclobutyl]-isoxazole-5-carboxamide (409 mg, 1.36 mmol, 1.00 eq.), 1-(1-methylpiperidin-4-yl)azetidine-3-carboxylic acid (270 mg, 1.36 mmol, 1.00 eq.), T₃P (4.3 g, 6.76 mmol, 5.00 eq., 50%) and TEA (688 mg, 6.80 mmol, 5.00 eq.) in tetrahydrofuran (10 mL) was stirred for 30 min at room temperature. The reaction was then quenched by the addition of water, extracted with ethyl acetate and the aqueous layers combined and concentrated under vacuum. The crude product was purified by Flash-Prep-HPLC with the following conditions (IntelFlash-1): Column, C18; mobile phase, methanol/H₂O=5:95 increasing to methanol/H₂O=95:5 within 30 min; Detector, UV 254 nm to give 220 mg (34%) of 3-phenyl-N-[trans-3-([[1-(1-methylpiperidin-4-yl)azetidin-3-yl]formohydrazido]carbonyl)cyclobutyl]-isoxazole-5-carboxamide as a light yellow solid; LC-MS (ES, m/z): [M+H]⁺=481.2.

Step 4: a solution of 3-phenyl-N-[trans-3-([[1-(1-methylpiperidin-4-yl)azetidin-3-yl]formohydrazido]carbonyl)cyclobutyl]-isoxazole-5-carboxamide (160 mg, 0.33 mmol, 1.00 eq.) in POCl3 (8 mL) was stirred for 1 hour at 100° C. The reaction was then quenched by the addition of water/ice, the pH value of the solution was adjusted to 8 with sodium bicarbonate aqueous. The resulting solution was extracted with dichloromethane and the organic layers combined, washed with brine, dried and concentrated under vacuum. The crude product was purified by Prep-HPLC with the following conditions (HPLC-10): Column, XBridge Shield RP18 OBD Column, Sum, 19*150 mm; mobile phase, water (0.05% NH₄HCO₃) and ACN (27.0% ACN up to 37.0% in 8 min); Detector, UV 254/220 nm to give 19.6 mg (13%) of front peak as a white solid and 4.2 mg (3%) of second peak as an off-white solid.

Front Peak: LC-MS (ES, m/z): [M+H]⁺=463.2; ¹H NMR (300 MHz, DMSO-d₆, ppm): δ 9.45-9.43 (d, 1H, J=7.5 Hz), 7.95-7.92 (m, 2H), 7.66 (s, 1H), 7.56-7.53 (m, 3H), 4.67-4.64 (m, 1H), 3.85-3.80 (m, 1H), 3.73-3.69 (m, 1H), 3.60-3.55 (m, 2H), 3.29-3.24 (m, 3H), 2.68-2.62 (m, 5H), 2.12 (s, 3H), 2.04-1.98 (m, 1H), 1.91-1.84 (m, 2H), 1.62-1.58 (m, 2H), 1.21-1.11 (m, 2H); HPLC purity: 97.8% at 254 nm.

Second Peak: LC-MS (ES, m/z): [M+H]⁺=463.2; ¹H NMR (300 MHz, DMSO-d₆, ppm): δ 9.47-9.44 (d, 1H, J=7.8 Hz), 7.95-7.92 (m, 2H), 7.66 (s, 1H), 7.56-7.54 (m, 3H), 4.72-4.64 (m, 1H), 3.77-3.74 (m, 1H), 3.62 (s, 2H), 3.29 (s, 3H), 2.71-2.66 (m, 6H), 2.40-2.30 (m, 1H), 2.12 (s, 3H), 1.89-1.75 (m, 4H), 1.29-1.25 (m, 2H); HPLC purity: 95.1% at 254 nm.

### Example 26: 3-phenyl-N-(trans-3-(5-(1-(2,2,2-trifluoroethyl)azetidin-3-yl)-1,3,4-oxadiazol-2-yl)cyclobutyl)isoxazole-5-carboxamide

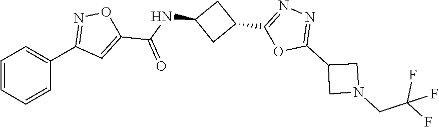 [SMILES: O=C(N[C@H]1C[C@H](c2nnc(C3CN(CC(F)(F)F)C3)o2)C1)c1cc(-c2ccccc2)no1]

The title compound was prepared using a similar method as shown in example 20.

### Example 27: N-(trans-3-(5-(1-(cyclobutylmethyl)azetidin-3-yl)-1,3,4-oxadiazol-2-yl)cyclobutyl)-3-phenylisoxazole-5-carboxamide

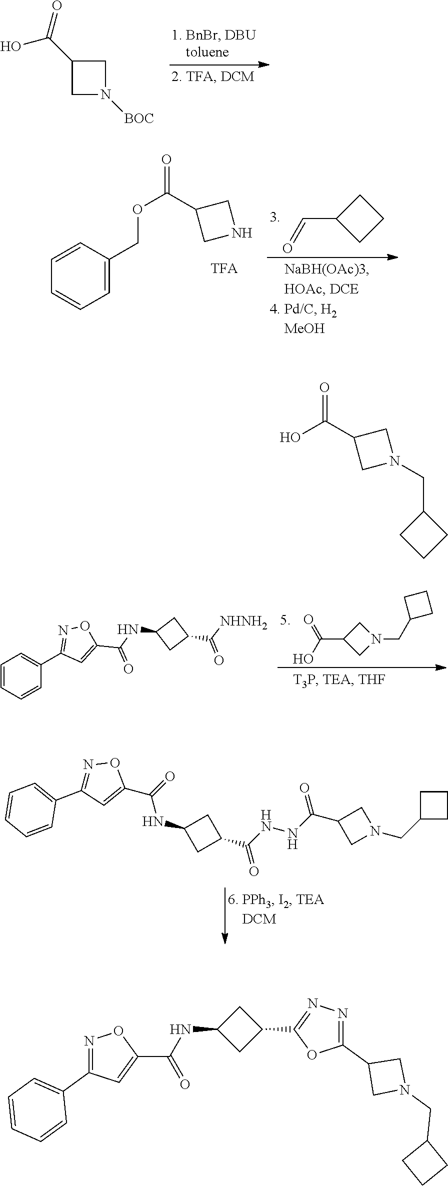

Step 1: 3-benzyl 1-tert-butyl azetidine-1,3-dicarboxylate: a solution of 1-[(tert-butoxy)carbonyl]azetidine-3-carboxylic acid (5 g, 24.85 mmol, 1.00 eq.), BnBr (4.65 g, 27.19 mmol, 1.10 eq.) and DBU (5.67 g, 37.24 mmol, 1.50 eq.) in toluene (80 mL) was stirred for 4 hours at room temperature. The reaction was then quenched by the addition of water, extracted with ethyl acetate and the organic layers combined. The resulting mixture was washed with brine, dried over anhydrous sodium sulfate and concentrated under vacuum. The residue was applied onto a silica gel column with ethyl acetate/petroleum ether (1:6) to give 5.4 g (75%) of 3-benzyl 1-tert-butyl azetidine-1,3-dicarboxylate as colorless oil; LC-MS (ES, m/z): [M+H−Boc]⁺=192.0.

Step 2: 2,2,2-trifluoroacetic acid benzyl azetidine-3-carboxylate: a solution of 3-benzyl 1-tert-butyl azetidine-1,3-dicarboxylate (5.4 g, 18.53 mmol, 1.00 eq.)) and trifluoroacetic acid (7 mL).in dichloromethane (50 mL) was stirred overnight at room temperature. The resulting mixture was concentrated under vacuum to give 7 g (crude) of 2,2,2-trifluoroacetic acid benzyl azetidine-3-carboxylate as light yellow oil; LC-MS (ES, m/z): [M+H−TFA]⁺=191.8.

Step 3: benzyl 1-(cyclobutylmethyl)azetidine-3-carboxylate: a solution of 2,2,2-trifluoroacetic acid cyclohexylmethyl azetidine-3-carboxylate (1.3 g, 4.18 mmol, 1.00 eq.), cyclobutanecarboxaldehyde (358 mg, 4.26 mmol, 1.00 eq.) and acetic acid (255 mg, 4.25 mmol, 1.00 eq.) in DCE (20 mL) was stirred for 30 min, and then NaBH(OAc)₃ (1.44 g, 6.79 mmol, 1.60 eq.) was added. The resulting solution was stirred for 2 hours at room temperature. The reaction was then quenched by the addition of water, extracted with dichloromethane and the organic layers combined. The resulting mixture was washed with brine, dried over anhydrous sodium sulfate and concentrated under vacuum. The residue was applied onto a silica gel column with dichloromethane/methanol (20:1) to give 650 mg (59%) of benzyl 1-(cyclobutylmethyl)azetidine-3-carboxylate as colorless oil; LC-MS (ES, m/z): [M+H]⁺=260.1.

Step 4: 1-(cyclobutylmethyl)azetidine-3-carboxylic acid: to a solution of benzyl 1-(cyclobutylmethyl)azetidine-3-carboxylate (650 mg, 2.51 mmol, 1.00 eq.) in methanol (10 mL) was added Palladium on carbon (65 mg) and the solution was degassed and back filled with hydrogen. The resulting solution was stirred for 2 hours at room temperature. The solids were filtered out and concentrated under vacuum to afford 425 mg (99%) of 1-(cyclobutylmethyl)azetidine-3-carboxylic acid as a white solid; LC-MS (ES, m/z): [M+H]⁺=170.1.

Step 5: 3-phenyl-N-[trans-3-([[1-(cyclobutylmethyl)azetidin-3-yl]formohydrazido]carbonyl)cyclobutyl]-isoxazole-5-carboxamide: a solution of 3-phenyl-N-[(trans-3-(hydrazinecarbonyl)cyclobutyl]-isoxazole-5-carboxamide (300 mg, 1.00 mmol, 1.00 eq.), 1-(cyclobutylmethyl)azetidine-3-carboxylic acid (200 mg, 1.20 mmol, 1.20 eq.), T₃P (3.18 g, 5.00 mmol, 5.00 eq., 50%) and TEA (505 mg, 4.99 mmol, 5.00 eq.) in tetrahydrofuran (10 mL) was stirred for 30 min at room temperature. The reaction was then quenched by the addition of water, extracted with ethyl acetate and the aqueous layers combined and concentrated under vacuum. The crude product was purified by Flash-Prep-HPLC with the following conditions (IntelFlash-1): Column, C18; mobile phase, MeCN/H₂O=5:95 increasing to MeCN/H₂O=95:5 within 30 min; Detector, UV 254 nm to afford 210 mg (47%) of 3-phenyl-N-[trans-3-([[1-(cyclobutylmethyl)azetidin-3-yl]formohydrazido]carbonyl)cyclobutyl]-isoxazole-5-carboxamide as a light yellow solid; LC-MS (ES, m/z): [M+H]⁺=452.1.

Step 6: 3-phenyl-N-[trans-3-[5-[1-(cyclobutylmethyl)azetidin-3-yl]-1,3,4-oxadiazol-2-yl]cyclobutyl]-isoxazole-5-carboxamide: I₂ (401 mg, 1.58 mmol, 2.50 eq.), TEA (415 mg, 4.10 mmol, 6.50 eq.) and 3-phenyl-N-[trans-3-([[1-(cyclobutylmethyl)azetidin-3-yl]formohydrazido]carbonyl)cyclobutyl]-isoxazole-5-carboxamide (285 mg, 0.63 mmol, 1.00 eq.) were added to a solution of Ph₃P (414 mg, 1.58 mmol, 2.50 eq.) in dichloromethane (20 mL) under N₂. The reaction mixture was stirred for 1 hour at room temperature, quenched with water and then extracted with ethyl acetate and the organic layers combined. The organic layer was washed with brine, dried over anhydrous sodium sulfate and concentrated under vacuum. The residue was applied onto a silica gel column with DCM/MeOH (25:1). The resulting crude product was purified by Prep-HPLC with the following conditions (HPLC-10): Column, X Bridge Prep C18 OBD Column, 19*150 mm, 5 um; mobile phase, water (0.05% NH₄HCO₃) and ACN (30% ACN up to 80% within 8 min); Detector, UV 254 nm to give 125.6 mg (46%) of 3-phenyl-N-[trans-3-[5-[1-(cyclobutylmethyl)azetidin-3-yl]-1,3,4-oxadiazol-2-yl]cyclobutyl]-isoxazole-5-carboxamide as a white solid; LC-MS (ES, m/z): [M+H]⁺=434.3; ¹H NMR (400 MHz, DMSO-d₆, ppm): δ 9.45-9.43 (d, 1H, J=7.6 Hz), 7.95-7.93 (m, 2H), 7.65 (s, 1H), 7.55-7.54 (m, 2H), 4.71-4.63 (m, 1H), 3.88-3.81 (m, 1H), 3.74-3.67 (m, 1H), 3.59-3.55 (t, 2H, J=7.2 Hz), 3.31 (s, 1H), 3.29-3.26 (d, 1H, J=6.8 Hz), 2.70-2.63 (m, 4H), 2.45-2.43 (m, 2H), 2.32-2.24 (m, 1H), 1.99-1.95 (m, 2H), 1.88-1.73 (m, 2H), 1.67-1.59 (m, 2H); HPLC purity: 99.3% at 254 nm.

### Examples 28 and 29: N-(cis-3-(5-(hydroxymethyl)-1H-1,2,3-triazol-1-yl)cyclobutyl)-3-phenylisoxazole-5-carboxamide and N-(cis-3-(4-(hydroxymethyl)-1H-1,2,3-triazol-1-yl)cyclobutyl)-3-phenylisoxazole-5-carboxamide

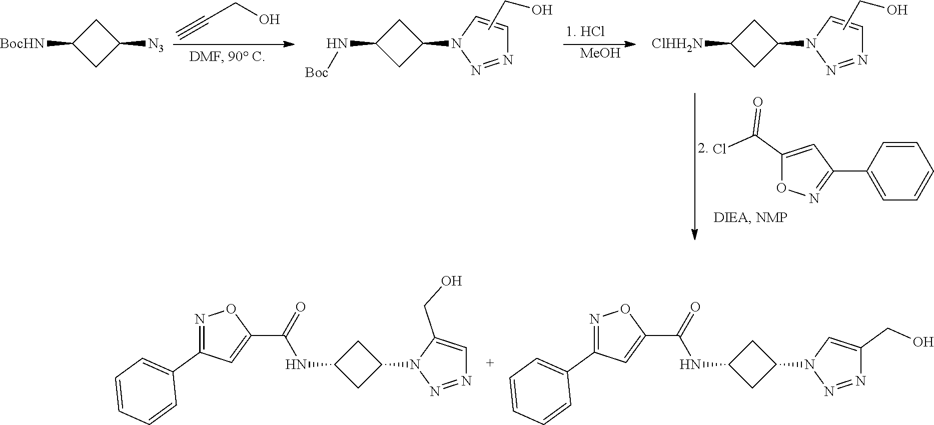

Step 1: 1-[cis-3-aminocyclobutyl]-1H-1,2,3-triazol-5-yl]methanol hydrochloride: a solution of tert-butyl N-[cis-3-[4/5-(hydroxymethyl)-1H-1,2,3-triazol-1-yl]cyclobutyl]carbamate (prepared using a procedure similar to example 36; 400 mg, 1.49 mmol, 1.00 eq.) in hydrogen chloride/MeOH (5 mL) was stirred for 18 hours at room temperature. The resulting mixture was concentrated under vacuum and diluted with 3 mL of dioxane. The solids were collected by filtration and dried in an oven under reduced pressure to give 301 mg (crude) of 1-[cis-3-aminocyclobutyl]-1H-1,2,3-triazol-5-yl]methanol hydrochloride as a white solid; LC-MS (ES, m/z): [M+1]⁺=167.1.

Step 2: 3-phenyl-N-[cis-3-[4-(hydroxymethyl)-1H-1,2,3-triazol-1-yl]cyclobutyl]isoxazole-5-carboxamide and 3-phenyl-N-[cis-3-[5-(hydroxymethyl)-1H-1,2,3-triazol-1-yl]cyclobutyl]isoxazole-5-carboxamide: DIEA (787 mg, 6.09 mmol, 3.00 eq.) was added dropwise to a cold solution (10° C.) of [1-[cis-3-aminocyclobutyl]-1H-1,2,3-triazol-4/5-yl]methanol hydrochloride (410 mg, 2.00 mmol, 1.00 eq.) in NMP (4 mL) and stirred for 30 min at 25° C., followed by the addition of a solution of 3-phenylisoxazole-5-carbonyl chloride (310 mg, 1.64 mmol, 1.00 eq.) in NMP (1 mL) dropwise with stirring at 0 to 10° C. The reaction was stirred for 30 min and then quenched by the addition of 0.5 mL of methanol. The mixture was stirred at 25° C. for 30 min then 40 mL of water was added. The crude solid was collected by filtration and purified by prep-HPLC: Column: XBridge BEH130 Prep C18 OBD Column 19*150 mm, 5 um, 13 nm; Mobile Phase A: water (10 mmol/L NH₄HCO₃), Mobile Phase B: ACN; Flow rate: 20 mL/min; Gradient: 22% B to 47% B in 8 min; 254 nm to give 152 mg (22%) of 3-phenyl-N-[cis-3-[5-(hydroxymethyl)-1H-1,2,3-triazol-1-yl]cyclobutyl]isoxazole-5-carboxamide as a white solid and 143.15 mg (28%) of 3-phenyl-N-[cis-3-[4-(hydroxymethyl)-1H-1,2,3-triazol-1-yl]cyclobutyl]isoxazole-5-carboxamide as a white solid.

3-phenyl-N-[cis-3-[5-(hydroxymethyl)-1H-1,2,3-triazol-1-yl]cyclobutyl]isoxazole-5-carboxamide: LC-MS (ES, m/z): [M+1]⁺=340.0; ¹H NMR (300 MHz, DMSO-d₆, ppm): δ 9.48-9.45 (d, J=7.5 Hz, 1H), 7.94-7.91 (m, 2H), 7.66-7.62 (m, 2H), 7.56-7.54 (m, 3H), 5.46-5.42 (m, 1H), 4.89-4.80 (m, 1H), 4.58-4.57 (d, J=5.4 Hz, 2H), 4.45-4.35 (m, 1H), 2.92-2.80 (m, 4H); HPLC purity: 99.2% at 254 nm.

3-phenyl-N-[cis-3-[4-(hydroxymethyl)-1H-1,2,3-triazol-1-yl]cyclobutyl]isoxazole-5-carboxamide: LC-MS (ES, m/z): [M+1]⁺=340.0; ¹H NMR (300 MHz, DMSO-d₆, ppm): δ 9.41-9.39 (d, J=8.4 Hz, 1H), 8.16 (s, 1H), 7.96-7.93 (m, 2H), 7.67 (s, 1H), 7.56-7.54 (m, 3H), 5.23-5.19 (t, J=5.6 Hz, 1H), 4.98-4.92 (m, 1H), 4.55-4.53 (d, J=5.4 Hz, 2H), 4.45-4.37 (m, 1H), 2.98-2.90 (m, 2H), 2.75-2.65 (m, 2H); HPLC purity: 99.3% at 254 nm.

### Examples 30 and 31: N-(trans-3-(5-(oxetan-3-yl)-1H-1,2,3-triazol-1-yl)cyclobutyl)-5-phenylisoxazole-3-carboxamide and N-(trans-3-(4-(oxetan-3-yl)-1H-1,2,3-triazol-1-yl)cyclobutyl)-5-phenylisoxazole-3-carboxamide

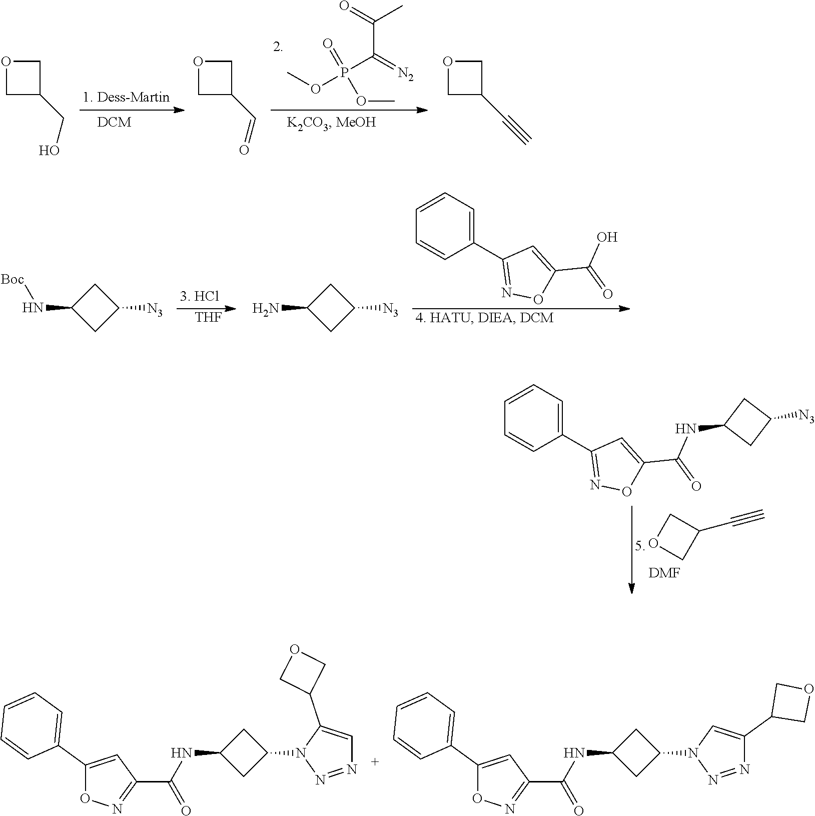

Step 1: oxetane-3-carbaldehyde: a solution of oxetan-3-ylmethanol (2 g, 22.70 mmol, 1.00 eq.) in dichloromethane (20 mL) and 1,1,1-triacetoxy-1,1-dihydro-1,2-benziodoxol-3(1H)-one (11.7 g, 27.59 mmol, 1.00 eq.) was stirred for 2 hours at 25° C. The solids were filtered out and the mixture was concentrated under vacuum to give 2.1 g (crude) of oxetane-3-carbaldehyde as yellow oil.

Step 2: 3-ethynyloxetane: a solution of oxetane-3-carbaldehyde (2.1 g, 24.39 mmol, 1.00 eq.), potassium carbonate (6.6 g, 47.75 mmol, 2.00 eq.) and dimethyl (1-diazo-2-oxopropyl)phosphonate (7 g, 36.44 mmol, 1.50 eq.) in methanol (30 mL) was stirred for 3 hours at 25° C. The resulting solution was diluted with 150 mL of water, extracted with ethyl acetate (2×100 mL) and the organic layers combined. The resulting mixture was washed with brine (2×100 mL), dried over anhydrous sodium sulfate and concentrated under vacuum to give 820 mg (41%) of 3-ethynyloxetane as colorless oil.

Step 3: trans-3-azidocyclobutan-1-amine: a solution of tert-butyl N-[trans-3-azidocyclobutyl]carbamate (1 g, 4.71 mmol, 1.00 eq.) in tetrahydrofuran (20 mL)/conc. HCl aqueous (5 mL) was stirred for 2 hours at 25° C. The resulting mixture was concentrated under vacuum to give 800 mg (crude) of cis-3-azidocyclobutan-1-amine as yellow oil.

Step 4: 3-phenyl-N-[trans-3-azidocyclobutyl]-isoxazole-5-carboxamide: HATU (1.37 g, 3.60 mmol, 1.50 eq.), DIEA (928 mg, 7.18 mmol, 3.00 eq.) and 3-phenyl-isoxazole-5-carboxylic acid (453 mg, 2.39 mmol, 1.00 eq.) were added to a solution of trans-3-azidocyclobutan-1-amine (800 mg, 7.13 mmol, 1.00 eq.) in dichloromethane (15 mL) and the mixture was stirred for 2 hours at 25° C. The resulting solution was diluted with 150 mL of H₂O, extracted with ethyl acetate (2×100 mL) and the organic layers combined. The organic layer was washed with brine (2×100 mL), dried over anhydrous sodium sulfate and concentrated under vacuum. The residue was applied onto a silica gel column with petroleum ether:ethyl acetate (10:1) to afford 390 mg (19%) of 3-phenyl-N-[trans-3-azidocyclobutyl]-isoxazole-5-carboxamide as a yellow solid; LC-MS (ES, m/z): [M+H]⁺=284.1.

Step 5: 5-phenyl-N-[trans-3-[4-(oxetan-3-yl)-1H-1,2,3-triazol-1-yl]cyclobutyl]isoxazole-3-carboxamide and 5-phenyl-N-[trans-3-[5-(oxetan-3-yl)-1H-1,2,3-triazol-1-yl]cyclobutyl]isoxazole-3-carboxamide: a solution of 3-phenyl-N-[(trans-3-azidocyclobutyl]isoxazole-5-carboxamide (283 mg, 1.00 mmol, 1.00 eq.) and 3-ethynyloxetane (410 mg, 4.99 mmol, 5.00 eq.) in DMF (10 mL) was stirred for 16 hours at 100° C. The resulting solution was diluted with 100 mL of H₂O, extracted with ethyl acetate (2×100 mL) and the combined organic layers were dried over anhydrous sodium sulfate and concentrated under vacuum. The crude product was purified by Prep-TLC (petroleum ether:ethyl acetate=1:5). The resulting isomers was separated by Chiral-Prep-HPLC with the following conditions (Prep-HPLC-032): Column, Phenomenex Lux 5u Cellulose-4 AXIA Packed, 250*21.2 mm, Sum; mobile phase, Hex and ethanol (hold 50.0% ethanol in 20 min); Detector, UV 254/220 nm to afford 16.8 mg (5%) of 5-phenyl-N-[trans-3-[5-(oxetan-3-yl)-1H-1,2,3-triazol-1-yl]cyclobutyl]isoxazole-3-carboxamide as a white solid and 29.1 mg (8%) of 3-phenyl-N-[trans-3-[4-(oxetan-3-yl)-1H-1,2,3-triazol-1-yl]cyclobutyl]isoxazole-5-carboxamide as a white solid.

5-phenyl-N-[trans-3-[5-(oxetan-3-yl)-1H-1,2,3-triazol-1-yl]cyclobutyl]isoxazole-3-carboxamide: LC-MS (ES, m/z): [M+H]⁺=366.1; ¹H NMR (300 MHz, DMSO-d₆) δ 9.51-9.49 (d, J=7.2 Hz, 1H), 7.96-7.94 (m, 3H), 7.68 (s, 1H), 7.57-7.56 (m, 3H), 4.97-4.93 (m, 3H), 4.75-4.70 (m, 1H), 4.66-4.62 (m, 2H), 4.48-4.42 (m, 1H), 2.86-2.74 (m, 4H); HPLC purity: 99.5% at 254 nm.

5-phenyl-N-[trans-3-[4-(oxetan-3-yl)-1H-1,2,3-triazol-1-yl]cyclobutyl]isoxazole-3-carboxamide: LC-MS (ES, m/z): [M+H]⁺=366.1; ¹H NMR (300 MHz, DMSO-d₆) δ 9.52-9.50 (d, J=6.9 Hz, 1H), 8.33 (s, 1H), 7.96-7.93 (m, 2H), 7.68 (s, 1H), 7.56-7.54 (m, 3H), 5.34-5.24 (m, 1H), 4.92-4.88 (m, 2H), 4.76-4.65 (m, 3H), 4.42-4.32 (m, 1H), 2.91-2.75 (m, 4H); HPLC purity: 98% at 254 nm.

### Examples 32 and 33: N-(trans-3-(4-(1-methylazetidin-3-yl)-1H-1,2,3-triazol-1-yl)cyclobutyl)-3-phenylisoxazole-5-carboxamide and N-(trans-3-(5-(1-methylazetidin-3-yl)-1H-1,2,3-triazol-1-yl)cyclobutyl)-3-phenylisoxazole-5-carboxamide

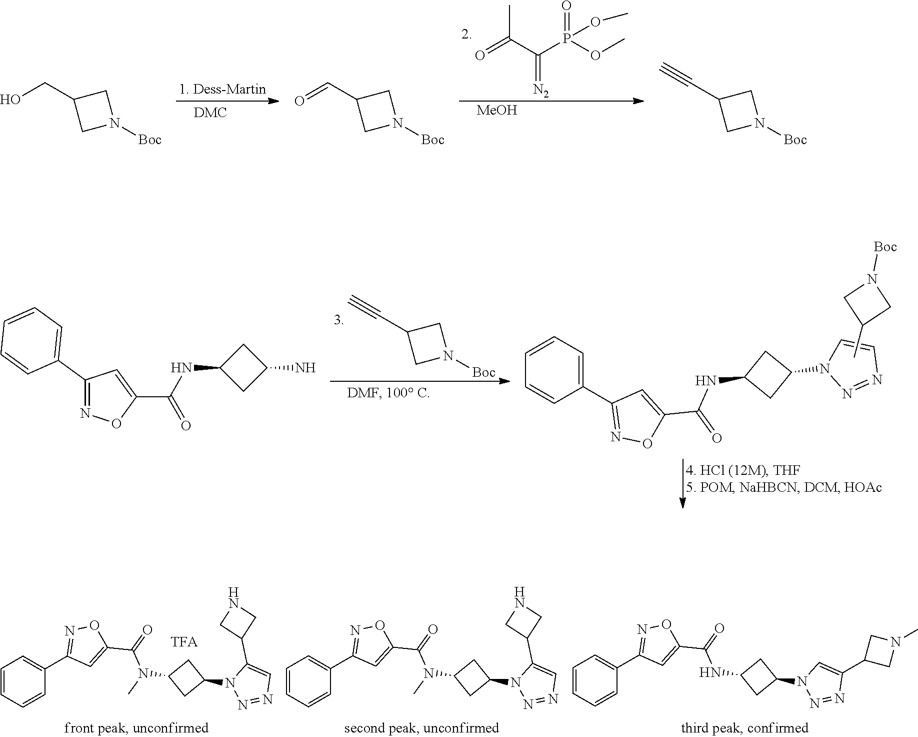

Step 1: tert-butyl 3-formylazetidine-1-carboxylate: a solution of tert-butyl 3-(hydroxymethyl)azetidine-1-carboxylate (3.74 g, 19.97 mmol, 1.00 equip), and Dess-Martin reagent (12.72 g, 30.00 mmol, 1.50 eq.) in dichloromethane (100 mL) was stirred for 2 hours at room temperature. The solids were filtered out, the resulting mixture was concentrated under vacuum to give 3.8 g (crude) of tert-butyl 3-formylazetidine-1-carboxylate as a white solid.

Step 2: tert-butyl 3-ethynylazetidine-1-carboxylate: a solution of tert-butyl 3-formylazetidine-1-carboxylate (3.7 g, 19.98 mmol, 1.00 eq.), potassium carbonate (8.28 g, 59.91 mmol, 3.00 eq.) and dimethyl (1-diazo-2-oxopropyl)phosphonate (5.76 g, 29.98 mmol, 1.50 eq.) in methanol (50 mL) was stirred for 3 hours at room temperature. The resulting solution was diluted with 200 mL of ether, washed with saturated sodium bicarbonate aqueous (2×200 mL), dried over anhydrous sodium sulfate and concentrated under vacuum to give 3.282 g (crude) of tert-butyl 3-ethynylazetidine-1-carboxylate as yellow oil.

Step 3: tert-butyl 3-[1-[trans-3-(3-phenylisoxazole-5-amido)cyclobutyl]-1H-1,2,3-triazol-4/5-yl]azetidine-1-carboxylate: a solution of 3-phenyl-N-[trans-3-azidocyclobutyl]isoxazole-5-carboxamide (327 mg, 1.15 mmol, 1.00 eq.) and tert-butyl 3-ethynylazetidine-1-carboxylate (627 mg, 3.46 mmol, 3.00 eq.) in DMF (4 mL) was placed in a microwave reactor for 6 hours at 140° C. The resulting mixture was concentrated under vacuum and the residue was applied onto a silica gel column with ethyl acetate/petroleum ether (1:1) to give 553 mg (crude) mixture of tert-butyl 3-[1-[trans-3-(3-phenylisoxazole-5-amido)cyclobutyl]-1H-1,2,3-triazol-5-yl]azetidine-1-carboxylate and tert-butyl 3-[1-[trans-3-(3-phenylisoxazole-5-amido)cyclobutyl]-1H-1,2,3-triazol-4-yl]azetidine-1-carboxylate as a yellow solid; LC-MS (ES, m/z): [M+H]⁺=465.3.

Step 4: 3-phenyl-N-[trans-3-[4/5-(azetidin-3-yl)-1H-1,2,3-triazol-1-yl]cyclobutyl]isoxazole-5-carboxamide hydrochloride: a solution of the mixture of tert-butyl 3-[1-trans-3-(3-phenylisoxazole-5-amido)cyclobutyl]-1H-1,2,3-triazol-4/5-yl]azetidine-1-carboxylate (553 mg, 1.19 mmol, 1.00 eq.) in tetrahydrofuran (10 mL)/hydrogen chloride aqueous (6N, 6 mL) was stirred for 2 hours at room temperature. The resulting mixture was concentrated under vacuum to give 551 mg (crude) of a mixture of 3-phenyl-N-[trans-3-[4/5-(azetidin-3-yl)-1H-1,2,3-triazol-1-yl]cyclobutyl]isoxazole-5-carboxamide hydrochloride as a brown solid; LC-MS (ES, m/z): [M−HCl+H]⁺=365.3.

Step 5: N-(trans-3-(4-(1-methylazetidin-3-yl)-1H-1,2,3-triazol-1-yl)cyclobutyl)-3-phenylisoxazole-5-carboxamide and N-(trans-3-(5-(1-methylazetidin-3-yl)-1H-1,2,3-triazol-1-yl)cyclobutyl)-3-phenylisoxazole-5-carboxamide: a solution of the mixture of 3-phenyl-N-[trans-3-[4/5-(azetidin-3-yl)-1H-1,2,3-triazol-1-yl]cyclobutyl]isoxazole-5-carboxamide hydrochloride, POM (302 mg, 6.86 mmol, 4.99 eq.) and acetic acid (165 mg, 2.75 mmol, 2.00 eq.) in DCM (20 mL) was stirred for 30 min at room temperature. NaBHCN (346 mg, 5.49 mmol, 4.00 eq.) was added to the reaction mixture and it was stirred for 3 hours at room temperature. The mixture was concentrated under vacuum and the crude product was purified by Prep-HPLC with the following conditions (HPLC-10): Column, XBridge C18 OBD Prep Column, 19 mm×250 mm; mobile phase, water (10 mmol/L NH₄HCO₃) and ACN (40.0% ACN up to 90.0% in 8 min); Detector, UV 254/220 nm. This resulted in 50 mg crude first peak, 20 mg (4%) of second peak as a white solid and 75 mg (15%) of third peak) as a white solid. Then the crude first peak was purified by Prep-HPLC with the following conditions (HPLC-10): Column, XBridge C18 OBD Prep Column, 19 mm×250 mm; mobile phase, water (0.05% TFA) and ACN (20.0% ACN up to 50.0% in 10 min); Detector, UV 254/220 nm to give 30 mg of product as a yellow oil.

First Peak (Putative Structure):

Second peak (putative structure): LC-MS (ES, m/z): [M+H]⁺=379.2; ¹H NMR (400 MHz, CD₃OD, ppm): 8.05 (s, 1H), 7.89-7.88 (d, J=2.8 Hz, 2H), 7.52-7.51 (m, 3H), 7.43 (s, 1H), 5.11 (br, 1H), 4.90-4.88 (m, 1H), 4.74-7.54 (m, 1H), 4.54-4.46 (m, 2H), 4.36-4.25 (m, 2H), 3.10-2.91 (m, 5H), 2.89-2.88 (m, 2H); HPLC purity: 99.4% at 254 nm.

Third peak: N-(trans-3-(4-(1-methylazetidin-3-yl)-1H-1,2,3-triazol-1-yl)cyclobutyl)-3-phenylisoxazole-5-carboxamide: LC-MS [M+H]⁺=379.3; ¹H NMR (400 MHz, DMSO-d₆, ppm): δ 9.53-9.51 (d, J=6.8 Hz, 1H), 8.23 (s, 1H), 7.96-7.74 (m, 2H), 7.69 (s, 1H), 7.56-7.54 (m, 3H), 5.28-5.24 (m, 1H), 4.72-4.67 (m, 1H), 3.64-3.56 (m, 3H), 3.12-3.09 (m, 2H), 2.88-2.75 (m, 4H), 2.08 (s, 3H); HPLC purity; 98.7% at 254 nm.

### Example 34: N-(trans-3-(5-(1-(methylsulfonyl)ethyl)-1H-1,2,3-triazol-1-yl)cyclobutyl)-3-phenylisoxazole-5-carboxamide

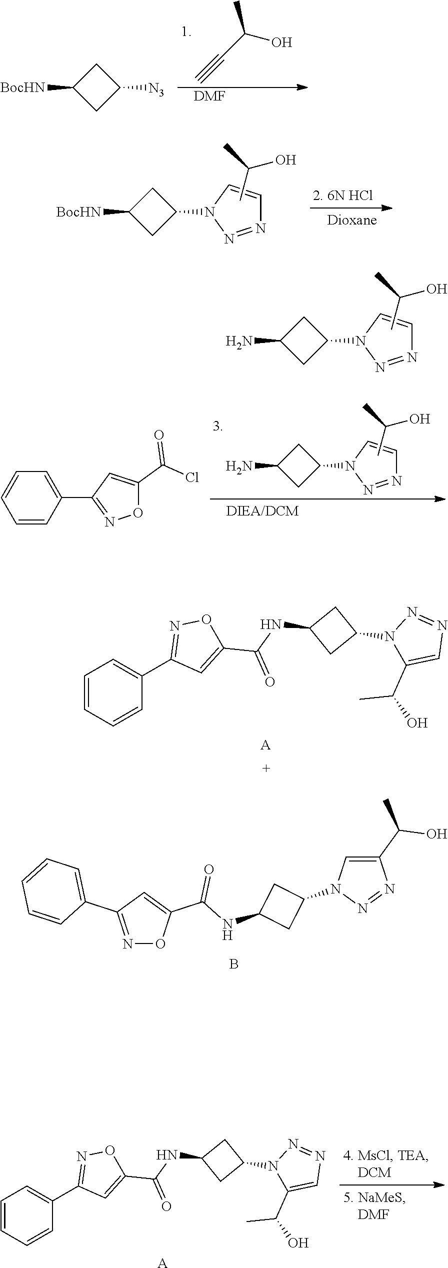

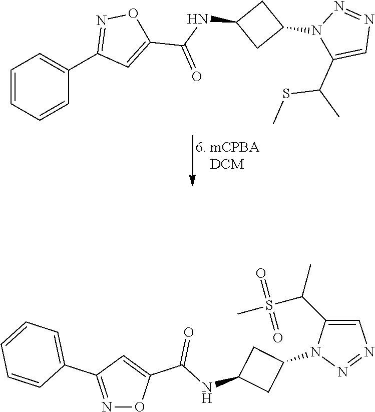 [SMILES: CSC(C)c1cnnn1[C@H]1C[C@H](NC(=O)c2cc(-c3ccccc3)no2)C1 ; CC(c1cnnn1[C@H]1C[C@H](NC(=O)c2cc(-c3ccccc3)no2)C1)S(C)(=O)=O]

### Preparation of Intermediates A and B

Step 1: N-[trans-3-[4/5-[(1R)-1-hydroxyethyl]-1H-1,2,3-triazol-1-yl]cyclobutyl]carbamate: a solution of tert-butyl N-[trans-3-azidocyclobutyl]carbamate (2 g, 9.42 mmol, 1.00 eq.) and (2R)-but-3-yn-2-ol (3.3 g, 47.08 mmol, 5.00 eq.) in DMF (5 mL) was stirred for overnight at 100° C. in an oil bath. The resulting mixture was concentrated under vacuum. The residue was applied onto a silica gel column with ethyl acetate/petroleum ether (3:1) to give 2.1 g (79%) of a mixture of tert-butyl N-[trans-3-[4/5-[(1R)-1-hydroxyethyl]-1H-1,2,3-triazol-1-yl]cyclobutyl]carbamate as a light yellow solid; LC-MS (ES, m/z): [M+H]⁺=283.2.

Step 2: (1R)-1-[1-[trans-3-aminocyclobutyl]-1H-1,2,3-triazol-4/5-yl]ethanol: a solution of the mixture of tert-butyl N-[trans-3-[4/5-[(1R)-1-hydroxyethyl]-1H-1,2,3-triazol-1-yl]cyclobutyl]carbamate in dioxane (10 mL)/hydrogen chloride aqueous (6N, 3 mL) was stirred for 2 hours at room temperature. The resulting mixture was concentrated under vacuum to give 1.45 g (crude) of a mixture of (1R)-1-[1-[trans-3-aminocyclobutyl]-1H-1,2,3-triazol-4/5-yl]ethanol as a light yellow solid; LC-MS-PH (ES, m/z): [M+H]⁺=183.1.

Step 3: N-(trans-3-(5-((R)-1-hydroxyethyl)-1H-1,2,3-triazol-1-yl)cyclobutyl)-3-phenylisoxazole-5-carboxamide (A) and N-(trans-3-(4-((R)-1-hydroxyethyl)-1H-1,2,3-triazol-1-yl)cyclobutyl)-3-phenylisoxazole-5-carboxamide (B): DIEA (2.55 g, 3.00 eq.) and 3-phenylisoxazole-5-carbonyl chloride (1.77 g, 8.53 mmol, 1.30 eq.) were added dropwise to a cold (0° C.) solution of a mixture of (1R)-1-[1-[trans-3-aminocyclobutyl]-1H-1,2,3-triazol-4/5-yl]ethanol in dichloromethane (20 mL) and the mixture was stirred for 2 hours at 0° C. The resulting mixture was washed with hydrogen chloride aqueous (2N) (1×50 mL) and potassium carbonate (5%) (1×100 mL), concentrated under vacuum, and the crude product was purified by prep-HPLC to give 0.236 g (10%) of N-(trans-3-(5-((R)-1-hydroxyethyl)-1H-1,2,3-triazol-1-yl)cyclobutyl)-3-phenylisoxazole-5-carboxamide and 0.333 g (14%) of N-(trans-3-(4-((R)-1-hydroxyethyl)-1H-1,2,3-triazol-1-yl)cyclobutyl)-3-phenylisoxazole-5-carboxamide as a white solid; LC-MS (ES, m/z): [M+H]⁺=354.2.

### Preparation of N-(trans-3-(5-(1-(methylsulfonyl)ethyl)-1H-1,2,3-triazol-1-yl)cyclobutyl)-3-phenylisoxazole-5-carboxamide

Step 4: N-(trans-3-(5-((R)-1-chloroethyl)-1H-1,2,3-triazol-1-yl)cyclobutyl)-3-phenylisoxazole-5-carboxamide: MsCl (81.3 mg, 2.00 eq.) was added dropwise to a 0° C. solution of 3-phenyl-N-[(trans-3-[5-[(1R)-1-hydroxyethyl]-1H-1,2,3-triazol-1-yl]cyclobutyl]isoxazole-5-carboxamide (126 mg, 0.36 mmol, 1.00 eq.) and TEA (108 mg, 3.00 eq.) in dichloromethane (20 mL) and the solution was stirred for 5 hours at room temperature. The mixture was diluted with 30 ml of dichloromethane, washed with CuSO₄ aqueous (2×30 mL) and concentrated under vacuum to give 151 mg (crude) of N-(trans-3-(5-((R)-1-chloroethyl)-1H-1,2,3-triazol-1-yl)cyclobutyl)-3-phenylisoxazole-5-carboxamide as a brown oil; LC-MS (ES, m/z): [M+H]⁺=372.1.

Step 5: a solution of N-(trans-3-(5-((R)-1-chloroethyl)-1H-1,2,3-triazol-1-yl)cyclobutyl)-3-phenylisoxazole-5-carboxamide (151 mg, 0.41 mmol, 1.00 eq.) and NaSMe (50 mg, 2.00 eq.) in DMF (5 mL) was stirred for 5 hours at 100° C. in an oil bath. The reaction was then quenched by the addition of 20 mL of water, extracted with ethyl acetate (3×20 mL) and the organic layers combined. The resulting mixture was washed with brine (2×10 mL) and concentrated under vacuum to give 189 mg (crude) of 3-phenyl-N-[trans-3-[5-[1-(methylsulfanyl)ethyl]-1H-1,2,3-triazol-1-yl]cyclobutyl]-isoxazole-5-carboxamide as brown oil; LC-MS (ES, m/z): [M+H]⁺=384.4.

Step 6: mCPBA (338 mg, 1.96 mmol, 4.00 eq.) was added in several batches to a 0° C. solution of 3-phenyl-N-[trans-3-[5-[1-(methylsulfanyl)ethyl]-1H-1,2,3-triazol-1-yl]cyclobutyl]isoxazole-5-carboxamide (189 mg, 0.49 mmol, 1.00 eq.) in dichloromethane (10 mL) and the mixture was stirred for 5 hours at room temperature. The reaction mixture was diluted with 50 mL of dichloromethane, washed with Na₂S₂O₃ aqueous (1×50 mL) and concentrated under vacuum. The crude product was purified by Prep-HPLC with the following conditions (Water): Column, Xbridge Prep C18, 5 um, 19*150 mm; mobile phase, water with 0.08% NH₄HCO₃ and CH₃CN (30% CH₃CN up to 70% CH₃CN in 10 min, up to 95% in 2 min and down to 30% in 2 min); Detector, UV 254 nm and 220 nm to give 23.3 mg (11%) of 3-phenyl-N-[trans-3-[5-[1-methanesulfonylethyl]-1H-1,2,3-triazol-1-yl]cyclobutyl]-isoxazole-5-carboxamide as a white solid; LC-MS (ES, m/z): [M+H]⁺=416.2; ¹H NMR (DMSO-d₆, 400 MHz): δ 9.52-9.49 (d, J=12.0 Hz, 1H), 7.95-7.93 (m, 3H), 7.69 (s, 1H), 7.56-7.54 (m, 3H), 5.36-5.29 (m, 1H), 4.93-4.87 (m, 1H), 4.85-4.76 (m, 1H), 3.01 (s, 3H), 2.92-2.78 (m, 4H), 1.69-1.67 (d, J=7.2 Hz, 3H); HPLC purity: 99.2% at 254 nm.

### Example 35: N-(trans-3-(4-(1-(methylsulfonyl)ethyl)-1H-1,2,3-triazol-1-yl)cyclobutyl)-3-phenylisoxazole-5-carboxamide

The title compound was prepared by a similar procedure as shown in example 34 using intermediate B as the starting material. The crude product was purified by Prep-HPLC with the following conditions (Water): Column, Xbridge Prep C18, 5 um, 19*150 mm; mobile phase, water with 0.08% NH₄HCO₃ and CH₃CN (30% CH₃CN up to 75% CH₃CN in 10 min, up to 95% in 2 min and down to 30% in 2 min); Detector, UV 254 nm and 220 nm to give 54.5 mg (17.6%) of 3-phenyl-N-[trans-3-[5-[1-methanesulfonylethyl]-1H-1,2,3-triazol-1-yl]cyclobutyl]-isoxazole-5-carboxamide as a white solid; LC-MS (ES, m/z): [M+H]⁺=416.2; ¹H NMR (DMSO-d₆, 400 MHz): δ 9.54-9.52 (d, J=7.2 Hz, 1H), 8.43 (s, 1H), 7.96-7.94 (m, 2H), 7.68 (s, 1H), 7.56-7.54 (m, 3H), 5.37-5.29 (m, 1H), 4.72-4.68 (m, 2H), 2.95 (s, 3H), 2.88-2.81 (m, 4H), 1.68-1.66 (d, J=7.2 Hz, 3H); HPLC purity: 98.4% at 254 nm.

### Example 36: 3-(4-fluorophenyl)-N-(trans-3-(5-((R)-1-hydroxyethyl)-1,3,4-oxadiazol-2-yl)cyclobutyl)isoxazole-5-carboxamide

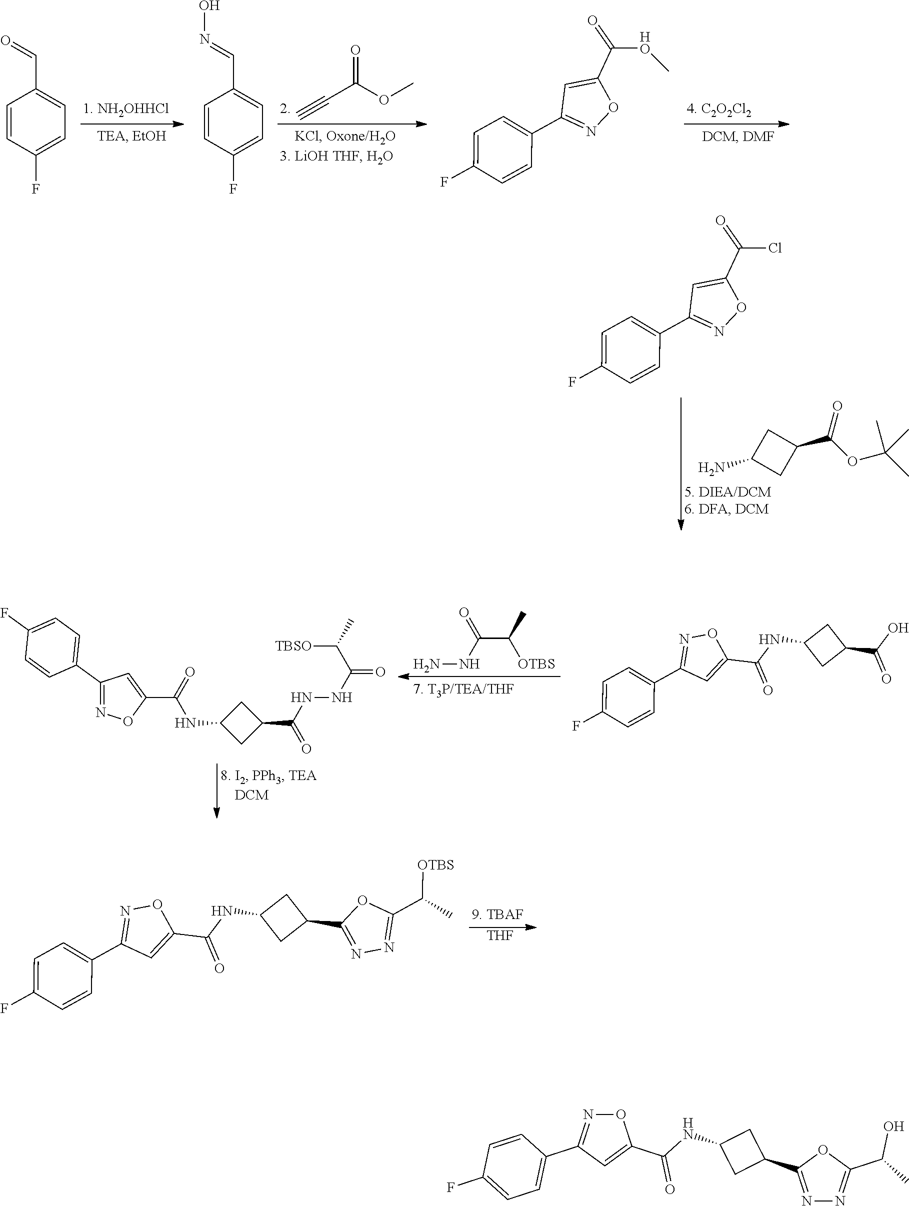

The title compound was prepared using a methodology similar to the one shown in example 13 and purified by Flash-Prep-HPLC with the following conditions (IntelFlash-1): Column, C18; mobile phase, H₂O/CH₃CN=100:1 increasing to H₂O/CH₃CN=1:100 within 30 min; Detector, UV 254 nm to give 37.7 mg (25%) as a white solid; LC-MS (ES, m/z): [M+1]⁺=373.0; ¹H NMR (400 MHz, DMSO-d₆): δ9.49-9.47 (d, J=7.6 Hz, 1H), 8.03-7.98 (m, 2H), 7.68 (s, 1H), 7.42-7.37 (m, 2H), 5.97-5.95 (d, J=6 Hz, 1H), 7.96-4.89 (m, 1H), 4.71-4.65 (m, 1H), 3.76-3.72 (m, 1H), 2.73-2.60 (m, 4H), 1.50-1.48 (d, J=6.4 Hz, 3H); HPLC purity: 99.8% at 254 nm.

### Examples 37 and 38: N-((1S,3s)-3-((5-((R)-1-hydroxyethyl)-1,3,4-oxadiazol-2-yl)methyl)cyclobutyl)-3-phenylisoxazole-5-carboxamide and N-((1R,3r)-3-((5-((R)-1-hydroxyethyl)-1,3,4-oxadiazol-2-yl)methyl)cyclobutyl)-3-phenylisoxazole-5-carboxamide

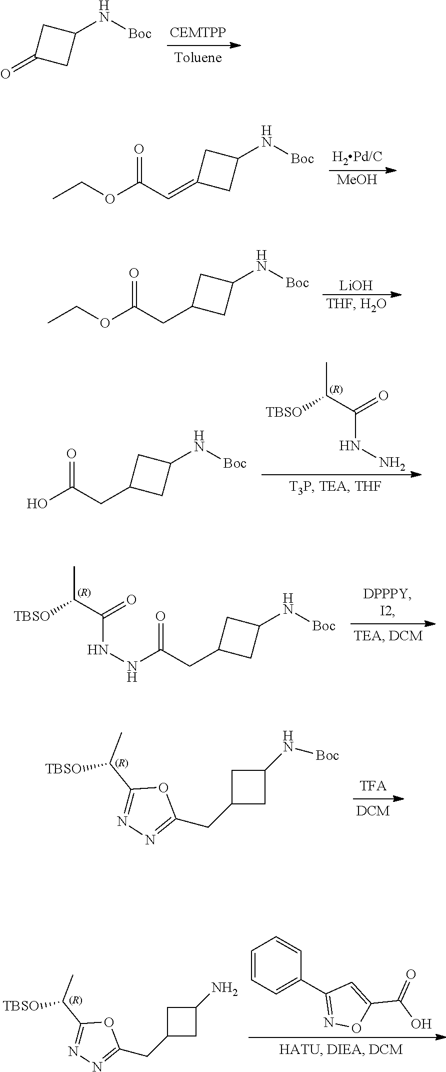

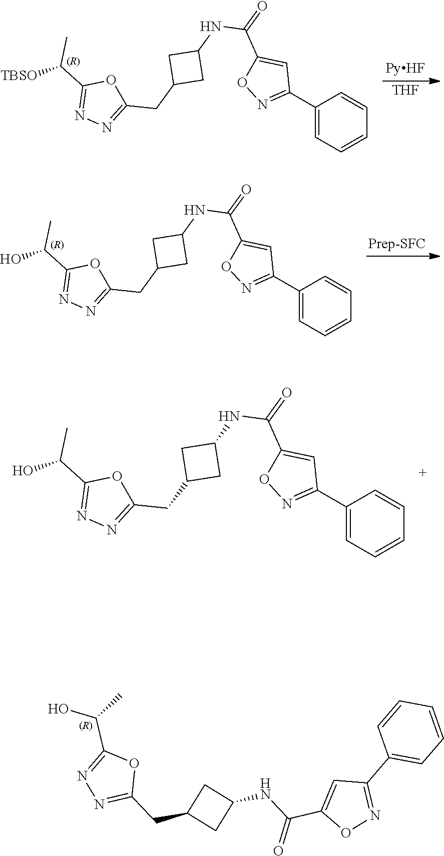

Step 1: Ethyl 2-(3-((tert-Butoxycarbonyl)amino)cyclobutylidene)acetate. To a 250-mL round-bottom flask was placed a solution of tert-butyl N-(3-oxocyclobutyl)carbamate (13 g, 70.19 mmol, 1.00 equiv) in toluene (100 mL), then (carbethoxymethylene)triphenylphosphorane (CEMTPP) (25.7 g, 73.77 mmol, 1.05 equiv) was added. The resulting solution was stirred for 2 h at 100° C. The resulting mixture was concentrated under vacuum then the residue was applied onto a silica gel column and eluted with EtOAc/petroleum ether (1:5) affording 16.7 g (93%) of ethyl 2-(3-[[(tert-butoxy)carbonyl]amino]cyclobutylidene) acetate as a white solid. LCMS (ES, m/z): [M+H]⁺=256.2.

Step 2: Ethyl 2-(3-((tert-Butoxycarbonyl)amino)cyclobutyl)acetate. To a 250-mL round-bottom flask, was placed a solution of ethyl 2-(3-[[(tert-butoxy)carbonyl]amino]cyclobutylidene)acetate (16.7 g, 65.41 mmol, 1.00 equiv, as prepared above) in MeOH (100 mL), then Pd on carbon (1 g) was added. The solution was degassed and back filled with hydrogen. The resulting solution was stirred for 3 h at RT. The solids were removed by filtration, then the resulting solution was concentrated under reduced pressure affording 15.5 g (92%) of ethyl 2-(3-[[(tert-butoxy)carbonyl]amino]cyclobutyl) acetate as colorless oil. LCMS (ES, m/z): [M+H]⁺=258.2.

Step 3: 2-(3-[[(tert-Butoxy)carbonyl]amino]cyclobutyl)acetic acid. To a 500-mL round-bottom flask was placed a solution of ethyl 2-(3-[[(tert-butoxy)carbonyl]amino]cyclobutyl)acetate (15.5 g, 60.23 mmol, 1.00 equiv) in THF/H₂O (150/50 mL) and LiOH (2.16 g, 90.20 mmol, 1.50 equiv). The resulting solution was stirred for 3 h at rt, then the resulting mixture was concentrated under reduced pressure. The resulting solution was diluted with 200 mL of aq.NaHSO₄, extracted with 3×150 mL of EtOAc, and then the organic extracts were combined. The solution was washed with 2×100 mL of brine, dried, and concentrated under reduced pressure, affording 13.8 g (crude) of 2-(3-[[(tert-butoxy)carbonyl]amino]cyclobutyl)acetic acid as colorless oil. LCMS (ES, m/z): [M+H]⁺=230.1.

Step 4: tert-Butyl N-(3-[2-[(2R)-2-[(tert-Butyldimethylsilyl)oxy]propanehydrazido]-2-oxoethyl]cyclobutyl)carbamate. To a 500-mL round-bottom flask was placed a solution of 2-(3-[[(tert-butoxy)carbonyl]amino]cyclobutyl)acetic acid (13 g, 56.70 mmol, 1.00 equiv) in THF (250 mL). To this solution were added (2R)-2-[(tert-butyldimethylsilyl)oxy]propanehydrazide (18.6 g, 85.18 mmol, 1.50 equiv), TEA (28.9 g, 285.60 mmol, 5.00 equiv) and T₃P (72 g, 113.21 mmol, 2.00 equiv). The reaction was stirred for 2 h at RT, then diluted with 400 mL of H₂O and extracted with EtOAc (3×300 mL). The organic extracts were combined, washed with brine (2×300 mL), dried over Na₂SO₄, and concentrated under reduced pressure. The residue was applied onto a silica gel column with petroleum ether/EtOAc (2:1) affording 14.5 g (60%) of tert-butyl N-(3-[2-[(2R)-2-[(tert-butyldimethylsilyl)oxy]propanehydrazido]-2-oxoethyl]cyclobutyl)carbamate as yellow oil. LCMS (ES, m/z): [M+H]⁺=430.3.

Step 5: tert-butyl N-[3-([5-[(1R)-1-[(tert-butyldimethylsilyl)oxy]ethyl]-1,3,4-oxadiazol-2-yl]methyl)cyclobutyl]carbamate. To a 250-mL 3-necked round-bottom flask purged and maintained with nitrogen was placed a solution of PPh₃ (2.84 g, 10.83 mmol, 2.00 equiv) in DCM (100 mL). To this solution were added I₂ (2.75 g, 10.83 mmol, 2.00 equiv), TEA (3.7 g, 36.56 mmol, 5.00 equiv) and tert-butyl N-[3-([N-[(2R)-2-[(tert-butyldimethylsilyl)oxy]propanoyl]hydrazinecarbonyl]methyl)cyclobutyl]carbamate (3.1 g, 7.22 mmol, 1.00 equiv). The resulting solution was stirred for 2 h at RT, then diluted with 150 mL of H₂O and extracted with EtOAc (2×150 mL). The organic extracts were combined, washed with brine (2×100 mL), dried over anhydrous Na₂SO₄, and concentrated under reduced pressure. The residue was applied onto a silica gel column and eluted with petroleum ether/EtOAc (5:1) affording 2 g (67%) of tert-butyl N-[3-([5-[(1R)-1-[(tert-butyldimethylsilyl)oxy]ethyl]-1,3,4-oxadiazol-2-yl]methyl)cyclobutyl]carbamate as yellow oil. LCMS (ES, m/z): [M+H]⁺=412.3. ¹H NMR (400 MHz, CDCl3): δ 5.11-5.02 (m, 1H), 4.15-4.08 (m, 1H), 3.02-2.92 (m, 2H), 2.59-2.52 (m, 1H), 2.26-2.19 (m, 1H), 2.14-2.08 (m, 1H), 1.70-1.62 (m, 2H), 1.58-1.56 (d, J=7.6 Hz, 2H), 1.43 (s, 9H), 0.88 (s, 9H), 0.11 (s, 3H), 0.04 (s, 3H).

Step 6: 3-([5-[(1R)-1-[(tert-butyldimethylsilyl)oxy]ethyl]-1,3,4-oxadiazol-2-yl]methyl)cyclobutan-1-amine. To a 100-mL round-bottom flask was placed a solution of tert-butyl N-[3-([5-(1R)-1-[(tert-butyldimethylsilyl)oxy]ethyl]-1,3,4-oxadiazol-2-yl]methyl)cyclobutyl]carbamate (2 g, 4.86 mmol, 1.00 equiv) in DCM (50 mL), then TFA (3 mL, 8.00 equiv) was added. The resulting solution was stirred for 2 h at RT then concentrated under reduced pressure affording 2.5 g (crude) of 3-([5-[(1R)-1-[(tert-butyldimethylsilyl)oxy]ethyl]-1,3,4-oxadiazol-2-yl]methyl)cyclobutan-1-amine as yellow crude oil. LCMS (ES, m/z): [M+H]⁺=312.2.

Step 7: N-[3-([5-[(1R)-1-[(tert-butyldimethylsilyl)oxy]ethyl]-1,3,4-oxadiazol-2-yl]methyl)cyclobutyl]-3-phenyl-1,2-oxazole-5-carboxamide. To a 100-mL round-bottom flask was placed a solution of 3-([5-[(1R)-1-[(tert-butyldimethylsilyl)oxy]ethyl]-1,3,4-oxadiazol-2-yl]methyl)cyclobutan-1-amine (1 g, crude) in DCM (50 mL), then 3-phenyl-1,2-oxazole-5-carboxylic acid (468 mg, 2.47 mmol, 1.00 equiv), HATU (1.28 g, 3.37 mmol, 1.20 equiv) and DIEA (1.1 mL, 2.80 equiv) were added. The resulting solution was stirred for 2 h at RT, washed with water (3×50 mL), and then concentrated under reduced pressure affording 680 mg (crude) of N-[3-([5-[(1R)-1-[(tert-butyldimethylsilyl)oxy]ethyl]-1,3,4-oxadiazol-2-yl]methyl)cyclobutyl]-3-phenyl-1,2-oxazole-5-carboxamide as yellow oil. LCMS (ES, m/z): [M+H]⁺=483.2.

Step 8: N-[3-([5-[(1R)-1-hydroxyethyl]-1,3,4-oxadiazol-2-yl]methyl)cyclobutyl]-3-phenyl-1,2-oxazole-5-carboxamide. To a 100-mL 3-necked round-bottom flask was placed a solution of N-[3-([5-[(1R)-1-[(tert-butyldimethylsilyl)oxy]ethyl]-1,3,4-oxadiazol-2-yl]methyl)cyclobutyl]-3-phenyl-1,2-oxazole-5-carboxamide (1 g, 2.07 mmol, 1.00 equiv) in THF (20 mL), then Py.HF (2.5 mL, 8.00 equiv) was added. The resulting solution was stirred for 2 h at 0° C. then quenched by the addition of 100 mL of brine. The resulting mixture was extracted with EtOAc (3×100 mL), then the organic extracts were combined, washed with NaHCO₃ (2×100 mL), brine (2×100 mL), and concentrated under reduced pressure. The residue was applied onto a silica gel column and eluted with petroleum ether/EtOAc (1:3) affording 460 mg of N-[3-([5-[(1R)-1-hydroxyethyl]-1,3,4-oxadiazol-2-yl]methyl)cyclobutyl]-3-phenyl-1,2-oxazole-5-carboxamide as light yellow oil. LCMS (ES, m/z): [M+H]⁺=369.2.

N-[3-([5-[(1R)-1-hydroxyethyl]-1,3,4-oxadiazol-2-yl]methyl)cyclobutyl]-3-phenyl-1,2-oxazole-5-carboxamide (520 mg, 1.41 mmol, 1.00 equiv) was purified by Prep-SFC with the following conditions: Column: Phenomenex Lux 5u Cellulose-4, 250*50 mm; Mobile Phase A:CO₂:50, Mobile Phase B: MeOH-Preparative: 50; Flow rate: 150 mL/min; 220 nm; RT1:6.38; RT2:7.33 affording 98.6 mg (19%) of 3-phenyl-N-[(1s,3s)-3-([5-[(1R)-1-hydroxyethyl]-1,3,4-oxadiazol-2-yl]methyl)cyclobutyl]-1,2-oxazole-5-carboxamide as a white solid and 78.7 mg (15%) of 3-phenyl-N-[(1r,3r)-3-([5-[(1R)-1-hydroxyethyl]-1,3,4-oxadiazol-2-yl]methyl)cyclobutyl]-1,2-oxazole-5-carboxamide as a white solid.

3-Phenyl-N-[(1s,3s)-3-([5-[(1R)-1-hydroxyethyl]-1,3,4-oxadiazol-2-yl]methyl)cyclobutyl]-1,2-oxazole-5-carboxamide: LCMS (ES, m/z): [M+H]⁺=369.0. ¹H NMR (300 MHz, DMSO-d₆): δ 9.23-9.20 (d, J=7.8 Hz, 1H), 7.94-7.91 (m, 2H), 7.62 (s, 1H), 7.55-7.53 (m, 3H), 5.92 (s, 1H), 4.92-4.85 (q, J=6.6 Hz, 1H), 4.35-4.27 (m, 1H), 2.99-2.97 (d, J=6.6 Hz, 2H), 2.45-2.35 (m, 3H), 1.98-1.92 (m, 2H), 1.47-1.44 (d, J=6.6 Hz, 3H). Purity (HPLC, 254 nm): 99.0%.

3-Phenyl-N-[(1r,3r)-3-([5-[(1R)-1-hydroxyethyl]-1,3,4-oxadiazol-2-yl]methyl)cyclobutyl]-1,2-oxazole-5-carboxamide: LCMS (ES, m/z): [M+H]⁺=369.0. ¹H NMR (300 MHz, DMSO-d₆): δ 9.23-9.20 (d, J=8.4 Hz, 1H), 7.94-7.91 (m, 2H), 7.62 (s, 1H), 7.55-7.53 (m, 3H), 5.92 (s, 1H), 4.92-4.85 (q, J=6.6 Hz, 1H), 4.35-4.28 (m, 1H), 2.99-2.97 (d, J=6.6 Hz, 2H), 2.45-2.35 (m, 3H), 1.98-1.92 (m, 2H), 1.47-1.44 (d, J=6.6 Hz, 3H). Purity (HPLC, 254 nm): 98.3%.

### Example 39 and 40: 3-(4-Fluorophenyl)-N-[(1s,3s)-3-([5-[(1R)-1-hydroxyethyl]-1,3,4-oxadiazol-2-yl]methyl)cyclobutyl]-1,2-oxazole-5-carboxamide and 3-(4-Fluorophenyl)-N-[(1r,3r)-3-([5-[(1R)-1-hydroxyethyl]-1,3,4-oxadiazol-2-yl]methyl)cyclobutyl]-1,2-oxazole-5-carboxamide

The title compounds were prepared using a methodology similar to the one shown in Example 37. The mixture was separated by Chiral-Prep-HPLC with the following conditions: Column: Repaired IA, 21.2*150 mm, 5 um; Mobile Phase A:Hex-HPLC, Mobile Phase B: EtOH-HPLC; Flow rate: 20 mL/min; Gradient: 50 B to 50 B in 11.5 min; 254/220 nm; RT1:7.21; RT2:8.75. This resulted in 95 mg (34%) of 3-(4-fluorophenyl)-N-[(1s,3s)-3-([5-[(1R)-1-hydroxyethyl]-1,3,4-oxadiazol-2-yl]methyl)cyclobutyl]-1,2-oxazole-5-carboxamide as a white solid and 79.6 mg (28%) of 3-(4-fluorophenyl)-N-[(1r,3r)-3-([5-[(1R)-1-hydroxyethyl]-1,3,4-oxadiazol-2-yl]methyl)cyclobutyl]-1,2-oxazole-5-carboxamide as a white solid.

3-(4-fluorophenyl)-N-[(1s,3s)-3-([5-[(1R)-1-hydroxyethyl]-1,3,4-oxadiazol-2-yl]methyl)cyclobutyl]-1,2-oxazole-5-carboxamide: LCMS (ES, m/z): [M+H]⁺=386.9. 1H NMR (300 MHz, DMSO-d₆): δ 9.23-9.20 (d, J=7.8 Hz, 1H), 8.02-7.97 (m, 2H), 7.63 (s, 1H), 7.42-7.36 (m, 2H), 5.92-5.90 (d, J=5.4 Hz 1H), 4.91-4.87 (m, 1H), 4.35-4.28 (m, 1H), 2.99-2.97 (d, J=6.9 Hz, 2H), 2.45-2.40 (m, 3H), 1.97-1.92 (m, 2H), 1.47-1.44 (d, J=6.9 Hz, 3H). Purity (HPLC, 254 nm): 99.3%.

3-(4-fluorophenyl)-N-[(1r,3r)-3-([5-[(1R)-1-hydroxyethyl]-1,3,4-oxadiazol-2-yl]methyl)cyclobutyl]-1,2-oxazole-5-carboxamide: LCMS (ES, m/z): [M+H]⁺=386. ¹H NMR (300 MHz, DMSO-d₆): δ 9.31-9.29 (d, J=7.2 Hz, 1H), 8.01-7.96 (m, 2H), 7.64 (s, 1H), 7.41-7.35 (m, 2H), 5.92-5.90 (d, J=5.7 Hz 1H), 4.93-4.84 (m, 1H), 4.58-4.51 (q, J=7.5 Hz, 1H), 3.10-3.07 (d, J=7.8 Hz, 2H), 2.70-2.64 (s, 1H), 2.38-2.29 (m, 2H), 2.18-2.09 (m, 2H), 1.46-1.44 (d, J=6.6 Hz, 3H). Purity (HPLC, 254 nm): 98.0%.

### Examples 41 and 42: 3-Phenyl-N-[(1s,3s)-3-([5-[(1R)-1-hydroxyethyl]-1,3,4-thiadiazol-2-yl]methyl)cyclobutyl]-1,2-oxazole-5-carboxamide and 3-Phenyl-N-[(1r,3r)-3-([5-[(1R)-1-hydroxyethyl]-1,3,4-thiadiazol-2-yl]methyl)cyclobutyl]-1,2-oxazole-5-carboxamide

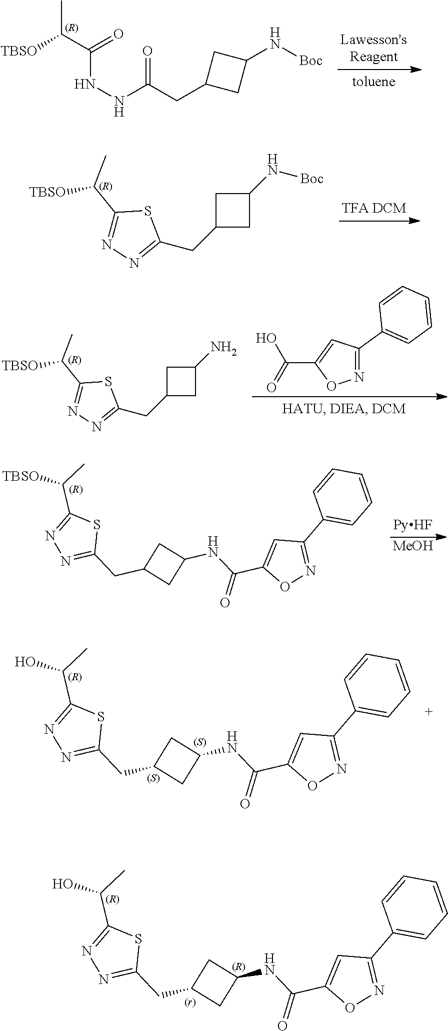

Step 1: tert-Butyl N-[3-([5-[(1R)-1-[(tert-Butyldimethylsilyl)oxy]ethyl]-1,3,4-thiadiazol-2-yl]methyl)cyclobutyl]carbamate. To a 250-mL round-bottom flask was placed a solution of tert-butyl N-(3-[2-[(2R)-2-[(tert-butyldimethylsilyl)oxy]propanehydrazido]-2-oxoethyl]cyclobutyl)carbamate (6 g, 13.97 mmol, 1.00 equiv) in toluene (100 mL) then Lawesson's reagent (8.5 g, 21.02 mmol, 1.50 equiv) was added. The resulting solution was stirred for 1.5 h at 80° C. then concentrated under reduced pressure. The resulting solution was diluted with 200 mL of H₂O and then extracted with EtOAc (3×200 mL). The organic extracts were combined, washed with brine (2×200 mL), dried over anhydrous Na₂SO₄, and concentrated under reduced pressure. The crude product was purified by Flash-Prep-HPLC (CombiFlash-1: Column, C18; mobile phase, X:H₂O (0.5% NH₄HCO₃), Y:CAN, X/Y=80/20 increasing to X/Y=5/95 within 40 min; Detector, UV 254 nm) affording 2.2 g (37%) of tert-butyl N-[3-([5-[(1R)-1-[(tert-butyldimethylsilyl)oxy]ethyl]-1,3,4-thiadiazol-2-yl]methyl)cyclobutyl]carbamate as yellow oil. LCMS (ES, m/z): [M+H−BOC]⁺=328.0.

Step 2: 3-([5-[(1R)-1-[(tert-butyldimethylsilyl)oxy]ethyl]-1,3,4-thiadiazol-2-yl]methyl)cyclobutan-1-amine. To a 50-mL round-bottom flask was placed a solution of tert-butyl N-[3-([5-[(1R)-1-[(tert-butyldimethylsilyl)oxy]ethyl]-1,3,4-thiadiazol-2-yl]methyl)cyclobutyl]carbamate (2.2 g, 5.14 mmol, 1.00 equiv) in DCM (20 mL) and TFA (4 mL). The resulting solution was stirred for 1 h at RT then concentrated under reduced pressure affording 3 g (crude) of 3-([5-[(1R)-1-[(tert-butyldimethylsilyl)oxy]ethyl]-1,3,4-thiadiazol-2-yl]methyl)cyclobutan-1-amine as yellow oil.

Step 3: N-[3-([5-[(1R)-1-[(tert-butyldimethylsilyl)oxy]ethyl]-1,3,4-thiadiazol-2-yl]methyl)cyclobutyl]-3-phenyl-1,2-oxazole-5-carboxamide. To a 50-mL round-bottom flask was placed a solution of 3-([5-[(1R)-1-[(tert-butyldimethylsilyl)oxy]ethyl]-1,3,4-thiadiazol-2-yl]methyl)cyclobutan-1-amine (500 mg, 1.53 mmol, 1.00 equiv) in DCM (20 mL), then HATU (753 mg, 1.98 mmol, 1.30 equiv), 3-phenyl-1,2-oxazole-5-carboxylic acid (317 mg, 1.68 mmol, 1.10 equiv) and DIEA (589 mg, 4.56 mmol, 3.00 equiv) were added. The resulting mixture was stirred for 2 h at RT then diluted with 100 mL of H₂O and extracted with EtOAc (2×50 mL). The organic extracts were combined, washed with brine (2×50 mL), dried over anhydrous Na₂SO₄, and concentrated under reduced pressure. The residue was applied onto a silica gel column and eluted with EtOAc/petroleum ether (1:5) affording 320 mg (42%) of N-[3-([5-[(1R)-1-[(tert-butyldimethylsilyl)oxy]ethyl]-1,3,4-thiadiazol-2-yl]methyl)cyclobutyl]-3-phenyl-1,2-oxazole-5-carboxamide as yellow oil. LCMS (ES, m/z): [M+H]⁺=499.1.

Step 4: 3-Phenyl-N-[(1s,3s)-3-([5-[(1R)-1-hydroxyethyl]-1,3,4-thiadiazol-2-yl]methyl)cyclobutyl]-1,2-oxazole-5-carboxamide and 3-Phenyl-N-[(1r,3r)-3-([5-[(1R)-1-hydroxyethyl]-1,3,4-thiadiazol-2-yl]methyl)cyclobutyl]-1,2-oxazole-5-carboxamide. To a 10-mL round-bottom flask was placed a solution of N-[3-([5-[(1R)-1-[(tert-butyldimethylsilyl)oxy]ethyl]-1,3,4-thiadiazol-2-yl]methyl)cyclobutyl]-3-phenyl-1,2-oxazole-5-carboxamide (320 mg, 0.64 mmol, 1.00 equiv) in MeOH (3 mL), then Py.HF (1 mL) was added. The resulting solution was stirred for 2 h at rt, diluted with 50 mL of H₂O, and extracted with EtOAc (2×50 mL). The organic extracts were combined, washed with brine (2×50 mL), dried over anhydrous Na₂SO₄, and concentrated under reduced pressure. The residue was applied onto a silica gel column and eluted with EtOAc/petroleum ether (1:1). The resulting isomers were separated by Chiral-Prep-HPLC (Prep-HPLC-004: Column, Phenomenex Lux 5u Cellulose-4AXIA Packed, 250*21.2 mm, 5 um; mobile phase, Hex and IPA (hold 50.0% IPA in 18 min); Detector, UV 254/220 nm) affording 88.7 mg (36%) of 3-phenyl-N-[(1s,3s)-3-([5-[(1R)-1-hydroxyethyl]-1,3,4-thiadiazol-2-yl]methyl)cyclobutyl]-1,2-oxazole-5-carboxamide as a white solid and 57.8 mg (23%) of 3-phenyl-N-[(1r,3r)-3-([5-[(1R)-1-hydroxyethyl]-1,3,4-thiadiazol-2-yl]methyl)cyclobutyl]-1,2-oxazole-5-carboxamide as a white solid.

3-Phenyl-N-[(1s,3s)-3-([5-[(1R)-1-hydroxyethyl]-1,3,4-thiadiazol-2-yl]methyl)cyclobutyl]-1,2-oxazole-5-carboxamide: LCMS (ES, m/z): [M+H]⁺=385.0. ¹H NMR (300 MHz, DMSO-d₆) δ 9.23-9.20 (d, J=7.5 Hz, 1H), 7.94-7.91 (m, 2H), 7.62 (s, 1H), 7.55-7.53 (m, 3H), 6.26-6.24 (d, J=5.1 Hz, 1H), 5.09-5.03 (m, 1H), 4.35-4.28 (m, 1H), 3.19-3.16 (d, J=7.2 Hz, 2H), 2.43-2.34 (m, 3H), 1.98-1.92 (m, 2H), 1.49-1.47 (d, J=6.3 Hz, 3H).). Purity (HPLC, 254 nm): 97.9%.

3-Phenyl-N-[(1r,3r)-3-([5-[(1R)-1-hydroxyethyl]-1,3,4-thiadiazol-2-yl]methyl)cyclobutyl]-1,2-oxazole-5-carboxamide: LCMS (ES, m/z): [M+H]⁺=385. ¹H NMR (300 MHz, DMSO-d₆): δ 9.31-9.29 (d, J=7.2 Hz, 1H), 7.94-7.91 (m, 2H), 7.64 (s, 1H), 7.55-7.53 (m, 3H), 6.25-6.24 (d, J=5.1 Hz, 1H), 5.09-5.01 (m, 1H), 4.60-4.52 (m, 1H), 3.29-3.26 (m, 2H), 2.66-2.62 (m, 1H), 2.37-2.27 (m, 2H), 2.18-2.12 (m, 2H), 1.49-1.47 (d, J=6.6 Hz, 3H). Purity (HPLC, 254 nm): 98.4%.

### Examples 43 and 44: 3-(4-Fluorophenyl)-N-[(1s,3s)-3-([5-[(1R)-1-hydroxyethyl]-1,3,4-thiadiazol-2-yl]methyl)cyclobutyl]-1,2-oxazole-5-carboxamide and 3-(4-Fluorophenyl)-N-[(1r,3r)-3-([5-[(1R)-1-hydroxyethyl]-1,3,4-thiadiazol-2-yl]methyl)cyclobutyl]-1,2-oxazole-5-carboxamide

The title compounds were prepared using a methodology similar to the one shown in Example 41. The resulting isomers were separated by Prep-SFC (Prep SFC100: Column, Phenomenex Lux 5u Cellulose-4AXIA Packed, 250*21.2 mm, 5 um; mobile phase, CO2 (60%), ETOH (0.2% DEA)-(40%); Detector, uv 220 nm) affording 125 mg (22%) of 3-(4-fluorophenyl)-N-[(1s,3s)-3-([5-[(1R)-1-hydroxyethyl]-1,3,4-thiadiazol-2-yl]methyl)cyclobutyl]-1,2-oxazole-5-carboxamide as a white solid and 110.8 mg (20%) of 3-(4-fluorophenyl)-N-[(1r,3r)-3-([5-[(1R)-1-hydroxyethyl]-1,3,4-thiadiazol-2-yl]methyl)cyclobutyl]-1,2-oxazole-5-carboxamide as a white solid.

3-(4-Fluorophenyl)-N-[(1s,3s)-3-([5-[(1R)-1-hydroxyethyl]-1,3,4-thiadiazol-2-yl]methyl)cyclobutyl]-1,2-oxazole-5-carboxamide: LCMS (ES, m/z): [M+H]⁺=403. ¹H NMR (400 MHz, DMSO-d₆) δ 9.24-9.22 (d, J=8.0 Hz, 1H), 8.01-7.98 (m, 2H), 7.63 (s, 1H), 7.41-7.37 (m, 2H), 6.25 (s, 1H), 5.05-5.04 (m, 1H), 4.34-4.28 (m, 1H), 3.18-3.16 (d, J=6.8 Hz, 2H), 2.45-2.36 (m, 3H), 1.94-1.92 (m, 2H), 1.48-1.47 (d, J=6.4 Hz, 3H). Purity (HPLC, 254 nm): 99.4%.

3-(4-Fluorophenyl)-N-[(1r,3r)-3-([5-[(1R)-1-hydroxyethyl]-1,3,4-thiadiazol-2-yl]methyl)cyclobutyl]-1,2-oxazole-5-carboxamide: LCMS (ES, m/z): [M+H]⁺=403.0. ¹H NMR (300 MHz, DMSO-d₆) δ 9.32-9.29 (d, J=7.5 Hz, 1H), 8.02-7.97 (m, 2H), 7.65 (s, 1H), 7.42-7.36 (m, 2H), 6.25-6.24 (d, J=5.1 Hz, 1H), 5.09-5.01 (m, 1H), 4.62-4.50 (m, 1H), 3.29-3.26 (d, J=8.1 Hz, 2H), 2.69-2.60 (m, 1H), 2.37-2.27 (m, 2H), 2.19-2.10 (m, 2H), 1.49-1.47 (d, J=6.6 Hz, 3H). Purity (HPLC, 254 nm): 96.3%.

### Examples 45 and 46: N-[(1s,3s)-3-([5-[(1R)-1-Hydroxyethyl]-1,3,4-oxadiazol-2-yl]methyl)cyclobutyl]-3-(thiophen-2-yl)-1,2-oxazole-5-carboxamide and N-[(1r,3r)-3-([5-[(1R)-1-Hydroxyethyl]-1,3,4-oxadiazol-2-yl]methyl)cyclobutyl]-3-(thiophen-2-yl)-1,2-oxazole-5-carboxamide

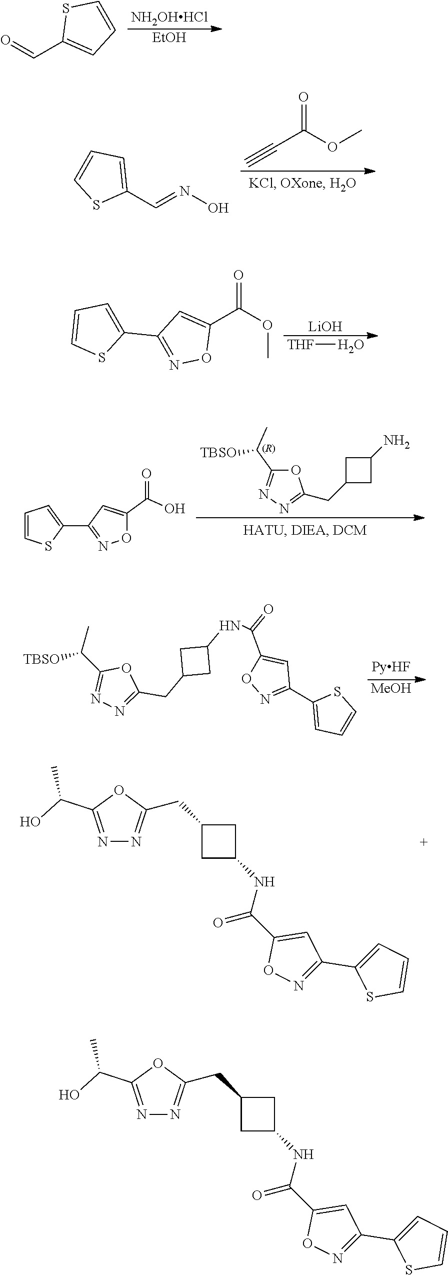

Step 1: N-(thiophen-2-ylmethylidene)hydroxylamine. To a 100-mL round-bottom flask was placed a solution of thiophene-2-carbaldehyde (5 g, 44.58 mmol, 1.00 equiv) in EtOH (50 mL) then NH₂OH.HCl (3.7 g, 1.20 equiv) was added. The resulting solution was stirred for 2 h at RT then the reaction was extracted with EtOAc. The organic extracts were combined, dried, and concentrated under reduced pressure affording 4.5 g (79%) of N-(thiophen-2-ylmethylidene)hydroxylamine as yellow oil. LCMS (ES, m/z): [M+H]⁺=128.0.

Step 2: Methyl 3-(Thiophen-2-yl)-1,2-oxazole-5-carboxylate. To a 100-mL round-bottom flask was placed a solution of N-(thiophen-2-ylmethylidene)hydroxylamine (4.5 g, 35.39 mmol, 1.00 equiv) in H₂O (50 mL), then methyl prop-2-ynoate (8 mL, 2.50 equiv), KCl (2.6 g, 1.00 equiv) and Oxone (14.4 g, 1.50 equiv) were added. The resulting solution was stirred for 2 h at RT then the reaction was extracted with EtOAc (3×100 mL). The organic extracts were combined, dried, and concentrated under reduced pressure affording 5.4 g (73%) of methyl 3-(thiophen-2-yl)-1,2-oxazole-5-carboxylate as a yellow solid. LCMS (ES, m/z): [M+H]⁺=210.0.

Step 3: 3-(thiophen-2-yl)-1,2-oxazole-5-carboxylic acid. To a 250-mL round-bottom flask was placed a solution of methyl 3-(thiophen-2-yl)-1,2-oxazole-5-carboxylate (5.4 g, 25.81 mmol, 1.00 equiv) in THF and H₂O (30 mL/10 mL), then LiOH (1.33 g, 55.53 mmol, 2.00 equiv) was added. The resulting solution was stirred for 1 h at RT. After concentrating under reduced pressure, the residue was diluted with 100 mL of H₂O then the resulting solution was washed with EtOAc (2×30 mL). The pH value of the aqueous layer was adjusted to 3 with HCl, then the solution was extracted with EtOAc (3×100 mL). The organic extracts were combined, dried over anhydrous Na₂SO₄, and concentrated under reduced pressure affording 3.2 g (64%) of 3-(thiophen-2-yl)-1,2-oxazole-5-carboxylic acid as a white solid. LCMS (ES, m/z): [M+H]⁺=196.1.

Step 4: N-[3-([5-[(1R)-1-[(tert-butyldimethylsilyl)oxy]ethyl]-1,3,4-oxadiazol-2-yl]methyl)cyclobutyl]-3-(thiophen-2-yl)-1,2-oxazole-5-carboxamide. To a 100-mL 3-necked round-bottom flask was placed a solution of 3-([5-[(1R)-1-[(tert-butyldimethylsilyl)oxy]ethyl]-1,3,4-oxadiazol-2-yl]methyl)cyclobutan-1-amine (1.25 g, 4.02 mmol, 1.00 equiv) in DCM (30 mL) then 3-(thiophen-2-yl)-1,2-oxazole-5-carboxylic acid (800 mg, 4.10 mmol, 1.02 equiv), HATU (2.3 g, 6.05 mmol, 1.50 equiv) and DIEA (3.1 g, 24.01 mmol, 6.00 equiv) were added. The resulting solution was stirred for 3 h at RT then washed with brine (2×60 mL), dried over anhydrous Na₂SO₄, and concentrated under reduced pressure. The residue was applied onto a silica gel column and eluted with EtOAc/petroleum ether (1:8 affording 2.3 g (crude) of N-[3-([5-[(1R)-1-[(tert-butyldimethylsilyl)oxy]ethyl]-1,3,4-oxadiazol-2-yl]methyl)cyclobutyl]-3-(thiophen-2-yl)-1,2-oxazole-5-carboxamide as yellow oil. LCMS (ES, m/z): [M+H]⁺=489.2.

Step 5: N-[(1s,3s)-3-([5-[(1R)-1-Hydroxyethyl]-1,3,4-oxadiazol-2-yl]methyl)cyclobutyl]-3-(thiophen-2-yl)-1,2-oxazole-5-carboxamide and N-[(1r,3r)-3-([5-[(1R)-1-Hydroxyethyl]-1,3,4-oxadiazol-2-yl]methyl)cyclobutyl]-3-(thiophen-2-yl)-1,2-oxazole-5-carboxamide. To a 100-mL 3-necked round-bottom flask was placed a solution of N-[3-([5-(1R)-1-[(tert-butyldimethylsilyl)oxy]ethyl]-1,3,4-oxadiazol-2-yl]methyl)cyclobutyl]-3-(thiophen-2-yl)-1,2-oxazole-5-carboxamide (1.1 g, 2.25 mmol, 1.00 equiv) in MeOH (50 mL) then Py.HF (6 mL) was added. The resulting solution was stirred for 1 h at RT then concentrated under reduced pressure. The residue was dissolved in EtOAc (60 mL), washed with NaHCO₃ solution (2×50 mL) and brine (2×50 mL), then dried over anhydrous Na₂SO₄, and concentrated under reduced pressure. The residue was applied onto a silica gel column and eluted with EtOAc/petroleum ether (1:3) affording 150 mg of a mixture of PH-PTS-005-0005 and PH-PTS-005-0017. The mixture was separated by Chiral-Prep-HPLC (Column: Phenomenex Lux 5u Cellulose-4, AXIA Packed, 250*21.2 mm, 5 um; Mobile Phase A: Hex, Mobile Phase B: EtOH; Flow rate: 20 m/min; Gradient: 30 B to 30 B in 27 min; 254/220 nm; RT1:19.83; RT2:23.28) affording 52.6 mg of N-[(1s,3s)-3-([5-[(1R)-1-hydroxyethyl]-1,3,4-oxadiazol-2-yl]methyl)cyclobutyl]-3-(thiophen-2-yl)-1,2-oxazole-5-carboxamide as a white solid and 51.3 mg of N-[(1r,3r)-3-([5-[(1R)-1-hydroxyethyl]-1,3,4-oxadiazol-2-yl]methyl)cyclobutyl]-3-(thiophen-2-yl)-1,2-oxazole-5-carboxamide as a white solid.

N-[(1s,3s)-3-([5-[(1R)-1-hydroxyethyl]-1,3,4-oxadiazol-2-yl]methyl)cyclobutyl]-3-(thiophen-2-yl)-1,2-oxazole-5-carboxamide: LC-MS-PH-PTS-005-0005-0: (ES, m/z): [M+H]⁺=375.0. ¹H NMR (300 MHz, DMSO-d₆): δ 9.24-9.20 (d, J=7.5 Hz, 1H), 7.80-7.78 (m, 2H), 7.59 (s, 1H), 7.26-7.23 (m, 1H), 5.92-5.90 (d, J=5.7 Hz, 1H), 4.93-4.84 (m, 1H), 4.35-4.27 (m, 1H), 2.99-2.96 (d, J=6.6 Hz, 2H), 2.47-2.37 (m, 3H), 1.97-1.91 (m, 2H), 1.46-1.44 (d, J=6.6 Hz, 3H). Purity (HPLC, 254 nm): 95.9%.

N-[(1r,3r)-3-([5-[(1R)-1-hydroxyethyl]-1,3,4-oxadiazol-2-yl]methyl)cyclobutyl]-3-(thiophen-2-yl)-1,2-oxazole-5-carboxamide: LCMS (ES, m/z): [M+H]⁺=375.0. ¹H NMR (300 MHz, DMSO-d₆): δ 9.32-9.30 (d, J=7.2 Hz, 1H), 7.80-7.78 (d, J=4.5 Hz, 2H), 7.60 (s, 1H), 7.26-7.23 (m, 1H), 5.93-5.91 (d, J=5.4 Hz, 1H), 4.93-4.85 (m, 1H), 4.58-4.51 (m, 1H), 3.10-3.08 (d, J=7.8 Hz, 2H), 2.72-2.65 (m, 1H), 2.83-2.29 (m, 2H), 2.18-2.11 (m, 2H), 1.47-1.44 (d, J=6.9 Hz, 3H). Purity (HPLC, 254 nm): 99.7%.

### Examples 47 and 48: N-((1r,3r)-3-((5-(Hydroxymethyl)-1H-1,2,3-triazol-1-yl)methyl)cyclobutyl)-3-phenylisoxazole-5-carboxamide and N-((1r,3r)-3-((4-(Hydroxymethyl)-1H-1,2,3-triazol-1-yl)methyl)cyclobutyl)-3-phenylisoxazole-5-carboxamide

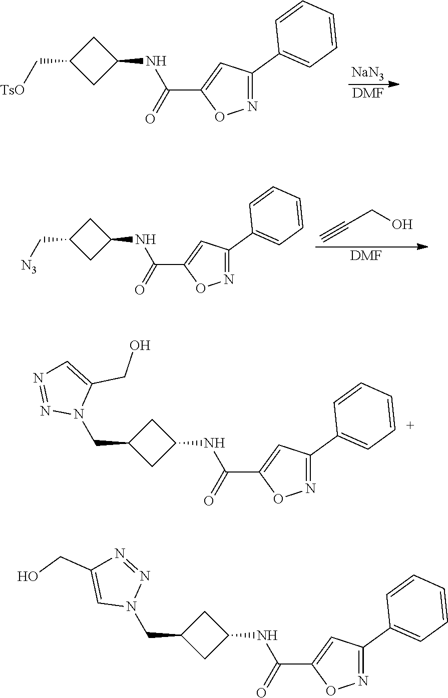

Step 1: N-((1r,3r)-3-(Azidomethyl)cyclobutyl)-3-phenylisoxazole-5-carboxamide. To a 50-mL round-bottom flask was placed a solution of ((1r,3r)-3-(3-phenylisoxazole-5-carboxamido)cyclobutyl)methyl 4-methylbenzenesulfonate (1.5 g, 3.52 mmol, 1.00 equiv) in DMF (15 mL) then NaN₃ (390 mg, 6.00 mmol, 1.50 equiv) was added. The resulting solution was stirred for 5 h at 80° C., quenched by the addition of 20 mL of ice/water, and extracted with DCM (2×30 mL). The organic extracts were combined, dried over anhydrous Na₂SO₄, and concentrated under reduced pressure. The residue was applied onto a silica gel column and eluted with EtOAc/petroleum ether (1:20) affording 0.9 g (86%) of N-((1r,3r)-3-(azidomethyl)cyclobutyl)-3-phenylisoxazole-5-carboxamide as a white solid. LCMS: (ES, m/z): [M+H]⁺=298.1.

Step 2: N-((1r,3r)-3-((5-(Hydroxymethyl)-1H-1,2,3-triazol-1-yl)methyl)cyclobutyl)-3-phenylisoxazole-5-carboxamide and N-((1r,3r)-3-((4-(Hydroxymethyl)-1H-1,2,3-triazol-1-yl)methyl)cyclobutyl)-3-phenylisoxazole-5-carboxamide. To a 25-mL round-bottom flask was placed a solution of N-((1r,3r)-3-(azidomethyl)cyclobutyl)-3-phenylisoxazole-5-carboxamide (700 mg, 2.35 mmol, 1.00 equiv) in DMF (5 mL) then prop-2-yn-1-ol (660 mg, 11.77 mmol, 5.00 equiv) was added. The resulting solution was stirred for 24 h at 80° C., then the solvent was removed under reduced pressure. The residue was applied onto a silica gel column and eluted with EtOAc/petroleum ether (1:5). The resulting mixture was separated by Prep-SFC (Column: Lux 5u Celluloes-3, AXIA Packed, 250*21.2 mm; Mobile Phase A:CO₂:70, Mobile Phase B: MeOH:30; Flow rate: 40 mL/min; 220 nm; RT1:4.47; RT2:5.32) affording 120 mg (27%) of N-((1r,3r)-3-((5-(hydroxymethyl)-1H-1,2,3-triazol-1-yl)methyl)cyclobutyl)-3-phenylisoxazole-5-carboxamide as a white solid and 119.8 mg (27%) of N-((1r,3r)-3-((4-(hydroxymethyl)-1H-1,2,3-triazol-1-yl)methyl)cyclobutyl)-3-phenylisoxazole-5-carboxamide as a white solid.

N-((1r,3r)-3-((5-(Hydroxymethyl)-1H-1,2,3-triazol-1-yl)methyl)cyclobutyl)-3-phenylisoxazole-5-carboxamide: LCMS (ES, m/z): [M+H]⁺=354.1. ¹H NMR (300 MHz, DMSO-d₆): δ 9.29-9.27 (d, J=7.2 Hz, 1H), 8.00 (s, 1H), 7.93-7.90 (m, 2H), 7.63 (s, 1H), 7.55-7.52 (m, 3H), 5.17-5.13 (t, J=5.7 Hz, 1H), 4.60-4.49 (m, 5H), 2.74-2.70 (m, 1H), 2.31-2.12 (m, 4H). Purity (HPLC, 254 nm): 98.8%.

N-((1r,3r)-3-((4-(Hydroxymethyl)-1H-1,2,3-triazol-1-yl)methyl)cyclobutyl)-3-phenylisoxazole-5-carboxamide: LCMS (ES, m/z): [M+H]⁺=354. ¹H NMR (300 MHz, DMSO-d₆): δ 9.30-9.27 (d, J=7.5 Hz, 1H), 7.93-7.90 (m, 2H), 7.63-7.60 (m, 2H), 7.57-7.52 (m, 5H), 5.51-5.47 (t, J=5.7 Hz, 1H), 4.66-4.60 (m, 3H), 4.53-4.47 (m, 2H), 2.86-2.78 (m, 1H), 2.30-2.15 (m, 4H). Purity (HPLC, 254 nm): 96.9%.

### Example 49: 3-Phenyl-N-[(1r,3r)-3-[5-(oxetan-2-yl)-1,3,4-oxadiazol-2-yl]cyclobutyl]-1,2-oxazole-5-carboxamide

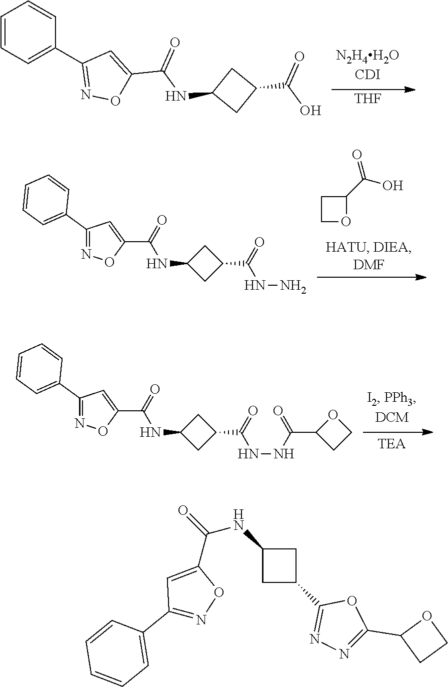 [SMILES: O=C(N[C@H]1C[C@H](C(=O)O)C1)c1cc(-c2ccccc2)no1 ; NNC(=O)[C@H]1C[C@H](NC(=O)c2cc(-c3ccccc3)no2)C1 ; O=C(O)C1CCO1 ; O=C(N[C@H]1C[C@H](C(=O)NNC(=O)C2CCO2)C1)c1cc(-c2ccccc2)no1 ; O=C(N[C@H]1C[C@H](c2nnc(C3CCO3)o2)C1)c1cc(-c2ccccc2)no1 ; "THF"]

Step 1: 3-Phenyl-N-[(1r,3r)-3-(hydrazinecarbonyl)cyclobutyl]-1,2-oxazole-5-carboxamide. To a 100-mL round-bottom flask was placed a solution of (1r,3r)-3-(3-phenyl-1,2-oxazole-5-amido)cyclobutane-1-carboxylic acid (2.87 g, 10.03 mmol, 1.00 equiv) in THF (50 mL), then CDI (3.24 g, 20.00 mmol, 2.00 equiv) was added. The resulting solution was stirred for 1 h at 25° C. and then N₂H4.H₂O (2.1 g, 30.00 mmol, 3.00 equiv) was added. The resulting solution was stirred for 16 h at RT then diluted with 300 mL of H₂O. The solids were collected by filtration and dried in an oven under reduced pressure affording 487 mg (16%) of 3-phenyl-N-[(1r,3r)-3-(hydrazinecarbonyl)cyclobutyl]-1,2-oxazole-5-carboxamide as a light yellow solid. LCMS (ES, m/z): [M+H]⁺=301.1.

Step 2: 3-Phenyl-N-[(1r,3r)-3-[(oxetan-2-ylformohydrazido)carbonyl]cyclobutyl]-1,2-oxazole-5-carboxamide. To a 25-mL round-bottom flask was placed a solution of 3-phenyl-N-[(1r,3r)-3-(hydrazinecarbonyl)cyclobutyl]-1,2-oxazole-5-carboxamide (280 mg, 0.93 mmol, 1.00 equiv) in DMF (5 mL) then HATU (570 mg, 1.50 mmol, 1.50 equiv), DIEA (361 mg, 2.79 mmol, 3.00 equiv) and oxetane-2-carboxylic acid (143 mg, 1.40 mmol, 1.50 equiv) were added. The resulting solution was stirred for 2 h at RT then diluted with 50 mL of H₂O and extracted with EtOAc (2×50 mL). The organic extracts were combined, washed with brine (2×50 mL), dried over anhydrous Na₂SO₄, and concentrated under reduced pressure. The crude product was purified by Prep-TLC (petroleum ether/ethyl acetate=1:2) affording 220 mg (61%) of 3-phenyl-N-[(1r,3r)-3-[(oxetan-2-ylformohydrazido)carbonyl]cyclobutyl]-1,2-oxazole-5-carboxamide as a yellow solid. LCMS (ES, m/z): [M+H]⁺=385.1.

Step 3: 3-Phenyl-N-[(1r,3r)-3-[5-(oxetan-2-yl)-1,3,4-oxadiazol-2-yl]cyclobutyl]-1,2-oxazole-5-carboxamide. To a 100-mL 3-necked round-bottom flask was placed a solution of PPh₃ (299 mg, 1.14 mmol, 2.00 equiv) in DCM (20 mL), then 12 (290 mg, 1.14 mmol, 2.00 equiv) and TEA (230 mg, 2.27 mmol, 4.00 equiv) were added. The resulting solution was stirred for 10 min at RT then 3-phenyl-N-[(1r,3r)-3-[(oxetan-2-ylformohydrazido)carbonyl]cyclobutyl]-1,2-oxazole-5-carboxamide (220 mg, 0.57 mmol, 1.00 equiv) was added and stirred for 1 h at rt. The reaction was diluted with 100 mL of H₂O and extracted with EtOAc (2×100 mL). The organic extracts were combined, washed with brine (2×100 mL), dried over anhydrous Na₂SO₄, and concentrated under reduced pressure. The residue was applied onto a silica gel column and eluted with EtOAc/petroleum ether (2:1) affording 174.8 mg (83%) of 3-phenyl-N-[(1r,3r)-3-[5-(oxetan-2-yl)-1,3,4-oxadiazol-2-yl]cyclobutyl]-1,2-oxazole-5-carboxamide as an off-white solid. LCMS (ES, m/z): [M+H]⁺=367.3. ¹H NMR (400 MHz, DMSO-d₆) δ 9.45 (d, J=7.2 Hz, 1H), 7.93-7.91 (m, 2H), 7.65 (s, 1H), 7.54-7.52 (m, 3H), 5.88-5.84 (t, J=7.6 Hz, 1H), 4.72-4.62 (m, 3H), 3.81-3.74 (m, 1H), 3.12-2.96 (m, 2H), 2.73-2.66 (m, 4H). Purity (HPLC, 254 nm): 99.5%.

### Example 50: 4-Fluoro-3-phenyl-N-[(1r,3r)-3-[5-[(1R)-1-hydroxyethyl]-1,3,4-oxadiazol-2-yl]cyclobutyl]-1,2-oxazole-5-carboxamide

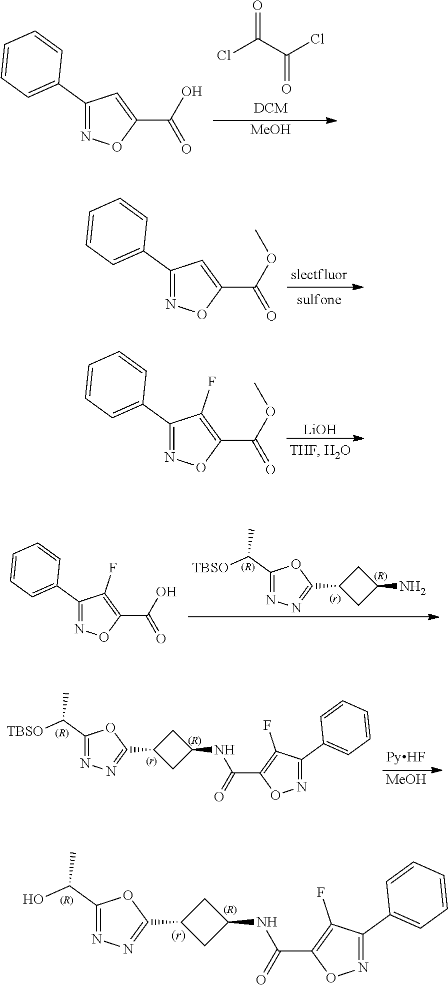

Step 1: Methyl 3-Phenyl-1,2-oxazole-5-carboxylate. To a 50-mL round-bottom flask was placed a solution of 3-phenyl-1,2-oxazole-5-carboxylic acid (1.89 g, 9.99 mmol, 1.00 equiv) in DCM (20 mL) then oxalyl chloride (1.9 g, 14.97 mmol, 1.50 equiv) and a drop of DMF were added. The resulting solution was stirred for 1 h at RT then MeOH (5 mL) was added. The reaction was stirred for 1 h at RT then concentrated under reduced pressure affording 1.9 g (94%) of methyl 3-phenyl-1,2-oxazole-5-carboxylate as a yellow solid.

Step 2: Methyl 4-Fluoro-3-phenyl-1,2-oxazole-5-carboxylate. To a 25-mL round-bottom flask was placed a solution of methyl 3-phenyl-1,2-oxazole-5-carboxylate (1 g, 4.92 mmol, 1.00 equiv) in sulfone (10 mL) then Selectfluor (3.54 g, 10.00 mmol, 2.00 equiv) was added. The resulting solution was stirred for 16 h at 120° C., diluted with 100 mL of H₂O, and extracted with EtOAc (2×100 mL). The organic extracts were combined, washed with brine (2×100 mL), dried over anhydrous Na₂SO₄, and concentrated under reduced pressure. The crude product was purified by Prep-TLC (petroleum ether/ethyl acetate=10:1) affording 250 mg (25%) of methyl 4-fluoro-3-phenyl-1,2-oxazole-5-carboxylate as a white solid. LCMS (ES, m/z): [M+H]⁺=222.0.

Step 3: 4-Fluoro-3-phenyl-1,2-oxazole-5-carboxylic acid. To a 25-mL round-bottom flask was placed a solution of methyl 4-fluoro-3-phenyl-1,2-oxazole-5-carboxylate (250 mg, 1.13 mmol, 1.00 equiv) in THF/H₂O (10/3 mL) then LiOH (82 mg, 3.42 mmol, 3.00 equiv) was added. The resulting solution was stirred for 2 h at RT and then diluted with 50 mL of H₂O. The pH of the solution was adjusted to 4-5 using concentrated 12M HCl, then extracted with EtOAc (2×50 mL). The organic extracts were combined, washed with brine (2×50 mL), dried over anhydrous Na₂SO₄, and concentrated under reduced pressure affording 210 mg (90%) of 4-fluoro-3-phenyl-1,2-oxazole-5-carboxylic acid as a white solid.

Step 3: 4-Fluoro-3-phenyl-N-[(1r,3r)-3-[5-[(1S)-1-[(tert-butyldimethylsilyl)oxy]ethyl]-1,3,4-oxadiazol-2-yl]cyclobutyl]-1,2-oxazole-5-carboxamide. To a 50-mL round-bottom flask was placed a solution of 4-fluoro-3-phenyl-1,2-oxazole-5-carboxylic acid (210 mg, 1.01 mmol, 1.00 equiv) in DCM (10 mL), then HATU (570 mg, 1.50 mmol, 1.50 equiv), (1r,3r)-3-5-[(1S)-1-[(tert-butyldimethylsilyl)oxy]ethyl]-1,3,4-oxadiazol-2-ylcyclobutan-1-amine (300 mg, 1.01 mmol, 1.00 equiv) and DIEA (387 mg, 2.99 mmol, 3.00 equiv) were added. The resulting solution was stirred for 1 h at RT, diluted with 100 mL of H₂O, and extracted with EtOAc (2×100 mL). The organic extracts were combined, washed with brine (2×100 mL), dried over anhydrous Na₂SO₄, and concentrated under reduced pressure. The residue was applied onto a silica gel column and eluted with EtOAc/petroleum ether (1:5) affording 360 mg (73%) of 4-fluoro-3-phenyl-N-[(1r,3r)-3-[5-[(1S)-1-[(tert-butyldimethylsilyl)oxy]ethyl]-1,3,4-oxadiazol-2-yl]cyclobutyl]-1,2-oxazole-5-carboxamide as a white solid. LCMS (ES, m/z): [M+H]⁺=487.3.

Step 4: 4-Fluoro-3-phenyl-N-[(1r,3r)-3-[5-[(1R)-1-hydroxyethyl]-1,3,4-oxadiazol-2-yl]cyclobutyl]-1,2-oxazole-5-carboxamide. To a 25-mL round-bottom flask was placed a solution of 4-fluoro-3-phenyl-N-[(1r,3r)-3-[5-[(1R)-1-[(tert-butyldimethylsilyl)oxy]ethyl]-1,3,4-oxadiazol-2-yl]cyclobutyl]-1,2-oxazole-5-carboxamide (360 mg, 0.74 mmol, 1.00 equiv) in methanol (6 mL) then Py.HF (2 mL) was added. The resulting solution was stirred for 1 h at RT then diluted with 50 mL of H₂O, and extracted with EtOAc (2×50 mL). The organic extracts were combined, washed with brine (3×50 mL), dried over anhydrous Na₂SO₄, and concentrated under reduced pressure. The crude product was purified by Prep-HPLC (HPLC-10: Column, X Bridge C18 OBD Prep Column, 19 mm×250 mm; mobile phase, Water (0.5% NH₄HCO₃) and ACN (30.0% ACN up to 50.0% in 8 min); Detector, UV 254/220 nm) affording 133.3 mg (48%) of 4-fluoro-3-phenyl-N-[(1r,3r)-3-[5-[(1R)-1-hydroxyethyl]-1,3,4-oxadiazol-2-yl]cyclobutyl]-1,2-oxazole-5-carboxamide as a white solid. LCMS (ES, m/z): [M+H]⁺=372.9. ¹H NMR (400 MHz, DMSO-d₆) δ 9.49-9.47 (d, J=7.2 Hz, 1H), 7.99-7.94 (m, 1H), 7.65-7.60 (m, 1H), 7.52 (s, 1H), 7.48-7.37 (m, 2H), 5.96-5.95 (d, J=6.4 Hz, 1H), 4.95-4.89 (m, 1H), 4.70-4.64 (m, 1H), 3.78-3.72 (m, 1H), 2.72-2.63 (m, 4H), 1.49-1.48 (d, J=6.8 Hz, 3H). Purity (HPLC, 254 nm): 99.7%.

### Examples 51 and 52: 3-Phenyl-N-[(1s,3s)-3-[5-[(1R)-1-hydroxyethyl]-1H-1,2,3-triazol-1-yl]cyclobutyl]-1,2-oxazole-5-carboxamide and 3-Phenyl-N-[(1s,3s)-3-[4-[(1R)-1-hydroxyethyl]-1H-1,2,3-triazol-1-yl]cyclobutyl]-1,2-oxazole-5-carboxamide

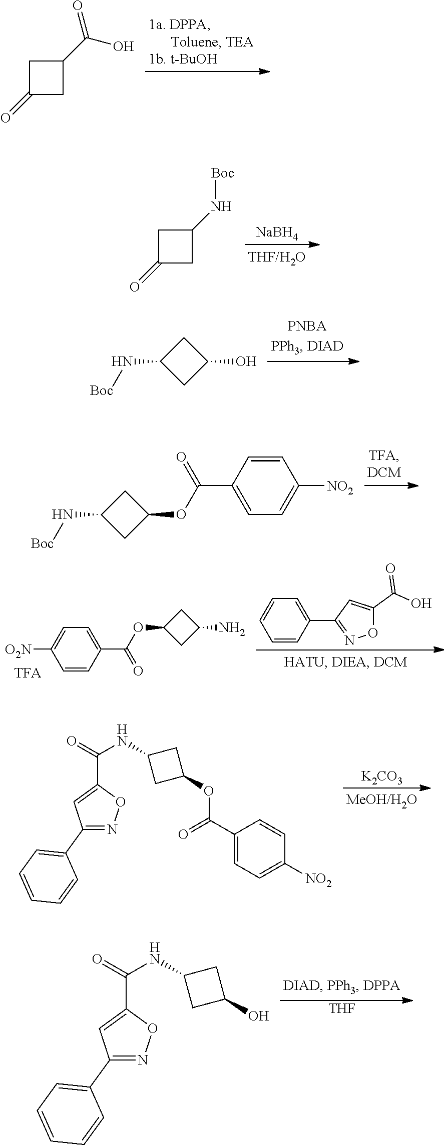

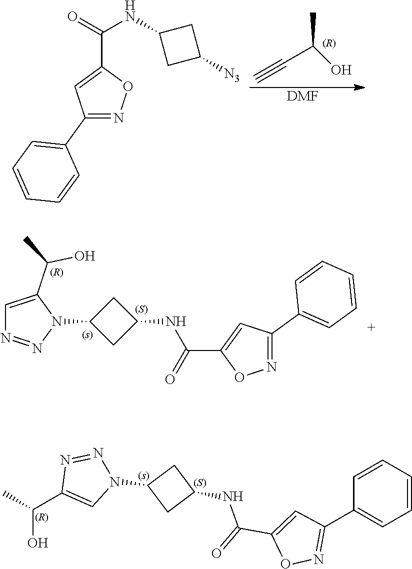

Step 1: tert-Butyl N-(3-Oxocyclobutyl)carbamate. To a 1000-mL 3-necked round-bottom flask was placed a solution of 3-oxocyclobutane-1-carboxylic acid (20 g, 175.29 mmol, 1.00 equiv) in toluene (400 mL), then TEA (19.5 g, 192.71 mmol, 1.10 equiv) and DPPA (53 g, 192.73 mmol, 1.10 equiv) were added. The resulting solution was stirred overnight at 0° C., then washed with saturated sodium bicarbonate aqueous (2×120 mL), H₂O (1×120 mL), and brine (1×60 mL) at 0-10° C. The solution was dried over anhydrous Na₂SO₄ and filtered. To this solution was added t-BuOH (100 mL) and then the reaction was stirred for 16 h at 100° C. The solvent was removed under reduced pressure then the residue was washed with TBME (60 mL) affording 8.3 g (26%) of tert-butyl N-(3-oxocyclobutyl)carbamate as a light white solid. ¹H NMR (400 MHz, CDCl₃) δ 4.94 (brs, 1H), 4.29 (brs, 1H), 3.48-3.36 (m, 2H), 3.13-3.01 (m, 2H), 1.48 (s, 9H).

Step 2: tert-Butyl N-[(1s,3s)-3-Hydroxycyclobutyl]carbamate. To a 250-mL round-bottom flask was placed a solution of tert-butyl N-(3-oxocyclobutyl)carbamate (8.3 g, 44.81 mmol, 1.00 equiv) in THF/H₂O=9:1 (100 mL) then NaBH₄ (830 mg, 22.54 mmol, 0.50 equiv) was added in portions at −70° C. The resulting solution was stirred for 1 h at −50° C. then the reaction was quenched by the addition of water. The mixture was extracted with EtOAc, the organic extracts were combined and the solvent was removed under reduced pressure. The residue was dissolved in 20 mL of toluene at 80° C., then the solution was cooled to RT and stirred for 1 h. The solids were collected by filtration affording 7.56 g (90%) of tert-butyl N-[(1s,3s)-3-hydroxycyclobutyl]carbamate as a white solid. ¹H NMR (400 MHz, CDCl₃) δ 4.67 (brs, 1H), 4.08-4.01 (m, 1H), 3.69-3.66 (m, 1H), 2.82-2.76 (m, 2H), 2.00 (brs, 1H), 1.88-1.75 (m, 2H), 1.46 (s, 9H).

Step 3: (1r,3r)-3-[[(tert-Butoxy)carbonyl]amino]cyclobutyl-4-nitrobenzoate. To a 250-mL 3-necked round-bottom flask purged and maintained with an inert atmosphere of nitrogen was placed a solution of tert-butyl N-[(1s,3s)-3-hydroxycyclobutyl]carbamate (7.56 g, 40.38 mmol, 1.00 equiv) in THF (100 mL), then PPh₃ (15.89 g, 60.58 mmol, 1.50 equiv) and PNBA (7.43 g, 1.10 equiv) were added. This was followed by the addition of DIAD (12.25 g, 60.58 mmol, 1.50 equiv) dropwise with stirring at 0° C. The resulting solution was stirred overnight at RT, then the reaction was quenched by the addition of water and extracted with EtOAc. The organic extracts were combined and then concentrated under reduced pressure. The residue was dissolved in 10 mL of EtOH and stirred for 2 h at RT. The solids were collected by filtration affording 10.8 g (80%) of (1r,3r)-3-[[(tert-butoxy)carbonyl]amino]cyclobutyl 4-nitrobenzoate as a white solid. ¹H NMR (300 MHz, CDCl₃) δ 8.28-8.17 (m, 4H), 5.36-5.32 (m, 1H), 4.77 (brs, 1H), 4.36 (brs, 1H), 2.65-2.56 (m, 2H), 2.47-2.38 (m, 2H), 1.43 (s, 9H).

Step 4: (1r,3r)-3-Aminocyclobutyl 4-nitrobenzoate trifluoroacetic acid salt. To a 100-mL round-bottom flask was placed a solution of (1r,3r)-3-[[(tert-butoxy)carbonyl]amino]cyclobutyl 4-nitrobenzoate (10.8 g, 32.11 mmol, 1.00 equiv) in DCM (25 mL) and TFA (7 mL). The resulting solution was stirred overnight at RT, then the solvent was removed under reduced pressure affording 10.3 g (92%) of (1r,3r)-3-aminocyclobutyl 4-nitrobenzoate trifluoroacetic acid salt as a white solid. ¹H NMR (300 MHz, CD₃OD) δ 8.28-8.25 (m, 4H), 5.52-5.44 (m, 1H), 4.09-4.00 (m, 1H), 2.85-2.62 (m, 4H).

Step 5: (1r,3r)-3-(3-Phenyl-1,2-oxazole-5-amido)cyclobutyl 4-nitrobenzoate. To a 250-mL round-bottom flask was placed a solution of (1r,3r)-3-aminocyclobutyl 4-nitrobenzoate trifluoroacetic acid salt (4 g, 11.42 mmol, 1.00 equiv), DIEA (7.4 g, 57.26 mmol, 5.00 equiv) and 3-phenyl-1,2-oxazole-5-carboxylic acid (2.6 g, 13.74 mmol, 1.20 equiv) in DCM (100 mL). To this solution was added HATU (6.5 g, 17.09 mmol, 1.50 equiv), then the reaction was stirred for 30 min at RT. The reaction was quenched with H₂O and extracted with EtOAc. The organic extracts were combined, washed with brine, dried over anhydrous Na₂SO₄, and concentrated under reduced pressure. The residue was applied onto a silica gel column and eluted with EtOAc/petroleum ether (1:5) affording 4.57 g (98%) of (1r,3r)-3-(3-phenyl-1,2-oxazole-5-amido)cyclobutyl 4-nitrobenzoate as a white solid. LCMS (ES, m/z): [M+H]⁺=408.1.

Step 6: 3-Phenyl-N-[(1r,3r)-3-hydroxycyclobutyl]-1,2-oxazole-5-carboxamide. To a 100-mL round-bottom flask was placed a solution of (1r,3r)-3-(3-phenyl-1,2-oxazole-5-amido)cyclobutyl 4-nitrobenzoate (4.4 g, 10.80 mmol, 1.00 equiv) in MeOH/H₂O=2:1 (30 mL), then K₂CO₃ (4.4 g, 31.83 mmol, 3.00 equiv) was added. The resulting mixture was stirred overnight at 40° C. The reaction was quenched with H₂O and then extracted with EtOAc. The organic extracts were combined, washed with brine, dried over Na₂SO₄, and then concentrated under reduced pressure affording 2.2 g (79%) of 3-phenyl-N-[(1r,3r)-3-hydroxycyclobutyl]-1,2-oxazole-5-carboxamide as a white solid. LCMS (ES, m/z): [M+H]⁺=259.1.

Step 7: 3-Phenyl-N-[(1s,3s)-3-azidocyclobutyl]-1,2-oxazole-5-carboxamide. To a 100-mL round-bottom flask was placed a solution of 3-phenyl-N-[(1r,3r)-3-hydroxycyclobutyl]-1,2-oxazole-5-carboxamide (2.2 g, 8.52 mmol, 1.00 equiv), DPPA (2.8 g, 10.17 mmol, 1.20 equiv) and PPh₃ (3.3 g, 12.58 mmol, 1.50 equiv) in THF (40 mL), then DIAD (2.6 g, 12.86 mmol, 1.50 equiv) was added dropwise. The reaction was stirred for 1 h at 30° C., quenched by the addition of brine, and extracted with EtOAc. The organic extracts were combined, dried over anhydrous Na₂SO₄, and concentrated under reduced pressure. The residue was applied onto a silica gel column and eluted with ethyl acetate/petroleum ether (1:10) affording 860 mg (36%) of 3-phenyl-N-[(1s,3s)-3-azidocyclobutyl]-1,2-oxazole-5-carboxamide as a white solid.

Step 8: 3-Phenyl-N-[(1s,3s)-3-[5-[(1R)-1-hydroxyethyl]-1H-1,2,3-triazol-1-yl]cyclobutyl]-1,2-oxazole-5-carboxamide and 3-Phenyl-N-[(1s,3s)-3-[4-[(1R)-1-hydroxyethyl]-1H-1,2,3-triazol-1-yl]cyclobutyl]-1,2-oxazole-5-carboxamide. To a 10-mL sealed tube was placed a solution of 3-phenyl-N-[(1s,3s)-3-azidocyclobutyl]-1,2-oxazole-5-carboxamide (550 mg, 1.94 mmol, 1.00 equiv) in DMF (2.5 mL), then (2R)-but-3-yn-2-ol (680 mg, 9.70 mmol, 5.00 equiv) was added. The resulting solution was stirred overnight at 100° C. After removing the solvent under reduced pressure, the residue was applied onto a silica gel column with ethyl acetate/petroleum ether (1:3). The resulting mixture was separated by Prep-SFC (Prep SFC80-1: Column, Chiralpak AD-H, 2*25 cm; mobile phase, CO₂ (50%) and ethanol (50%); Detector, UV 220 nm) affording 170.0 mg (25%) of 3-phenyl-N-[(1s,3s)-3-[5-[(1R)-1-hydroxyethyl]-1H-1,2,3-triazol-1-yl]cyclobutyl]-1,2-oxazole-5-carboxamide as a white solid and 222 mg (32%) of 3-phenyl-N-[(1s,3s)-3-[4-[(1R)-1-hydroxyethyl]-1H-1,2,3-triazol-1-yl]cyclobutyl]-1,2-oxazole-5-carboxamide as a white solid.

3-Phenyl-N-[(1s,3s)-3-[5-[(1R)-1-hydroxyethyl]-1H-1,2,3-triazol-1-yl]cyclobutyl]-1,2-oxazole-5-carboxamide: LCMS (ES, m/z): [M+H]⁺=354. ¹H NMR (300 MHz, DMSO-d₆) δ 9.50-9.47 (d, J=7.2 Hz, 1H), 7.94-7.90 (m, 2H), 7.66 (s, 1H), 7.61 (s, 1H), 7.56-7.54 (m, 3H), 5.52-5.50 (d, J=6.0 Hz, 1H), 4.95-4.85 (m, 2H), 4.45-4.31 (m, 1H), 2.94-2.80 (m, 4H), 1.45-1.43 (d, J=6.6 Hz, 3H). Purity (HPLC, 254 nm): 99.4%.

3-Phenyl-N-[(1s,3s)-3-[4-[(1R)-1-hydroxyethyl]-1H-1,2,3-triazol-1-yl]cyclobutyl]-1,2-oxazole-5-carboxamide: LCMS (ES, m/z): [M+H]⁺=354. ¹H NMR (300 MHz, DMSO-d₆) δ 9.41-9.39 (d, J=8.4 Hz, 1H), 8.13 (s, 1H), 7.96-7.93 (m, 2H), 7.68 (s, 1H), 7.56-7.54 (m, 3H), 5.30-5.28 (d, J=4.8 Hz, 1H), 5.00-4.80 (m, 2H), 4.48-4.35 (m, 1H), 2.98-2.89 (m, 2H), 2.74-2.64 (m, 2H), 1.43-1.41 (d, J=6.6 Hz, 3H).

### Examples 53 and 54: 3-Phenyl-N-[(1s,3s)-3-[5-[(1S)-1-hydroxyethyl]-1H-1,2,3-triazol-1-yl]cyclobutyl]-1,2-oxazole-5-carboxamide and 3-Phenyl-N-[(1s,3s)-3-[4-[(1S)-1-hydroxyethyl]-1H-1,2,3-triazol-1-yl]cyclobutyl]-1,2-oxazole-5-carboxamide

Step 1: 3-Phenyl-N-[(1s,3s)-3-[5-[(1S)-1-hydroxyethyl]-1H-1,2,3-triazol-1-yl]cyclobutyl]-1,2-oxazole-5-carboxamide and 3-Phenyl-N-[(1s,3s)-3-[4-[(1S)-1-hydroxyethyl]-1H-1,2,3-triazol-1-yl]cyclobutyl]-1,2-oxazole-5-carboxamide. To a 10-mL sealed tube, was placed a solution of 3-phenyl-N-[(1s,3s)-3-azidocyclobutyl]-1,2-oxazole-5-carboxamide (500 mg, 1.77 mmol, 1.00 equiv) in DMF (2.5 mL), then (2S)-but-3-yn-2-ol (618 mg, 8.82 mmol, 5.00 equiv) was added. The reaction was stirred overnight at 100° C. then concentrated under reduced pressure. The residue was applied onto a silica gel column with EtOAc/petroleum ether (1:3). The resulting mixture was separated by Prep-SFC (Prep SFC80-1: Column, Chiralpak AD-H, 2*25 cm; mobile phase, CO2(55%) and methanol(45%); Detector, UV 220 nm) affording 106.1 mg (17%) of 3-phenyl-N-[(1s,3s)-3-[5-[(1S)-1-hydroxyethyl]-1H-1,2,3-triazol-1-yl]cyclobutyl]-1,2-oxazole-5-carboxamide as a white solid and 192.2 mg (31%) of 3-phenyl-N-[(1s,3s)-3-[4-[(1S)-1-hydroxyethyl]-1H-1,2,3-triazol-1-yl]cyclobutyl]-1,2-oxazole-5-carboxamide as a white solid.

3-Phenyl-N-[(1s,3s)-3-[5-[(1S)-1-hydroxyethyl]-1H-1,2,3-triazol-1-yl]cyclobutyl]-1,2-oxazole-5-carboxamide: LCMS (ES, m/z): [M+H]⁺=354. ¹H NMR (300 MHz, DMSO-d₆) δ 9.50-9.47 (d, J=7.5 Hz, 1H), 7.94-7.90 (m, 2H), 7.66 (s, 1H), 7.61 (s, 1H), 7.56-7.54 (m, 3H), 5.52-5.50 (d, J=6.0 Hz, 1H), 4.95-4.85 (m, 2H), 4.45-4.31 (m, 1H), 2.94-2.80 (m, 4H), 1.45-1.43 (d, J=6.3 Hz, 3H). Purity (HPLC, 254 nm): 96.0%.

3-Phenyl-N-[(1s,3s)-3-[4-[(1S)-1-hydroxyethyl]-1H-1,2,3-triazol-1-yl]cyclobutyl]-1,2-oxazole-5-carboxamide: LCMS (ES, m/z): [M+H]⁺=354. ¹H NMR (300 MHz, DMSO-d₆) δ 9.41-9.39 (d, J=8.1 Hz, 1H), 8.13 (s, 1H), 7.96-7.93 (m, 2H), 7.68 (s, 1H), 7.56-7.54 (m, 3H), 5.30-5.28 (d, J=4.5 Hz, 1H), 5.00-4.92 (m, 1H), 4.88-4.80 (m, 1H), 4.48-4.35 (m, 1H), 2.98-2.89 (m, 2H), 2.74-2.50 (m, 2H), 1.43-1.41 (d, J=6.6 Hz, 3H). Purity (HPLC, 254 nm): 97.7%.

### Examples 55 and 56: 3-Phenyl-N-[(1s,3s)-3-([5-[(1R)-1-hydroxyethyl]-1H-1,2,3-triazol-1-yl]methyl)cyclobutyl]-1,2-oxazole-5-carboxamide and 3-Phenyl-N-[(1s,3s)-3-([4-[(1R)-1-hydroxyethyl]-1H-1,2,3-triazol-1-yl]methyl)cyclobutyl]-1,2-oxazole-5-carboxamide

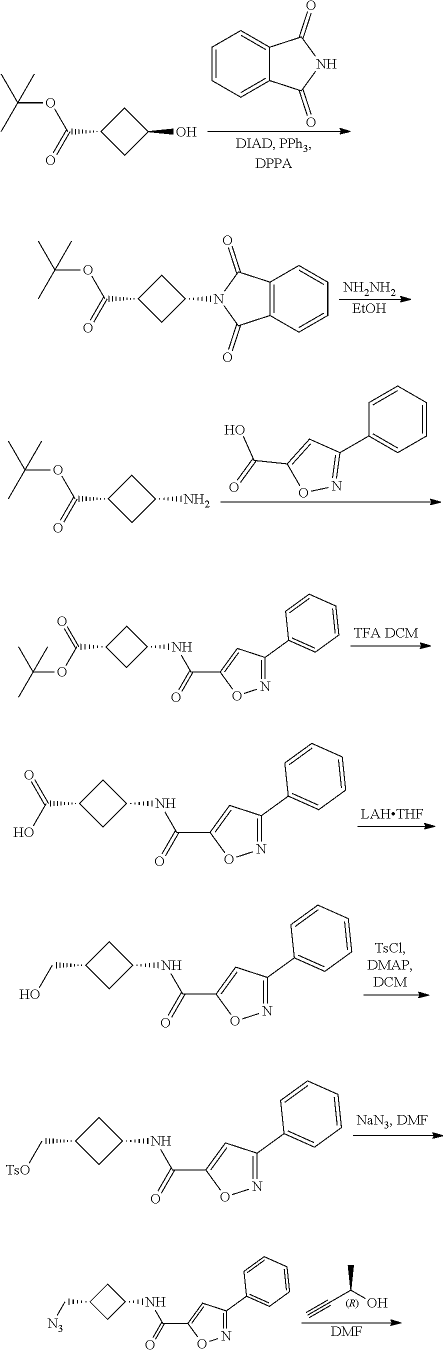

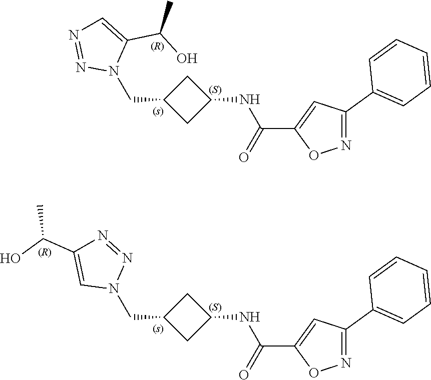

Step 1: tert-Butyl (1s,3s)-3-(1,3-Dioxo-2,3-dihydro-1H-isoindol-2-yl)cyclobutane-1-carboxylate. To a 250-mL 3-necked round-bottom flask was placed a solution of tert-butyl (1r,3r)-3-hydroxycyclobutane-1-carboxylate (1.1 g, 5.87 mmol, 1.00 equiv), 2,3-dihydro-1H-isoindole-1,3-dione (1.04 g, 7.07 mmol, 1.19 equiv), and PPh₃ (2.5 g) in THF (60 mL). This was followed by the addition of DIAD (300 mg) dropwise with stirring at 0° C. The resulting solution was stirred for 1 h at RT then the reaction was quenched by the addition of 50 mL of water. The resulting solution was extracted with EtOAc (3×50 mL) and the organic layers combined. The resulting mixture was washed with brine (2×50 mL), dried over anhydrous Na₂SO₄, and concentrated under reduced pressure. The residue was applied onto a silica gel column and eluted with EtOAc/petroleum ether (1:40) affording 810 mg of tert-butyl (1s,3s)-3-(1,3-dioxo-2,3-dihydro-1H-isoindol-2-yl)cyclobutane-1-carboxylate as a white solid. LCMS (ES, m/z): [M+H]⁺=302.2.

Step 2: tert-Butyl (1s,3s)-3-Aminocyclobutane-1-carboxylate. To a 2000-mL round-bottom flask was placed a solution of tert-butyl (1s,3s)-3-(1,3-dioxo-2,3-dihydro-1H-isoindol-2-yl)cyclobutane-1-carboxylate (810 mg, 2.64 mmol, 1.00 equiv) in EtOH (50 mL) and then N₂H₄.H₂O (400 mg, 3.00 equiv) was added. The resulting solution was stirred for 4 h at RT, then the solids were removed by filtration. The filtrate was concentrated under reduced pressure affording 500 mg of crude tert-butyl (1s,3s)-3-aminocyclobutane-1-carboxylate as light yellow oil. LCMS [M+H]⁺=172.1

Step 3: tert-Butyl (1s,3s)-3-(3-Phenyl-1,2-oxazole-5-amido)cyclobutane-1-carboxylate. To a 100-mL round-bottom flask was placed a solution of tert-butyl (1s,3s)-3-aminocyclobutane-1-carboxylate (1.7 g, 9.93 mmol, 1.00 equiv) in DCM (50 mL), then 3-phenyl-1,2-oxazole-5-carboxylic acid (1.9 g, 10.04 mmol, 1.00 equiv), HATU (5.7 g, 14.99 mmol, 1.50 equiv) and DIEA (3.9 g, 30.18 mmol, 3.00 equiv) were added. The resulting solution was stirred for 1 h at RT, then quenched by the addition of water and extracted with EtOAc. The organic extracts were combined, washed with brine, dried over anhydrous Na₂SO₄, and concentrated under reduced pressure. The residue was applied onto a silica gel column and eluted with EtOAc/petroleum ether (1:7) affording 2 g (59%) of tert-butyl (1s,3s)-3-(3-phenyl-1,2-oxazole-5-amido)cyclobutane-1-carboxylate as a white solid. LCMS (ES, m/z): [M+H]⁺=343.2.

Step 4: (1s,3s)-3-(3-Phenyl-1,2-oxazole-5-amido)cyclobutane-1-carboxylic acid. To a 25-mL round-bottom flask was placed a solution of tert-butyl (1s,3s)-3-(3-phenyl-1,2-oxazole-5-amido)cyclobutane-1-carboxylate (830 mg, 2.42 mmol, 1.00 equiv) in DCM (10 mL) and TFA (3 mL). The resulting solution was stirred for 2 h at rt, then the reaction was concentrated under reduced pressure affording 680 mg (98%) of (1s,3s)-3-(3-phenyl-1,2-oxazole-5-amido)cyclobutane-1-carboxylic acid as a light yellow solid.

Step 5: 3-Phenyl-N-[(1s,3s)-3-(hydroxymethyl)cyclobutyl]-1,2-oxazole-5-carboxamide. To a 100-mL 3-necked round-bottom flask was placed a solution of (1s,3s)-3-(3-phenyl-1,2-oxazole-5-amido)cyclobutane-1-carboxylic acid (1.2 g, 4.19 mmol, 1.00 equiv) in THF (50 mL) followed by the addition of LiAlH₄ (319 mg, 8.41 mmol, 2.00 equiv) in portions at 0° C. over 5 min. The resulting solution was stirred for 2 h at RT, then quenched by the addition of 100 mL of 2N HCl, and extracted with EtOAc (2×100 mL). The organic extracts were combined, washed with brine (2×100 mL), dried over anhydrous Na₂SO₄, and concentrated under reduced pressure affording 860 mg (75%) of 3-phenyl-N-[(1s,3s)-3-(hydroxymethyl)cyclobutyl]-1,2-oxazole-5-carboxamide as a light yellow solid. LCMS (ES, m/z): [M+H]⁺=273.1.

Step 6: [(1s,3s)-3-(3-Phenyl-1,2-oxazole-5-amido)cyclobutyl]methyl 4-methylbenzene-1-sulfonate. To a 50-mL round-bottom flask was placed a solution of 3-phenyl-N-[(1s,3s)-3-(hydroxymethyl)cyclobutyl]-1,2-oxazole-5-carboxamide (860 mg, 3.16 mmol, 1.00 equiv) in DCM (20 mL) then DMAP (781 mg, 6.39 mmol, 2.00 equiv) and TsCl (779 mg, 4.09 mmol, 1.30 equiv) were added. The resulting solution was stirred for 16 h at RT, diluted with 100 mL of H₂O, and extracted with EtOAc (2×100 mL). The organic extracts were combined, washed with brine (2×100 mL), dried over anhydrous Na₂SO₄, and concentrated under reduced pressure affording 1.1 g (82%) of [(1s,3s)-3-(3-phenyl-1,2-oxazole-5-amido)cyclobutyl]methyl 4-methylbenzene-1-sulfonate as a yellow solid. LCMS (ES, m/z): [M+H]⁺=427.2.

Step 7: 3-Phenyl-N-[(1s,3s)-3-(azidomethyl)cyclobutyl]-1,2-oxazole-5-carboxamide. To a 25-mL round-bottom flask was placed a solution of [(1s,3s)-3-(3-phenyl-1,2-oxazole-5-amido)cyclobutyl]methyl 4-methylbenzene-1-sulfonate (1.1 g, 2.58 mmol, 1.00 equiv) in DMF (10 mL), then NaN₃ (254 mg, 3.91 mmol, 1.50 equiv) was added. The resulting solution was stirred for 1 h at 80° C., diluted with 100 mL of H₂O, and extracted with EtOAc (2×100 mL). The organic extracts were combined, washed with brine (5×100 mL), dried over anhydrous Na₂SO₄, and concentrated under reduced pressure affording 750 mg (98%) of 3-phenyl-N-[(1s,3s)-3-(azidomethyl)cyclobutyl]-1,2-oxazole-5-carboxamide as a yellow solid.

Step 8: 3-Phenyl-N-[(1s,3s)-3-([5-[(1R)-1-hydroxyethyl]-1H-1,2,3-triazol-1-yl]methyl)cyclobutyl]-1,2-oxazole-5-carboxamide and 3-Phenyl-N-[(1s,3s)-3-([4-[(1R)-1-hydroxyethyl]-1H-1,2,3-triazol-1-yl]methyl)cyclobutyl]-1,2-oxazole-5-carboxamide. To a 25-mL round-bottom flask was placed a solution of 3-phenyl-N-[(1s,3s)-3-(azidomethyl)cyclobutyl]-1,2-oxazole-5-carboxamide (350 mg, 1.18 mmol, 1.00 equiv) in DMF (5 mL), then (2R)-but-3-yn-2-ol (420 mg, 5.99 mmol, 5.00 equiv) was added. The resulting solution was stirred for 16 h at 80° C., then diluted with 50 mL of H₂O, and extracted with EtOAc (3×50 mL). The organic extracts were combined, washed with brine (3×50 mL), dried over anhydrous Na₂SO₄, and concentrated under reduced pressure. The residue was applied onto a silica gel column and eluted with EtOAc/petroleum ether (5:1). The pure isomers were separated by Chiral-Prep-HPLC (Prep-HPLC-009: Column, Chiralpak IB, 2*25 cm, 5 um; mobile phase, Hex and ethanol (hold 15.0% ethanol in 29 min); Detector, UV 254/220 nm) affording 29.4 mg (7%) of 3-phenyl-N-[(1s,3s)-3-([5-[(1R)-1-hydroxyethyl]-1H-1,2,3-triazol-1-yl]methyl)cyclobutyl]-1,2-oxazole-5-carboxamide as a light yellow solid and 31.6 mg (7%) of 3-phenyl-N-[(1s,3s)-3-([4-[(1R)-1-hydroxyethyl]-1H-1,2,3-triazol-1-yl]methyl)cyclobutyl]-1,2-oxazole-5-carboxamide as a white solid.

3-Phenyl-N-[(1s,3s)-3-([5-[(1R)-1-hydroxyethyl]-1H-1,2,3-triazol-1-yl]methyl)cyclobutyl]-1,2-oxazole-5-carboxamide: LCMS (ES, m/z): [M+H]⁺=368.1. ¹H NMR (300 MHz, DMSO-d₆) δ 9.25-9.22 (d, J=7.8 Hz, 1H), 7.94-7.91 (m, 2H), 7.63-7.60 (d, J=6.9 Hz, 2H), 7.55-7.53 (m, 3H), 5.53-7.51 (d, J=6.0 Hz, 1H), 4.93-4.85 (m, 1H), 4.43-4.40 (d, J=7.2 Hz, 2H), 4.35-4.27 (m, 1H), 2.64-2.55 (m, 1H), 2.39-2.30 (m, 2H), 2.03-1.94 (m, 2H), 1.48-1.46 (d, J=6.3 Hz, 3H). Purity (HPLC, 254 nm): 95.2%.

3-Phenyl-N-[(1s,3s)-3-([4-[(1R)-1-hydroxyethyl]-1H-1,2,3-triazol-1-yl]methyl)cyclobutyl]-1,2-oxazole-5-carboxamide: LCMS (ES, m/z): [M+H]⁺=368.1. ¹H NMR (300 MHz, DMSO-d₆) δ 9.22-9.19 (d, J=7.8 Hz, 1H), 7.90-7.76 (m, 2H), 7.84 (s, 1H), 7.59 (s, 1H), 7.55-7.46 (m, 3H), 5.19-5.18 (d, J=4.5 Hz, 1H), 4.80-4.76 (m, 1H), 4.35-4.24 (m, 3H), 2.60-2.50 (m, 1H), 2.33-2.25 (m, 2H), 1.95-1.88 (m, 2H), 1.37-1.35 (d, J=6.3 Hz, 3H). Purity (HPLC, 254 nm): 95.0%.

### Examples 57 and 58: 3-Phenyl-N-[(1s,3s)-3-([5-[(1S)-1-hydroxyethyl]-1H-1,2,3-triazol-1-yl]methyl)cyclobutyl]-1,2-oxazole-5-carboxamide and 3-Phenyl-N-[(1s,3s)-3-([4-[(1S)-1-hydroxyethyl]-1H-1,2,3-triazol-1-yl]methyl)cyclobutyl]-1,2-oxazole-5-carboxamide

Into a 25-mL round-bottom flask, was placed a solution of 3-phenyl-N-[(1s,3s)-3-(azidomethyl)cyclobutyl]-1,2-oxazole-5-carboxamide (270 mg, 0.91 mmol, 1.00 equiv) in toluene (5 mL), then (2S)-but-3-yn-2-ol (315 mg, 4.49 mmol, 5.00 equiv) was added. The resulting solution was stirred for 16 h at 100° C. and then concentrated under reduced pressure. The crude product was purified by Prep-TLC (petroleum ether/EtOAc=1:5). The resulting mixture was separated by Chiral-Prep-HPLC (2#-Gilson Gx 281(HPLC-09): Column: Chiralpak IB, 2*25 cm, 5 um; Mobile Phase A: hexane, Mobile Phase B: EtOH; Flow rate: 20 m/min; Gradient: 30 B to 30 B in 15 min; 254/220 nm; RT1:7.642; RT2:10.588) affording 32.8 mg (10%) of 3-phenyl-N-[(1s,3s)-3-([5-[(1S)-1-hydroxyethyl]-1H-1,2,3-triazol-1-yl]methyl)cyclobutyl]-1,2-oxazole-5-carboxamide as a white solid and 68.5 mg (21%) of 3-phenyl-N-[(1s,3s)-3-([4-[(1S)-1-hydroxyethyl]-1H-1,2,3-triazol-1-yl]methyl)cyclobutyl]-1,2-oxazole-5-carboxamide as a white solid.

3-Phenyl-N-[(1s,3s)-3-([5-[(1S)-1-hydroxyethyl]-1H-1,2,3-triazol-1-yl]methyl)cyclobutyl]-1,2-oxazole-5-carboxamide: LCMS (ES, m/z): [M+H]⁺=368.2. ¹H NMR (400 MHz, DMSO-d₆) δ 9.24-9.22 (d, J=7.6 Hz, 1H), 7.94-7.92 (m, 2H), 7.62-7.60 (d, J=8.4 Hz, 1H), 7.55-7.53 (m, 3H), 5.52-5.50 (d, J=6.0 Hz, 1H), 4.95-4.83 (m, 1H), 4.43-4.40 (m, 2H), 4.35-4.31 (m, 3H), 2.54-2.52 (m, 1H), 2.36-2.33 (m, 2H), 2.05-1.98 (m, 2H), 1.48-1.46 (d, J=6.4 Hz, 3H). Purity (HPLC, 254 nm): 93.1%.

3-Phenyl-N-[(1s,3s)-3-([4-[(1S)-1-hydroxyethyl]-1H-1,2,3-triazol-1-yl]methyl)cyclobutyl]-1,2-oxazole-5-carboxamide: LCMS (ES, m/z): [M+H]⁺=368.2. ¹H NMR (400 MHz, DMSO-d₆) δ 9.24-9.22 (d, J=7.6 Hz, 1H), 7.94-7.92 (m, 2H), 7.87 (s, 1H), 7.62 (s, 1H), 7.55-7.53 (m, 3H), 5.22-5.21 (d, J=4.8 Hz, 1H), 4.85-4.79 (m, 1H), 4.38-4.37 (d, J=7.2 Hz, 2H), 4.34-4.28 (m, 1H), 2.54-2.46 (m, 1H), 2.39-2.32 (m, 2H), 2.01-1.93 (m, 2H), 1.41-1.39 (d, J=6.4 Hz, 3H). Purity (HPLC, 254 nm): 98.6%.

### Examples 59 and 60: 3-Phenyl-N-[(1r,3r)-3-([5-[(1R)-1-hydroxyethyl]-1H-1,2,3-triazol-1-yl]methyl)cyclobutyl]-1,2-oxazole-5-carboxamide and 3-Phenyl-N-[(1r,3r)-3-([4-[(1R)-1-hydroxyethyl]-1H-1,2,3-triazol-1-yl]methyl)cyclobutyl]-1,2-oxazole-5-carboxamide

Step 1: 3-Phenyl-N-[(1r,3r)-3-(azidomethyl)cyclobutyl]-1,2-oxazole-5-carboxamide. To a 25-mL round-bottom flask was placed a solution of [(1r,3r)-3-(3-phenyl-1,2-oxazole-5-amido)cyclobutyl]methyl 4-methylbenzene-1-sulfonate (920 mg, 2.16 mmol, 1.00 equiv) in DMF (10 mL), then NaN₃ (169 mg, 2.60 mmol, 1.20 equiv) was added. The resulting solution was stirred for 2 h at 80° C., then diluted with 100 mL of H₂O, and extracted with EtOAc (2×50 mL). The organic extracts were combined, washed with brine (2×50 mL), dried over anhydrous Na₂SO₄, and concentrated under reduced pressure affording 600 mg (94%) of 3-phenyl-N-[(1r,3r)-3-(azidomethyl)cyclobutyl]-1,2-oxazole-5-carboxamide as a light yellow solid.

Step 2: 3-Phenyl-N-[(1r,3r)-3-([5-[(1R)-1-hydroxyethyl]-1H-1,2,3-triazol-1-yl]methyl)cyclobutyl]-1,2-oxazole-5-carboxamide and 3-phenyl-N-[(1r,3r)-3-([4-[(1R)-1-hydroxyethyl]-1H-1,2,3-triazol-1-yl]methyl)cyclobutyl]-1,2-oxazole-5-carboxamide. To a 5-mL round-bottom flask was placed a solution of 3-phenyl-N-[(1r,3r)-3-(azidomethyl)cyclobutyl]-1,2-oxazole-5-carboxamide (300 mg, 1.01 mmol, 1.00 equiv) in DMF (5 mL), then (2R)-but-3-yn-2-ol (210 mg, 3.00 mmol, 3.00 equiv) was added. The resulting solution was stirred for 16 h at 100° C., then diluted with 50 mL of H₂O, and extracted with EtOAc (2×50 mL). The organic extracts were combined, washed with brine (2×50 mL), dried over anhydrous Na₂SO₄, and concentrated under reduced pressure. The crude product was purified by Prep-TLC (petroleum ether/ethyl acetate=1:5). The resulting mixture was separated by Chiral-Prep-HPLC (Prep-HPLC-004: Column, Chiralpak IA, 2*25 cm, 5 um; mobile phase, Hex and IPA (hold 30.0% IPA in 15 min); Detector, UV 254/220 nm) affording 103.5 mg (28%) of 3-phenyl-N-[(1r,3r)-3-([5-[(1R)-1-hydroxyethyl]-1H-1,2,3-triazol-1-yl]methyl)cyclobutyl]-1,2-oxazole-5-carboxamide as a white solid and 127.1 mg (38%) of 3-phenyl-N-[(1r,3r)-3-([4-[(1R)-1-hydroxyethyl]-1H-1,2,3-triazol-1-yl]methyl)cyclobutyl]-1,2-oxazole-5-carboxamide as a light yellow solid.

3-Phenyl-N-[(1r,3r)-3-([5-[(1R)-1-hydroxyethyl]-1H-1,2,3-triazol-1-yl]methyl)cyclobutyl]-1,2-oxazole-5-carboxamide: LCMS (ES, m/z): [M+H]⁺=368.2. ¹H NMR (400 MHz, DMSO-d₆) δ 9.29-9.27 (d, J=7.2 Hz, 1H), 7.92-7.90 (m, 2H), 7.62-7.59 (m, 2H), 7.53-7.52 (m, 3H), 5.53-5.52 (d, J=6.0 Hz, 1H), 4.92-4.89 (m, 1H), 4.62-4.58 (m, 1H), 4.56-4.48 (m, 2H), 2.85-2.81 (m, 1H), 2.27-2.17 (m, 4H), 1.46-1.44 (d, J=6.4 Hz, 3H). Purity (HPLC, 254 nm): 95.0%.

3-Phenyl-N-[(1r,3r)-3-([4-[(1R)-1-hydroxyethyl]-1H-1,2,3-triazol-1-yl]methyl)cyclobutyl]-1,2-oxazole-5-carboxamide: LCMS (ES, m/z): [M+H]⁺=368.2. ¹H NMR (400 MHz, DMSO-d₆) δ 9.27-9.25 (d, J=7.2 Hz, 1H), 7.94 (s, 1H), 7.93-7.89 (m, 2H), 7.62 (s, 1H), 7.53-7.51 (m, 3H), 5.21-5.20 (d, J=4.8 Hz, 1H), 4.83-4.80 (m, 1H), 4.59-4.51 (m, 1H), 4.48-4.46 (d, J=7.6 Hz, 2H), 2.74-2.66 (m, 1H), 2.28-2.21 (m, 2H), 2.17-2.11 (m, 2H), 1.39-1.37 (d, J=6.8 Hz, 3H). Purity (HPLC, 254 nm): 96.9%.

### Examples 61 and 62: 3-Phenyl-N-[(1r,3r)-3-([5-[(1S)-1-hydroxyethyl]-1H-1,2,3-triazol-1-yl]methyl)cyclobutyl]-1,2-oxazole-5-carboxamide and 3-Phenyl-N-[(1r,3r)-3-([4-[(1S)-1-hydroxyethyl]-1H-1,2,3-triazol-1-yl]methyl)cyclobutyl]-1,2-oxazole-5-carboxamide

To a 25-mL round-bottom flask was placed a solution of 3-phenyl-N-[(1r,3r)-3-(azidomethyl)cyclobutyl]-1,2-oxazole-5-carboxamide (210 mg, 0.71 mmol, 1.00 equiv) in toluene (5 mL), then (2S)-but-3-yn-2-ol (245 mg, 3.50 mmol, 5.00 equiv) was added. The resulting solution was stirred for 16 h at 100° C., then concentrated under reduced pressure. The crude product was purified by Prep-TLC (petroleum ether/EtOAc=1:5). The resulting mixture was separated by Chiral-Prep-HPLC (Prep-HPLC-004: Column, Chiralpak IC, 2*25 cm, 5 um; mobile phase, Hex and ethanol (hold 50.0% ethanol in 15 min); Detector, UV 254/220 nm) affording 44.2 mg (17%) of 3-phenyl-N-[(1r,3r)-3-([5-[(1S)-1-hydroxyethyl]-1H-1,2,3-triazol-1-yl]methyl)cyclobutyl]-1,2-oxazole-5-carboxamide as a white solid and 78.5 mg (30%) of 3-phenyl-N-[(1r,3r)-3-([4-[(1S)-1-hydroxyethyl]-1H-1,2,3-triazol-1-yl]methyl)cyclobutyl]-1,2-oxazole-5-carboxamide as a white solid.

3-Phenyl-N-[(1r,3r)-3-([5-[(1S)-1-hydroxyethyl]-1H-1,2,3-triazol-1-yl]methyl)cyclobutyl]-1,2-oxazole-5-carboxamide: LCMS (ES, m/z): [M+H]⁺=368.2. ¹H NMR (400 MHz, DMSO-d₆) δ 9.31-9.29 (d, J=7.2 Hz, 1H), 7.94-7.91 (m, 2H), 7.64-7.60 (m, 2H), 7.55-7.52 (m, 3H), 5.55-5.53 (d, J=6.0 Hz, 1H), 4.95-4.89 (m, 1H), 4.64-4.47 (m, 3H), 2.88-2.82 (m, 1H), 2.30-2.19 (m, 4H), 1.48-1.46 (d, J=6.4 Hz, 3H). Purity (HPLC, 254 nm): 97.5%.

3-Phenyl-N-[(1r,3r)-3-([4-[(1S)-1-hydroxyethyl]-1H-1,2,3-triazol-1-yl]methyl)cyclobutyl]-1,2-oxazole-5-carboxamide: LCMS (ES, m/z): [M+H]⁺=368.2. ¹H NMR (400 MHz, DMSO-d₆) δ 9.30-9.28 (d, J=7.6 Hz, 1H), 7.96 (s, 1H), 7.94-7.91 (m, 2H), 7.64 (s, 1H), 7.55-7.53 (m, 3H), 5.23-5.21 (d, J=4.8 Hz, 1H), 4.85-4.79 (m, 1H), 4.58-4.53 (m, 1H), 4.50-4.48 (d, J=7.6 Hz, 2H), 2.75-2.71 (m, 1H), 2.30-2.23 (m, 2H), 2.18-2.12 (m, 2H), 1.41-1.39 (d, J=6.4 Hz, 3H). Purity (HPLC, 254 nm): 99.2%.

### Examples 63 and 64: 3-Phenyl-N-[(1r,3r)-3-([3-[(1R)-1-hydroxyethyl]-1H-pyrazol-1-yl]methyl)cyclobutyl]-1,2-oxazole-5-carboxamide and 3-Phenyl-N-[(1r,3r)-3-([3-[(1S)-1-hydroxyethyl]-1H-pyrazol-1-yl]methyl)cyclobutyl]-1,2-oxazole-5-carboxamide

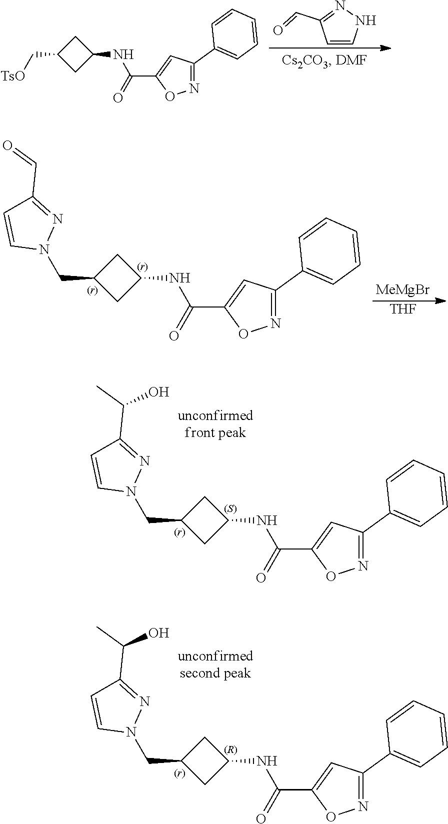

Step 1: 3-Phenyl-N-[(1r,3r)-3-[(3-formyl-1H-pyrazol-1-yl)methyl]cyclobutyl]-1,2-oxazole-5-carboxamide. To a 50-mL round-bottom flask was placed a solution of [(1r,3r)-3-(3-phenyl-1,2-oxazole-5-amido)cyclobutyl]methyl 4-methylbenzene-1-sulfonate (1.28 g, 3.00 mmol, 1.00 equiv) in DMF (20 mL), then Cs₂CO₃ (1.95 g, 5.98 mmol, 2.00 equiv) and 1H-pyrazole-3-carbaldehyde (432 mg, 4.50 mmol, 1.50 equiv) were added. The resulting solution was stirred for 3 h at 100° C., then the solids were removed by filtration. The filtrate was purified by Flash-Prep-HPLC (CombiFlash-1: Column, C18; mobile phase, X: H₂O (0.5% NH₄HCO₃), Y: CAN, X/Y=90/10 increasing to X/Y=5/95 within 40 min; Detector, UV 254 nm) affording 450 mg (43%) of 3-phenyl-N-[(1r,3r)-3-[(3-formyl-1H-pyrazol-1-yl)methyl]cyclobutyl]-1,2-oxazole-5-carboxamide as a yellow solid. LCMS (ES, m/z): [M+H]⁺=351.2.

Step 2: 3-Phenyl-N-[(1r,3r)-3-([3-[(1R)-1-hydroxyethyl]-1H-pyrazol-1-yl]methyl)cyclobutyl]-1,2-oxazole-5-carboxamide (front peak) and 3-phenyl-N-[(1r,3r)-3-([3-[(1S)-1-hydroxyethyl]-1H-pyrazol-1-yl]methyl)cyclobutyl]-1,2-oxazole-5-carboxamide (second peak). To a 50-mL 3-necked round-bottom flask was placed a solution of 3-phenyl-N-[(1r,3r)-3-[(3-formyl-1H-pyrazol-1-yl)methyl]cyclobutyl]-1,2-oxazole-5-carboxamide (450 mg, 1.28 mmol, 1.00 equiv) in THF (20 mL). The solution was cooled to 0° C., then MeMgBr (1.3 mL, 3.00 equiv, 3 mol/L) was added dropwise with stirring at 0° C. over 10 min. The reaction was stirred for 2 h at RT, then quenched by the addition of 10 mL of 2N HCl and 50 mL of H₂O, and extracted with EtOAc (3×50 mL). The organic extracts were combined, washed with brine (3×50 mL), dried over anhydrous Na₂SO₄, and concentrated under reduced pressure. The residue was applied onto a silica gel column and eluted with EtOAc/petroleum ether (2:1). The resulting mixture was separated by Chiral-Prep-HPLC (Prep-HPLC-004: Column, Chiralpak IC, 2*25 cm, 5 um; mobile phase, Hex and ethanol (hold 50.0% ethanol in 13 min); Detector, UV 254/220 nm) affording 126.1 mg (27%) of 3-phenyl-N-[(1r,3r)-3-([3-[(1R)-1-hydroxyethyl]-1H-pyrazol-1-yl]methyl)cyclobutyl]-1,2-oxazole-5-carboxamide (front peak) as a light yellow solid and 136.9 mg (29%) of 3-phenyl-N-[(1r,3r)-3-([3-[(1S)-1-hydroxyethyl]-1H-pyrazol-1-yl]methyl)cyclobutyl]-1,2-oxazole-5-carboxamide (second peak) as a white solid.

3-Phenyl-N-[(1r,3r)-3-([3-[(1R)-1-hydroxyethyl]-1H-pyrazol-1-yl]methyl)cyclobutyl]-1,2-oxazole-5-carboxamide: LCMS (ES, m/z): [M+H]⁺=367.0. ¹H NMR (300 MHz, DMSO-d₆) δ 9.28-9.25 (d, J=7.2 Hz, 1H), 7.94-7.91 (m, 2H), 7.65-7.63 (m, 2H), 7.55-7.53 (m, 3H), 6.15-6.14 (d, J=1.8 Hz, 1H), 4.95-4.93 (d, J=4.8 Hz, 1H), 4.72-4.64 (m, 1H), 4.58-4.45 (m, 1H), 4.19-4.16 (d, J=7.8 Hz, 2H), 2.72-2.64 (m, 1H), 2.27-2.12 (m, 4H), 1.34-1.32 (d, J=6.3 Hz, 3H). Purity (HPLC, 254 nm): 98.9%.

3-Phenyl-N-[(1r,3r)-3-([3-[(1S)-1-hydroxyethyl]-1H-pyrazol-1-yl]methyl)cyclobutyl]-1,2-oxazole-5-carboxamide: LCMS (ES, m/z): [M+H]⁺=367.0. ¹H NMR (300 MHz, DMSO-d₆) δ 9.28-9.25 (d, J=7.5 Hz, 1H), 7.94-7.91 (m, 2H), 7.65-7.63 (m, 2H), 7.55-7.53 (m, 3H), 6.15-6.14 (d, J=2.1 Hz, 1H), 4.95-4.93 (d, J=5.1 Hz, 1H), 4.72-4.63 (m, 1H), 4.55-4.48 (m, 1H), 4.19-4.16 (d, J=7.5 Hz, 2H), 2.69-2.64 (m, 1H), 2.27-2.11 (m, 4H), 1.34-1.32 (d, J=6.3 Hz, 3H). Purity (HPLC, 254 nm): 98.3%.

### Examples 65 and 66: 3-Phenyl-N-[(1s,3s)-3-([3-[(1S)-1-hydroxyethyl]-1H-pyrazol-1-yl]methyl)cyclobutyl]-1,2-oxazole-5-carboxamide (Front Peak) and 3-Phenyl-N-[(1s,3s)-3-([3-[(1R)-1-hydroxyethyl]-1H-pyrazol-1-yl]methyl)cyclobutyl]-1,2-oxazole-5-carboxamide (Second Peak)

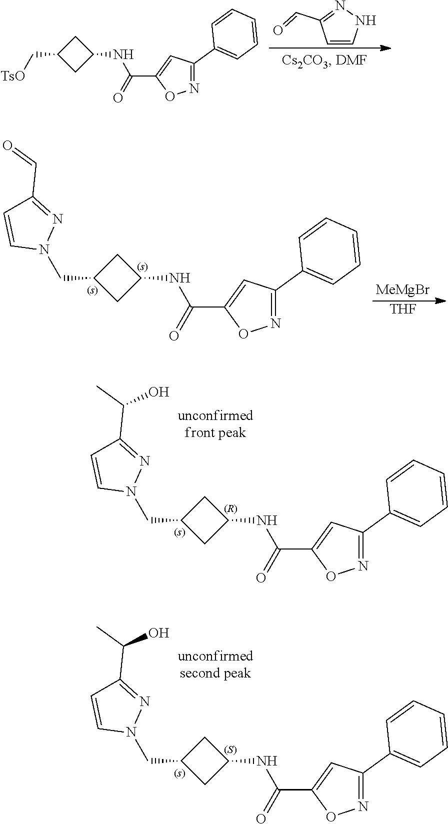

Step 1: 3-Phenyl-N-[(1s,3s)-3-[(3-formyl-1H-pyrazol-1-yl)methyl]cyclobutyl]-1,2-oxazole-5-carboxamide. To a 50-mL round-bottom flask was placed a solution of [(1s,3s)-3-(3-phenyl-1,2-oxazole-5-amido)cyclobutyl]methyl 4-methylbenzene-1-sulfonate (1.3 g, 3.05 mmol, 1.00 equiv) in DMF (15 mL), then Cs₂CO₃ (1.96 g, 6.02 mmol, 2.00 equiv) and 1H-pyrazole-3-carbaldehyde (432 mg, 4.50 mmol, 1.50 equiv) were added. The resulting solution was stirred for 3 h at 100° C., then the solids were removed by filtration. The filtrate was purified by Flash-Prep-HPLC (CombiFlash-1: Column, C18; mobile phase, X: H₂O (0.5% NH₄HCO₃), Y: ACN, X/Y=90/10 increasing to X/ACN=5/95 within 40 min; Detector, UV 254 nm) affording 430 mg (40%) of 3-phenyl-N-[(1s,3s)-3-[(3-formyl-1H-pyrazol-1-yl)methyl]cyclobutyl]-1,2-oxazole-5-carboxamide as a yellow solid. LCMS (ES, m/z): [M+H]⁺=351.2.

Step 2: 3-Phenyl-N-[(1s,3s)-3-([3-[(1S)-1-hydroxyethyl]-1H-pyrazol-1-yl]methyl)cyclobutyl]-1,2-oxazole-5-carboxamide (front peak) and 3-phenyl-N-[(1s,3s)-3-([3-[(1R)-1-hydroxyethyl]-1H-pyrazol-1-yl]methyl)cyclobutyl]-1,2-oxazole-5-carboxamide (second peak). To a 100-mL 3-necked round-bottom flask was placed a solution of 3-phenyl-N-[(1s,3s)-3-[(3-formyl-1H-pyrazol-1-yl)methyl]cyclobutyl]-1,2-oxazole-5-carboxamide (430 mg, 1.23 mmol, 1.00 equiv) in THF (30 mL), then the solution was cooled to 0° C. and MeMgBr (1.2 mL, 3 mol/L, 3.00 equiv) was added dropwise with stirring at 0° C. over 5 min. The reaction was stirred for 3 h at RT, then quenched by the addition of 2N HCl (10 mL) and 50 mL of H₂O, and extracted with EtOAc (3×50 mL). The organic extracts were combined, washed with brine (3×50 mL), dried over anhydrous Na₂SO₄, and concentrated under reduced pressure. The residue was applied onto a silica gel column and eluted with EtOAc/petroleum ether (2:1). The pure isomers were separated by Chiral-Prep-HPLC (Prep-HPLC-009: Column, Phenomenex Lux 5u Cellulose-3, 5*25 cm, 5 um; mobile phase, Hex and IPA (hold 50.0% IPA-in 17 min); Detector, UV 220/254 nm) affording 89.5 mg (20%) of 3-phenyl-N-[(1s,3s)-3-([3-[(1S)-1-hydroxyethyl]-1H-pyrazol-1-yl]methyl)cyclobutyl]-1,2-oxazole-5-carboxamide (front peak) as a white solid and 65.5 mg (15%) of 3-phenyl-N-[(1s,3s)-3-([3-[(1R)-1-hydroxyethyl]-1H-pyrazol-1-yl]methyl)cyclobutyl]-1,2-oxazole-5-carboxamide (second peak) as a white solid.

3-Phenyl-N-[(1s,3s)-3-([3-[(1S)-1-hydroxyethyl]-1H-pyrazol-1-yl]methyl)cyclobutyl]-1,2-oxazole-5-carboxamide (front peak): LCMS (ES, m/z): [M+H]⁺=367.1. ¹H NMR (300 MHz, DMSO-d₆) δ 9.21-9.18 (d, J=7.5 Hz, 1H), 7.94-7.92 (m, 2H), 7.62 (s, 1H), 7.55-7.53 (m, 4H), 6.15 (d, J=2.1 Hz, 1H), 4.94-4.92 (d, J=4.8 Hz, 1H), 4.71-4.63 (m, 1H), 4.34-4.26 (m, 1H), 4.09-4.06 (d, J=6.9 Hz, 2H), 2.48-2.29 (m, 3H), 1.98-1.89 (m, 2H), 1.34-1.32 (d, J=6.3 Hz, 3H). Purity (HPLC, 254 nm): 98.2%.

3-Phenyl-N-[(1s,3s)-3-([3-[(1R)-1-hydroxyethyl]-1H-pyrazol-1-yl]methyl)cyclobutyl]-1,2-oxazole-5-carboxamide (second peak): LCMS (ES, m/z): [M+H]⁺=367.2. ¹H NMR (300 MHz, DMSO-d₆) δ 9.21-9.18 (d, J=7.5 Hz, 1H), 7.93-7.90 (m, 2H), 7.61 (s, 1H), 7.54-7.52 (m, 4H), 6.14 (d, J=2.1 Hz, 1H), 4.94-4.93 (d, J=4.8 Hz, 1H), 4.70-4.62 (m, 1H), 4.35-4.22 (m, 1H), 4.08-4.05 (d, J=6.9 Hz, 2H), 2.46-2.28 (m, 3H), 1.97-1.88 (m, 2H), 1.33-1.31 (d, J=6.6 Hz, 3H). Purity (HPLC, 254 nm): 97.9%.

### Examples 67 and 68: 3-Phenyl-N-[(1r,3r)-3-([4-[(1S)-1-hydroxyethyl]-1H-pyrazol-1-yl]methyl)cyclobutyl]-1,2-oxazole-5-carboxamide (Front Peak) and 3-Phenyl-N-[(1r,3r)-3-([4-[(1R)-1-hydroxyethyl]-1H-pyrazol-1-yl]methyl)cyclobutyl]-1,2-oxazole-5-carboxamide (Second Peak)

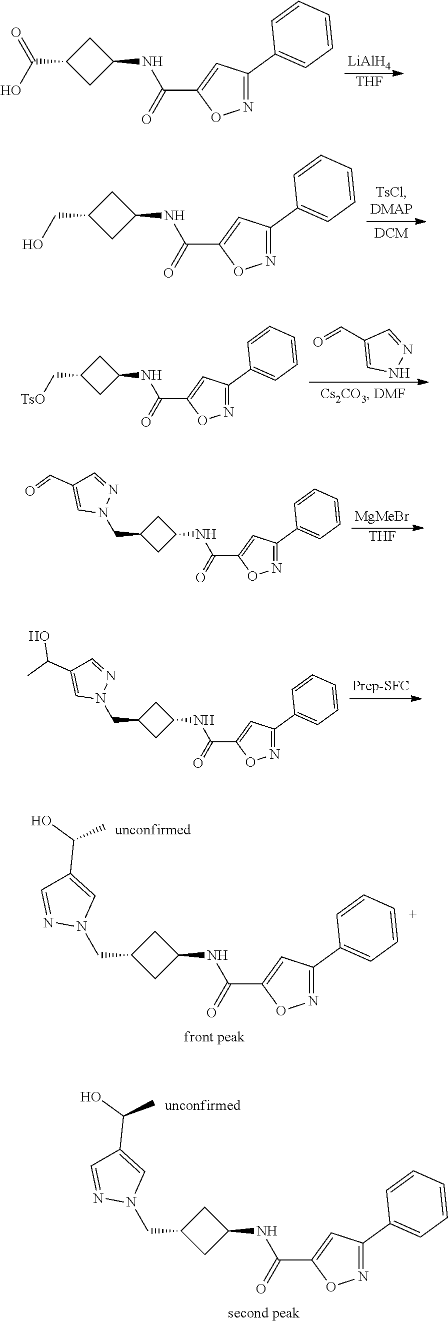

Step 1: 3-Phenyl-N-[(1r,3r)-3-(hydroxymethyl)cyclobutyl]-1,2-oxazole-5-carboxamide. To a 250-mL 3-necked round-bottom flask was placed a solution of (1r,3r)-3-(3-phenyl-1,2-oxazole-5-amido)cyclobutane-1-carboxylic acid (2 g, 6.99 mmol, 1.00 equiv) in THF (40 mL), then the solution was cooled to 0° C. and LiAlH₄ (800 mg, 21.08 mmol, 3.00 equiv) was added. The resulting solution was stirred for 2 h at 5° C., then quenched by the addition of Na₂SO₄.10H₂O. The solids were removed by filtration, then the filtrate was concentrated under reduced pressure affording 850 mg (45%) of 3-phenyl-N-[(1r,3r)-3-(hydroxymethyl)cyclobutyl]-1,2-oxazole-5-carboxamide as a yellow oil. LCMS (ES, m/z): [M+H]⁺=273.1.

Step 2: [(1r,3r)-3-(3-Phenyl-1,2-oxazole-5-amido)cyclobutyl]methyl 4-methylbenzene-1-sulfonate. To a 50-mL round-bottom flask was placed a solution of 3-phenyl-N-[(1r,3r)-3-(hydroxymethyl)cyclobutyl]-1,2-oxazole-5-carboxamide (850 mg, 3.12 mmol, 1.00 equiv) and DMAP (762 mg, 6.24 mmol, 1.20 equiv) in DCM (20 mL). To this solution was added TsCl (712 mg, 3.73 mmol, 1.20 equiv) then the mixture was stirred for 24 h at RT. The reaction was diluted with 50 mL of water/ice and extracted with DCM. The organic extracts were combined, dried over anhydrous Na₂SO₄, and concentrated under reduced pressure affording 980 mg (crude) of [(1r,3r)-3-(3-phenyl-1,2-oxazole-5-amido)cyclobutyl]methyl 4-methylbenzene-1-sulfonate as a light yellow solid. LCMS (ES, m/z): [M+H]⁺=427.1.

Step 3: 3-Phenyl-N-[(1r,3r)-3-[(4-formyl-1H-pyrazol-1-yl)methyl]cyclobutyl]-1,2-oxazole-5-carboxamide. To a 50-mL round-bottom flask was placed a solution of [(1r,3r)-3-(3-phenyl-1,2-oxazole-5-amido)cyclobutyl]methyl 4-methylbenzene-1-sulfonate (980 mg, 2.30 mmol, 1.00 equiv) in DMF (20 mL), then 1H-pyrazole-4-carbaldehyde (331 mg, 3.44 mmol, 1.50 equiv) and Cs₂CO₃ (1.1 g, 3.37 mmol, 1.50 equiv) were added. The resulting solution was stirred for 3 h at 70° C., diluted with 50 mL of H₂O, filtered, and then extracted with EtOAc. The organic extracts were combined, dried over anhydrous Na₂SO₄, and concentrated under reduced pressure. The residue was applied onto a silica gel column and eluted with EtOAc/petroleum ether (1:3) affording 400 mg (50%) of 3-phenyl-N-[(1r,3r)-3-[(4-formyl-1H-pyrazol-1-yl)methyl]cyclobutyl]-1,2-oxazole-5-carboxamide as a white solid. LCMS (ES, m/z): [M+H]⁺=351.1.

Step 4: 3-Phenyl-N-[(1r,3r)-3-[[4-(1-hydroxyethyl)-1H-pyrazol-1-yl]methyl]cyclobutyl]-1,2-oxazole-5-carboxamide. To a 150-mL round-bottom flask was placed a solution of 3-phenyl-N-[(1r,3r)-3-[(4-formyl-1H-pyrazol-1-yl)methyl]cyclobutyl]-1,2-oxazole-5-carboxamide (600 mg, 1.71 mmol, 1.00 equiv) in THF (20 mL) then the solution was cooled to 5° C. To this solution was added MeMgBr (1M in hexane, 1.79 mL, 1.79 mmol, 4.00 equiv) at 5° C. under nitrogen. The resulting solution was stirred for 3 h at 5° C. The pH value of the solution was adjusted to 3 with 1M HCl, then the resulting solution was extracted with EtOAc. The organic extracts were combined, dried over anhydrous Na₂SO₄, and concentrated under reduced pressure. The residue was applied onto a silica gel column and eluted with EtOAc/petroleum ether (1:3) affording 440 mg (70%) of 3-phenyl-N-[(1r,3r)-3-[[4-(1-hydroxyethyl)-1H-pyrazol-1-yl]methyl]cyclobutyl]-1,2-oxazole-5-carboxamide as a yellow solid. LCMS (ES, m/z): [M+H]⁺=367.2.

Step 5: 3-Phenyl-N-[(1r,3r)-3-([4-[(1S)-1-hydroxyethyl]-1H-pyrazol-1-yl]methyl)cyclobutyl]-1,2-oxazole-5-carboxamide (front peak) and 3-phenyl-N-[(1r,3r)-3-([4-[(1R)-1-hydroxyethyl]-1H-pyrazol-1-yl]methyl)cyclobutyl]-1,2-oxazole-5-carboxamide (second peak). The crude N-(3-[[4-(1-hydroxyethyl)-1H-pyrazol-1-yl]methyl]cyclobutyl)-3-phenyl-1,2-oxazole-5-carboxamide (440 mg, 1.20 mmol, 1.00 equiv) was separated by Prep-SFC (Column: Phenomenex Lux 5u Cellulose-4£¬, AXIA Packed, 250*21.2 mm, 5 um; Mobile Phase A: CO2: 60, Mobile Phase B: Hex: 40; Flow rate: 40 m/min; 220 nm; RT1:5.12; RT2:6.06) affording 141.7 mg (32%) of 3-phenyl-N-[(1r,3r)-3-([4-[(1S)-1-hydroxyethyl]-1H-pyrazol-1-yl]methyl)cyclobutyl]-1,2-oxazole-5-carboxamide (front peak) as a white solid and 146.5 mg (33%) of 3-phenyl-N-[(1r,3r)-3-([4-[(1R)-1-hydroxyethyl]-1H-pyrazol-1-yl]methyl)cyclobutyl]-1,2-oxazole-5-carboxamide (second peak) as a red solid.

3-Phenyl-N-[(1r,3r)-3-([4-[(1S)-1-hydroxyethyl]-1H-pyrazol-1-yl]methyl)cyclobutyl]-1,2-oxazole-5-carboxamide (front peak): LCMS (ES, m/z): [M+H−H₂O]⁺=349.1. ¹H NMR (300 MHz, DMSO-d₆): δ 9.26-9.23 (d, J=7.5 Hz, 1H), 7.93-7.90 (m, 2H), 7.62-7.60 (m, 2H), 7.54-7.52 (m, 3H), 7.32 (s, 1H), 4.85-4.83 (d, J=4.8 Hz, 1H), 4.71-4.63 (m, 1H), 4.55-4.47 (m, 1H), 4.20-4.17 (d, J=7.8 Hz, 2H), 2.68-2.64 (m, 1H), 2.27-2.10 (m, 4H), 1.33-1.31 (d, J=6.3 Hz, 3H). Purity (HPLC, 254 nm): 98.2%.

3-Phenyl-N-[(1r,3r)-3-([4-[(1R)-1-hydroxyethyl]-1H-pyrazol-1-yl]methyl)cyclobutyl]-1,2-oxazole-5-carboxamide (second peak): LCMS (ES, m/z): [M+H−H₂O]⁺=349.1. ¹H NMR (300 MHz, DMSO-d₆): δ 9.26-9.24 (d, J=7.2 Hz, 1H), 7.93-7.90 (m, 2H), 7.64-7.60 (m, 2H), 7.54-7.52 (m, 3H), 7.32 (s, 1H), 4.85-4.84 (d, J=4.8 Hz, 1H), 4.71-4.63 (m, 1H), 4.58-4.45 (m, 1H), 4.20-4.17 (d, J=7.8 Hz, 2H), 2.68-2.64 (m, 1H), 2.27-2.10 (m, 4H), 1.33-1.31 (d, J=6.3 Hz, 3H). Purity (HPLC, 254 nm): 97.0%.

### Examples 69 and 70: 3-Phenyl-N-[(1s,3s)-3-([4-[(1R)-1-hydroxyethyl]-1H-pyrazol-1-yl]methyl)cyclobutyl]-1,2-oxazole-5-carboxamide (Front Peak) and 3-phenyl-N-[(1s,3s)-3-([4-[(1S)-1-hydroxyethyl]-1H-pyrazol-1-yl]methyl)cyclobutyl]-1,2-oxazole-5-carboxamide (Second Peak)

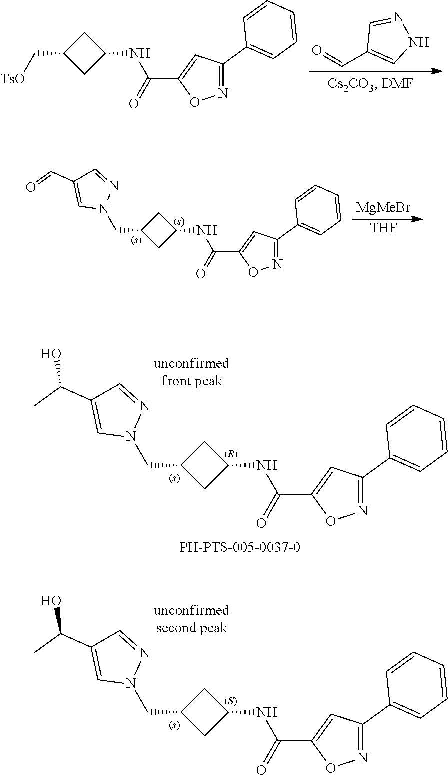

Step 1: 3-Phenyl-N-[(1s,3s)-3-[(4-formyl-1H-pyrazol-1-yl)methyl]cyclobutyl]-1,2-oxazole-5-carboxamide. To a 50-mL round-bottom flask was placed a solution of [(1s,3s)-3-(3-phenyl-1,2-oxazole-5-amido)cyclobutyl]methyl 4-methylbenzene-1-sulfonate (1 g, 2.34 mmol, 1.00 equiv) in DMF (15 mL) then Cs₂CO₃ (1.5 g, 4.60 mmol, 2.00 equiv) and 1H-pyrazole-4-carbaldehyde (338 mg, 3.52 mmol, 1.50 equiv) were added. The resulting solution was stirred for 3 h at 100° C. then the solids were removed by filtration. The crude product was purified by Flash-Prep-HPLC (CombiFlash-1: Column, C18 silica gel; mobile phase, X: H₂O (0.5% NH₄HCO₃), Y: ACN, X/Y=90/10 increasing to X/Y=5/95 within 40 min; Detector, UV 254 nm) affording 460 mg (56%) of 3-phenyl-N-[(1s,3s)-3-[(4-formyl-1H-pyrazol-1-yl)methyl]cyclobutyl]-1,2-oxazole-5-carboxamide as a yellow solid. LCMS (ES, m/z): [M+H]⁺=351.1.

Step 2: 3-Phenyl-N-[(1s,3s)-3-([4-[(1R)-1-hydroxyethyl]-1H-pyrazol-1-yl]methyl)cyclobutyl]-1,2-oxazole-5-carboxamide (front peak) and 3-phenyl-N-[(1s,3s)-3-([4-[(1S)-1-hydroxyethyl]-1H-pyrazol-1-yl]methyl)cyclobutyl]-1,2-oxazole-5-carboxamide (second peak). To a 100-mL 3-necked round-bottom flask was placed a solution of 3-phenyl-N-[(1s,3s)-3-[(4-formyl-1H-pyrazol-1-yl)methyl]cyclobutyl]-1,2-oxazole-5-carboxamide (460 mg, 1.31 mmol, 1.00 equiv) in THF (30 mL) then the solution was cooled to 0° C. To this solution was added MeMgBr (1.3 mL, 3.00 equiv) dropwise with stirring at 0° C. over 10 min. The resulting solution was stirred for 3 h at RT, quenched with 2N HCl (10 mL) and 50 mL of H₂O, and extracted with EtOAc (3×50 mL). The organic extracts were combined, washed with brine (3×50 mL), dried over anhydrous Na₂SO₄, and concentrated under reduced pressure. The residue was applied onto a silica gel column and eluted with EtOAc/petroleum ether (2:1). The product was purified by Chiral-Prep-HPLC (Prep-HPLC-004: Column, Chiralpak IB, 2*25 cm, 5 um; mobile phase, Hex and ethanol (hold 10.0% ethanol in 41 min); Detector, uv 254/220 nm) affording 132.3 mg (28%) of 3-phenyl-N-[(1s,3s)-3-([4-[(1R)-1-hydroxyethyl]-1H-pyrazol-1-yl]methyl)cyclobutyl]-1,2-oxazole-5-carboxamide front peak) as an off-white solid and 139.4 mg (29%) of 3-phenyl-N-[(1s,3s)-3-([4-[(1S)-1-hydroxyethyl]-1H-pyrazol-1-yl]methyl)cyclobutyl]-1,2-oxazole-5-carboxamide (second peak) as an off-white solid.

3-Phenyl-N-[(1s,3s)-3-([4-[(1R)-1-hydroxyethyl]-1H-pyrazol-1-yl]methyl)cyclobutyl]-1,2-oxazole-5-carboxamide (front peak): LCMS (ES, m/z): [M+H]⁺=367.1. ¹H NMR (300 MHz, DMSO-d₆) δ 9.21-9.18 (d, J=7.5 Hz, 1H), 7.94-7.91 (m, 2H), 7.62 (s, 1H), 7.55-7.51 (m, 3H), 7.48 (s, 1H), 7.32 (s, 1H), 4.85 (brs, 1H), 4.70-4.64 (q, J=6.6 Hz, 1H), 4.36-4.23 (m, 1H), 4.10-4.07 (d, J=6.9 Hz, 2H), 2.46-2.29 (m, 3H), 1.99-1.89 (m, 2H), 1.33-1.31 (d, J=6.3 Hz, 3H). Purity (HPLC, 254 nm): 96.4%.

3-Phenyl-N-[(1s,3s)-3-([4-[(1S)-1-hydroxyethyl]-1H-pyrazol-1-yl]methyl)cyclobutyl]-1,2-oxazole-5-carboxamide (second peak): LCMS (ES, m/z): [M+H]⁺=367.1. ¹H NMR (300 MHz, DMSO-d₆) δ 9.21-9.18 (d, J=7.5 Hz, 1H), 7.92-7.87 (m, 2H), 7.68 (s, 1H), 7.61-7.48 (m, 4H), 7.31 (s, 1H), 4.85 (brs, 1H), 4.73-4.65 (m, 1H), 4.36-4.28 (m, 1H), 4.09-4.07 (d, J=6.6 Hz, 2H), 2.43-2.32 (m, 3H), 1.95-1.85 (m, 2H), 1.32-1.31 (d, J=5.1 Hz, 3H). Purity (HPLC, 254 nm): 96.0%.

### Example 71: 3-Phenyl-N-[(1r,3r)-3-(4-fluorophenoxymethyl)cyclobutyl]-1,2-oxazole-5-carboxamide

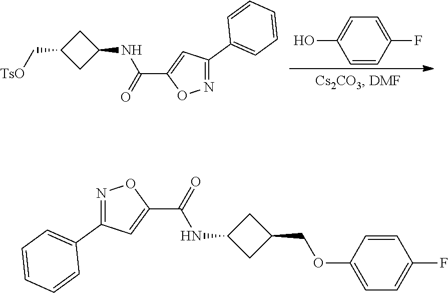

To a 50-mL round-bottom flask was placed a solution of [(1r,3r)-3-(3-phenyl-1,2-oxazole-5-amido)cyclobutyl]methyl 4-methylbenzene-1-sulfonate (550 mg, 1.29 mmol, 1.00 equiv) in DMF (10 mL), then 4-fluorophenol (217 mg, 1.94 mmol, 1.50 equiv) and Cs₂CO₃ (631 mg, 1.93 mmol, 1.50 equiv) were added. The resulting solution was stirred for 3 h at 70° C., then diluted with H₂O, and extracted with EtOAc. The organic extracts were combined, dried over anhydrous Na₂SO₄, and concentrated under reduced pressure. The residue was applied onto a silica gel column and eluted with EtOAc/petroleum ether (1:4). The resulting crude product was further purified by Prep-HPLC (Waters: Column, X Bridge Shield RP18 OBD Column, 5 um, 19*150 mm; mobile phase, water with 0.03% TFA and CH₃CN (10.0% CH₃CN up to 30% CH₃CN in 8 min, up to 100% in 4 min and down to 10% in 3 min); Detector, uv 254 nm and 220 nm) affording 152.3 mg (53%) of 3-phenyl-N-[(1r,3r)-3-(4-fluorophenoxymethyl)cyclobutyl]-1,2-oxazole-5-carboxamide as a white solid. LCMS (ES, m/z): [M+H]⁺=367.1. ¹H NMR (300 MHz, DMSO-d₆): δ 9.31-9.29 (d, J=7.2 Hz, 1H), 7.92-7.91 (m, 2H), 7.63 (s, 1H), 7.55-7.54 (m, 3H), 7.15-7.09 (m, 2H), 6.99-6.95 (m, 2H), 4.61-4.53 (m, 1H), 4.05-4.03 (d, J=6.9 Hz, 2H), 2.69-2.63 (m, 1H), 2.38-2.29 (m, 2H), 2.23-2.17 (m, 2H). Purity (HPLC, 254 nm): 97.4%.

### Example 72: 3-phenyl-N-[(1s,3s)-3-(4-fluorophenoxymethyl)cyclobutyl]-1,2-oxazole-5-carboxamide

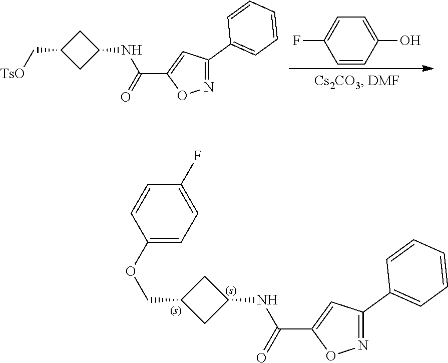

To a 25-mL round-bottom flask was placed a solution of [(1s,3s)-3-(3-phenyl-1,2-oxazole-5-amido)cyclobutyl]methyl 4-methylbenzene-1-sulfonate (213 mg, 0.50 mmol, 1.00 equiv) in DMF (5 mL), then Cs₂CO₃ (326 mg, 1.00 mmol, 2.00 equiv) and 4-fluorophenol (84 mg, 0.75 mmol, 1.50 equiv) were added. The resulting solution was stirred for 3 h at 100° C., then diluted with 50 mL of H₂O, and extracted with EtOAc (2×50 mL). The organic extracts were combined, washed with brine (2×50 mL), dried over anhydrous Na₂SO₄, and concentrated under reduced pressure. The crude product was purified by Prep-TLC (petroleum ether/ethyl acetate=1:2) affording 56.9 mg (31%) of 3-phenyl-N-[(1s,3s)-3-(4-fluorophenoxymethyl)cyclobutyl]-1,2-oxazole-5-carboxamide as a white solid. LCMS (ES, m/z): [M+H]⁺=367.1. ¹H NMR (300 MHz, DMSO-d₆) δ 9.23-9.21 (d, J=7.5 Hz, 1H), 7.94-7.90 (m, 2H), 7.62 (s, 1H), 7.55-7.48 (m, 3H), 7.14-7.07 (m, 2H), 6.97-6.93 (m, 2H), 4.40-4.32 (m, 1H), 3.94-3.92 (d, J=5.1 Hz, 2H), 2.43-2.39 (m, 3H), 2.00-1.94 (m, 2H). Purity (HPLC, 254 nm): 99.5%.

### Example 73: 3-phenyl-N-[(1r,3r)-3-(4-cyanophenoxymethyl)cyclobutyl]-1,2-oxazole-5-carboxamide

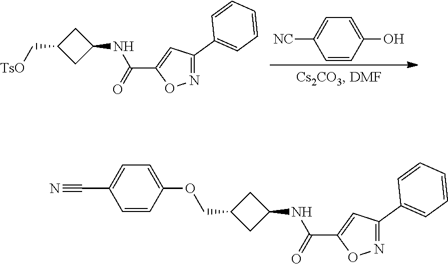

To a 50-mL round-bottom flask was placed a solution of [(1r,3r)-3-(3-phenyl-1,2-oxazole-5-amido)cyclobutyl]methyl 4-methylbenzene-1-sulfonate (560 mg, 1.31 mmol, 1.00 equiv) in DMF (10 mL), then 4-hydroxybenzonitrile (235 mg, 1.97 mmol, 1.50 equiv) and Cs₂CO₃ (643 mg, 1.97 mmol, 1.50 equiv) were added. The resulting solution was stirred for 2 h at 110° C., then diluted by the addition of water, and extracted with EtOAc. The organic extracts were combined, was washed with H₂O, dried over anhydrous Na₂SO₄, and concentrated under reduced pressure. This residue was purified by Prep-HPLC (Waters: Column, X Bridge Prep C18 Sum, 19*150 mm; mobile phase, water with 0.03% TFA and CH₃CN (10.0% CH₃CN up to 30% CH₃CN in 6 min, up to 100% in 5 min and down to 10% in 2 min); Detector, uv 254 nm and 220 nm) affording 129.9 mg (87%) of 3-phenyl-N-[(1r,3r)-3-(4-cyanophenoxymethyl)cyclobutyl]-1,2-oxazole-5-carboxamide as a white solid. LCMS-PH-PTS (ES, m/z): [M+H]⁺=374.1. ¹H NMR (300 MHz, DMSO-d₆): δ 9.32-9.30 (d, J=7.5 Hz, 1H), 7.94-7.91 (m, 2H), 7.78-7.76 (d, J=8.7 Hz, 2H), 7.63 (s, 1H), 7.55-7.54 (m, 3H), 7.15-7.12 (m, J=8.7 Hz, 2H), 4.63-4.55 (m, 1H), 4.19-4.17 (d, J=6.9 Hz, 2H), 2.72-2.66 (m, 1H). 2.40-2.30 (m, 2H), 2.24-2.18 (m, 2H). Purity (HPLC, 254 nm): 97.3%.

### Example 74: 3-Phenyl-N-[(1s,3s)-3-(4-cyanophenoxymethyl)cyclobutyl]-1,2-oxazole-5-carboxamide

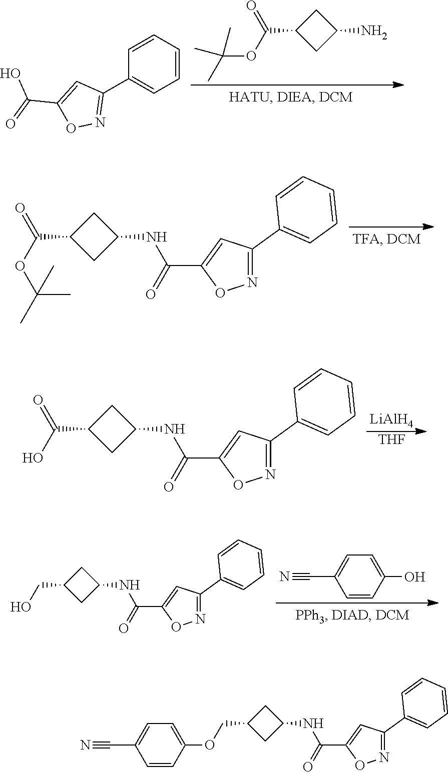

Step 1: tert-Butyl (1s,3s)-3-(3-Phenyl-1,2-oxazole-5-amido)cyclobutane-1-carboxylate. To a 100-mL round-bottom flask was placed a solution of tert-butyl (1s,3s)-3-aminocyclobutane-1-carboxylate (1.7 g, 9.93 mmol, 1.00 equiv) in DCM (50 mL), then 3-phenyl-1,2-oxazole-5-carboxylic acid (1.9 g, 10.04 mmol, 1.00 equiv), HATU (5.7 g, 14.99 mmol, 1.50 equiv) and DIEA (3.9 g, 30.18 mmol, 3.00 equiv) were added. The resulting solution was stirred for 1 h at RT, then quenched by the addition of water, and extracted with EtOAc. The organic extracts were combined, washed with brine, dried over anhydrous Na₂SO₄, and concentrated under reduced pressure. The residue was applied onto a silica gel column and eluted with EtOAc/petroleum ether (1:7) affording 2 g (59%) of tert-butyl (1s,3s)-3-(3-phenyl-1,2-oxazole-5-amido)cyclobutane-1-carboxylate as a white solid. LCMS (ES, m/z): [M+Na]⁺=365.1.

Step 2: (1s,3s)-3-(3-Phenyl-1,2-oxazole-5-amido)cyclobutane-1-carboxylic acid. To a 100-mL round-bottom flask was placed a solution of tert-butyl (1s,3s)-3-(3-phenyl-1,2-oxazole-5-amido)cyclobutane-1-carboxylate (2 g, 5.84 mmol, 1.00 equiv) in DCM (20 mL) and TFA (7 mL). The resulting solution was stirred for 4 h at RT, then the solvent was removed under reduced pressure affording 1.8 g (crude) of (1s,3s)-3-(3-phenyl-1,2-oxazole-5-amido)cyclobutane-1-carboxylic acid as an off-white solid. LCMS (ES, m/z): [M+H]⁺=286.8.

Step 3: 3-Phenyl-N-[(1s,3s)-3-(hydroxymethyl)cyclobutyl]-1,2-oxazole-5-carboxamide. To a 100-mL round-bottom flask was placed a solution of (1s,3s)-3-(3-phenyl-1,2-oxazole-5-amido)cyclobutane-1-carboxylic acid (1 g, 2.79 mmol, 1.00 equiv, 80%) in THF (25 mL), then the solution was cooled to 0° C. To this solution was added LiAlH₄ (425 mg, 11.18 mmol, 4.00 equiv) in portions at 0° C., then the resulting solution was stirred for 1 h at 10° C. The reaction was quenched by the addition of Na₂SO₄.10H₂O, then the solids were removed by filtration, and the filtrate was concentrated under reduced pressure. The residue was applied onto a silica gel column and eluted with EtOAc/petroleum ether (1:2) affording 420 mg (55%) of 3-phenyl-N-[(1s,3s)-3-(hydroxymethyl)cyclobutyl]-1,2-oxazole-5-carboxamide as a white solid. LCMS (ES, m/z): [M+H]⁺=273.1.

Step 4: 3-Phenyl-N-[(1s,3s)-3-(4-cyanophenoxymethyl)cyclobutyl]-1,2-oxazole-5-carboxamide. To a 100-mL 3-necked round-bottom flask purged and maintained with an inert atmosphere of nitrogen, was placed a solution of 3-phenyl-N-[(1s,3s)-3-(hydroxymethyl)cyclobutyl]-1,2-oxazole-5-carboxamide (420 mg, 1.31 mmol, 1.00 equiv, 85%), 4-hydroxybenzonitrile (320 mg, 2.69 mmol, 2.00 equiv) and PPh₃ (1.08 g, 4.12 mmol, 3.00 equiv) in THF (10 mL). This was followed by the addition of DIAD (840 mg, 4.15 mmol, 3.00 equiv) dropwise with stirring at 0° C. The resulting solution was stirred for 2 h at RT. The reaction was quenched by the addition of water, then extracted with EtOAc. The organic extracts were combined, washed with brine, dried over anhydrous Na₂SO₄, and concentrated under reduced pressure. The crude product was purified by Flash-Prep-HPLC (IntelFlash-1: Column, C18; mobile phase, MeCN/H₂O=5:95 increasing to MeCN/H₂O=50:50 within 20 min; Detector, UV 254 nm) affording 148.5 mg (30%) of 3-phenyl-N-[(1s,3s)-3-(4-cyanophenoxymethyl)cyclobutyl]-1,2-oxazole-5-carboxamide as a white solid. LCMS (ES, m/z): [M+H]⁺=374.2. ¹H NMR (400 MHz, DMSO-d₆): δ 9.25-9.24 (d, J=7.6 Hz, 1H), 7.94-7.92 (m, 2H), 7.79-7.76 (m, 2H), 7.64 (s, 1H), 7.55-7.53 (m, 3H), 7.16-7.12 (m, 2H), 4.43-4.35 (p, J=8.0 Hz, 1H), 4.08-4.06 (d, J=6.0 Hz, 2H), 2.45-2.40 (m, 3H), 2.02-1.97 (m, 2H). Purity (HPLC, 254 nm): 98.2%.

### Examples 75 and 76: 3-(5-Fluorothiophen-2-yl)-N-[(1s,3s)-3-([5-[(1R)-1-hydroxyethyl]-1,3,4-oxadiazol-2-yl]methyl)cyclobutyl]-1,2-oxazole-5-carboxamide and 3-(5-Fluorothio phen-2-yl)-N-[(1r,3r)-3-([5-[(1R)-1-hydroxyethyl]-1,3,4-oxadiazol-2-yl]methyl)cyclobutyl]-1,2-oxazole-5-carboxamide

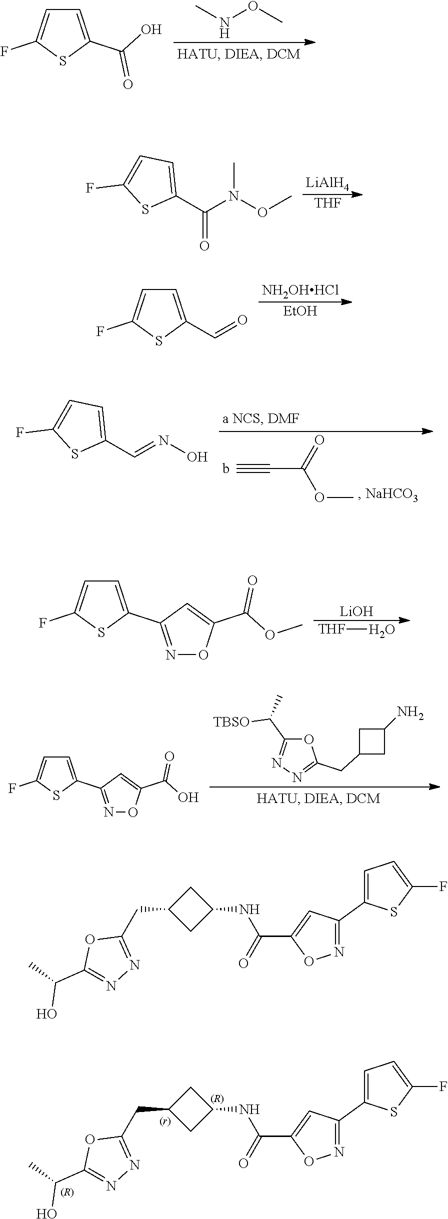

Step 1: 5-Fluoro-N-methoxy-N-methylthiophene-2-carboxamide. To a 100-mL round-bottom flask was placed a solution of 5-fluorothiophene-2-carboxylic acid (1 g, 6.84 mmol, 1.00 equiv) in DCM (50 mL), then methoxy(methyl)amine hydrochloride (730 mg, 7.53 mmol, 1.10 equiv), HATU (3.9 g, 10.26 mmol, 1.50 equiv), and DIEA (2.82 mL, 3.00 equiv) were added. The reaction was stirred for 3 h at room temperature, diluted with H₂O, and extracted with DCM (2×100 mL). The organic extracts were combined, washed with brine (2×50 mL), dried over anhydrous Na₂SO₄, and concentrated under reduced pressure. The residue was applied onto a silica gel column and eluted with EtOAc/petroleum ether (1:4) affording 1.14 g (88%) of 5-fluoro-N-methoxy-N-methylthiophene-2-carboxamide as a yellow liquid. LCMS (ES, m/z): [M+H]⁺=190.0.

Step 2: (E)-N-[(5-Fluorothiophen-2-yl)methylidene]hydroxylamine. To a 50-mL round-bottom flask was placed a solution of 5-fluoro-N-methoxy-N-methylthiophene-2-carboxamide (1.14 g, 6.03 mmol, 1.00 equiv) in THF (20 mL), then LiAlH₄ (342 mg, 9.01 mmol, 1.20 equiv) was added. The action was stirred for 1 h at room temperature, then quenched by the addition of 20 mL of H₂O/ice, and extracted with EtOAc (2×20 mL). The organic extracts were dried and used directly in the next step.

To a 250-mL round-bottom flask was placed a solution of 5-fluorothiophene-2-carbaldehyde (780 mg, 5.99 mmol, 1.00 equiv) in EtOH/EtOAc (120 mL), then NH₂OH.HCl (0.5 g, 1.20 equiv) was added. The resulting solution was stirred for 3 h at room temperature then the solvent was removed under reduced pressure. The residue was dissolved in H₂O (50 mL), then the resulting solution was extracted with EtOAc (3×100 mL). The organic extracts were combined, washed with brine (2×100 mL), dried over anhydrous Na₂SO₄, and concentrated under reduced pressure affording 650 mg (75%) of (E)-N-[(5-fluorothiophen-2-yl)methylidene]hydroxylamine as a yellow solid. LCMS (ES, m/z): [M+H]⁺=146.0.

Step 3: Methyl 3-(5-Fluorothiophen-2-yl)-1,2-oxazole-5-carboxylate. To a 25-mL round-bottom flask was placed a solution of (E)-N-[(5-fluorothiophen-2-yl)methylidene]hydroxylamine (300 mg, 2.07 mmol, 1.00 equiv) DMF (5 mL), then NCS (414 mg, 3.11 mmol, 1.50 equiv) was added in small portions. The resulting solution was stirred for 1 h at room temperature, then methyl prop-2-ynoate (0.27 mL, 2.00 equiv) was added followed by Na₂CO₃ (260 mg, 3.09 mmol, 1.50 equiv) in small portions. The reaction was stirred for 2 h at room temperature, diluted with 50 mL of H₂O, and extracted with EtOAc (3×100 mL). The organic extracts were combined, washed with brine (2×50 mL), dried over anhydrous Na₂SO₄, and concentrated under vacuum. The residue was purified by prep TLC (ethyl acetate/petroleum ether=1/3) affording 200 mg (43%) of methyl 3-(5-fluorothiophen-2-yl)-1,2-oxazole-5-carboxylate as a yellow solid.

Step 4: 3-(5-fluorothiophen-2-yl)-1,2-oxazole-5-carboxylic acid. To a 50-mL round-bottom flask was placed a solution of methyl 3-(5-fluorothiophen-2-yl)-1,2-oxazole-5-carboxylate (254 mg, 1.12 mmol, 1.00 equiv) in THF-H₂O (3:1, 10 mL), then LiOH (52 mg, 2.17 mmol, 2.00 equiv) was added. The reaction was stirred for 1 h at room temperature, diluted with H₂O (20 mL), and washed with ethyl acetate (2×50 mL). The pH of the aqueous layer was adjusted to 3 with 1M HCl, then the resulting solution was extracted with EtOAc (3×50 mL). The organic extracts were combined, was washed with brine (2×50 mL), dried over anhydrous Na₂SO₄, and concentrated under reduced pressure affording 170 mg (71%) of 3-(5-fluorothiophen-2-yl)-1,2-oxazole-5-carboxylic acid as a yellow solid. LCMS (ES, m/z): [M+H]⁺=214.1.

Step 5: 3-(5-Fluorothiophen-2-yl)-N-[(1s,3s)-3-([5-[(1R)-1-hydroxyethyl]-1,3,4-oxadiazol-2-yl]methyl)cyclobutyl]-1,2-oxazole-5-carboxamide and 3-(5-Fluorothiophen-2-yl)-N-[(1r,3r)-3-([5-[(1R)-1-hydroxyethyl]-1,3,4-oxadiazol-2-yl]methyl)cyclobutyl]-1,2-oxazole-5-carboxamide. To a 50-mL round-bottom flask was placed a solution of 3-(5-fluorothiophen-2-yl)-1,2-oxazole-5-carboxylic acid (170 mg, 0.80 mmol, 1.00 equiv) in DCM (20 mL), then 3-([5-[(1R)-1-[(tert-butyldimethylsilyl)oxy]ethyl]-1,3,4-oxadiazol-2-yl]methyl)cyclobutan-1-amine (273 mg, 0.88 mmol, 1.10 equiv), HATU (455 mg, 1.20 mmol, 1.50 equiv), and DIEA (0.33 mL, 3.00 equiv) were added. The reaction was stirred for 3 h at room temperature, diluted with H₂O, and extracted with DCM. The organic extracts were combined, washed with brine (2×30 mL), dried over anhydrous Na₂SO₄, and concentrated under reduced pressure. The residue was purified by Prep-TLC (EtOAc/petroleum ether=1/4), then the resulting pure isomers were separated by Chiral-Prep-HPLC (Prep-HPLC-032: Column, Lux 5u Cellulose-4, AXIA Packed, 250*21.2 mm; mobile phase, Hex and IPA (hold 30.0% IPA in 21 min); Detector, UV 254/220 nm) affording 37.2 mg (19%) of 3-(5-fluorothiophen-2-yl)-N-[(1s,3s)-3-([5-[(1R)-1-hydroxyethyl]-1,3,4-oxadiazol-2-yl]methyl)cyclobutyl]-1,2-oxazole-5-carboxamide as a white solid and 9.4 mg (5%) of 3-(5-fluorothiophen-2-yl)-N-[(1r,3r)-3-([5-[(1R)-1-hydroxyethyl]-1,3,4-oxadiazol-2-yl]methyl)cyclobutyl]-1,2-oxazole-5-carboxamide as a white solid.

3-(5-Fluorothiophen-2-yl)-N-[(1s,3s)-3-([5-[(1R)-1-hydroxyethyl]-1,3,4-oxadiazol-2-yl]methyl)cyclobutyl]-1,2-oxazole-5-carboxamide: LCMS (ES, m/z): [M+H]⁺=393.1. ¹H NMR (300 MHz, DMSO-d₆): δ 9.23-9.20 (d, J=7.8 Hz, 1H), 7.61 (s, 1H), 7.57-7.55 (t, J=3.9 Hz, 2H), 6.94-6.92 (m, 1H), 5.91-5.89 (d, J=5.7 Hz, 1H), 4.92-4.83 (m, 1H), 4.33-4.23 (m, 1H), 2.97-2.95 (d, J=6.3 Hz, 2H), 2.46-2.33 (m, 3H), 1.96-1.90 (m, 2H), 1.45-1.43 (d, J=6.6 Hz, 3H). Purity (HPLC, 254 nm): 98.8%.

3-(5-Fluorothiophen-2-yl)-N-[(1r,3r)-3-([5-[(1R)-1-hydroxyethyl]-1,3,4-oxadiazol-2-yl]methyl)cyclobutyl]-1,2-oxazole-5-carboxamide: LCMS (ES, m/z): [M+H]⁺=393.1. ¹H NMR (300 MHz, DMSO-d₆): δ 9.33-9.31 (d, J=7.2 Hz, 1H), 7.62 (s, 1H), 7.57-7.54 (t, J=4.2 Hz, 1H), 6.94-6.92 (m, 1H), 5.92-5.90 (d, J=5.7 Hz, 1H), 4.92-4.84 (m, 1H), 4.57-4.49 (m, 1H), 3.09-3.06 (d, J=7.8 Hz, 2H), 2.72-2.64 (m, 1H), 2.37-2.27 (m, 2H), 2.17-2.12 (m, 2H), 1.46-1.43 (d, J=6.6 Hz, 3H). Purity (HPLC, 254 nm): 99.3%.

### Example 77

Chronic Obstructive Pulmonary Disease (COPD) Assay

A COPD model assay for compound modulation of the phenotypes associated with the COPD is conducted by exposing human bronchial epithelial (HBE) cells to cigarette smoke extract (CSE). One or more assays to determine a restoration of normal function in this COPD model in response to compounds disclosed herein such as compound A or A′ are used. The determination of restoration of normal function can then be detected for example by any one of a number of methodologies including one or more of short-circuit current measurements of chloride transport to evaluate CFTR function in response to CSE and in response to treatment with compounds; equivalent current measurements of chloride transport to evaluate CFTR function in response to CSE and in response to treatment with compounds; immunoblotting, immunoblotting, western blotting and/or ELISA.

### Example 78

Assay for Increased Transcript Levels of CFTR

As stably transfected human airway cell line (CFBEs) or primary human lung epithialial cells (hBEs) are differentiated for a minimum of 4 weeks in an air-liquid interface on SnapWell filter plates prior to transcript analysis. CFBEs or hBEs of a given genotype are incubated for 24 h at 37° C. and 5% CO₂ in differentiated media containing the indicated concentration of compounds disclosed herein such as compound A or DMSO, all at a final concentration of 0.1% DMSO. Following the incubation, the media is aspirated away, and the cells are rapidly frozen in a dry-ice ethanol bath. The cells are thawed into Quantigene Plex 2.0 Lysis Mixture containing Proteinase K and lysed at an estimated concentration of 200 cells/microliters. Lysates (80 microliters) are used in the Quantigene Plex 2.0 gene expression assay according to the manufacturer's instructions. The CFTR transcript levels presented are adjusted to the levels of RPL13A transcript, used to control for differences in lysate loading. Such assays measure the changes in levels of CFTR transcript in compound-treated cells, relative to DMSO-treated cells, for a given genotype of the CFTR alleles present in human bronchial epithelial cells.

### Example 79

Table 2 indicates mutation type and activity with compounds/combination with compound A. ## indicates activity at 30 uM of 50% to <100% of the indicated relative activity treatment, #### indicates activity at 30 uM of ≥150% of the indicated relative activity treatment, + indicates activity at 10 uM of 15% to <50% of the indicated relative activity treatment, ++ indicates activity at 10 uM of 50% to <100% of the indicated relative activity treatment, +++ indicates activity at 10 uM of 100% to <150% of the indicated relative activity treatment, ++++ indicates activity at 10 uM of ≥150% of the indicated relative activity treatment. NB124 is used at 250 ug/ml, ivacaftor is used at 1 uM, lumacaftor is used at 3 uM, and VX-661 is used at 3 uM.

TABLE 2

| | | | Combin- | Combin- | Combin- | Combin- |
|---|---|---|---|---|---|---|
| | Relative | | ation | ation with | ation with | ation |
| | Activity | Stand | with | ivacaftor and | ivacaftor | with |
| Genotype | of 100% | Alone | ivacaftor | lumacaftor | and VX-661 | NB124 |
| Non-CF | Vehicle | +++ | | | | |
| (“wild- | treated | | | | | |
| type”/ | | | | | | |
| “wild- | | | | | | |
| type” | | | | | | |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating chronic obstructive pulmonary disease, bronchitis, or asthma in a patient in need thereof, or in a patient at risk of developing chronic obstructive pulmonary disease, comprising a) administering an effective amount of a compound represented by Formula III or IV and b) optionally administering an effective amount of one or more of an additional active agent, wherein Formula II and IV are: 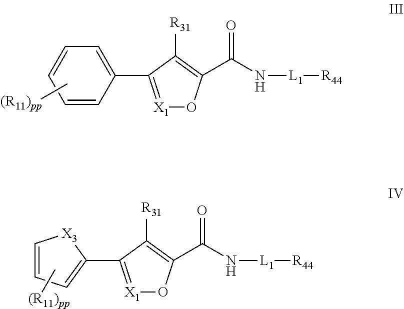
and pharmaceutically acceptable salts, stereoisomers, and prodrugs thereof, wherein:
X₁ is N or CR₃₃;
X₃ is selected from the group consisting of NRₕₕ, O, and S;
pp is 1, 2, or 3;
R₁₁ is independently selected for each occurrence from the group consisting of hydrogen, halogen, and C₁₋₄ alkyl (optionally substituted by one, two or three halogens);
R₃₁ is selected from the group consisting of hydrogen, halogen, and C₁₋₄ alkyl;
R₃₃ is selected from the group consisting of H, halogen, C₁₋₄ alkyl, and —NR′R″ wherein R′ and R″ are each independently selected for each occurrence from H and C₁₋₄ alkyl or taken together with the nitrogen to which they are attached form a heterocyclic ring;
L₁ is selected from the group consisting of C₁₋₆ alkylene, C₃₋₆ cycloalkylene, C₃₋₆ cycloalkylene-C₁₋₄ alkylene, C₁₋₃ alkylene-NRₕₕ—S(O)_{w}—, —C₁₋₃ alkylene-S(O)_{w}—NRₕₕ—, C₃₋₆ cycloalkylene-C₀₋₂ alkylene-S(O)_{w}—NRₕₕ, and C₃₋₆ cycloalkylene-C₀₋₂ alkylene NRₕₕ—S(O)_{w}—, wherein L₁ may be optionally substituted by one, two or three substituents selected from the group consisting of halogen, hydroxyl, and C₁₋₃ alkyl (optionally substituted by one, two or three substituents each selected independently from R_{ff});
R₄₄ is selected from the group consisting of H, halogen, hydroxyl, C₁₋₃ alkoxy, phenyl, —O-phenyl, —NR′-phenyl, heterocycle, and a 5-6 membered monocyclic or 8-10 membered bicyclic heteroaryl having one, two or three heteroatoms each selected from O, N, and S; wherein phenyl, —O-phenyl, —NR′-phenyl, heterocycle and heteroaryl may be optionally substituted by one or two substituents each selected independently from R_{gg};
R_{ff} is selected for each occurrence from group consisting of halogen, hydroxyl, C₁₋₄ alkyl, C₁₋₄ alkyoxy, C₂₋₄ alkenyl, C₃₋₆ cycloalkyl, —NR′R″, —NR′—S(O)_{w}—C₁₋₃ alkyl, S(O)_{w}—NR′R″, and —S(O)_{w}—C₁₋₃ alkyl, where w is 0, 1, or 2, wherein C₁₋₄ alkyl, C₁₋₄ alkyoxy, C₂₋₄ alkenyl and C₃₋₆ cycloalkyl may be optionally substituted by one, two or three substituents each independently selected from the group consisting of halogen, hydroxyl, —NR′R″, —NR′—S(O)_{w}—C₁₋₃ alkyl, S(O)_{w}—NR′R″, and —S(O)_{w}—C₁₋₃ alkyl;
R_{gg} is selected for each occurrence from the group consisting of halogen, hydroxyl, cyano, —NR′R″, —NR′—S(O)_{w}—C₁₋₃ alkyl, —S(O)_{w}—NR′R″, and —S(O)_{w}—C₁₋₃ alkyl, where w is 0, 1, or 2; heterocycle, C₁₋₆ alkyl, C₃₋₆ cycloalkyl, and C₁₋₆ alkenyl, wherein C₁₋₆ alkyl, C₃₋₆ cycloalkyl, and C₁₋₆ alkenyl are optionally substituted by one, two, or three substituents each independently selected from Rⱼⱼ; and heterocycle is optionally substituted by one, two, or three substituents each independently selected from Rₗₗ;
Rⱼⱼ is selected for each occurrence from the group consisting of halogen, hydroxyl, C₁₋₆ alkoxy (optionally substituted by one, two, or three substituents each independently selected from Rₖₖ); C₃₋₆ cycloalkyl, C₃₋₆ cycloalkoxy, heterocycle, C(O)OH, —C(O)OC₁₋₆ alkyl, —NR′R″, —NR′—S(O)_{w}—C₁₋₃ alkyl, —S(O)_{w}—NR′R″, and —S(O)_{w}—C₁₋₃ alkyl, where w is 0, 1, or 2;
Rₖₖ is selected for each occurrence from the group consisting of halogen, hydroxyl, C₁₋₆ alkyl (optionally substituted by one, two, or three substituents each independently selected from halogen, hydroxyl, C₃₋₆ cycloalkyl, and heterocycle (optionally substituted by C₁₋₆ alkyl)), C₃₋₆ cycloalkyl (optionally substituted by one, two, or three substituents each independently selected from halogen, hydroxyl, and C₁₋₆ alkyl), phenyl, heterocycle (optionally substituted by one, two or three substituents independently selected from halogen, hydroxyl, and C₁₋₆ alkyl), and heteroaryl;
Rₗₗ is selected for each occurrence from the group consisting of halogen, hydroxyl, C₁₋₆ alkyl (optionally substituted by one, two, or three substituents each independently selected from halogen, hydroxyl, and C₃₋₆ cycloalkyl) and heterocycle (optionally substituted by one, two or three substituents independently selected from halogen, hydroxyl, and C₁₋₆ alkyl);
R′ and R″ are each independently selected for each occurrence from H, C₁₋₄ alkyl, phenyl and heterocycle;
w is 0, 1 or 2; and
Rₕₕ is selected for each occurrence from the group consisting of H, C₁₋₆ alkyl and C₃₋₆ cycloalkyl.

2. The method of claim 1, wherein L₁ is C₁₋₃ alkylene, C₃₋₅ cycloalkylene, or C₃₋₆ cycloalkylene-C₁₋₄ alkylene.

3. The method of claim 1 or 2, wherein R₃₁ is H or F.

4. The method of any one of claims 1-3, wherein R_{gg} is selected from the group consisting of: 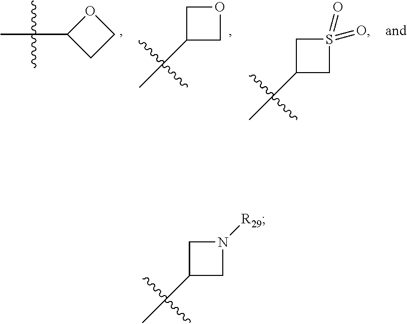
wherein R₂₉ is selected from C₁₋₆ alkyl (optionally substituted by one, two or three substituents each independently selected from the group consisting of halogen, hydroxyl, C₁₋₆ alkoxy, and cycloalkyl) and heterocycle (optionally substituted by one, two or three substituents each independently selected from the group consisting of halogen, hydroxyl, C₁₋₆ alkyl and C₁₋₆ alkoxy).

5. The method of claim 4, wherein R₂₉ is selected from the group consisting of:

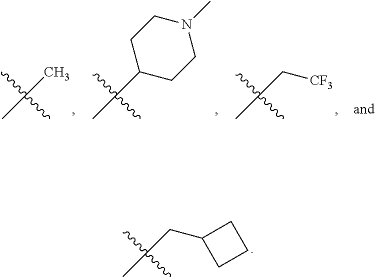

6. The method of any one of claims 1-5, wherein the compound is represented by: 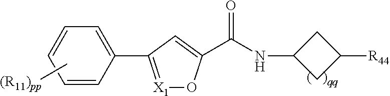
wherein qq is 0 or 1.

7. The method of any one of claims 1-6, wherein the compound is represented by:

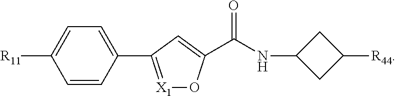

8. The method of any one of claims 1-7, wherein R₄₄ is selected from the group consisting of: pyrrolidinyl, piperidinyl, tetrahydropyranyl, and tetrahydrofuranyl.

9. The method of any one of claims 1-7, wherein R₄₄ is selected from the group consisting of: 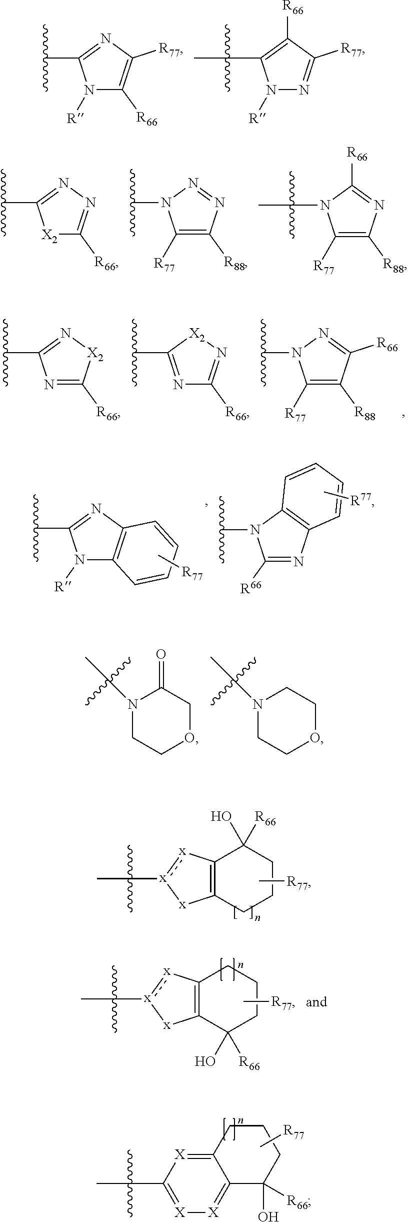
wherein X independently for each occurrence is selected from the group consisting of O, S, NRₕₕ, C, C(R₈₈), and C(R₈₈)(R₉₉); X₂ independently for each occurrence is selected from the group consisting of O, S and NRₕₕ; R″ is H or C₁₋₄alkyl; and each R₆₆, R₇₇, R₈₈ and R₉₉ is independently selected for each occurrence from H and R_{gg}, and n is 0, 1, 2, or 3.

10. The method of claim 9, wherein each R₆₆, R₇₇, R₈₈ and R₉₉ is independently selected for each occurrence from the group consisting of hydrogen, halogen, hydroxyl, C₁₋₆ alkyl, C₃₋₆ cycloalkyl, and heterocycle, wherein C₁₋₆ alkyl, C₃₋₆ cycloalkyl, and heterocycle are optionally substituted by one, two or three substituents each independently selected from the group consisting of hydroxyl, C₁₋₆ alkyl, C₁₋₆ alkoxy (optionally substituted by C₃₋₆cycloalkyl, heterocycle, —C₁₋₂alkyl-heterocycle and C₁₋₂alkyl-C₃₋₆cycloalkyl), —S(O)_{w}—C₁₋₃ alkyl (w is 0, 1, or 2) and —NR′S(O)₂C₁₋₆ alkyl; and
R′ is independently selected for each occurrence from H and C₁₋₄ alkyl.

11. The method of any one of claims 1-10, wherein pp is 0, 1 or 2, and R₁₁ is selected from H, F, or methyl.

12. The method of any one of claims 1-12, wherein the chronic obstructive pulmonary disease is emphysema.

13. The method of any one of claims 1-12, wherein the additional active agent is selected from the group consisting of: β₂ agonists, muscarinic antagonists, anticholinergics, corticosteroids, methylxanthine compounds, antihistamines, decongestants, anti-tussive drug substances, PDE I-VI inhibitors, prostacycline analogs, mucolytics, calcium blockers and CFTR modulators.

14. The method of claim 13, wherein the corticosteroid is selected from the group consisting of: dexamethasone, budesonide, beclomethasone, triamcinolone, dexamethasone, mometasone, ciclesonide, fluticasone, flunisolide, dexamethasone sodium phosphate and pharmaceutically acceptable salts and esters thereof.

15. The method of any one of claims 1-14, wherein the additional active agent is selected from the group consisting of interferon γ1β; bosentan, entanercept, and imatinib mesylate.

16. The method of claim 15, wherein the β-agonist is a long acting β-agonist.

17. The method of claim 15, wherein the β-agonist is selected from the group consisting of: albuterol, formoterol, pirbuterol, metapoterenol, salmeterol, arformoterol, indacaterol, levalbuterol, terbutaline and pharmaceutically acceptable salts thereof.

18. The method of claim 15, wherein the corticosteroid is selected from budesonide or beclomethasone dipropionate.

19. The method of any one of claims 1-18 wherein at least two additional active agents are administered and are each selected from the group consisting of vilanterol, umeclidine, formoterol, salmeterol, budesone, fluticasone and pharmaceutically acceptable salts thereof.

20. The method of claims 1-19, wherein the at least one additional active agent is a long acting muscarinic antagonist selected from the group consisting of tiotropium, glycopyrronium, aclidinium and pharmaceutically acceptable salts thereof.

21. The method of any one of claims 1-20 wherein at least two additional active agents are administered.

22. The method of any one of claims 1-21, wherein at least one additional active agent is a CFTR corrector or potentiator.

23. The method of any one of claims 1-22 wherein the risk factor for developing chronic obstructive pulmonary disorder in a patient is a history of smoking or having mesothelioma.

24. The method of any one of claims 1-22, wherein the risk factor for developing chronic obstructive pulmonary disorder is air pollution.

25. The method of any one of claims 1-24, wherein administering an effective amount of a compound is orally or by inhalation.

26. The method of any one of claims 1-25, wherein administering an effective amount of additional active agent is oral or inhalation administration.

27. The method of any one of claims 1-26, wherein the compound of Formula III or IV is selected from the group consisting of: 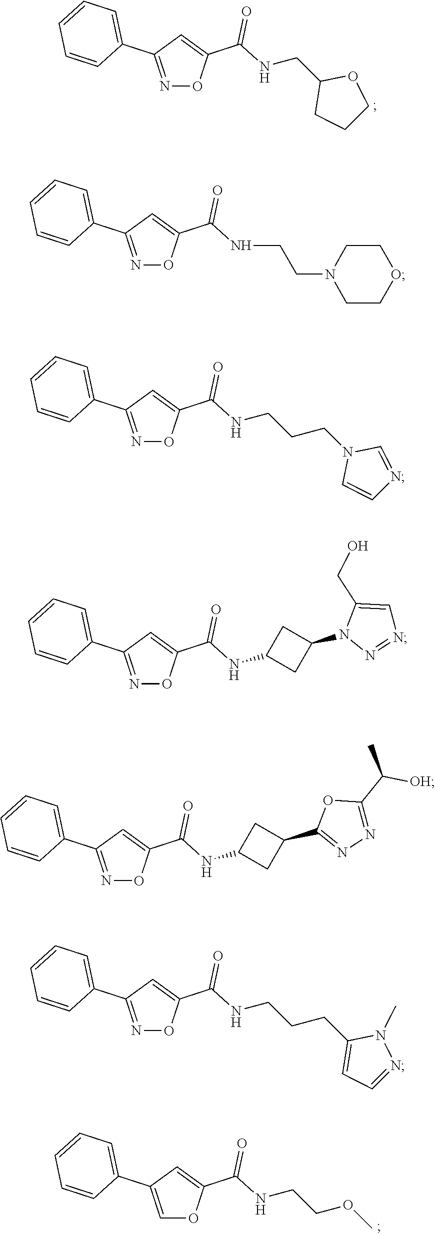 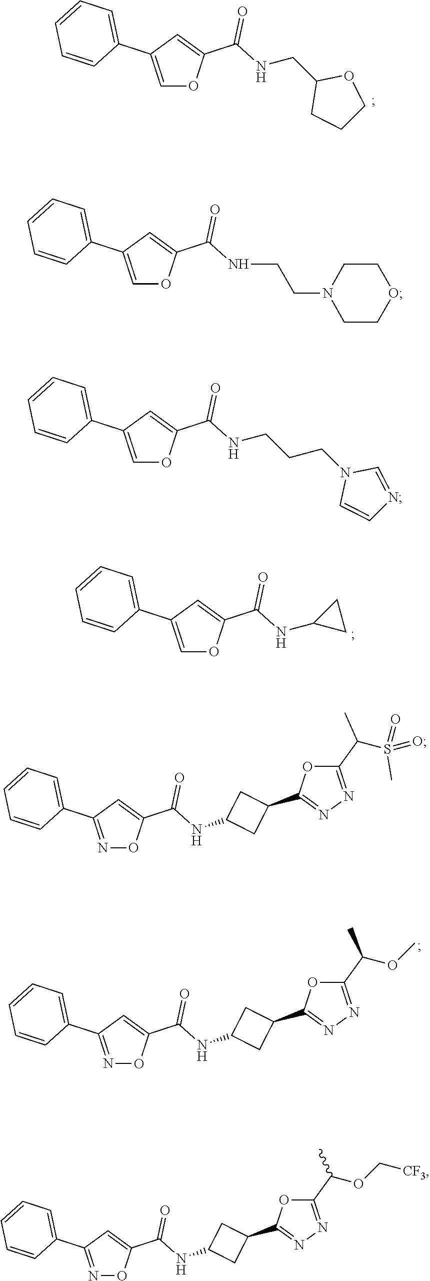 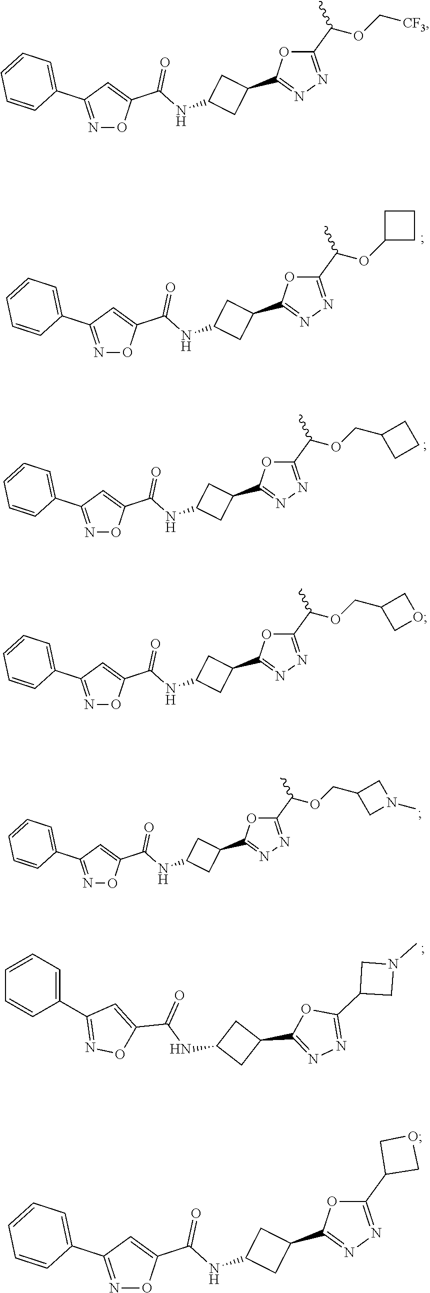 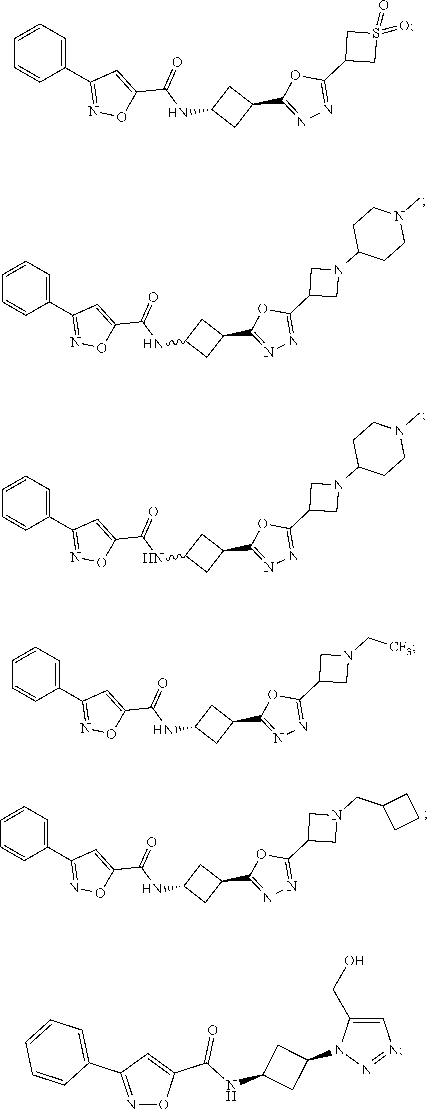 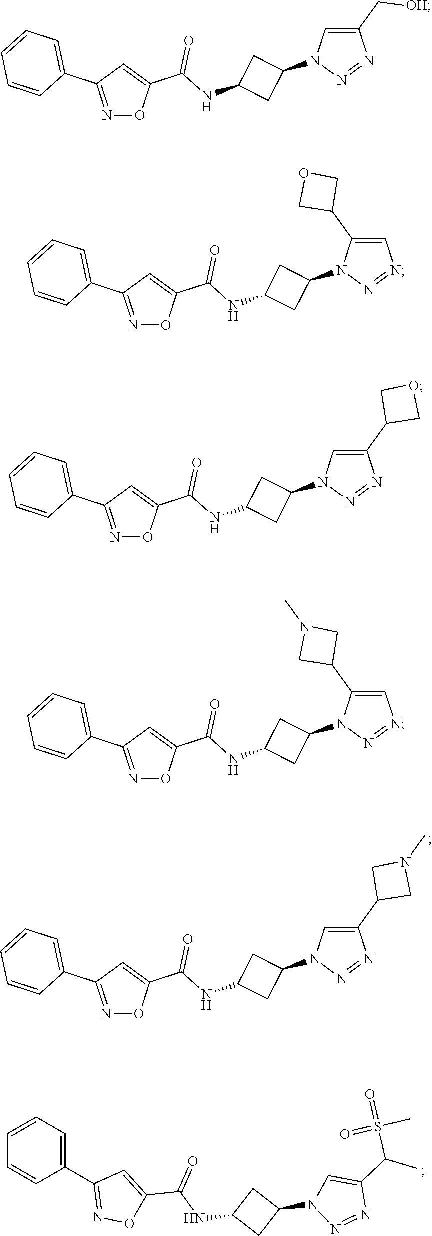 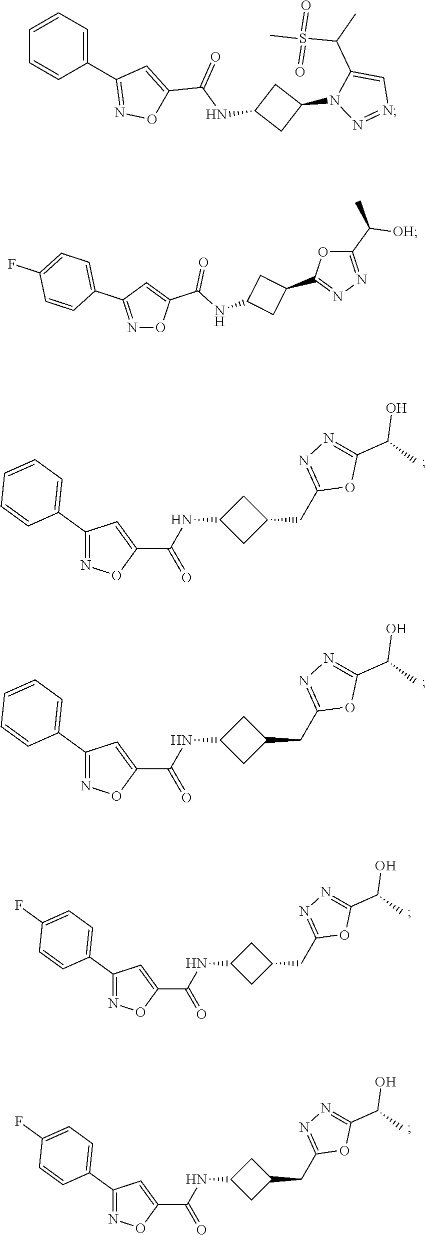 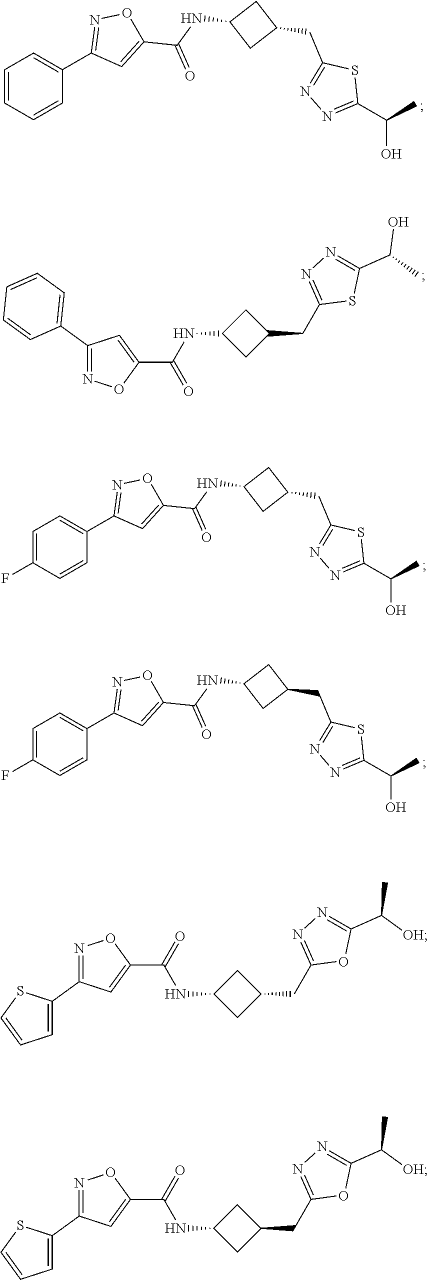 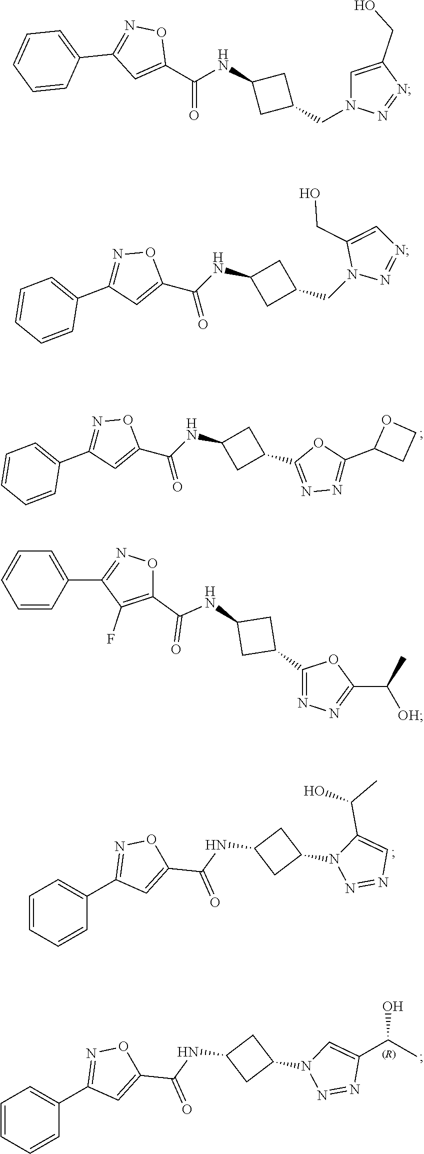 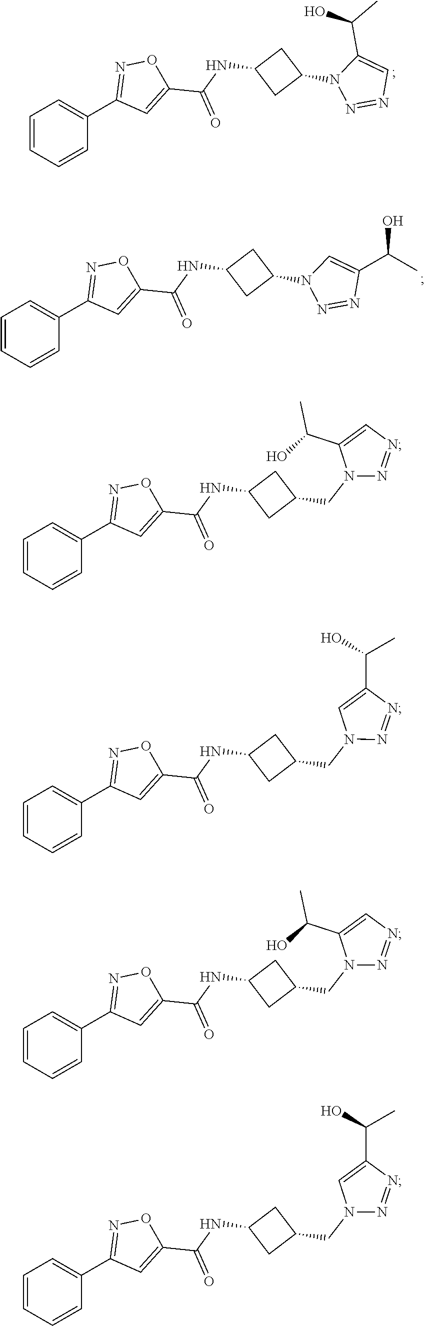 [SMILES: C[C@H](O)c1cnnn1[C@H]1C[C@@H](NC(=O)c2cc(-c3ccccc3)no2)C1 ; C[C@H](O)c1cn([C@H]2C[C@@H](NC(=O)c3cc(-c4ccccc4)no3)C2)nn1 ; C[C@@H](O)c1cnnn1C[C@H]1C[C@@H](NC(=O)c2cc(-c3ccccc3)no2)C1 ; C[C@@H](O)c1cn(C[C@H]2C[C@@H](NC(=O)c3cc(-c4ccccc4)no3)C2)nn1 ; C[C@H](O)c1cnnn1C[C@H]1C[C@@H](NC(=O)c2cc(-c3ccccc3)no2)C1 ; C[C@H](O)c1cn(C[C@H]2C[C@@H](NC(=O)c3cc(-c4ccccc4)no3)C2)nn1] 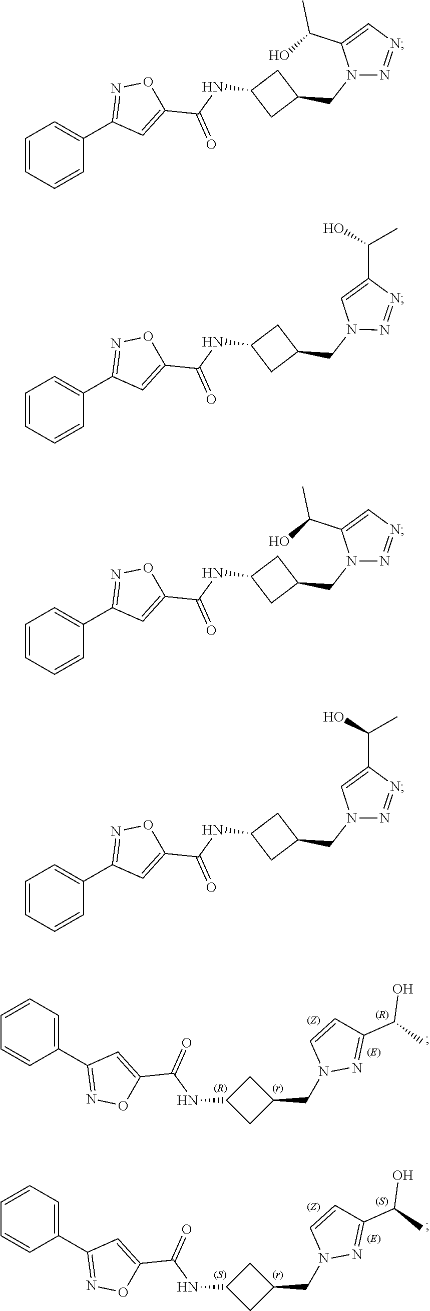 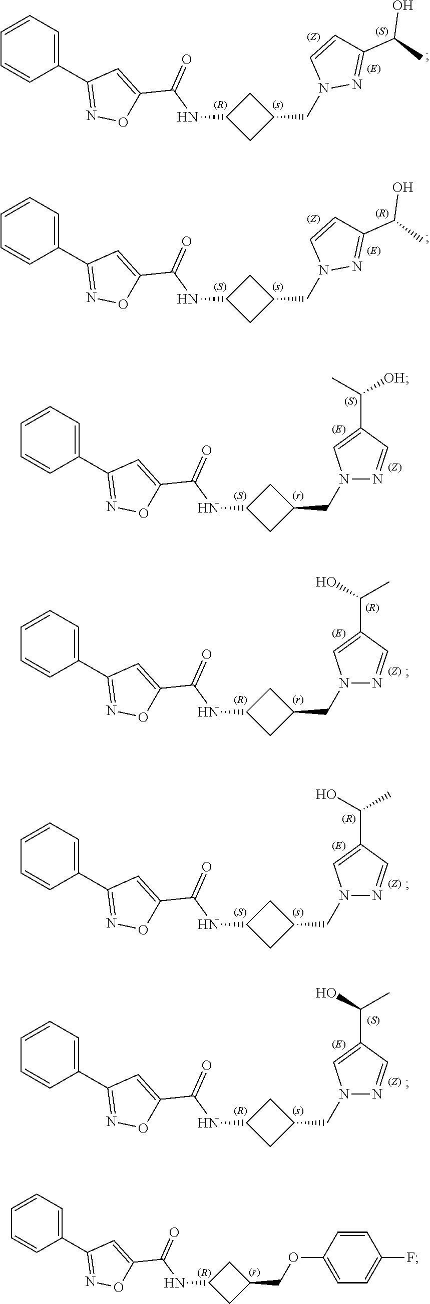 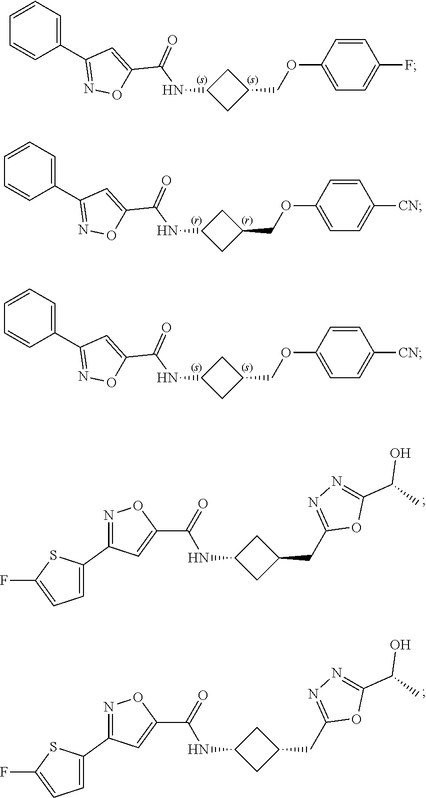
and pharmaceutically acceptable salts thereof.
